SEQUENCE LISTING (101 sequences)

SEQ ID NO: 1 - RNA, 22 nt, Artificial Sequence
  hsa-let-7a-5p MIMAT0000062 Sense
ugagguaguagguuguauaguu

SEQ ID NO: 2 - RNA, 22 nt, Artificial Sequence
  hsa-let-7a-5p MIMAT0000062 Anti-sense
acuccaucauccaacauaucaa

SEQ ID NO: 3 - RNA, 22 nt, Artificial Sequence
  hsa-let-7c-5p MIMAT0000064 Sense
ugagguaguagguuguaugguu

SEQ ID NO: 4 - RNA, 22 nt, Artificial Sequence
  hsa-let-7c-5p MIMAT0000064 Anti-sense
acuccaucauccaacauaccaa

SEQ ID NO: 5 - RNA, 22 nt, Artificial Sequence
  hsa-miR-99a-5p MIMAT0000097 Sense
aacccguagauccgaucuugug

SEQ ID NO: 6 - RNA, 22 nt, Artificial Sequence
  hsa-miR-99a-5p MIMAT0000097 Anti-sense
uugggcaucuaggcuagaacac

SEQ ID NO: 7 - RNA, 22 nt, Artificial Sequence
  hsa-miR-100-5p MIMAT0000098 Sense
aacccguagauccgaacuugug

SEQ ID NO: 8 - RNA, 22 nt, Artificial Sequence
  hsa-miR-100-5p MIMAT0000098 Anti-sense
uugggcaucuaggcuugaacac

SEQ ID NO: 9 - RNA, 22 nt, Danio rerio
  misc_feature (0)...(0): dre-let-7a-5p
ugagguaguagguuguauaguu

SEQ ID NO: 10 - RNA, 22 nt, Mus musculus
  misc_feature (0)...(0): mmu-let-7a-5p
ugagguaguagguuguauaguu

SEQ ID NO: 11 - RNA, 22 nt, Rattus norvegicus
  misc_feature (0)...(0): rno-let-7a-5p
ugagguaguagguuguauaguu

SEQ ID NO: 12 - RNA, 22 nt, Sus scrofa
  misc_feature (0)...(0): ssc-let-7a-5p
ugagguaguagguuguauaguu

SEQ ID NO: 13 - RNA, 22 nt, Pan troglodytes
  misc_feature (0)...(0): ptr-let-7a-5p
ugagguaguagguuguauaguu

SEQ ID NO: 14 - RNA, 22 nt, Homo sapiens
  misc_feature (0)...(0): hsa-let-7a-5p
ugagguaguagguuguauaguu

SEQ ID NO: 15 - RNA, 22 nt, Canis familiaris
  misc_feature (0)...(0): cfa-let-7a-5p
ugagguaguagguuguauaguu

SEQ ID NO: 16 - RNA, 22 nt, Danio rerio
  misc_feature (0)...(0): dre-let-7c-5p
ugagguaguagguuguaugguu

SEQ ID NO: 17 - RNA, 22 nt, Mus musculus
  misc_feature (0)...(0): mmu-let-7c-5p
ugagguaguagguuguaugguu

SEQ ID NO: 18 - RNA, 22 nt, Rattus norvegicus
  misc_feature (0)...(0): rno-let-7c-5p
ugagguaguagguuguaugguu

SEQ ID NO: 19 - RNA, 22 nt, Sus scrofa
  misc_feature (0)...(0): ssc-let-7c-5p
ugagguaguagguuguaugguu

SEQ ID NO: 20 - RNA, 22 nt, Pan troglodytes
  misc_feature (0)...(0): ptr-let-7c-5p
ugagguaguagguuguaugguu

SEQ ID NO: 21 - RNA, 22 nt, Homo sapiens
  misc_feature (0)...(0): hsa-let-7c-5p
ugagguaguagguuguaugguu

SEQ ID NO: 22 - RNA, 22 nt, Canis familiaris
  misc_feature (0)...(0): cfa-let-7c-5p
ugagguaguagguuguaugguu

SEQ ID NO: 23 - RNA, 22 nt, Danio rerio
  misc_feature (0)...(0): dre-miR-99a-5p
aacccguagauccgaucuugug

SEQ ID NO: 24 - RNA, 22 nt, Mus musculus
  misc_feature (0)...(0): mmu-miR-99a-5p
aacccguagauccgaucuugug

SEQ ID NO: 25 - RNA, 22 nt, Rattus norvegicus
  misc_feature (0)...(0): rno-miR-99a-5p
aacccguagauccgaucuugug

SEQ ID NO: 26 - RNA, 21 nt, Canis familiaris
  misc_feature (0)...(0): cfa-miR-99a
aacccguagauccgaucuugu

SEQ ID NO: 27 - RNA, 22 nt, Sus scrofa
  misc_feature (0)...(0): ssc-miR-99a
aacccguagauccgaucuugug

SEQ ID NO: 28 - RNA, 22 nt, Pan troglodytes
  misc_feature (0)...(0): ptr-miR-99a
aacccguagauccgaucuugug

SEQ ID NO: 29 - RNA, 22 nt, Homo sapiens
  misc_feature (0)...(0): hsa-miR-99a-5p
aacccguagauccgaucuugug

SEQ ID NO: 30 - RNA, 22 nt, Danio rerio
  misc_feature (0)...(0): dre-miR-100-5p
aacccguagauccgaacuugug

SEQ ID NO: 31 - RNA, 22 nt, Mus musculus
  misc_feature (0)...(0): mmu-miR-100-5p
aacccguagauccgaacuugug

SEQ ID NO: 32 - RNA, 22 nt, Rattus norvegicus
  misc_feature (0)...(0): rno-miR-100-5p
aacccguagauccgaacuugug

SEQ ID NO: 33 - RNA, 22 nt, Sus scrofa
  misc_feature (0)...(0): ssc-miR-100
aacccguagauccgaacuugug

SEQ ID NO: 34 - RNA, 22 nt, Pan troglodytes
  misc_feature (0)...(0): ptr-miR-100
aacccguagauccgaacuugug

SEQ ID NO: 35 - RNA, 22 nt, Homo sapiens
  misc_feature (0)...(0): hsa-miR-100-5p
aacccguagauccgaacuugug

SEQ ID NO: 36 - DNA, 19 bp, Artificial Sequence
  JRX0100
ccatacaacctactacctc

SEQ ID NO: 37 - DNA, 19 bp, Artificial Sequence
  JRX0101
ctatacaacctactacctc

SEQ ID NO: 38 - DNA, 18 bp, Artificial Sequence
  JRX0102
catacaacctactacctc

SEQ ID NO: 39 - DNA, 18 bp, Artificial Sequence
  JRX0103
tatacaacctactacctc

SEQ ID NO: 40 - DNA, 17 bp, Artificial Sequence
  JRX0104
atacaacctactacctc

SEQ ID NO: 41 - DNA, 17 bp, Artificial Sequence
  JRX0105
atacaacctactacctc

SEQ ID NO: 42 - DNA, 16 bp, Artificial Sequence
  JRX0106
tacaacctactacctc

SEQ ID NO: 43 - DNA, 16 bp, Artificial Sequence
  JRX0107
tacaacctactacctc

SEQ ID NO: 44 - DNA, 15 bp, Artificial Sequence
  JRX0108
acaacctactacctc

SEQ ID NO: 45 - DNA, 15 bp, Artificial Sequence
  JRX0109
acaacctactacctc

SEQ ID NO: 46 - DNA, 19 bp, Artificial Sequence
  JRX0110
caagttcggatctacgggt

SEQ ID NO: 47 - DNA, 19 bp, Artificial Sequence
  JRX0111
caagatcggatctacgggt

SEQ ID NO: 48 - DNA, 18 bp, Artificial Sequence
  JRX0112
aagatcggatctacgggt

SEQ ID NO: 49 - DNA, 18 bp, Artificial Sequence
  JRX0113
aagttcggatctacgggt

SEQ ID NO: 50 - DNA, 17 bp, Artificial Sequence
  JRX0114
agatcggatctacgggt

SEQ ID NO: 51 - DNA, 17 bp, Artificial Sequence
  JRX0115
agttcggatctacgggt

SEQ ID NO: 52 - DNA, 16 bp, Artificial Sequence
  JRX0116
gatcggatctacgggt

SEQ ID NO: 53 - DNA, 16 bp, Artificial Sequence
  JRX0117
gttcggatctacgggt

SEQ ID NO: 54 - DNA, 15 bp, Artificial Sequence
  JRX0118
atcggatctacgggt

SEQ ID NO: 55 - DNA, 15 bp, Artificial Sequence
  JRX0119
ttcggatctacgggt

SEQ ID NO: 56 - DNA, 7 bp, Artificial Sequence
  Loop sequence
tgtgctt

SEQ ID NO: 57 - DNA, 21 bp, Artificial Sequence
  Forward primer for mouse U6 promoter
tcgcacagacttgtgggagaa

SEQ ID NO: 58 - DNA, 28 bp, Artificial Sequence
  Reverse primer mouse U6 promoter
cgcacattaagcctctatagttactagg

SEQ ID NO: 59 - DNA, 57 bp, Artificial Sequence
  let-7a-5p sequence in vector JBT-miR1
gtgaggtagtaggttgtatagtttcaagagaactatacaacctactacctcattttt

SEQ ID NO: 60 - DNA, 57 bp, Artificial Sequence
  miR-99a-5p sequence in vector JBT-miR1
gaacccgtagatccgatcttgtgtcaagagcacaagatcggatctacgggttttttt

SEQ ID NO: 61 - DNA, 120 bp, Artificial Sequence
  (H1-)let-7a-5p & (U6)-miR-99a-5p in vector JBT-miR1
gtgaggtagtaggttgtatagtttcaagagaactatacaacctactacctcatttttgagctcaaaaaaacccgtagatccgatcttgtgctcttgacacaagatcggatctacgggttc

SEQ ID NO: 62 - DNA, 57 bp, Artificial Sequence
  let-7c-5p sequence in vector JBT-miR1
gtgaggtagtaggttgtatggtttcaagagaaccatacaacctactacctcattttt

SEQ ID NO: 63 - DNA, 57 bp, Artificial Sequence
  miR-100-5p sequence in vector JBT-miR1
gaacccgtagatccgaacttgtgtcaagagcacaagttcggatctacgggttttttt

SEQ ID NO: 64 - DNA, 120 bp, Artificial Sequence
  (H1-)let-7C-5p & (U6)-miR-100-5p in vector JBT-miR1
gtgaggtagtaggttgtatggtttcaagagaaccatacaacctactacctcatttttgagctcaaaaaaacccgtagatccgaacttgtgctcttgacacaagttcggatctacgggttc

SEQ ID NO: 65 - DNA, 19 bp, Artificial Sequence
  let-7c inhibitor sequence
ccatacaacctactacctc

SEQ ID NO: 66 - DNA, 19 bp, Artificial Sequence
  let-7a inhibitor sequence
ctatacaacctactacctc

SEQ ID NO: 67 - DNA, 18 bp, Artificial Sequence
  let-7c inhibitor sequence
catacaacctactacctc

SEQ ID NO: 68 - DNA, 18 bp, Artificial Sequence
  let-7a inhibitor sequence
tatacaacctactacctc

SEQ ID NO: 69 - DNA, 17 bp, Artificial Sequence
  let-7a/c inhibitor sequence
atacaacctactacctc

SEQ ID NO: 70 - DNA, 17 bp, Artificial Sequence
  let-7a/c inhibitor sequence
atacaacctactacctc

SEQ ID NO: 71 - DNA, 16 bp, Artificial Sequence
  let-7a/c inhibitor sequence
tacaacctactacctc

SEQ ID NO: 72 - DNA, 16 bp, Artificial Sequence
  let-7a/c inhibitor sequence
tacaacctactacctc

SEQ ID NO: 73 - DNA, 15 bp, Artificial Sequence
  let-7a/c inhibitor sequence
acaacctactacctc

SEQ ID NO: 74 - DNA, 15 bp, Artificial Sequence
  let-7a/c inhibitor sequence
acaacctactacctc

SEQ ID NO: 75 - DNA, 19 bp, Artificial Sequence
  miR-100 inhibitor sequence
caagttcggatctacgggt

SEQ ID NO: 76 - DNA, 19 bp, Artificial Sequence
  miR-99 inhibitor sequence
caagatcggatctacgggt

SEQ ID NO: 77 - DNA, 18 bp, Artificial Sequence
  miR-99 inhibitor sequence
aagatcggatctacgggt

SEQ ID NO: 78 - DNA, 18 bp, Artificial Sequence
  miR-100 inhibitor sequence
aagttcggatctacgggt

SEQ ID NO: 79 - DNA, 17 bp, Artificial Sequence
  miR-99 inhibitor sequence
agatcggatctacgggt

SEQ ID NO: 80 - DNA, 17 bp, Artificial Sequence
  miR-100 inhibitor sequence
agttcggatctacgggt

SEQ ID NO: 81 - DNA, 16 bp, Artificial Sequence
  miR-99 inhibitor sequence
gatcggatctacgggt

SEQ ID NO: 82 - DNA, 16 bp, Artificial Sequence
  miR-100 inhibitor sequence
gttcggatctacgggt

SEQ ID NO: 83 - DNA, 15 bp, Artificial Sequence
  miR-99 inhibitor sequence
atcggatctacgggt

SEQ ID NO: 84 - DNA, 15 bp, Artificial Sequence
  miR-100 inhibitor sequence
ttcggatctacgggt

SEQ ID NO: 85 - DNA, 6558 bp, Artificial Sequence
  viral vector JBT-miR1
cggcctcagtgagcgagcgcgcagagagggagtggccaactccatcactaggggttcctgcggccgcacgcgtctagttattaatagtaatcgaattcgtgttactcatagcagatcaccgctgctcgtcaactgaggcgcccaggcagtcaggggacttcgtcgcccggcctcagtgagcgagcgcgcagcgagggagtggcccacttcatcactaggcgttcctgcggccgcacgcgtctagttcactcatagtaatcgaacttcgcgttactcataacagtgaacgctgacgtcatcaacccgctccaaggaatcgcgggcccagtgtcactaggcggggaacacccagcgcgcgtgcgccctggcaggaagatggctgcgagggacaggggagtggcgccccgcaatattttccatgtcggctatgtgttcttgggaaatcaccataaacgtgaaatgtctttggatttgggaatcttataagtttctgtatgagaccactcggatccgtgaggtagtaggttgtatagtttcaagagaactatacaacctactacctcatttttgagctcaaaaaaaacccgtagatccgatcttgtgctcttgacacaagatcggatctacgggtttcggtgttcgcgtcctttccacaagatatataaacccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttctgtcccctccaccccacgtcgacattaatgaagcttggcgactagtaatactgtaatagtaatcaattacggggtcattagttcatagcccatatatggagttccgcgttacataacttacggtaaatggcccgcctggctgaccgcccaacgacccccgcctatatgcatggccccaggaaatcggaaatagttcaagttacggtaagcatatgatagtccattttaaaacattattttaaactgcaaactacccaagaaaattatactttctacgtcacgtattttgtactaatatctttgtgtttacagtcaaattattctaattatctctcctaacagccttgtatcgtatatgcaaatatgaacggaatcatgggaaataggccctcttcctgcccgaccttctgtcccctccaccccacgtcgacgacaggattggtgacagagaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactcggatccggtgaggtagtaggttgtatggtttcaagagaaccatacaacctactacctcatttttgagctcaaaaaaaaacccgtagatccgaacttgtgctcttgacacaagttcggatctacgggttcggtgttcgcgtcctttccacaagatatataaacccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttaagcttggcgactagtaatactgtaatagtaatcaattacggggtcattagttcatagcccatatatggagttccgcgttacataacttacggtaaatggcccgcctggctgaccgcccaacgacccccgccccatgatgccctagtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctggtttagtgaaccgtcagatccgctagagatccggtaccgaggagatctgccgccgcgatcgccggcgcgccagatctcacgcttaactagctagcggaccgacgcgtacgcggccgctcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgaggattataaggatgacgacgataaattcgtcgagcaccaccaccaccaccactaataaggtttatccgatccaccggatctagataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 86 - DNA, 120 bp, Artificial Sequence
  let-7a-5p sequence in vector JBT-miR2
gacggcgctaggatcatcaacaactatacaaccaatgtactacctcacaagtattctggtcacagaatacaacaactatacaaccaatgtactacctcacaagatgatcctagcgccgtc

SEQ ID NO: 87 - DNA, 120 bp, Artificial Sequence
  let-7a-5p Reverse Complement in vector JBT-miR2
gacggcgctaggatcatcttgtgaggtagtacattggttgtatagttgttgtattctgtgaccagaatacttgtgaggtagtacattggttgtatagttgttgatgatcctagcgccgtc

SEQ ID NO: 88 - DNA, 120 bp, Artificial Sequence
  miR-99a-5p sequence in vector JBT-miR2
gacggcgctaggatcatcaaccacaagatcggaaatgtctacgggtacaagtattctggtcacagaatacaaccacaagatcggaaatgtctacgggtacaagatgatcctagcgccgtc

SEQ ID NO: 89 - DNA, 120 bp, Artificial Sequence
  miR-99a-5p Reverse Complement in vector JBT-miR2
gacggcgctaggatcatcttgtacccgtagacatttccgatcttgtggttgtattctgtgaccagaatacttgtacccgtagacatttccgatcttgtggttgatgatcctagcgccgtc

SEQ ID NO: 90 - RNA, 22 nt, Artificial Sequence
  hsa-let-7a
ugagguaguagguuguauaguu

SEQ ID NO: 91 - RNA, 22 nt, Artificial Sequence
  hsa-let-7c
ugagguaguagguuguaugguu

SEQ ID NO: 92 - RNA, 22 nt, Artificial Sequence
  hsa-miR-100
aacccguagauccgaacuugug

SEQ ID NO: 93 - RNA, 22 nt, Artificial Sequence
  hsa-miR-99a
aacccguagauccgaucuugug

SEQ ID NO: 94 - DNA, 24 bp, Artificial Sequence
  HsU6R
gctaatcttctctgtatcgttcca

SEQ ID NO: 95 - DNA, 21 bp, Artificial Sequence
  HsU6F
ggatcagcgtttgagtaagag

SEQ ID NO: 96 - DNA, 22 bp, Artificial Sequence
  Human-U6-52
gcctatttcccatgattccttc

SEQ ID NO: 97 - DNA, 19 bp, Artificial Sequence
  Human-U6-32
ggtgtttcgtcctttccac

SEQ ID NO: 98 - DNA, 1096 bp, Artificial Sequence
  TUD cassette inserted into pAV-U6-GFP
  U6 promoter (1)..(241)
  miR-99a TuD (412)..(531)
  let-7a/c TuD (550)..(669)
  H1 Promoter (678)..(892)
  ITR (950)..(1036)
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgaacaccgacggcgctaggatcatcttgtacccgtagacatttccgatcttgtggttgtattctgtgaccagaatacttgtacccgtagacatttccgatcttgtggttgatgatcctagcgccgtcttttttgagctcaaaaaagacggcgctaggatcatcaacaactatacaaccaatgtactacctcacaagtattctggtcacagaatacaacaactatacaaccaatgtactacctcacaagatgatcctagcgccgtcggatccgagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcactagttatgagtaacacgaattcgattactattaataactagacgcgtgcggccgcaggaacccctagtgatggagttggccactccctctctgcgcgctcgctcactgaggccgggcgaccaaaggtcgcccgacgcccgggcggcctcagtgagcgagcgagcgcgcagctgcctgcaggacatgtgagcaaaaggccagc

SEQ ID NO: 99 - DNA, 2308 bp, Artificial Sequence
  Albumin Stuffer Design 1
  ITR (154)..(180)
  U6 Promoter (340)..(580)
  miR-99a (751)..(870)
  let-7a/c (889)..(1008)
  H1 Promoter (1017)..(1231)
  ITR (2168)..(2308)
ccactccctctatgcgcgctcgctcactcactcggccctggagaccaaaggtctccagactgccggcctctggccggcagggccgagtgagtgagcgagcgcgcatagagggagtgggtacctccatcatctaggtttgcccctgcaggcagctgcgcgctcgctcgctcactgaggccgcccgggcgtcgggcgacctttggtcgcccggcctcagtgagcgagcgcgcagagagggagtggccaactccatcactaggggttcctgcggccgcacgcgtctagttattaatagtaatcgaattcgtgttactcataactagtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgaacaccgacggcgctaggatcatcttgtacccgtagacatttccgatcttgtggttgtattctgtgaccagaatacttgtacccgtagacatttccgatcttgtggttgatgatcctagcgccgtcttttttgagctcaaaaaagacggcgctaggatcatcaacaactatacaaccaatgtactacctcacaagtattctggtcacagaatacaacaactatacaaccaatgtactacctcacaagatgatcctagcgccgtcggatccgagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcactagttatgagtaacacgaattcgattactattaataactagacgcgtgcggccgcaggaacccctagtgatggagttggccactccctctctgcgcgctcgctcactgaggccgggcgaccaaaggtcgcccgacgcccgggcggcctcagtgagcgagcgagcgcgcagctgcctgcaggacatgtgagcaaaaggccagccaacaaaatcagcagctaatgaaggcaagtcagcaggtcactcatcattttccacttcggcaatgcagtgggattattccaacagaggtttttcacagcattccttcagttaactggagatcgaatcttgattttcacagatatacttggcaaggtccgccctgtcatcagcacattcaagcagatctccatggcagcattccgtgtggacttaggtaagatctgtcactaacttggaaacttctgcaaactcagcttagggaaatctctggctcaggcgagctactgcctcagcttagaaagctctttctccaaattattggagactggcacacttaagtccctgttaggcagacgaagccttcccttcatcccgaagttcatcgagctttggcaacaggcaggcagctttatcagcagcttggcaacattctgtaaaagcagctttatacctttaagcaaagaaaaggagttccggggcataaaagtaaggatgtcttctggcaatttataataagtattttttcaaaaatgtctcttcattgtcatgaaaagcagtgcatcacacatcaacctctggtctcaccaatcgggggaggtttgggttgtttacttagtgttgcaagaattattttattctctcaggttcttgttttgcacagcagtcagctcattcaccataggtttcacgaagagttgcacgcggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 100 - DNA, 3593 bp, Artificial Sequence
  ADD Stuffer Design 2
  ITR (154)..(180)
  U6 Promoter (340)..(580)
  miR-99a (751)..(870)
  let-7a/c (889)..(1008)
  H1 Promoter (1017)..(1231)
  ITR (3453)..(3593)
ccactccctctatgcgcgctcgctcactcactcggccctggagaccaaaggtctccagactgccggcctctggccggcagggccgagtgagtgagcgagcgcgcatagagggagtgggtacctccatcatctaggtttgcccctgcaggcagctgcgcgctcgctcgctcactgaggccgcccgggcgtcgggcgacctttggtcgcccggcctcagtgagcgagcgcgcagagagggagtggccaactccatcactaggggttcctgcggccgcacgcgtctagttattaatagtaatcgaattcgtgttactcataactagtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgaacaccgacggcgctaggatcatcttgtacccgtagacatttccgatcttgtggttgtattctgtgaccagaatacttgtacccgtagacatttccgatcttgtggttgatgatcctagcgccgtcttttttgagctcaaaaaagacggcgctaggatcatcaacaactatacaaccaatgtactacctcacaagtattctggtcacagaatacaacaactatacaaccaatgtactacctcacaagatgatcctagcgccgtcggatccgagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcactagttatgagtaacacgaattcgattactattaataactagacgcgtgcggccgcaggaacccctagtgatggagttggccactccctctctgcgcgctcgctcactgaggccgggcgaccaaaggtcgcccgacgcccgggcggcctcagtgagcgagcgagcgcgcagctgcctgcaggacatgtgagcaaaaggccagccgtttagtgaaccgtcagatcgcctggagacgccatccacgctgttttgacctccatagaagacaccgggaccgatccagcctccggactctagagagacgtacaaaaaagagcaagaagctaaaaaagatttaaaaattatttttagcgcagttaatggaacaggaactaaatttaccccaaaaatattacgtgaatcaggatataacgttattgaggttgaagagcatgcatttgaagatgaaacatttaaaaatgttgtaaatccaaatccagaatttgatcctgcatgaaaaataccgcttgaatatggtattaaacatgatgcagatattattattatgaatgacccagatgctgacagatttggaatggcaataaaacatgatggtcattttgtaagattagatggaaatcaaacaggaccaattttaattgattgaaaattatcaaatctaaaacgcttaaatagcattccaaaaaatccggctctatattcaagttttgtaacaagtgatttgggtgatagaatcgctcatgaaaaatatggagttaatattgtaaaaactttaactggatttaaatgaatgggtagagaaattgctaaagaagaagataacggattaaattttgtttttgcttatgaagaaagttatggatatgtaattgatgactcagctagagataaagatggaatacaagcttctatattaatagcagaggctgcttgattttataaaaaacaaaataaaacattagtagactatttagaagatttatttaaagaaatgggtgcatattacactttcactttaaacttgaattttaaaccagaagaaaagaaattaaaaattgaaccattaatgaaatcattgagagcaacacccttaactcaaattgctggacttaaagttgttaatgttgaagactacatcgatggaatgtataatatgccaggacaagacttactaaaattttatttagaagataagtcatgatttgctgttcgcccaagtggaactgaacctaaactaaaaatttattttataggtgttggtgaatctgttcaaaacgctaaagttaaagtagacgaaattattaaagaattaaaattaaaaatgaatatataggagaaaaaatgaaactaaacaaatatatagatcacacattattaaaacaagatgctacgaaagctgaaattaaacaattatgtgatgaagcaattgaatttgattttgcaacagtttgtgttaattcatattgaacaagctattgtaaagaattattaaaaggcacaaatgtaggaataacaaatgttgtaggttttcctctaggtgcatgcacaacagctacaaaagcattcgaagtttctgaagcaattaaagatggtgcaacagaaattgatatggtattaaatattggtgcattaaaagacaaaaattatgaattagttttagaagacatgaaagctgtaaaaaaagcagctggatcacatgttgttaaatgtattatggaaaattgtttattaacaaaagaagaaatcatgaaagcttgtgaaatagctgttgaagctggattagaatttgttaaaacatcaacaggattttcaaaatcaggtgcaacatttgaagatgttaaactaatgaagtcagttgttaaagacaatgctttagttaaagcagctggtggagttagaacatttgaagatgctcaaaaaatgattgaagcaggagctgaccgcttaggaacaagtggtggagtagctattattaaaggtgaagaaaacaacgcgagttactaaaactagcgtttttttattttgctcatttttattaaaagtttgcaaaaaggaacataaaaattctaattattgatactaaagttattaaaaagaagattttggttgattttataaaggtcatagaatataatattttagcatgtgtattttgtgtgctcatttacaaccgtctcgcggccgcgggacgcggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 101 - DNA, 1604 bp, Artificial Sequence
  Partial JBT-miR2 viral sequence
  U6 Promoter (251)..(491)
  miR-99a (501)..(620)
  let-7a/c (639)..(758)
  H1 Promoter (767)..(981)
agtttgttttggcaccaaaatcaacgggactttccaaaatgtcgtaacaactccgccccattgacgtcaatgggcgggggtcgttgggcggtcagccaggcgggccatttaccgtaagttatgtaacgcggaactccatatatgggctatgaactaatgaccccgtaattgattactattacagtattactagtcgccaagcttcattaatgtcgacgtggggtggaggggacagaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgaacaccgacggcgctaggatcatcttgtacccgtagacatttccgatcttgtggttgtattctgtgaccagaatacttgtacccgtagacatttccgatcttgtggttgatgatcctagcgccgtcttttttgagctcaaaaaagacggcgctaggatcatcaacaactatacaaccaatgtactacctcacaagtattctggtcacagaatacaacaactatacaaccaatgtactacctcacaagatgatcctagcgccgtcggatccgagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcactagttatgagtaacacgaattcgattactattaataactagacgcgtgcggccgcaggaacccctagtgatggagttggccactccctctctgcgcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaactgagggcctatttcc

(19) United States
(12) Patent Application Publication
(10) Pub. No.: US 2022/0307028 A1
(43) Pub. Date: Sep. 29, 2022

(54) COMPOSITIONS AND METHODS FOR MITIGATION OF ISCHEMIC REPERFUSION INJURY

(71) Applicant: JAAN BIOTHERAPEUTICS LLC, SAN DIEGO, CA (US)

(72) Inventor: BHAWANJIT KAUR BRAR, San Diego, CA (US)

(21) Appl. No.: 17/770,031

(22) PCT Filed: Oct. 20, 2020
(86) PCT No.: PCT/US2020/056536

§ 371 Date: Apr. 18, 2022

Related U.S. Application Data
(60) Provisional application No. 62/923,612, filed on Oct. 21, 2019.
(60) Provisional application No. 62/937,429, filed on Nov. 19, 2019.
(60) Provisional application No. 62/961,418, filed on Jan. 15, 2020.
(60) Provisional application No. 62/981,531, filed on Feb. 26, 2020.

(51) Int. Cl. C12N 15/113; A61P 13/12; A61P 9/00

(52) U.S. Cl. CPC C12N 15/113; A61P 13/12; A61P 9/00; C12N 2310/113; C12N 2310/315; C12N 2310/3231

(57) ABSTRACT

There are provided, in some embodiments, therapeutic compositions and methods for preventing, inhibiting, reducing, or treating cardiac ischemic reperfusion injury. The therapeutic composition can comprise a plurality of microRNA (miR) antagonists. In some embodiments, the method comprises administering a therapeutic composition to a subject before, during, and/or after a cardiac ischemic event. The method can comprise reperfusion of ischemic cardiac tissue.

### RELATED APPLICATIONS

The present application claims priority to U.S. Provisional Application No. 62/981,531, filed Feb. 26, 2020; U.S. Provisional Application No. 62/961,418, filed Jan. 15, 2020; U.S. Provisional Application No. 62/937,429, filed Nov. 19, 2019; and U.S. Provisional Application No. 62/923,612, filed Oct. 21, 2019. The entire contents of these applications are hereby expressly incorporated by reference in their entireties.

### STATEMENT REGARDING FEDERALLY SPONSORED R&D

This invention was made with government support under contract no. 1R43HL137416-01A1 awarded by the National Institutes of Health, and under grant no. 1R41HL134387-01A1 awarded by the National Institutes of Health. The government has certain rights in the invention.

### REFERENCE TO SEQUENCE LISTING

The present application is being filed along with a Sequence Listing in electronic format. The Sequence Listing is provided as a file entitled Sequence_Listing_68BX-306236-WO, created Oct. 20, 2020, which is 40.0 kilobytes in size. The information in the electronic format of the Sequence Listing is incorporated herein by reference in its entirety.

### BACKGROUND

### Field

The present disclosure relates generally to the fields of biochemistry and medicine. More particularly, disclosed herein are methods of preventing, inhibiting, reducing, or treating cardiac ischemic reperfusion injury.

### Description of the Related Art

Heart diseases encompass a family of disorders, including, but not limited to cardiomyopathies, myocardial infarction, and ischemic heart disease where cardiac muscle regeneration is required. Ischemic heart disease is a leading cause of morbidity and mortality in the industrialized world. Disorders within the heart disease spectrum are understood to arise from pathogenic changes in distinct cell types, such as cardiomyocytes, via alterations in a complex set of biochemical pathways. For example, certain pathological changes linked with heart disease can be accounted for by alterations in cardiomyocyte gene expression that lead to cardiomyocyte hypertrophy and impaired cardiomyocyte survival and contraction. Thus, an ongoing challenge in the development of heart disease treatments has been to identify effective therapies suitable for various types of heart diseases by, for example, promoting endogenous cardiac myocytes within the heart to divide and repair the damaged cardiac muscle.

Cardiac ischemia, a condition characterized by reduced blood flow and oxygen to the heart muscle, or myocardium, is one hallmark of cardiovascular disease that can ultimately lead to a heart attack, or myocardial infarction. Cardiovascular disease can also result in restricted blood flow and reduced oxygen supply to other areas of the body resulting in ischemic injuries to various organs and tissues, including the brain, which can lead to stroke. Re-establishment of blood flow, or reperfusion, and re-oxygenation of the affected area following an ischemic episode is critical to limit irreversible damage. However, reperfusion also brings potentially damaging consequences, such as reperfusion injury, which is caused by the restoration of coronary blood flow after an ischemic episode and results from the generation and accumulation of reactive oxygen and nitrogen species during reperfusion. Ischemia-reperfusion injury is biochemically characterized by a depletion of oxygen during an ischemic event, a resultant increase in intracellular calcium levels, followed by reoxygenation and the concomitant generation of reactive oxygen species during reperfusion. Reperfusion injury may be responsible for as much as 50% of the damage to the heart following a myocardial infarction. The prevalence of cardiovascular disease in the United States, and throughout the world, necessitates the development of methods and compositions that can effectively prevent, reduce, or counteract ischemia and ischemia-reperfusion injury resulting from a cardiac ischemic event. There a significant need for new and more effective therapies and therapeutic agents for the treatment of ischemia and ischemia-reperfusion injuries resulting from cardiovascular disease and other conditions.

### SUMMARY

Disclosed herein include methods of preventing, inhibiting, reducing, or treating cardiac ischemic reperfusion injury. In some embodiments, the method comprises: administering a therapeutic composition to a subject before, during, and/or after a cardiac ischemic event, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b). The method can comprise: reperfusion of ischemic cardiac tissue.

Disclosed herein include methods of treating myocardial infarction. In some embodiments, the method comprises: administering a therapeutic composition to a subject before, during, and/or after reperfusion of ischemic cardiac tissue, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b). In some embodiments, myocardial infarction is a cardiac ischemic event.

Disclosed herein include methods of inducing cardiomyocyte regeneration, cardiac repair, vasculogenesis and/or cardiomyocyte differentiation following a cardiac ischemic event. In some embodiments, the method comprises: administering a therapeutic composition to a subject before, during, or after reperfusion of ischemic cardiac tissue, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

Disclosed herein include methods of treating a disease or disorder associated with dysregulation of FHL1 and/or TNNT2. In some embodiments, the method comprises: administering a therapeutic composition to a subject in need thereof, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

Disclosed herein include methods of treating a kidney condition of a subject and/or protecting a kidney of a subject from injury. In some embodiments, the method comprises: administering a therapeutic composition to the subject, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

In some embodiments, at least one of the one or more miR-99a antagonists comprises an anti-miR-99a comprising a nucleotide sequence having at least 80%, 85%, 90%, 95%, 96%, 97, 98%, 99% or 100% identity to a sequence selected from the group consisting of SEQ ID NOs 47, 48, 50, 52, and 54. In some embodiments, at least one of the one or more miR-100-5p antagonists comprises an anti-miR-100-5p comprising a nucleotide sequence having at least 80%, 85%, 90%, 95%, 96%, 97, 98%, 99% or 100% identity to a sequence selected from the group consisting of SEQ ID NOs 46, 49, 51, 53, and 55. In some embodiments, at least one of the one or more Let-7a-5p antagonists comprises an anti-miR-Let-7a-5p comprising a nucleotide sequence having at least 80%, 85%, 90%, 95%, 96%, 97, 98%, 99% or 100% identity to a sequence selected from the group consisting of SEQ ID NOs: 37, 39, and 40-45. In some embodiments, at least one of the one or more Let-7c-5p antagonists comprises an anti-miR-Let-7c-5p comprising a nucleotide sequence having at least 80%, 85%, 90%, 95%, 96%, 97, 98%, 99% or 100% identity to a sequence selected from the group consisting of SEQ ID NOs: 36, 38, and 40-45.

In some embodiments, at least one of the one or more miR-99a antagonists comprises an anti-miR-99a comprising a nucleotide sequence having one or more mismatched nucleobases with respect to a sequence selected from the group consisting of SEQ ID NOs: 47, 48, 50, 52, and 54. In some embodiments, at least one of the one or more miR-100-5p antagonists comprises an anti-miR-100-5p comprising a nucleotide sequence having one or more mismatched nucleobases with respect to a sequence selected from the group consisting of SEQ ID NOs: 46, 49, 51, 53, and 55. In some embodiments, at least one of the one or more Let-7a-5p antagonists comprises an anti-miR-Let-7a-5p comprising a nucleotide sequence having one or more mismatched nucleobases with respect to a sequence selected from the group consisting of SEQ ID NOs: 37, 39, and 40-45. In some embodiments, at least one of the one or more Let-7c-5p antagonists comprises an anti-miR-Let-7c-5p comprising a nucleotide sequence having one or more mismatched nucleobases with respect to a sequence selected from the group consisting of SEQ ID NOs: 36, 38, and 40-45.

In some embodiments, at least one of the anti-miRs comprises one or more chemical modifications selected from the group consisting of a modified internucleoside linkage, a modified nucleotide, and a modified sugar moiety, and combinations thereof. In some embodiments, the one or more chemical modifications comprises a modified internucleoside linkage. In some embodiments, the modified internucleoside linkage is selected from the group consisting of a phosphorothioate, 2′-Omethoxyethyl (MOE), 2′-fluoro, alkylphosphonate, phosphorodithioate, alkylphosphonothioate, phosphoramidate, carbamate, carbonate, phosphate triester, acetamidate, carboxymethyl ester, and combinations thereof. In some embodiments, the modified internucleoside linkage comprises a phosphorothioate internucleoside linkage. In some embodiments, at least one of the one or more chemical modifications comprises a modified nucleotide. The modified nucleotide can comprise a locked nucleic acid (LNA). The locked nucleic acid (LNA) can be incorporated at one or both ends of the modified anti-miR. In some embodiments, the modified nucleotide comprises a locked nucleic acid (LNA) chemistry modification, a peptide nucleic acid (PNA), an arabino-nucleic acid (FANA), an analogue, a derivative, or a combination thereof. In some embodiments, at least one of the one or more chemical modifications comprises a modified sugar moiety. In some embodiments, the modified sugar moiety is a 2′-O-methoxyethyl modified sugar moiety, a 2′-methoxy modified sugar moiety, a 2′-O-alkyl modified sugar moiety, a bicyclic sugar moiety, or a combination thereof. In some embodiments, the modified sugar moiety comprises a 2′-O-methyl sugar moiety.

In some embodiments, the cloning or expression vector is a viral vector. In some embodiments, the viral vector is a lentiviral vector or an adeno-associated viral (AAV) vector. In some embodiments, the cloning or expression vector comprises: (a) a nucleotide sequence having at least 80%, 85%, 90%, 95%, 96%, 97, 98%, 99% or 100% identity to each of the nucleotide sequences set forth in SEQ ID NOs: 59-64; (b) a nucleotide sequence having at least 80%, 85%, 90%, 95%, 96%, 97, 98%, 99% or 100% identity to each of the nucleotide sequences set forth in SEQ ID NOs: 86-89; or (c) a nucleotide sequence having at least 80%, 85%, 90%, 95%, 96%, 97, 98%, 99% or 100% identity to each of the nucleotide sequences set forth in the SEQ ID NOs indicated in (a) and (b). In some embodiments, the cloning or expression vector comprises a nucleotide sequence having least 80%, 85%, 90%, 95%, 96%, 97, 98%, 99% or 100% identity to the nucleotide sequence of SEQ ID NO: 85. In some embodiments, the plurality of miR antagonists are encoded by the same expression cassette or vector. In some embodiments, the plurality of miR antagonists are encoded by different expression cassettes or vectors.

In some embodiments, the cloning or expression vector comprises a nucleotide sequence having least 80%, 85%, 90%, 95%, 96%, 97, 98%, 99% or 100% identity to the nucleotide sequence of SEQ ID NO: 101. In some embodiments, the expression cassette comprises a tough decoy (TuD) cassette comprising a nucleotide sequence encoding one or more miR-99a antagonists. In some embodiments, the TuD cassette comprises one or more promoter sequences operably linked to the nucleotide sequence encoding one or more miR-99a antagonists, optionally the one or more promoter sequences comprise a H1 promoter and/or a U6 promoter. In some embodiments, the cloning or expression vector comprises two or more TuD cassettes. In some embodiments, an effective dose of a therapeutic composition comprising a cloning or expression vector comprising two or more TuD cassettes is at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) less than an effective dose of a therapeutic composition comprising a cloning or expression vector comprising one TuD cassette. In some embodiments, the TuD cassette comprises a nucleotide sequence having least 80%, 85%, 90%, 95%, 96%, 97, 98%, 99% or 100% identity to the nucleotide sequence of SEQ ID NO: 98. In some embodiments, the cloning or expression vector comprises a nucleotide sequence having least 80%, 85%, 90%, 95%, 96%, 97, 98%, 99% or 100% identity to the nucleotide sequence of SEQ ID NO: 99. In some embodiments, the cloning or expression vector comprises a nucleotide sequence having least 80%, 85%, 90%, 95%, 96%, 97, 98%, 99% or 100% identity to the nucleotide sequence of SEQ ID NO: 100.

In some embodiments, the therapeutic composition is a pharmaceutical composition. In some embodiments, administering the therapeutic composition occurs before the onset of the cardiac ischemic event. In some embodiments, administering the therapeutic composition occurs during the cardiac ischemic event. In some embodiments, the therapeutic composition is administered about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, about 72 hours, or about 96 hours prior to reperfusion of ischemic cardiac tissue. In some embodiments, administering the therapeutic composition occurs concurrent with reperfusion of ischemic cardiac tissue. In some embodiments, administering the therapeutic composition occurs after reperfusion of ischemic cardiac tissue. In some embodiments, the therapeutic composition is administered about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, about 72 hours, about 96 hours, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, or about 20 days, after reperfusion of ischemic cardiac tissue.

In some embodiments, the therapeutic composition comprises a plurality of microRNA (miR) antagonists, wherein the administration comprises subcutaneous administration, systemic administration, and/or intra-coronary administration. In some embodiments, the therapeutic composition is administered at a dose of about 0.08 mg/kg, about 0.24 mg/kg, about 0.81 mg/kg, about 1.22 mg/kg, about 2.44 mg/kg, about 3.25 mg/kg, about 4.06 mg/kg, about 4.89 mg/kg, about 5.69 mg/kg, about 6.50 mg/kg, about 7.32 mg/kg, or about 8.13 mg/kg. In some embodiments, the therapeutic composition comprises a plurality of microRNA (miR) antagonists, wherein the administration comprises intra-ventricular administration and/or intra-myocardial administration. In some embodiments, the therapeutic composition is administered at a dose of about 0.004 mg/kg, about 0.012 mg/kg, about 0.0405 mg/kg, about 0.061 mg/kg, about 0.122 mg/kg, about 0.1625 mg/kg, about 0.203 mg/kg, about 0.2445 mg/kg, about 0.2845 mg/kg, about 0.325 mg/kg, about 0.366 mg/kg, or about 0.4065 mg/kg. In some embodiments, subcutaneous administration of the therapeutic composition yields increased survival and reduced incidence of cardiac thrombus as compared to intravenous administration of the therapeutic composition. In some embodiments, the therapeutic composition comprises a viral vector, wherein the administration comprises intravenous systemic administration and/or intra-coronary administration at a dose of about 2.5×10¹² vg (viral genome)/kg, about 2.5×10¹³ vg/kg, about 2.5×10¹⁴ vg/kg, or about 2.5×10¹⁵ vg/kg. In some embodiments, the therapeutic composition comprises a viral vector, wherein the administration comprises intra-ventricular administration and/or intra-myocardial administration. In some embodiments, the therapeutic composition is administered at a dose of about 0.125×10¹² vg/kg, about 0.125×10¹³ vg/kg, about 0.125×10¹⁴ vg/kg, or about 0.125×10¹⁵ vg/kg. In some embodiments, the dose is administered in a single administration. In some embodiments, the dose is administered over multiple administrations.

The method can comprise: repeated administration of the therapeutic composition to the subject. The repeated administration can comprise administration of one or more additional doses of the therapeutic composition to the subject. In some embodiments, the repeated administration comprises administration of one or more additional doses of the therapeutic composition to the subject about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, about 72 hours, about 96 hours, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, and/or about 20 days, after reperfusion of ischemic cardiac tissue.

The method can comprise: administrating an effective amount of at least one additional therapeutic agent or at least one additional therapy to the subject for a combination therapy. In some embodiments, each of the therapeutic composition and the at least one additional therapeutic agent or therapy is administered in a separate formulation or are administered together in a single formulation. In some embodiments, the therapeutic composition and the at least one additional therapeutic agent or therapy are administered sequentially, are administered concomitantly, and/or are administered in rotation. In some embodiments, the at least one additional therapeutic agent or therapeutic therapy is selected from the group consisting of Idebenone, Eplerenone, VECTTOR, AVI-4658, Ataluren/PTC124/Translarna, BMN044/PRO044, CAT-1004, microDystrophin AAV gene therapy (SGT-001), Galectin-1 therapy (SB-002), LTBB4 (SB-001), rAAV2.5-CMV-minidystrophin, glutamine, NFKB inhibitors, sarcoglycan, delta (35 kDa dystrophin-associated glycoprotein), insulin like growth factor-1 (IGF-1) expression, genome editing through the CRISPR/Cas9 system, any gene delivery therapy aimed at reintroducing a functional recombinant version of the dystrophin gene, Exon skipping therapeutics, read-through strategies for nonsense mutations, cell-based therapies, utrophin upregulation, myostatin inhibition, anti-inflammatories/anti-oxidants, mechanical support devices, a biologic drug, a gene therapy or therapeutic gene modulation agent, any standard therapy for muscular dystrophy, and combinations thereof. In some embodiments, the at least one additional therapeutic agent or therapeutic therapy is selected from the group comprising a percutaneous coronary intervention, coronary artery bypass grafting, thrombolytic therapy, anti-platelet therapy, heparin, warfarin, fibrinolytics, oxygen therapy, a vasodilator, pain medication, a beta blocker, an angiotensin-converting enzyme (ACE) inhibitor, an angiotensin receptor blocker (ARB), a glycoprotein antagonist, a statin, an aldosterone antagonist, an implantable cardiac defibrillator (ICD), or any combination thereof.

In some embodiments, reperfusion of ischemic cardiac tissue comprises a percutaneous coronary intervention, coronary artery bypass grafting, thrombolytic therapy, anti-platelet therapy, heparin, warfarin, fibrinolytics, oxygen therapy, a vasodilator, pain medication, a beta blocker, an angiotensin-converting enzyme (ACE) inhibitor, an angiotensin receptor blocker (ARB), a glycoprotein antagonist, a statin, an aldosterone antagonist, an implantable cardiac defibrillator (ICD), or any combination thereof. In some embodiments, the subject is a mammal (e.g., a human) In some embodiments, the subject has or is suspected of having a cardiac disease, wherein the cardiac disease is myocardial infarction, ischemic heart disease, dilated cardiomyopathy, heart failure (e.g., congestive heart failure), ischemic cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis, aortic stenosis, aortic regurgitation, mitral stenosis, mitral regurgitation, mitral prolapse, pulmonary stenosis, pulmonary regurgitation, tricuspid stenosis, tricuspid regurgitation, congenital disorder, genetic disorder, or any combination thereof. In some embodiments, the subject is affected by a condition selected from the group comprising alcoholic cardiomyopathy, coronary artery disease, congenital heart disease, nutritional diseases affecting the heart, ischemic cardiomyopathy, hypertensive cardiomyopathy, valvular cardiomyopathy, inflammatory cardiomyopathy, cardiomyopathy secondary to a systemic metabolic disease, dilated cardiomyopathy (DCM), hypertrophic cardiomyopathy (HCM), arrhythmogenic right ventricular cardiomyopathy (ARVC), restrictive cardiomyopathy (RCM), noncompaction cardiomyopathy, supravalvular aortic stenosis (SVAS), vascular scarring, atherosclerosis, chronic progressive glomerular disease, glomerulosclerosis, progressive renal failure, vascular occlusion, hypertension, stenosis, diabetic retinopathy, or any combination thereof. In some embodiments, the cardiac ischemic reperfusion injury comprises cardiac ischemic damage, cardiac reperfusion injury, or a combination thereof.

In some embodiments, the administration reduces cardiac ischemic damage, cardiac reperfusion injury, or a combination thereof, as compared to a control subject. In some embodiments, the administration reduces creatine kinase levels as compared to a control subject by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) at a time point about 5 minutes to about 365 days after administration (e.g., about 5 minutes, about 10 minutes, about 15 minutes, about 20 minutes, about 25 minutes, about 30 minutes, about 40 minutes, about 50 minutes, or about 60 minutes, about 1 day, about 2 days, about 4 days, about 6 days, about 8 days, about 10 days, about 20 days, about 30 days, about 40 days, about 50 days, about 60 days, about 80 days, about 100 days, about 120 days, about 140 days, about 160 days, about 180 days, about 200 days, about 220 days, about 240 days, about 260 days, about 280 days, about 300 days, about 320 days, about 340 days, about 360 days, about 365 days, or a number or a range between any of these values). In some embodiments, the cardiac ischemic reperfusion injury comprises injuries caused by the cardiac ischemia event, reperfusion injuries, or a combination thereof. In some embodiments, the cardiac ischemic event comprises one or more of myocardial infarction, coronary artery bypass grafting (CABG), cardiac bypass surgery, cardiac transplantation, and angioplasty. In some embodiments, the cardiac ischemic event comprises a vascular interventional procedure employing a stent, laser catheter, atherectomy catheter, angioscopy device, beta or gamma radiation catheter, rotational atherectomy device, coated stent, radioactive balloon, heatable wire, heatable balloon, biodegradable stent strut, a biodegradable sleeve, or any combination thereof.

In some embodiments, the administration results in one or more of (1) increased survival as compared to a control subject, (2) improved kidney function of the subject as compared to a control subject, (3) a decrease in blood urea nitrogen (BUN) levels as compared to a control subject, (4) a reduced scarring in the left ventricle of the subject and/or improved regional wall motion in the left ventricle of the subject as compared to a control subject, (5) a decrease in end diastolic volume and/or end systolic volume as compared to a control subject, (6) an increase in ejection fraction as compared to a control subject, (7) an increase in the number of cardiomyocytes and/or mRNAs encoding proteins that are involved in differentiated cardiomyocyte muscle structure and function as compared to a control subject, (8) an increase in the mRNA levels and/or protein levels of one or more of Ank2, Kdm6a, Grk6, K1h115, Adam22, Pfkp, Gorasp2, Ralgps1, Inppl1, Kdm3a, Kit, Sort1, Dv12, Sema6d, Tead1, B4galnt2, Ltbp4, Osbp19, Nfe2I1, Tnnt2, and Fhl1 as compared to a control subject, and (9) a decrease in the mRNA levels and/or protein levels of one or more of Asph, Map6, Zfp120, Ctnndl, Eya3, Tnnt2, Kdm3a, Myo18a, Ncoa6, Slc25a13, Rpe, Ralgps1, Gimap1, Myo5a, Zeb2, Arap1, Nt5c2, Phldb1, Ttn, Camta2, Mef2c, Slk, Uimc1, Mthfd1I, Mtus1, Ythdc1, and Eif2ak4 as compared to a control subject, and (10) an increase in one of more of cardiomyocyte formation, cardiomyocyte proliferation, cardiomyocyte cell cycle activation, mitotic index of cardiomyocytes, myofilament density, borderzone wall thickness, or any combination thereof, as compared to a control subject, by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) at a time point about 5 minutes to about 365 days after administration (e.g., about 5 minutes, about 10 minutes, about 15 minutes, about 20 minutes, about 25 minutes, about 30 minutes, about 40 minutes, about 50 minutes, or about 60 minutes, about 1 day, about 2 days, about 4 days, about 6 days, about 8 days, about 10 days, about 20 days, about 30 days, about 40 days, about 50 days, about 60 days, about 80 days, about 100 days, about 120 days, about 140 days, about 160 days, about 180 days, about 200 days, about 220 days, about 240 days, about 260 days, about 280 days, about 300 days, about 320 days, about 340 days, about 360 days, about 365 days, or a number or a range between any of these values). In some embodiments, the administration induces endogenous cardiomyocyte regeneration. In some embodiments, the administration enhances cardiac function in the subject as compared to a control subject. Enhancing cardiac function can comprise one or more of (i) improving left ventricular function, (ii) improving fractional shortening, (iii) improving ejection fraction, (iv) reducing end-diastolic volume, (v) decreasing left ventricular mass, and (v) normalizing of heart geometry, or (vi) a combination thereof. In some embodiments, the administration has no significant effect on body weight and/or heart weight. In some embodiments, the administration does not cause one or more of arrhythmia, after contractions (AC), and contraction failure (CF).

In some embodiments, the therapeutic composition increases the mRNA levels and/or protein levels of FHL1 and/or TNNT2. In some embodiments, the disease or disorder is associated with one or more FHL1 mutations and/or one or more TNNT2 mutations. In some embodiments, the disease or disorder is a muscular dystrophy disorder or a muscular dystrophy-like muscle disorder. The muscular dystrophy disorder can be associated with Amyotrophic Lateral Sclerosis (ALS), Charcot-Marie-Tooth Disease (CMT), Congenital Muscular Dystrophy (CMD), Duchenne Muscular Dystrophy (DMD), Emery-Dreifuss Muscular Dystrophy (EDMD), Inherited and Endocrine Myopathies, Metabolic Diseases of Muscle, Mitochondrial Myopathies (MM), Myotonic Muscular Dystrophy (MMD), Spinal-Bulbar Muscular Atrophy (SBMA), or a combination thereof. In some embodiments, the disease or disorder is Limb girdle muscular dystrophy, X-linked myopathy with postural muscle atrophy (XMPMA), Reducing body myopathy (RBM), Scapuloperoneal (SP) syndrome, or any combination thereof. In some embodiments, the disease or disorder is hypertrophic cardiomyopathy (HCM), restrictive cardiomyopathy (RCM), dilated cardiomyopathy (DCM), or any combination thereof. The hypertrophic cardiomyopathy can be familial hypertrophic cardiomyopathy.

In some embodiments, the kidney condition is associated with a function of the subject's kidneys. In some embodiments, the kidney condition is selected from the group consisting of acute kidney diseases and disorders (AKD), acute kidney injury, acute and rapidly progressive glomerulonephritis, acute presentations of nephrotic syndrome, acute pyelonephritis, acute renal failure, idiopathic chronic glomerulonephritis, secondary chronic glomerulonephritis, chronic heart failure, chronic interstitial nephritis, chronic kidney disease (CKD), chronic liver disease, chronic pyelonephritis, diabetes, diabetic kidney disease, fibrosis, focal segmental glomerulosclerosis, Goodpasture's disease, diabetic nephropathy, hereditary nephropathy, interstitial nephropathy, hypertensive nephrosclerosis, IgG4-related renal disease, interstitial inflammation, lupus nephritis, nephritic syndrome, partial obstruction of the urinary tract, polycystic kidney disease, progressive renal disease, renal cell carcinoma, renal fibrosis, graft versus host disease after renal transplant, and vasculitis. In some embodiments, the injury is associated with one or more of surgery, radiocontrast imaging, radiocontrast nephropathy, cardiovascular surgery, cardiopulmonary bypass, extracorporeal membrane oxygenation (ECMO), balloon angioplasty, induced cardiac or cerebral ischemic-reperfusion injury, organ transplantation, kidney transplantation, sepsis, shock, low blood pressure, high blood pressure, kidney hypoperfusion, chemotherapy, drug administration, nephrotoxic drug administration, blunt force trauma, puncture, poison, or smoking.

In some embodiments, the therapeutic composition is administered in combination with a renal therapeutic agent is selected from the group consisting of dexamethasone, a steroid, budesonide, triamcinolone acetonide, an anti-inflammatory agent, an antioxidant, deferoxamine, feroxamine, a tin complex, a tin porphyrin complex, a metal chelator, ethylenediaminetetraacetic acid (EDTA), an EDTA-Fe complex, dimercapto succinic acid (DMSA), 2,3-dimercapto-1-propanesulfonic acid (DMPS), penicillamine, minocycline, prednisone, azathioprine, mycophenolate mofetil, mycophemolic acid, sirolimius, cyclorsporine, or tacrolimusan antibiotic, an iron chelator, a porphyrin, hemin, vitamin B 12, an Nrf2 pathway activator, bardoxolone, ACE inhibitors, enalapril, glycine polymers, antioxidants, glutathione, N acetyl cysteine, a chemotherapeutic, QPI-1002, QM56, SVT016426 (QM31), 16/86 (third generation ferrostatin), BASP siRNA, CCX140, BIIB023, CXA-10, alkaline phosphatase, Dnmtl inhibitor, THR-184, lithium, formoterol, IL-22, EPO, EPO derivative, agents that stimulate erthyropoietin, epoeitn alfa, darbepoietin alfa, PDGF inhibitor, CRMD-001, Atrasentan, Tolvaptan, RWJ-676070, Abatacept, Sotatercept, an anti-infective agent, an antibiotic, an anti-viral agent, an anti-fungal agent, an aminoglycoside, a nonsteroidal anti-inflammatory drug (NSAID), a diuretic drug, a statin, a senolytic, a corticosteroid, a glucocorticoid, a liposome, renin, angiotensin, ACE inhibitor, mediator of apoptosis, mediator of fibrosis, drug that targets p53, Apaf-1 inhibitor, RIPK1 inhibitor, RIPK3 inhibitor, inhibitor of IL17, inhibitor of IL6, inhibitor of IL23, inhibitor of CCR2, nitrated fatty acids, angiotensin blockers, agonists of the ALK3 receptor, and retinoic acid.

In some embodiments, the therapeutic composition is administered in combination with a renal protective agent or a renal prophylactic agent selected from the group consisting of thiazide, bemetanide, ethacrynic acid, furosemidem torsemide, glucose, mannitol, amiloride, spironolactone, eplerenone, triamterene, potassium canrenoate, bendroflumethiazide, hydrochlorothiazide, vasopressin, amphotericin B, acetazolamide, tovaptan, conivaptan, dopamine, dorzolamide, bendrolumethiazide, hydrochlorothiazide, caffeine, theophylline, theobromine, a statin, a senolytic, navitoclax obatoclax, a corticosteroid, prednisone, betamethasone, fludrocortisone, deoxycorticosterone, aldosterone, cortisone, hydrocortisone, belcometasone, mometasone, fluticasone, prednisolone, methylprednisolone, triamcinolone acetonide, a glucocorticoid, dexamethasone, a steroid, budesonide, triamcinolone acetonide, an anti-inflammatory agent, an antioxidant, a nonsteroidal anti-inflammatory drug (NSAID), deferoxamine, iron, tin, a metal, a metal chelate, ethylenediaminetetraacetic acid (EDTA), dimercap to succinic acid (DMSA), 2,3-dimercapto-1-propanesulfonic acid (DMPS), penicillamine, an antibiotic, an aminoglycoside, an iron chelator, a porphyrin, an Nrf2 pathway activator, bardoxolone, ACE inhibitors, enalapril, glycine polymers, antioxidants, glutathione, N-acetyl cysteine, a PDGF inhibitor, lithium, ferroptosis inhibitors, vitamin B 12QPI-1002, QM56, SVT016426 (QM31), 16/86 (third generation ferrostatin), BASP siRNA, CCX140, BIIB023, CXA-10, alkaline phosphatase, Dnmtl inhibitor, THR-184, lithium, formoterol, IL-22, EPO, EPO derivative, agents that stimulate erthyropoietin, epoeitn alfa, darbepoietin alfa, PDGF inhibitor, CRMD-001, Atrasentan, Tolvaptan, RWJ-676070, Abatacept, Sotatercept, an anti-infective agent, an antibiotic, an anti-viral agent, an antifungal agent, an aminoglycoside, a nonsteroidal anti-inflammatory drug (NSAID), a diuretic drug, a statin, a senolytic, a corticosteroid, a glucocorticoid, a liposome, renin, angiotensin, ACE inhibitor, mediator of apoptosis, mediator of fibrosis, drug that targets p53, Apaf-1 inhibitor, RIPK1 inhibitor, RIPK3 inhibitor, inhibitor of IL17, inhibitor of IL6, inhibitor of IL23, inhibitor of CCR2, nitrated fatty acids, angiotensin blockers, agonists of the ALK3 receptor, SGLT2 modulator, and retinoic acid.

In some embodiments, the therapeutic composition improves one or more markers of kidney function in the subject selected from the group comprising reduced blood urea nitrogen (BUN) in the subject, reduced creatinine in the blood of the subject, improved creatinine clearance in the subject, reduced proteinuria in the subject, reduced albumin:creatinine ratio in the subject, improved glomerular filtration rate in the subject, reduced NAG in the urine of the subject, reduced NGAL in the urine of the subject, reduced KIM-1 in the urine of the subject, reduced IL-18 in the urine of the subject, reduced MCP1 in the urine of the subject, reduced CTGF in the urine of the subject; reduced collagen IV fragments in the urine of the subject; reduced collagen III fragments in the urine of the subject; and reduced podocyte protein levels in the urine of the subject, wherein the podocyte protein is selected from nephrin and podocin, reduced cystatin C protein in the blood of a subject, reduced β-trace protein (BTP) in the blood of a subject, and reduced 2-microglobulin (B2M) in the blood of a subject.

### BRIEF DESCRIPTION OF THE DRAWINGS

FIGS. 1A-1H show non-limiting exemplary designs of the compositions and methods provided herein, as well as data related thereto. FIG. 1A depicts Viral Inhibitor Design JBT-miR2. Without being bound by any particular theory, in some embodiments, TuDs were artificial strands of RNA with miRNA-binding domains that were thought to sequester the miRNA into stable complexes through complementary base pairing, disabling a particular RNA interference pathway. In short, they were single strands of RNA with one antisense miRNA binding domain (Decoy) or a stabilized stem-loop with two miRNA binding domains. FIG. 1B depicts the expression of JBT-miR2 In vitro in Hela cells as visualized with GFP expression. Six hour infection in serum free medium. 5% serum added after six hour infection Imaged at day 7 after infection. FIGS. 1C-1E depicts the Average Values of Normalized/β-gal Fold miRNA AntagomiR Activity on pMIR-REPORT miRNA Expression Reporter System in Hela Cells: JRX0116 activity on (FIG. 1C) miR-99 Binding Site; (FIG. 1D) JRX0104 on Let-7a binding site; (FIG. 1E) JRX0104 activity on Let-7c binding site. Graphs are representative of mean of two experiments with duplicate samples. FIGS. 1F-1H depicts the Average Values of Normalized/β-gal Fold miRNA AntagomiR Activity on pMIR-REPORT miRNA Expression Reporter System in neonatal rat ventricular cardiomyocytes: JRX0116 activity on (FIG. 1F) miR-99 Binding Site; (FIG. 1G) JRX0104 on Let-7a binding site; (FIG. 1H) JRX0104 activity on Let-7c binding site. Graphs are representative of mean of two experiments with duplicate samples/wells per experiment.

FIGS. 2A-2C depict non-limiting schematic representations of the experimental procedures described herein. FIGS. 2A-2B depict non-limiting schematic representations of the procedures in mice given JBT-miR2 or scrambled control virus at reperfusion (Group 1; FIG. 2A) and two weeks after reperfusion (Group 2; FIG. 2B). FIG. 2C depicts a non-limiting schematic representation of procedures in mice given JN-101 or Vehicle at reperfusion.

FIGS. 3A-3B depict non-limiting exemplary ECHO data obtained using the methods and compositions provided herein. FIG. 3A: Group 1: representative Echocardiograph Images of mice treated with JBT-miR2 at the time of reperfusion (Group 1). FIG. 3B: Group 1: composite regional strain of 50 nodes evenly distributed around the left ventricle at 2-weeks and 8-weeks Post IR showing enhancement of stretch at 8-weeks between nodes 21-38 which corresponds to the infarcted were of the left ventricle.

FIGS. 4A-4C depict data related to MRI experiments performed on mice treated with JBT-miR2 or control. FIG. 4A: using MRI, the LV endocardial shape was reconstructed from 9 separate, stacked slices, taken at a spatial resolution of 0.5 mm, from base to apex. The shape at end-diastole (ED) and end-systole (ES) were both fitted, by a least squares routine, to a prolate spheroid, with 300 equidistant nodes on its surface. Displacement in space of each node is then calculated between ED and ES, and then normalized for overall LV size by the end-diastolic surface area (EDSA) providing a finite element measure of myocardial shortening (contraction). FIG. 4B: composite images were obtained by averaging data at each of the corresponding nodes defined by the prolate spheroid fit. The LV ES shape is color-coded topographically to reflect low to high degrees of nodal displacement. Note the low displacement values (less dark blue) in the area of the antero-apical infarction of mice 2-weeks after a single administration of JBT-miR2. FIG. 4C: the data is represented graphically with red nodal displacement normalized to ED surface area (EDSA) shifted above the line of identity with JBT-miR2 treatment.

FIGS. 5A-5D depict data related to MRI experiments performed on mice treated with JN-101 or vehicle. FIG. 5A depicts representative Echocardiograph Images of mice treated with JN-101 at the time of reperfusion Imaged at 2-weeks (Imaged at Diastole). FIG. 5B depicts composite regional strain of 50 nodes evenly distributed around the left ventricle at 2-weeks and 8-weeks Post IR showing enhancement of stretch at 8-weeks between nodes 21-38 which corresponds to the infarcted were of the left ventricle. FIG. 5C depicts composite images obtained by averaging data at each of the corresponding nodes defined by the prolate spheroid fit. The LV ES shape is color-coded topographically to reflect low to high degrees of nodal displacement. Note the low displacement values (less dark blue) in the area of the antero-apical infarction of mice 4-weeks after a single administration of JN-101. FIG. 5D depicts the data represented graphically with red nodal displacement normalized to ED surface area (EDSA) shifted above the line of identity with JN-101 treatment.

FIGS. 6A-6D depict data related to histological analyses. FIG. 6A: tissues received in formalin were examined grossly, dissected for placement into cassettes and processed into paraffin. Six sections at 4 μM were stained with Masson's Trichrome (#2 and #5) or 3-Plex IF (MHC, DAPI and H3P). Sections #1 and #4 were unstained. FIG. 6B: the upper panel shows serial sections of a mouse treated with Control at reperfusion (Group 1), compared with mice treated with JBT-miR2 at reperfusion. FIG. 6C: ImageDX quantification of scar size mm2 of tissue area across 6 slices/heart/mouse (N=3 Control, N=5 JBT-miR2 treated). FIG. 6D: the top panels depict 2× and 10× magnified images of sections of heart taken from a mouse subject to IR injury and treated with Control virus at reperfusion, with histology conducted at 8-weeks post administration, and the lower panel consists of a mouse similarly treated with JBT-miR2. Increased MHC positive green cells, indicative of increased differentiated cardiomyocytes were evident in JBT-miR2 treated sections compared to mice treated with Control virus.

FIGS. 7A-7B depict data related to metabolic blood function tests. Terminal blood collected from mice treated with Control (N=4) or JBT-miR2 (N=4) virus was evaluated for comprehensive metabolic blood function tests. Circulating levels of both Blood Urea Nitrogen (BUN) (FIG. 7A) and Creatine Kinase (CK) (FIG. 7B) were significantly reduced 6-weeks after JBT-miR2 virus administration. FIGS. 22A-22B depict comprehensive metabolic blood function analysis.

FIGS. 8A-8L depict data related to NGS experiments performed using the methods and compositions described herein. Total RNA was isolated from the left ventricle of hearts from mice treated with Control (N=2) or JBT-miR2 (N=2) Virus and subjected to NGS as described herein. FIGS. 8A-8D depict data related to mRNA expression changes in heart. FIG. 8A depicts a Volcano plot shows that 64 known mRNAs were upregulated in JBT-miR2 treated hearts compared with mice treated with Control virus. Eight-six mRNAs were down regulated in JBT-miR2 treated hearts compared with Control virus. FIG. 8B depicts a heat map demonstrating consistent mRNA expression changes in duplicate JBT-miR2 and Control virus treated hearts. FIG. 8C shows Kyoto Encyclopedia of genes and genome (KEGG) UP for mRNA and FIG. 8D shows Kyoto Encyclopedia of genes and genome (KEGG) Down for mRNA. FIGS. 8E-8H depict data related to lncRNA expression changes in the heart. FIGS. 8I-8L depict data related to TUCP expression changes in the heart. Additional expression change data is depicted in FIGS. 23A-23F.

FIGS. 9A-9L depict hemodynamic data related to mice treated with JBT-miR2 or scrambled control in group 1 (FIGS. 9A-9F) and group 2 (FIGS. 9G-9L).

FIGS. 10A-10F depict hemodynamic data related to JN-101 administered at reperfusion.

FIGS. 11A-11G depict hemodynamic data related to JN-101 administered 2-weeks after reperfusion. A correlation with reduced infarct size was observed. This data suggests the formation of functional, electrically coupled myocytes. These data indicate that JN-101 normalizes heart function under stress after heart attack.

FIG. 12 depicts H and E stained heart sections from group 1 mice.

FIG. 13 depicts data related to QPCR of Human U6 Promoter.

FIGS. 14A-14F depict data related to the body weight and heart weight of JBT-miR2 treated mice.

FIG. 15 depicts data related to the arrhythmogenic potential of JBT-miR2 on human ventricular cardiomyocytes.

FIGS. 16A-16D depict data related to the effects of JN-101 on body weight at sacrifice.

FIGS. 17A-17D depict data related to the effects of JN-101 on heart weight at sacrifice.

FIGS. 18A-18D depict data related to the effects of JN-101 on heart weight to body weight ratio at sacrifice.

FIGS. 19A-19C depict data related to the effects of JN-101 on body weight, heart weight to body weight ratio.

FIGS. 20A-20D depict data related to cardiac myocyte cell area and indication of cytokinesis. As shown in FIG. 20A, Cardiac Myocyte Cell Size is the Same in JN-101 (N=4 Mice, 10 mg/kg) Vs. Vehicle Treated Mice (N=4 Mice) at Day 25. Hematoxylin and Eosin Staining and Cell Area Calculated by ImageDx at 40× Magnification in Approximately 15,000 Cardiac Myocytes/Slide/Mouse. As shown in FIG. 20C, Day 15 Post JN-101 Injection (15 mg/kg) the Numbers of Dual labelled H3P/ARK Positive cells increased (N=4) mice, compared with Vehicle Treated Mice (N=4).

FIGS. 21A-21D depict data related to survival curves of mice with subcutaneous (SC) administration of JN-101 and intravenous (IV) administration of JN-101.

FIGS. 22A-22B depict data related to metabolic blood function tests of JBT-miR2 treated mice of group 1 (FIG. 22A) and group 2 (FIG. 22B).

FIGS. 23A-23F depict data related to NGS experiments performed using the methods and compositions described herein, showing Select mRNA expression changes up (FIG. 23A), showing select mRNA expression changes down (FIG. 23B), showing lncRNA expression changes up (FIG. 23C), showing lncRNA expression changes down (FIG. 23D), showing TUCP expression changes up (FIG. 23E), and showing TUCP expression changes down (FIG. 23F).

FIG. 24 depicts data related to metabolic blood function tests in uninjured mice.

FIG. 25 depicts data related to quantification of 1-Plex, 2-Plex and 3-Plex positive cells.

FIG. 26 depicts data related to quantification of cardiac myocyte cell area.

FIG. 27 depicts non-limiting exemplary schematic illustrations of the methods and compositions provided herein.

FIGS. 28A-28C depict data related to the effect of JBT-miR2 on Regional Normalized Displacement (Displ/EDSA) compared to control virus and untreated mice: protocol with treatment (vertical) vs scrambled control (horizontal) (FIG. 28A), repeat protocol without treatment (FIG. 28B), and Superimposed Plots (FIG. 28C).

FIGS. 29A-29F depict data related to single AAV2/9 delivering decoys to target MicroRNAs facilitating global recovery in a mouse model of ischemic reperfusion.

FIGS. 30A-30B depict data related to JN-101 facilitating global recovery in a mouse model of ischemic reperfusion.

FIGS. 31A-31F depict non-limiting exemplary schematics regarding the design of compositions provided herein. FIG. 31A depicts the pAV-U6-GFP vector and insert employed in some of the compositions provided herein (e.g., JBT-miR2). FIG. 31B depicts non-limiting exemplary sequences employed in the design of TuDs provided herein (SEQ ID NOS: 86 and 89).

FIG. 31C depicts a non-limiting exemplary TuD cassette that was inserted into pAV-U6 GFP (SEQ ID NO: 98). One or more of the TUD cassettes can be inserted into a cloning or expression vector described herein (e.g., cloned between the two ITR sequences). FIG. 31D depicts Albumin Stuffer Design 1 (SEQ ID NO: 99) and FIG. 31E depicts ADD Stuffer Design 2 (SEQ ID NO: 100). FIG. 31F depicts a portion of the nucleotide sequence of JBT-miR2 (SEQ ID NO: 101).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

There are provided, in some embodiments, methods of preventing, inhibiting, reducing, or treating cardiac ischemic reperfusion injury. In some embodiments, the method comprises: administering a therapeutic composition to a subject before, during, and/or after a cardiac ischemic event, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b). The method can comprise: reperfusion of ischemic cardiac tissue.

There are provided, in some embodiments, methods of treating myocardial infarction. In some embodiments, the method comprises: administering a therapeutic composition to a subject before, during, and/or after reperfusion of ischemic cardiac tissue, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b). Myocardial infarction can be a cardiac ischemic event.

There are provided, in some embodiments, methods of inducing cardiomyocyte regeneration, cardiac repair, vasculogenesis and/or cardiomyocyte differentiation following a cardiac ischemic event. In some embodiments, the method comprises: administering a therapeutic composition to a subject before, during, or after reperfusion of ischemic cardiac tissue, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

There are provided, in some embodiments, methods of treating a disease or disorder associated with dysregulation of FHL1 and/or TNNT2. In some embodiments, the method comprises: administering a therapeutic composition to a subject in need thereof, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

There are provided, in some embodiments, methods of treating a kidney condition of a subject and/or protecting a kidney of a subject from injury. In some embodiments, the method comprises: administering a therapeutic composition to the subject, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

Disclosed herein include methods increasing heart function, reducing mortality, reducing cardiac volumes and/or reducing scar size following ischemic reperfusion injury. In some embodiments, the method comprises: administering a therapeutic composition to a subject before, during, and/or after a cardiac ischemic event, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b). The method can comprise: reperfusion of ischemic cardiac tissue.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

The singular form “a”, “an”, and “the” include plural references unless the context clearly dictates otherwise. For example, the term “a molecule” includes one or more molecules, including mixtures thereof. As used in this disclosure and the appended claims, the term “and/or” can be singular or inclusive. For example, “A and/or B” is used herein to include all of the following alternatives: “A”, “B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

“Administering” means providing a pharmaceutical agent or composition to a subject, and includes, but is not limited to, administering by a medical professional and self-administering.

“Parenteral administration,” means administration through injection or infusion. Parenteral administration includes, but is not limited to, subcutaneous administration, intravenous administration, intramuscular administration, intra-arterial administration, and intracranial administration. “Subcutaneous administration” means administration just below the skin. “Intravenous administration” means administration into a vein. “Intraarterial administration” means administration into an artery.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and 0-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Antisense compound” means a compound having a nucleobase sequence that will allow hybridization to a target nucleic acid. In certain embodiments, an antisense compound is an oligonucleotide having a nucleobase sequence complementary to a target nucleic acid.

The terms “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing. The terms “protein”, “peptide”, and “polypeptide” are used interchangeably to denote an amino acid polymer or a set of two or more interacting or bound amino acid polymers. These terms, as used herein, encompass amino acid polymers in which one or more amino acid residue is an artificial chemical mimetic of a corresponding naturally occurring amino acid, as well as to naturally occurring amino acid polymers and non-naturally occurring amino acid polymer.

The phrase “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical nucleotide sequences. Because of the degeneracy of the genetic code, a large number of functionally identical nucleic acids can encode any given protein. For instance, the codons GCA, GCC, GCG and GCU all encode the amino acid alanine. Thus, at every position where an alanine is specified by a codon, the codon can be altered to any of the corresponding codons described above without altering the encoded polypeptide. Such nucleic acid variations are “silent variations,” which are one species of conservatively modified variations. Any one of the nucleic acid sequences described herein which encodes a polypeptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, all silent variations of a nucleic acid which encodes a polypeptide are implicit in each of the described sequences with respect to its expression product, but not with respect to actual probe sequences. In addition or alternatively, a variant can comprises deletions, substitutions, additions of one or more nucleotides at the 5′ end, 3′ end, and/or one or more internal sites in comparison to the reference polynucleotide. Similarities and/or differences in sequences between variants and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variants of a particular polynucleotide disclosed herein, including, but not limited to, a miRNA, will have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisan.

The terms “identical” or “percent identity”, in the context of two or more nucleic acids or proteins, refer to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a test sequence. This definition also includes sequences that have deletions and/or additions, as well as those that have substitutions. Sequence identity typically exists over a region that is at least about 50 amino acids or nucleotides in length, or over a region that is 50-100 amino acids or nucleotides in length, or over the entire length of a given sequence.

As used herein, the term “construct” is intended to mean any recombinant nucleic acid molecule such as an expression cassette, plasmid, cosmid, virus, autonomously replicating polynucleotide molecule, phage, or linear or circular, single-stranded or double-stranded, DNA or RNA polynucleotide molecule, derived from any source, capable of genomic integration or autonomous replication, comprising a nucleic acid molecule where one or more nucleic acid sequences has been linked in a functionally operative manner, e.g. operably linked.

The term “transfection” or “transfecting” is defined as a process of introducing a nucleic acid molecule to a cell using non-viral or viral-based methods. The nucleic acid molecule can be a sequence encoding complete proteins or functional portions thereof. Typically, a nucleic acid vector comprises the elements necessary for protein expression (e.g., a promoter, transcription start site, etc.). Non-viral methods of transfection include any appropriate transfection method that does not use viral DNA or viral particles as a delivery system to introduce the nucleic acid molecule into the cell. Exemplary non-viral transfection methods include, but are not limited to, calcium phosphate transfection, liposomal transfection, nucleofection, sonoporation, transfection through heat shock, magnetifection, and electroporation. For viral-based methods, any one of useful viral vectors known in the art can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures known in the art.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “gene” is used broadly to refer to any segment of nucleic acid molecule that encodes a protein or that can be transcribed into a functional RNA. Genes may include sequences that are transcribed but are not part of a final, mature, and/or functional RNA transcript, and genes that encode proteins may further comprise sequences that are transcribed but not translated, for example, 5′ untranslated regions (5′-UTR), 3′ untranslated regions (3′-UTR), introns, etc. Further, genes may optionally further comprise regulatory sequences required for their expression, and such sequences may be, for example, sequences that are not transcribed or translated. Genes can be obtained from a variety of sources, including cloning from a source of interest or synthesizing from known or predicted sequence information, and may include sequences designed to have desired parameters.

The term “internucleoside linkage” means a covalent linkage between adjacent nucleosides.

The term “nucleobase” means a heterocyclic moiety capable of non-covalently pairing with another nucleobase.

“Nucleoside” means a nucleobase linked to a sugar. “Linked nucleosides” means nucleosides joined by a covalent linkage. “Nucleotide” means a nucleoside having a phosphate group covalently linked to the sugar portion of a nucleoside.

“miR antagonist” means an agent designed to interfere with or inhibit the activity of a miRNA. In certain embodiments, a miR antagonist comprises an antisense compound targeted to a miRNA. In certain embodiments, a miR antagonist comprises a modified oligonucleotide having a nucleobase sequence that is complementary to the nucleobase sequence of a miRNA, or a precursor thereof. In certain embodiments, a miR antagonist comprises a small molecule, or the like that interferes with or inhibits the activity of an miRNA.

“miR-9a-5p antagonist” means an agent designed to interfere with or inhibit the activity of miR-9a-5p. “miR-100-5p antagonist” means an agent designed to interfere with or inhibit the activity of miR-100-5p. “Let-7a-5p antagonist” means an agent designed to interfere with or inhibit the activity of Let-7a-5p. “Let-7c-5p antagonist” means an agent designed to interfere with or inhibit the activity of Let-7c-5p.

“Modified oligonucleotide” means an oligonucleotide having one or more chemical modifications relative to a naturally occurring terminus, sugar, nucleobase, and/or internucleoside linkage.

“Modified internucleoside linkage” means any change from a naturally occurring internucleoside linkage.

“Phosphorothioate internucleoside linkage” means a linkage between nucleosides where one of the non-bridging atoms is a sulfur atom.

“Modified sugar” means substitution and/or any change from a natural sugar.

“Modified nucleobase” means any substitution and/or change from a natural nucleobase.

“5-methylcytosine” means a cytosine modified with a methyl group attached to the 5′ position.

“2′-O-methyl sugar” or “2′-0Me sugar” means a sugar having an O-methyl modification at the 2′ position.

“2′-O-methoxyethyl sugar” or “2′-MOE sugar” means a sugar having an 0-methoxyethyl modification at the 2′ position.

“2′-O-fluoro sugar” or “2′-F sugar” means a sugar having a fluoro modification of the 2′ position.

“Bicyclic sugar moiety” means a sugar modified by the bridging of two non-geminal ring atoms.

“2′-O-methoxyethyl nucleoside” means a 2′-modified nucleoside having a 2′-0-methoxyethyl sugar modification.

“2′-fluoro nucleoside” means a 2′-modified nucleoside having a 2′-fluoro sugar modification.

“2′-O-methyl” nucleoside means a 2′-modified nucleoside having a 2′-O-methyl sugar modification.

“Bicyclic nucleoside” means a 2′-modified nucleoside having a bicyclic sugar moiety.

As used herein, the terms “miR,” “mir,” and “miRNA” are used interchangeably and to refer to microRNA, a class of small RNA molecules that are capable of hybridizing to and regulating the expression of a coding RNA. In certain embodiments, a miRNA is the product of cleavage of a pre-miRNA by the enzyme Dicer. These terms as provided herein refer to a nucleic acid that forms a double stranded RNA which has the ability to reduce or inhibit expression of a gene or target gene when expressed in the same cell as the gene or target gene. The complementary portions of the nucleic acid that hybridize to form the double stranded molecule typically have substantial or complete identity. In one embodiment, a “microRNA” refers to a nucleic acid that has substantial or complete identity to a target gene and forms a double stranded miRNA. In some embodiments, the miRNA of the disclosure inhibits gene expression by interacting with a complementary cellular mRNA thereby interfering with the expression of the complementary mRNA. In some embodiments, the double stranded miRNA of the present disclosure is at least about 15-50 nucleotides in length (e.g., each complementary sequence of the double stranded miRNA is 15-50 nucleotides in length, and the double stranded miRNA is about 15-50 base pairs in length). In some embodiments, the length is 20-30 base nucleotides, preferably about 20-25 or about 24-29 nucleotides in length, e.g., 20, 21, 22, 23, 24, 25, 26, 27, 28, 29, or 30 nucleotides in length. In some embodiments of the disclosure, the microRNA is selected from, or substantially similar to a microRNA selected from, the group consisting of miR-9a-5p, miR-100-5p, Let-7a-5p, and Let-7c-5p.

As used herein, the term “anti-miRNA” is used interchangeably with the term “anti-miR”, which refers to an oligonucleotide capable of interfering with or inhibiting one or more activities of one or more target microRNAs. In some embodiments, the anti-miRNA is a chemically synthesized oligonucleotide. In some embodiments, the anti-miRNA is a small molecule. In some embodiments, the anti-miRNA is a miR antisense molecule. “Seed region” means nucleotides 2 to 6 or 2 to 7 from the 5′-end of a mature miRNA sequence.

The term “miRNA precursor” means a transcript that originates from a genomic DNA and that comprises a non-coding, structured RNA comprising one or more miRNA sequences. For example, in certain embodiments a miRNA precursor is a pre-miRNA. In certain embodiments, a miRNA precursor is a pri-miRNA.

“Pre-miRNA” or “pre-miR” means a non-coding RNA having a hairpin structure, which contains a miRNA. In certain embodiments, a pre-miRNA is the product of cleavage of a pri-miR by the double-stranded RNA-specific ribonuclease known as Drosha. Without wishing to be bound by any particular theory, it is believed that in the cytoplasm, the pre-miRNA hairpin is cleaved by the RNase III enzyme Dicer. This endoribonuclease interacts with 5′ and 3′ ends of the hairpin and cuts away the loop joining the 3′ and 5′ arms, yielding an imperfect miRNA:miRNA duplex of about 22 nucleotides in length. Although either strand of the duplex may potentially act as a functional miRNA, it is believed that only one strand is usually incorporated into the RNA-induced silencing complex (RISC) where the miRNA and its mRNA target interact. The remaining strand—sense strand—is degraded. The RNA-induced silencing complex, or RISC, is a multiprotein complex, specifically a ribonucleoprotein, which incorporates one strand of a single-stranded RNA (ssRNA) fragment, such as microRNA microRNA), or double-stranded small interfering RNA (siRNA).

“Modulation” means to a perturbation of function or activity. In certain embodiments, modulation means an increase in gene expression. In certain embodiments, modulation means a decrease in gene expression. The term “microRNA modulator” as used herein refers to an agent capable of modulating the level of expression of a microRNA (e.g., let-7 a, let-7 c, miR-100, miR-99). In some embodiments, the microRNA modulator is encoded by a nucleic acid. In other embodiments, the microRNA modulator is a small molecule (e.g., a chemical compound or synthetic microRNA molecule). In some embodiments, the microRNA modulator decreases the level of expression of a microRNA compared to the level of expression in the absence of the microRNA modulator. Where the microRNA modulator decreases the level of expression of a microRNA relative to the absence of the modulator, the microRNA modulator is an antagonist of the micro RNA. In some embodiments, the microRNA modulator increases the level expression of a micro RNA compared to the level of expression in the absence of the microRNA modulator. Where the microRNA modulator increases the level of expression of a micro RNA relative to the absence of the modulator, the microRNA modulator is an agonist of the microRNA.

As used herein, the term “myocardial cell” includes any cell that is obtained from, or present in, myocardium such as a human myocardium and/or any cell that is associated, physically and/or functionally, with myocardium. In some embodiments disclosed herein, a myocardial cell is a cardiomyocyte.

The term “nucleotide” covers naturally occurring nucleotides as well as non-naturally occurring nucleotides. Thus, “nucleotides” includes not only the known purine and pyrimidine heterocycles-containing molecules, but also heterocyclic analogues and tautomers thereof. Non-limiting examples of other types of nucleotides are molecules containing adenine, guanine, thymine, cytosine, uracil, purine, xanthine, diaminopurine, 8-oxo-N6-methyladenine, 7-deazaxanthine, 7-deazaguanine, N4,N4-ethanocytosin, N6,N6-ethano-2,6-diaminopurine, 5-methylcytosine, 5-(C3-C6)-alkynylcytosine, 5-fluorouracil, 5-bromouracil, pseudoisocytosine, 2-hydroxy-5-methyl-4-triazolopyridin, isocytosine, isoguanine, inosine and the “non-naturally occurring” nucleotides described in U.S. Pat. No. 5,432,272. The term “nucleotide” is intended to cover every and all of these examples as well as analogues and tautomers thereof.

The term “nucleic acid” and “polynucleotide” are used interchangeably herein and refer to deoxyribonucleotides or ribonucleotides and polymers thereof in either single- or double-stranded form, and complements thereof. The term “polynucleotide” include linear sequences of nucleotides. The term “nucleotide” typically refers to a single unit of a poly-nucleotide, e.g., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, and 2′-O-methyl ribonucleotides. As such, the term “nucleic acid” and “polynucleotide” encompass nucleic acids comprising phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil.

The term “operably linked”, as used herein, denotes a functional linkage between two or more sequences. For example, an operably linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements may be contiguous or non-contiguous.

The terms “promoter”, “promoter region”, or “promoter sequence”, as used interchangeably herein, refer to a nucleic acid sequence capable of binding RNA polymerase to initiate transcription of a gene in a 5′ to 3′ (“downstream”) direction. The specific sequence of the promoter typically determines the strength of the promoter. For example, a strong promoter leads to a high rate of transcription initiation. A gene is “under the control of” or “regulated by” a promoter when the binding of RNA polymerase to the promoter is the proximate cause of said gene's transcription. The promoter or promoter region typically provides a recognition site for RNA polymerase and other factors necessary for proper initiation of transcription. A promoter may be isolated from the 5′ untranslated region (5′ UTR) of a genomic copy of a gene. Alternatively, a promoter may be synthetically produced or designed by altering known DNA elements. Also considered are chimeric promoters that combine sequences of one promoter with sequences of another promoter. A promoter can be used as a regulatory element for modulating expression of an operably linked polynucleotide molecule such as, for example, a coding sequence of a polypeptide or a functional RNA sequence. Promoters may contain, in addition to sequences recognized by RNA polymerase and, preferably, other transcription factors, regulatory sequence elements such as cis-elements or enhancer domains that affect the transcription of operably linked genes. In some embodiments, a promoter can be “constitutive.” In some embodiments, a promoter may be regulated in a “tissue-specific” or “tissue-preferred” manner, such that it is only active in transcribing the operable linked coding region in a specific tissue type or types. In some embodiments, for therapeutic purposes, the promoter can be a tissue-specific promoter which supports transcription in cardiac and skeletal muscle cell. Further information in this regard can be found in, for example, PCT Patent Publication WO2004041177A2, which is hereby incorporated by reference in its entirety. In some embodiments, a promoter may comprise “naturally-occurring” or “synthetically” assembled nucleic acid sequences.

Expression of a transfected gene can occur transiently or stably in a host cell. During “transient expression” the transfected nucleic acid is not integrated into the host cell genome, and is not transferred to the daughter cell during cell division. Since its expression is restricted to the transfected cell, expression of the gene can be lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as in subsequent excision.

The terms “inhibitor,” “repressor” or “antagonist” or “downregulator”, as used interchangeably herein, refer to a substance, agent, or molecule that results in a detectably lower expression or activity level of a target gene as compared to a control. The inhibited expression or activity can be 10%, 20%, 30%, 40%, 50%, 60%, 70%, 80%, 90% or less than that in a control. In some embodiments, the inhibition is 1.5-fold, 2-fold, 3-fold, 4-fold, 5-fold, 10-fold, or more in comparison to a control. In some embodiments, an antagonist is an anti-miR.

As used herein, “treatment” refers to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Subjects in need of treatment include those already affected by a disease or disorder or undesired physiological condition as well as those in which the disease or disorder or undesired physiological condition is to be prevented. In some embodiments of the disclosure, the terms “treatment,” “therapy,” and “amelioration” refer to any reduction in the severity of symptoms, e.g., of a neurodegenerative disorder or neuronal injury. As used herein, the terms “treat” and “prevent” are not intended to be absolute terms. Treatment can refer to any delay in onset, amelioration of symptoms, and improvement in patient survival, increase in survival time or rate, etc., or a combination thereof. The effect of treatment can be compared to an individual or pool of individuals not receiving the treatment, or to the same patient prior to treatment or at a different time during treatment. In some embodiments, the severity of disease or disorder in an individual can be reduced by at least 10%, as compared, e.g., to the individual before administration or to a control individual not undergoing treatment. In some embodiments, the severity of disease or disorder in an individual is reduced by at least 25%, 50%, 75%, 80%, or 90%, or in some embodiments, no longer detectable using standard diagnostic techniques.

As used herein, the term “effective amount” or “therapeutically effective amount” refers to an amount sufficient to effect beneficial or desirable biological and/or clinical results. In some embodiments, the term refers to that amount of the therapeutic agent sufficient to ameliorate a given disorder or symptoms. For example, for the given parameter, a therapeutically effective amount can show an increase or decrease of at least 5%, 10%, 15%, 20%, 25%, 40%, 50%, 60%, 75%, 80%, 90%, or at least 100% compared to a control. Therapeutic efficacy can also be expressed as “-fold” increase or decrease. For example, a therapeutically effective amount can have at least a 1.2-fold, 1.5-fold, 2-fold, 5-fold, or more effect over a control.

The terms “subject,” “patient,” “individual in need of treatment” and like terms are used interchangeably and refer to, except where indicated, an mammal subject that is the object of treatment, observation, or experiment. As used herein, “mammal” refers to a subject belonging to the class Mammalia and includes, but not limited to, humans, domestic and farm animals, zoo animals, sports and pet animals. Non-limiting examples of mammals include humans, and non-human primates, mice, rats, sheep, dogs, horses, cats, cows, goats, pigs, and other mammalian species. In some embodiments, the mammal is a human. However, in some embodiments, the mammal is not a human. The term does not necessarily indicate that the subject has been diagnosed with a particular disease or disorder, but typically refers to a subject under medical supervision. “Subject suspected of having” means a subject exhibiting one or more clinical indicators of a disease or condition. In certain embodiments, the disease or condition is a muscular dystrophy (MD) disorder.

“Target nucleic acid,” “target RNA,” “target RNA transcript” and “nucleic acid target” all mean a nucleic acid capable of being targeted by antagonists. “Targeting” means the process of design and selection of nucleobase sequence that will hybridize to a target nucleic acid and induce a desired effect. “Targeted to” means having a nucleobase sequence that will allow hybridization to a target nucleic acid to induce a desired effect. In certain embodiments, a desired effect is reduction of a target nucleic acid.

As used herein, the term “variant” refers to a polynucleotide (or polypeptide) having a sequence substantially similar to a reference polynucleotide (or polypeptide). In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by one of ordinary skill in the art.

As used herein, “comprising” is synonymous with “including,” “containing,” or “characterized by,” and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

In some embodiments of the methods or processes described herein, the steps can be carried out in any order, except when a temporal or operational sequence is explicitly recited. Furthermore, in some embodiments, the specified steps can be carried out concurrently unless explicit claim language recites that they be carried out separately. For example, in some embodiments a claimed step of doing X and a claimed step of doing Y can be conducted simultaneously within a single operation, and the resulting process will fall within the literal scope of the claimed process.

The section headings, e.g., (a), (b), (i) etc., are presented merely for ease of reading the specification and claims, as they are used herein for organizational purposes only and are not to be construed as limiting the subject matter described. The use of headings in the specification or claims does not require the steps or elements be performed in alphabetical or numerical order or the order in which they are presented. All documents, or portions of documents, cited in the application including, without limitation, patents, patent applications, articles, books, manuals, and treatises are hereby expressly incorporated by reference in their entireties.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

I. Cardiac Diseases and Micro-Ribonucleic Acid (miRNA)

Cardiac disease or heart disease is a disease for which several classes or types exist (e.g., Ischemic Cardiomyopathy (ICM), Dilated Cardiomyopathy (DCM), Aortic Stenosis (AS)) and, many require unique treatment strategies. Thus, heart disease is not a single disease, but rather a family of disorders arising from distinct cell types (e.g., myocardial cells) by distinct pathogenetic mechanisms. The challenge of heart disease treatment has been to target specific therapies to particular heart disease types, to maximize effectiveness and to minimize toxicity. Improvements in heart disease categorization (classification) have thus been central to advances in heart disease treatment. As used herein, cardiac disease encompasses the following non-limiting examples: heart failure (e.g., congestive heart failure), ischemic cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis, aortic stenosis, aortic regurgitation, mitral stenosis, mitral regurgitation, mitral prolapse, pulmonary stenosis, pulmonary regurgitation, tricuspid stenosis, tricuspid regurgitation, congenital disorder, genetic disorder, or a combination thereof.

Heart cell regeneration: Throughout the 20th century the human heart was believed to be a terminally differentiated post mitotic organ, unable to be repaired after an injury. This was challenged in 2001 when mitosis in cardiomyocytes was evident after a myocardial infarction. Studies by others confirmed that adult mammalian hearts can elicit a primitive regeneration response upon injury with mature differentiated mononuclear mammalian cardiomyocytes re-entering the cell cycle upon application of chemical compounds that target specific signaling pathways.

miRNAs (also referred to as miRs) are small non-coding RNA molecules conserved in plants, animals, and some viruses, which function in RNA silencing and post-transcriptional regulation of gene expression. Identified in 1993, they are a vital and evolutionarily component of genetic regulation. They function via base-pairing and silencing complementary sequences within mRNA molecules thereby modulating target protein expression and downstream signaling pathways. There are 1000 known miRs in the human genome that can target 60% of human genes. In animals, miRNAs are processed from larger primary transcripts (pri-miRNA or pri-miR) through an approximate 60-bp hairpin precursor (pre-miRNA or pre-miR) into the mature forms (miRNA) by two RNAse III enzymes Drosha and Dicer. The mature miRNA is loaded into the 50 ribonucleoprotein complex (RISC), where it typically guides the downregulation of target mRNA through base pair inter-actions. Pri-miRNAs are transcribed by RNA polymerase II and predicted to be regulated by transcription factors in an inducible manner. While some miRNAs show ubiquitous expression, others exhibit only limited developmental stage-, tissue- or cell type-specific patterns of expression.

As described in greater detail below, measurements previously made in myocardial tissue have suggested the miRNAs play a regulatory role in myocardial growth, fibrosis, and remodeling. In particular, ribonucleic acid interference (RNAi) technology is an area of intense research for the development of new therapies for heart disease, with studies demonstrating the utility of adeno-associated virus (AAV) for delivering oligonucleotides in vivo. Two separate AAV2/9 virus' expressing antagonists of microRNAs (miRs) let-7a/let-c and miR-99/100 can induce proliferation of cardiomyocytes in the ischemic mouse heart for up to 3 months following a single injection. Transcriptomic and translational analysis on mice heart cells and tissues treated with viral delivered miR antagonists showed differences in the expression of genes and proteins involved in cardiac development, proliferation and muscle structure and function, implying that a similar regenerative effect, through targeting of these miRs, may occur in human cardiac myocytes and models of DMD.

RNAi technology can take many forms, but it is typically implemented within a cell in the form of a base-pair short hairpin (sh) RNA (shRNA), which is processed into an approximately 20 base pair small interfering RNA through the endogenous miR pathway. Viral delivery of complementary sequences to miRs is a common approach. AAV vectors are optimal in cardiovascular muscle gene delivery since they a) contain no viral protein-coding sequences to stimulate an immune response, b) do not require active cell division for expression to occur and c) have a significant advantage over adenovirus vectors because of their stable, long-term expression of recombinant genes in myocytes in vivo. Viral delivery of genes are in development for the treatment of DMD and include AAV1-gamma-sarcoglycan vector as a therapy for LGMD, recombinant (r) AAV2.5 vector for delivery of mini dystrophin, and rAAV, rhesus serotype 74.

As described herein, the mechanism by which the miRNA antagonist functions to inhibit the activity of the target miRNA is not limited in any way. For example, a nucleic acid-based antagonist, in some embodiments, may form a duplex with the target miRNA sequences and prevent proper processing of the mature miRNA product from its precursor, or may prevent the mature miRNA from binding to its target gene, or may lead to degradation of pri-, pre-, or mature miRNA, or may act through some other mechanism.

let-7a/c and miR-100/99: By studying the mechanisms of heart regeneration in zebrafish and neonatal mice, scientists have found that heart regeneration is a primarily cardiomyocyte-mediated process that occurs by dedifferentiation of mature cardiomyocytes followed by proliferation and further re-differentiation. Epigenetic remodeling and cell cycle control are two key steps controlling this regenerative process. Aguirre et al (Cell Stem Cell. 2014; 15(5):589-604) reported a very relevant study, which investigated the underlying mechanism of heart regeneration and identified a series of miRs strongly involved in zebrafish heart regeneration. Focus on those miRs that present significant expression changes and that were conserved across vertebrates, both in sequence and 3′ UTR binding sites, led to the identification of two miR families (miR-99/100, let-7a/c) clustered in two well-defined genomic locations. This finding was supported by a common role for the miR-99a/Let-7c-5p cluster in regulating vertebrate cardiomyogenesis. MIRANDA-based miR-UTR binding predictions showed a strong interaction for miR-99/100 with zebrafish FNTβ (beta subunit of farnesyl-transferase) and SMARCA5 (SWI/SNF-related matrix associated actin-dependent regulator of chromatin subfamily a, member 5), linking the miR families to cell cycle and epigenetic control in cardiomyocytes. Interestingly, miR-99/100 and let-7a/c levels are low during early mammalian heart development and promote quick cardiac mass growth, but increase exponentially during late development, with a corresponding decrease in FNTβ and SMARCA5 protein levels to block further cardiomyocyte proliferation. Postmortem analysis of injured human heart tissue, suggests that these miRs constitute a conserved roadblock to cardiac regeneration in adults. RNA-seq transcriptomic analysis on neonatal mouse cardiomyocytes transduced two viral delivered antagonists to let-7a/c and miR-99/100 revealed differences in genes involved in epigenetic remodeling, demethylation, cardiac development, proliferation, and unexpectedly, metabolic pathways and muscle structural and function. Indeed, miR-let 7a/c and miR-99/100 inhibition targets 1072 and 47 genes, respectively.

### II. Compositions of the Disclosure

### MicroRNA Antagonists

Disclosed herein includes embodiments of compositions that include a plurality of microRNA (miR) antagonists. As used herein, “miR antagonist” refers to an agent designed to interfere with or inhibit the activity of a miRNA. In certain embodiments, a miR antagonist comprises an antisense compound targeted to a miRNA. In certain embodiments, a miR antagonist comprises a modified oligonucleotide having a nucleotide sequence that is complementary to the nucleotide sequence of a miRNA, or a precursor thereof. In other embodiments, a miR antagonist comprises a small molecule, or the like that interferes with or inhibits the activity of a miRNA. In some embodiments, a miR antagonist is a miR-99a antagonist. In some embodiments, a miR antagonist is a miR-100-5p antagonist. In some embodiments, a miR antagonist is a miR-Let-7a-5p antagonist. In some embodiments, a miR antagonist is a miR-Let-7c-5p antagonist. The miR antagonists disclosed herein are useful, for example, in providing compositions and methods to prevent, inhibit, or reduce target gene expression in, for example, myocardium (e.g., myocardial tissue, myocardial cells). Thus, some of the embodiments disclosed herein relate to the use of the miR antagonists of the disclosure in methods for evaluation and therapy of cardiac diseases, including heart failure.

Implementations of embodiments of the compositions according to this aspect and other aspects of the disclosure can include one or more of the following features. In some embodiments, the plurality of miR antagonists includes 4, 5, 6, 7, 8, 9, 10, 15, 20, 30, 40, 50 miR antagonists or a number of antagonists that is within a range defined by any two of the aforementioned values. In some embodiments, the plurality of miR antagonists includes one or more selected from miR-99a antagonists, miR-100-5p antagonists, miR-Let-7a-5p antagonists, miR-Let-7c-5p antagonists, and combinations thereof. In some embodiments, the plurality of miR antagonists includes one or more miR-99a antagonists, one or more miR-100-5p antagonists, one or more miR-Let-7a-5p antagonists, and one or more miR-Let-7c-5p antagonists. In some embodiments, the numbers of each miR antagonist group are the same in the plurality of miR antagonists. In some embodiments, the numbers of each miR antagonist group are not the same in the plurality of miR antagonists.

Accordingly, in some embodiments, the plurality of miR antagonists includes at least one miR antagonist comprising a nucleotide sequence having, or having about, 85%, 90%, 91%, 92%, 93%, 94%, 95%, 96%, 97%, 98%, 99%, 100%, or a range between any two of these values, sequence identity to one or more of the miR antagonists disclosed herein. For example, in some embodiments, the miR antagonist comprises, or consists of, a nucleotide sequence having at least about 85%, at least about 90%, at least about 95%, at least about 96%, at least about 97%, at least about 98%, at least about 99%, or more, sequence identity to one or more of the miR antagonists disclosed herein. In some embodiments, the miR antagonist comprises, or consists of, a nucleotide sequence having at least about 85%, at least about 90%, at least about 95%, at least about 96%, at least about 97%, at least about 98%, at least about 99%, or more, sequence identity to one or more of the miR antagonists disclosed herein. In some embodiments, the miR antagonist comprises, or consists of, a nucleotide sequence having about 85%, about 90%, about 95%, about 96%, about 97%, about 98%, about 99%, about 100%, or a range between any two of these values, sequence identity to one or more of the miR antagonists disclosed herein.

In some embodiments, at least one of the one or more miR-99a antagonists includes an anti-miR-99a comprising a nucleotide sequence having at least about, or having about, 80%, 85%, 90%, 95%, 96%, 97, 98%, 99% or 100%, or a range between any two of these values, sequence identity to a sequence selected from the group consisting of SEQ ID NOs 47, 48, 50, 52, and 54. In some embodiments, at least one of the one or more miR-100-5p antagonists includes an anti-miR-100-5p comprising a nucleotide sequence having at least about, or having about, 80%, 85%, 90%, 95%, 96%, 97, 98%, 99% or 100%, or a range between any two of these values, sequence identity to a sequence selected from the group consisting of SEQ ID NOs 46, 49, 51, 53, and 55. In some embodiments, at least one of the one or more Let-7a-5p antagonists includes an anti-miR-Let-7a-5p comprising a nucleotide sequence having at least about, or having about, 80%, 85%, 90%, 95%, 96%, 97, 98%, 99% or 100%, or a range between any two of these values, sequence identity to a sequence selected from the group consisting SEQ ID NOs: 37, 39, and 40-45. In some embodiments, at least one of the one or more Let-7c-5p antagonists includes an anti-miR-Let-7c-5p comprising a nucleotide sequence having at least about, or having about, 80%, 85%, 90%, 95%, 96%, 97, 98%, 99% or 100%, or a range between any two of these values, sequence identity to a sequence selected from the group consisting of SEQ ID NOs: 36, 38, and 40-45.

In some embodiments of the compositions disclosed herein, one or more of the followings applies. In some embodiments, at least one of the one or more miR-99a antagonists includes an anti-miR-99a comprising a nucleotide sequence having one or more mismatched nucleobases with respect to a sequence selected from the group consisting of SEQ ID NOs: 47, 48, 50, 52, and 54. In some embodiments, at least one of the one or more miR-100-5p antagonists includes an anti-miR-100-5p comprising a nucleotide sequence having one or more mismatched nucleobases with respect to a sequence selected from the group consisting of SEQ ID NOs: 46, 49, 51, 53, and 55. In some embodiments, at least one of the one or more Let-7a-5p antagonists includes an anti-miR-Let-7a-5p comprising a nucleotide sequence having one or more mismatched nucleobases with respect to a sequence selected from the group consisting of SEQ ID NOs: 37, 39, and 40-45. In some embodiments, at least one of the one or more Let-7c-5p antagonists includes an anti-miR-Let-7c-5p comprising a nucleotide sequence having one or more mismatched nucleobases with respect to a sequence selected from the group consisting of SEQ ID NOs: 36, 38, and 40-45.

In some embodiments, the plurality of miR antagonists includes at least one miR antagonist comprising a nucleotide sequence having, or having about, 1, 2, 3, 4, 5, 6, 7, 8, 9, 10, or a range between any two of these values, mismatched nucleobases with respect to the nucleotide sequence of one or more of the miR antagonists disclosed herein. For example, in some embodiments, the miR antagonist comprises, or consists of, a nucleotide sequence having at least about 1, at least about 2, at least about 3, at least about 4, at least about 5, or more, mismatched nucleobases with respect to the nucleotide sequence of one or more of the miR antagonists disclosed herein. In some embodiments, the miR antagonist comprises, or consists of, a nucleotide sequence having at least about 6, at least about 7, at least about 8, at least about 9, at least about 10, or more, mismatched nucleobases with respect to the nucleotide sequence of one or more of the miR antagonists disclosed herein.

Accordingly, in some embodiments, at least one of the one or more miR-99a antagonists includes an anti-miR-99a comprising a nucleotide sequence having, or having about, 1, 2, 3, 4, 5, 6, 7, 8, 9, 10, or a range between any two of these values, mismatched nucleobases with respect to a nucleotide sequence selected from the group consisting of SEQ ID NOs: 47, 48, 50, 52, and 54. In some embodiments, at least one of the one or more miR-100-5p antagonists includes an anti-miR-100-5p comprising a nucleotide sequence having, or having about, 1, 2, 3, 4, 5, 6, 7, 8, 9, 10, or a range between any two of these values, mismatched nucleobases with respect to a nucleotide sequence selected from the group consisting of SEQ ID NOs: 46, 49, 51, 53, and 55. In some embodiments, at least one of the one or more Let-7a-5p antagonists includes an anti-miR-Let-7a-5p comprising a nucleotide sequence having, or having about, 1, 2, 3, 4, 5, 6, 7, 8, 9, 10, or a range between any two of these values, mismatched nucleobases with respect to a nucleotide sequence selected from the group consisting of SEQ ID NOs: 37, 39, and 40-45. In some embodiments, at least one of the one or more Let-7c-5p antagonists includes an anti-miR-Let-7c-5p comprising a nucleotide sequence having, or having about, 1, 2, 3, 4, 5, 6, 7, 8, 9, 10, or a range between any two of these values, mismatched nucleobases with respect to a nucleotide sequence selected from the group consisting of SEQ ID NOs: 36, 38, and 40-45.

In various embodiments of the compositions disclosed herein, at least one of the anti-miRs includes one or more chemical modifications described herein. Suitable chemical modifications include, but are not limited to, modifications to a nucleobase, a sugar, and/or an internucleoside linkage. A modified nucleobase, sugar, and/or internucleoside linkage may be selected over an unmodified form because of desirable properties such as, for example, enhanced cellular uptake, enhanced affinity for other oligonucleotides or nucleic acid targets and increased stability in the presence of nucleases. Accordingly, in some embodiments of the compositions disclosed herein, at least one of the anti-miRs includes one or more chemical modifications selected from the group consisting of a modified internucleoside linkage, a modified nucleotide, and a modified sugar moiety, and combinations thereof.

In some embodiments, the one or more chemical modifications includes a modified internucleoside linkage. Generally, a modified internucleoside linkage can be any internucleoside linkage known in the art. Non-limiting examples of suitable modified internucleoside linkage include a phosphorothioate, 2′-Omethoxyethyl (MOE), 2′-fluoro, alkylphosphonate, phosphorodithioate, alkylphosphonothioate, phosphoramidate, carbamate, carbonate, phosphate triester, acetamidate, carboxymethyl ester, and combinations thereof. In some embodiments, the modified internucleoside linkage comprises a phosphorus atom. In some embodiments, the modified internucleoside linkage does not comprise a phosphorus atom. In certain such embodiments, an internucleoside linkage is formed by a short chain alkyl internucleoside linkage. In certain such embodiments, an internucleoside linkage is formed by a cycloalkyl internucleoside linkages. In certain such embodiments, an internucleoside linkage is formed by a mixed heteroatom and alkyl internucleoside linkage. In certain such embodiments, an internucleoside linkage is formed by a mixed heteroatom and cycloalkyl internucleoside linkages. In certain such embodiments, an internucleoside linkage is formed by one or more short chain heteroatomic internucleoside linkages. In certain such embodiments, an internucleoside linkage is formed by one or more heterocyclic internucleoside linkages. In certain such embodiments, an internucleoside linkage has an amide backbone. In certain such embodiments, an internucleoside linkage has mixed N, O, S and CH₂ component parts. In some embodiments, at least one of the anti-miRs includes a modified internucleoside linkage which is a phosphorothioate internucleoside linkage.

In some embodiments, at least one of the one or more chemical modifications includes a modified nucleotide. A modified nucleotide can generally be any modified nucleotide and can be for example, a locked nucleic acid (LNA) chemistry modification, a peptide nucleic acid (PNA), an arabino-nucleic acid (FANA), an analogue, a derivative, or a combination thereof. In some embodiments, the modified nucleotide comprises 5-methylcytosines. In some embodiments, a modified nucleotide is selected from 5-hydroxymethyl cytosine, 7-deazaguanine and 7-deazaadenine. In certain embodiments, the modified nucleotide is selected from 7-deaza-adenine, 7-deazaguanosine, 2-aminopyridine and 2-pyridone. In certain embodiments, the modified nucleotide is selected from 5-substituted pyrimidines, 6-azapyrimidines and N-2, N-6 and 0-6 substituted purines, including 2 aminopropyladenine, 5-propynyluracil and 5-propynylcytosine. In certain embodiments, a modified nucleotide comprises a polycyclic heterocycle. In certain embodiments, a modified nucleotide comprises a tricyclic heterocycle. In certain embodiments, a modified nucleotide comprises a phenoxazine derivative. In certain embodiments, the phenoxazine can be further modified to form a nucleobase known in the art as a G-clamp.

In some embodiments, the modified nucleotide includes a locked nucleic acid (LNA). In some embodiments, the one or more chemical modifications includes at least one locked nucleic acid (LNA) chemistry modifications to enhance the potency, specificity and duration of action and broaden the routes of administration of oligonucleotides. This can be achieved by substituting some of the nucleobases in a base nucleotide sequence by LNA nucleobases. The LNA modified nucleotide sequences may have a size similar to the parent nucleobase or may be larger or preferably smaller. In some embodiments, the LNA-modified nucleotide sequences contain less than about 70%, less than about 65%, more preferably less than about 60%, less than about 55%, most preferably less than about 50%, less than about 45% LNA nucleobases and that their sizes are between about 5 and 25 nucleotides, more preferably between about 12 and 20 nucleotides. In some embodiments, the locked nucleic acid (LNA) is incorporated at one or both ends of the modified anti-miR.

In some embodiments, the one or more chemical modifications include at least one modified sugar moiety. In some embodiments, In certain embodiments, a sugar modified nucleoside is a 2′-modified nucleoside, wherein the sugar ring is modified at the 2′ carbon from natural ribose or 2′-deoxy-ribose. In some embodiments, a 2′-modified nucleoside has a bicyclic sugar moiety. In certain such embodiments, the bicyclic sugar moiety is a D sugar in the alpha configuration. In certain such embodiments, the bicyclic sugar moiety is a D sugar in the beta configuration. In certain such embodiments, the bicyclic sugar moiety is an L sugar in the alpha configuration. In certain such embodiments, the bicyclic sugar moiety is an L sugar in the beta configuration.

In some embodiments, the bicyclic sugar moiety comprises a bridge group between the 2′ and the 4′-carbon atoms. In certain such embodiments, the bridge group comprises from 1 to 8 linked biradical groups. In certain embodiments, the bicyclic sugar moiety comprises from 1 to 4 linked biradical groups. In certain embodiments, the bicyclic sugar moiety comprises 2 or 3 linked biradical groups. In certain embodiments, the bicyclic sugar moiety comprises 2 linked biradical groups. In certain embodiments, a linked biradical group is selected from —O—, —S—, —N(R₁)—, —C(R₁)(R₂)—, —C(R₁)═C(R₁)—, —C(R₁)═N—, —C(═NR₁)—, —Si(R₁)(R₂)—, —S(═O)₂—, —S(═O)—, —C(═O)— and —C(═S)—; where each R₁ and R₂ is, independently, H, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, substituted oxy (—O—), amino, substituted amino, azido, carboxyl, substituted carboxyl, acyl, substituted acyl, CN, thiol, substituted thiol, sulfonyl (S(═O)₂—H), substituted sulfonyl, sulfoxyl (S(═O)—H) or substituted sulfoxyl; and each substituent group is, independently, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, amino, substituted amino, acyl, substituted acyl, C₁-C₁₂ aminoalkyl, C₁-C₁₂ aminoalkoxy, substituted C₁-C₁₂ aminoalkyl, substituted C₁-C₁₂ aminoalkoxy or a protecting group.

In some embodiments, the bicyclic sugar moiety is bridged between the 2′ and 4′ carbon atoms with a biradical group selected from —O—(CH₂)ₚ—, —O—CH₂—, —O—CH₂CH₂—, —O—CH(alkyl)-, —NH—(CH₂)ₚ—, —N(alkyl)-(CH₂)ₚ—, —O—CH(alkyl)-, —(CH(alkyl))—(CH₂)ₚ—, —NH—O—(CH₂)ₚ—, —N(alkyl)-O—(CH₂)ₚ—, or —O—N(alkyl)-(CH₂)ₚ—, wherein p is 1, 2, 3, 4 or 5 and each alkyl group can be further substituted. In certain embodiments, p is 1, 2 or 3.

In some embodiments, a 2′-modified nucleoside comprises a 2′-substituent group selected from halo, allyl, amino, azido, SH, CN, OCN, CF₃, OCF₃, O-, S-, or N(Rₘ)-alkyl; O-, S-, or N(Rₘ)-alkenyl; O-, S- or N(Rₘ)-alkynyl; O-alkylenyl-O-alkyl, alkynyl, alkaryl, aralkyl, O-alkaryl, O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In some embodiments, a 2′-modified nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), —O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In some embodiments, a 2′-modified nucleoside comprises a 2′-substituent group selected from F, OCF₃, O—CH3, OCH2CH2OCH3, 2′-O(CH2)2SCH3, O—(CH2)2-O—N(CH3)2, O(CH2)2O(CH2)2N—, (CH3)2, and O—CH2-C(═O)—N(H)CH3.

In some embodiments, a 2′-modified nucleoside comprises a 2′-substituent group selected from F, O—CH₃, and OCH₂CH₂OCH₃.

In some embodiments, a sugar-modified nucleoside is a 4′-thio modified nucleoside. In certain embodiments, a sugar-modified nucleoside is a 4′-thio-2′-modified nucleoside. A 4′-thio modified nucleoside has a β-D-ribonucleoside where the 4′-O replaced with 4′-S. A 4′-thio-2′-modified nucleoside is a 4′-thio modified nucleoside having the 2′-OH replaced with a 2′-substituent group. Suitable 2′-substituent groups include 2′-OCH₃, 2′-O—(CH₂)₂—OCH₃, and 2′-F.

Accordingly, in some embodiments of the disclosure, the modified sugar moiety is a 2′-O-methoxyethyl modified sugar moiety, a 2′-methoxy modified sugar moiety, a 2′-O-alkyl modified sugar moiety, a bicyclic sugar moiety, or a combination thereof. In some embodiments, the modified sugar moiety comprises a 2′-O-methyl sugar moiety.

### Expression Cassettes

In some embodiments, one or more of the miR antagonists described herein are encoded by and expressed from expression cassettes. Thus, in one aspect, some embodiments of the present disclosure related to expression cassettes that include a nucleotide sequence encoding one or more miR antagonists described herein. As used herein, “expression” refers to the process of converting genetic information of a polynucleotide into RNA through transcription, which is typically catalyzed by an enzyme, RNA polymerase, and, where the RNA encodes a polypeptide, into protein, through translation of mRNA on ribosomes to produce the encoded protein. The term “expression cassette” as used herein, refers to a nucleic acid construct that encodes a gene, a protein, or a functional RNA operably linked to expression control elements, such as a promoter, and optionally, any or a combination of other nucleic acid sequences that affect the transcription or translation of the gene, such as, but not limited to, a transcriptional terminator, a ribosome binding site, a splice site or splicing recognition sequence, an intron, an enhancer, a polyadenylation signal, an internal ribosome entry site, etc.

### Cloning Vectors and Expression Vectors

In a related aspect, one or more of the miR antagonists described herein can be encoded by and/or expressed from a cloning vector or an expression vector. Accordingly, some embodiments of the present application are directed to a cloning vector or expression vector that includes an expression cassette as disclosed herein. As used herein, the term “vector” refers to a nucleic acid construct, typically a plasmid or a virus, used to transmit genetic material to a host cell. Vectors can be, for example, viruses, plasmids, cosmids, or phage. A vector as used herein can be composed of either DNA or RNA. In some embodiments, a vector is composed of DNA. In some embodiments, a vector is composed of RNA. The term “vector” includes cloning vectors and expression vectors, as well as viral vectors and integrating vectors. An “expression vector” is a vector that is capable of directing the expression of a gene, or protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

Accordingly, in some embodiments, the cloning vector or expression vector disclosed herein includes an expression cassette including a nucleotide sequence which encodes one or more miR antagonists described herein. In some embodiments, the cloning vector or expression vector disclosed herein includes an expression cassette including a nucleotide sequence which encodes one or more miR-99a antagonists, one or more miR-100-5p antagonists, one or more miR-Let-7a-5p antagonists, and one or more miR-Let-7c-5p antagonists.

In some embodiments, the cloning vector or expression vector is a viral vector. As used herein, a “viral vector” is a viral-derived nucleic acid molecule that is capable of transporting another nucleic acid into a cell. A viral vector is capable of directing expression of a gene, a protein or proteins encoded by one or more genes carried by the vector when it is present in the appropriate environment. Examples for viral vectors include, but are not limited to retroviral vectors, adenoviral vectors, lentiviral vectors, and adeno-associated viral vectors.

Accordingly, in some embodiments, the viral vector is a lentiviral vector or an adeno-associated viral (AAV) vector or any serotype. As used herein, the term “serotype” or “serovar” is a distinct variation within a species of bacteria or virus or among immune cells of different individuals. These microorganisms, viruses, or cells are classified together based on their cell surface antigens, allowing the epidemiologic classification of organisms to the sub-species level. Generally, the AAV vector can be any existing AAV vectors and can be, for example, an AAV vector selected from the group consisting of serotype 1, 2, 3, 4, 5, 6, 7, 8, 9, and 10 or chimeric AAV derived thereof, which will be even better suitable for high efficiency transduction in the tissue of interest. Upon transfection, AAV elicits only a minor immune reaction (if any) in the host. Therefore, AAV vector is highly suited for gene therapy approaches. It has been reported that, for transduction in mice, AAV serotype 6 and AAV serotype 9 are particularly suitable. For gene transfer into a human, AAV serotypes 1, 6, 8 and 9 are generally preferred. It has been also assumed that the capacity of AAV for packaging a therapeutic gene is limited to approximately 4.9 kb, while longer sequences lead to truncation of AAV particles. In some embodiments, the AAV vector is an AAV2/9 vector, e.g., AAV2 inverted terminal repeat (ITR) sequences cross-packaged into AAV capsid.

In some embodiments, disclosed herein are cloning or expression vectors having, or having about, 85%, 90%, 91%, 92%, 93%, 94%, 95%, 96%, 97%, 98%, 99%, 100%, or a range between any two of these values, sequence identity to one or more of the vectors disclosed herein. For example, in some embodiments, the cloning or expression vector comprises, or consists of, a nucleotide sequence having at least about 85%, at least about 90%, at least about 95%, at least about 96%, at least about 97%, at least about 98%, at least about 99%, or more, sequence identity to the full sequence of JBT-miR1 (SEQ ID NO: 85). In some embodiments, the vector comprises, or consists of, a nucleotide sequence having at least about 85%, at least about 90%, at least about 95%, at least about 96%, at least about 97%, at least about 98%, at least about 99%, or more, sequence identity to the nucleotide sequence of JBT-miR2. In some embodiments, the vector comprises, or consists of, a nucleotide sequence having about 85%, about 90%, about 95%, about 96%, about 97%, about 98%, about 99%, about 100%, or a range between any two of these values, sequence identity to the full sequence of JBT-miR1 (SEQ ID NO: 85) or JBT-miR2.

In some embodiments, the cloning vector or expression vector disclosed herein includes a nucleotide sequence having, or having about, 85%, 90%, 91%, 92%, 93%, 94%, 95%, 96%, 97%, 98%, 99%, 100%, or a range between any two of these values, sequence identity to each of the nucleotide sequences set forth in SEQ ID NOs: 59-64. In some embodiments, the cloning vector or expression vector disclosed herein includes a nucleotide sequence having, or having about, 85%, 90%, 91%, 92%, 93%, 94%, 95%, 96%, 97%, 98%, 99%, 100%, or a range between any two of these values, sequence identity to each of the nucleotide sequences set forth in SEQ ID NOs: 86-89. In some embodiments, the cloning vector or expression vector disclosed herein includes a nucleotide sequence having, or having about, 85%, 90%, 91%, 92%, 93%, 94%, 95%, 96%, 97%, 98%, 99%, 100%, or a range between any two of these values, sequence identity to each of the nucleotide sequences set forth in SEQ ID NOs: 59-64 and SEQ ID NOs: 86-89. In some embodiments, the cloning vector or expression vector disclosed herein includes a nucleotide sequence having, or having about, 85%, 90%, 91%, 92%, 93%, 94%, 95%, 96%, 97%, 98%, 99%, 100%, or a range between any two of these values, sequence identity to the nucleotide sequence of SEQ ID NO: 8

### Therapeutic Compositions and Pharmaceutical Formulations

In another aspect, disclosed herein are embodiments of a therapeutic composition that includes an effective amount of at least one therapeutic agent, and one or more of the followings: a) a composition comprising a plurality of microRNA (miR) antagonists as disclosed herein; b) an expression cassette as disclosed herein; and a cloning or expression vector as disclosed herein.

While it is possible for the agents to be administered as the raw substances, it is preferable, in view of their potency, to present them as a pharmaceutical formulation. Thus, in some embodiments of the compositions disclosed herein, the composition is further formulated into a pharmaceutical formulation. The term “pharmaceutical formulation”, as used herein, refers to a composition suitable for administering to an individual that includes a pharmaceutical agent. For example, a pharmaceutical formulation according to some aspects and embodiments of the present disclosure may comprise an anti-miR antagonist disclosed herein and a sterile aqueous solution. For example, the pharmaceutical formulations of the present disclosure for human use comprise the agent, together with one or more acceptable carriers therefor and optionally other therapeutic ingredients. The carrier(s) must be “acceptable” in the sense of being compatible with the other ingredients of the formulation and not deleterious to the recipient thereof or deleterious to the inhibitory function of the active agent. Desirably, the pharmaceutical formulations should not include oxidizing agents and other substances with which the agents are known to be incompatible.

Accordingly, some embodiments disclosed herein relate to pharmaceutical formulations that include a therapeutic composition described herein and a pharmaceutically acceptable carrier. The formulations can also comprise additional ingredients such as diluents, stabilizers, excipients, and adjuvants. As used herein, “pharmaceutically acceptable” carriers, excipients, diluents, adjuvants, or stabilizers are the ones nontoxic to the cell or subject being exposed thereto (preferably inert) at the dosages and concentrations employed or that have an acceptable level of toxicity as determined by the skilled practitioner.

Buffers may also be included in the pharmaceutical formulations to provide a suitable pH value for the formulation. Suitable such materials include sodium phosphate and acetate. Sodium chloride or glycerin may be used to render a formulation isotonic with the blood. If desired, the formulation may be filled into the containers under an inert atmosphere such as nitrogen or may contain an anti-oxidant, and are conveniently presented in unit dose or multi-dose form, for example, in a sealed ampoule.

The carriers, diluents and adjuvants can include antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

Generally, the pharmaceutical formulations disclosed herein can be prepared by any one of the methods and techniques known in the art. For example, solid dosage forms can be prepared by wet granulation, dry granulation, direct compression, and the like. In some embodiments, the solid dosage forms of the present disclosure may be coated or otherwise compounded to provide a dosage form affording the advantage of prolonged action. For example, the tablet or pill can comprise an inner dosage and an outer dosage component, the latter being in the form of an envelope over the former. In some embodiments, the two components can be separated by an enteric layer, which serves to resist disintegration in the stomach and permit the inner component to pass intact into the duodenum or to be delayed in release. In these instances, a variety of materials can be used for such enteric layers or coatings, such materials including a number of polymeric acids and mixtures of polymeric acids with such materials as shellac, cetyl alcohol, and cellulose acetate.

Titers of the expression vector and/or one or more of the miRNA antagonists to be administered will vary depending, for example, on the particular expression vector, the mode of administration, the treatment goal, the individual, and the cell type(s) being targeted, and can be determined by methods standard in the art.

As will be readily apparent to one of ordinary skill in the art, the useful in vivo dosage of the expression vectors and/or one or more of the miRNA antagonists to be administered and the particular mode of administration will vary depending upon the age, weight, the severity of the affliction, and animal species treated, the particular expression vector that is used, and the specific use for which the expression vector and/or one or more of the miRNA antagonists is employed. The determination of effective dosage levels, that is the dosage levels necessary to achieve the desired result, can be accomplished by one of ordinary skill in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

For example, dosage regimens may be adjusted to provide the optimum desired response. For example, a single dose may be administered, or several divided doses may be administered over time, or the dose may be proportionally reduced or increased as indicated by the exigencies of the therapeutic situation. It is especially advantageous to formulate parenteral compositions and formulations in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure are dictated by and directly dependent on (a) the unique characteristics of the therapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of therapeutic agents are well-known in the relevant art and would be understood to be encompassed by the skilled artisan once provided the teachings disclosed herein.

The expression vectors and/or the miRNA antagonists disclosed herein can be administered to a subject (e.g., a human) in need thereof. The route of the administration is not particularly limited. For example, a therapeutically effective amount of the recombinant viruses can be administered to the subject by via routes standard in the art. Non-limiting examples of the route include intramuscular, intravaginal, intravenous, intraperitoneal, subcutaneous, epicutaneous, intradermal, rectal, intraocular, pulmonary, intracranial, intraosseous, oral, buccal, or nasal. In some embodiments, the recombinant virus is administered to the subject by intramuscular injection. In some embodiments, the recombinant virus is administered to the subject by intravaginal injection. In some embodiments, the expression vectors and/or the miRNA antagonists is administered to the subject by the parenteral route (e.g., by intravenous, intramuscular or subcutaneous injection), by surface scarification or by inoculation into a body cavity of the subject. In some embodiments, the expression vectors and/or the miRNA antagonists are administered to muscle cells such as, cardiac muscle cells.

When administering these small miR oligonucleotide antagonists by injection, the administration may be by continuous infusion, or by single or multiple boluses. The dosage of the administered miR antagonist will vary depending upon such factors as the patient's age, weight, sex, general medical condition, and previous medical history. Typically, it is desirable to provide the recipient with a dosage of the molecule which is in the range of from about 1 μg/kg to 10 mg/kg (amount of agent/body weight of patient), although a lower or higher dosage may also be administered,

In some embodiments, it may be desirable to target delivery of a therapeutic to the heart, while limiting delivery of the therapeutic to other organs. This may be accomplished by any one of a number of methods known in the art. In some embodiments, delivery to the heart of a therapeutic composition or pharmaceutical formulation described herein comprises coronary artery infusion. In certain embodiments, coronary artery infusion involves inserting a catheter through the femoral artery and passing the catheter through the aorta to the beginning of the coronary artery. In yet some other embodiments, targeted delivery of a therapeutic to the heart involves using antibody-protamine fusion proteins, such as those previously describe (Song E et al., Nature Biotechnology, 2005), to deliver the small miR oligonucleotide antagonists disclosed herein.

Actual administration of the expression vectors and/or the miRNA antagonists can be accomplished by using any physical method that will transport the expression vectors and/or the miRNA antagonists into the target tissue of the subject. For example, the expression vectors and/or the miRNA antagonists can be injected into muscle, the bloodstream, and/or directly into the liver. Pharmaceutical formulations can be prepared as injectable formulations or as topical formulations to be delivered to the muscles by transdermal transport.

For intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of the expression vectors and/or the miRNA antagonists as a free acid (DNA contains acidic phosphate groups) or a pharmacologically acceptable salt can be prepared in water suitably mixed with a surfactant such as hydroxpropylcellulose. A dispersion of the expression vectors and/or the miRNA antagonists can also be prepared in glycerol, liquid polyethylene glycols and mixtures thereof and in oils. Under ordinary conditions of storage and use, these preparations contain a preservative to prevent the growth of microorganisms.

The expression vectors and/or the miRNA antagonists to be used can be utilized in liquid or freeze-dried form (in combination with one or more suitable preservatives and/or protective agents to protect the virus during the freeze-drying process). For gene therapy (e.g., of neurological disorders which may be ameliorated by a specific gene product) a therapeutically effective dose of the recombinant virus expressing the therapeutic protein is administered to a host in need of such treatment. The use of the expression vectors and/or the miRNA antagonists disclosed herein in the manufacture of a medicament for inducing immunity in, or providing gene therapy to, a host is within the scope of the present application.

In instances where human dosages for the expression vectors and/or the miRNA antagonists have been established for at least some condition, those same dosages, or dosages that are between about 0.1% and 500%, more preferably between about 25% and 250% of the established human dosage can be used. Where no human dosage is established, as will be the case for newly-discovered pharmaceutical formulations, a suitable human dosage can be inferred from ED₅₀ or ID₅₀ values, or other appropriate values derived from in vitro or in vivo studies, as qualified by toxicity studies and efficacy studies in animals

A therapeutically effective amount of the expression vectors and/or the miRNA antagonists can be administered to a subject at various points of time. For example, the expression vectors and/or the miRNA antagonists can be administered to the subject prior to, during, or after the infection by a virus. The expression vectors and/or the miRNA antagonists can also be administered to the subject prior to, during, or after the occurrence of a disease (e.g., cancer). In some embodiments, the expression vectors and/or the miRNA antagonists is administered to the subject during cancer remission. In some embodiments, the expression vectors and/or the miRNA antagonists is administered prior to infection by the virus for immunoprophylaxis.

Alternatively or in addition, the dosing frequency of the expression vectors and/or the miRNA antagonists can vary. For example, the expression vectors and/or the miRNA antagonists can be administered to the subject about once every week, about once every two weeks, about once every month, about one every six months, about once every year, about once every two years, about once every three years, about once every four years, about once every five years, about once every six years, about once every seven years, about once every eight years, about once every nine years, about once every ten years, or about once every fifteen years. In some embodiments, the expression vectors and/or the miRNA antagonists is administered to the subject at most about once every week, at most about once every two weeks, at most about once every month, at most about one every six months, at most about once every year, at most about once every two years, at most about once every three years, at most about once every four years, at most about once every five years, at most about once every six years, at most about once every seven years, at most about once every eight years, at most about once every nine years, at most about once every ten years, or at most about once every fifteen years.

In some embodiments, a pharmaceutical kit is provided, wherein the kit comprises: any of the forgoing the therapeutic compositions and pharmaceutical formulations, and written information (a) indicating that the formulation is useful for inhibiting, in myocardial cells, such as, for example cardiomyocytes, the function of a gene associated with the heart disease and/or (b) providing guidance on administration of the pharmaceutical formulation.

### III. Methods of the Disclosure

There are provided, in some embodiments, methods of preventing, inhibiting, reducing, or treating cardiac ischemic reperfusion injury. In some embodiments, the method comprises: administering a therapeutic composition to a subject before, during, and/or after a cardiac ischemic event, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b). The method can comprise: reperfusion of ischemic cardiac tissue.

Disclosed herein include methods increasing heart function, reducing mortality, reducing cardiac volumes and/or reducing scar size following ischemic reperfusion injury. In some embodiments, the method comprises: administering a therapeutic composition to a subject before, during, and/or after a cardiac ischemic event, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b). The method can comprise: reperfusion of ischemic cardiac tissue.

As used herein, the term “ischemia-reperfusion injury” (IRI), shall be given its ordinary meaning, and shall also refer to tissue damage (e.g., injury) caused by ischemia, reperfusion, or ischemia followed by reperfusion. Thus, the term “ischemia-reperfusion injury” includes injuries caused by ischemia, reperfusion injuries, and injuries caused by ischemia followed by reperfusion. Myocardial infarction (MI) is a type of cardiac ischemia event that can result in IR injury of the heart tissues. As used herein, the “injury resulting from ischemia,” “injury caused by ischemia” and “ischemic injury” can refer to an injury to a cell, tissue or organ caused by ischemia, or an insufficient supply of blood (e.g., due to a blocked artery), and, thus, oxygen, resulting in damage or dysfunction of the tissue or organ. In some embodiments, the term “ischemia-reperfusion injury” refers to an injury resulting from the restoration of blood flow to an area of a tissue or organ that had previously experienced deficient blood flow due to an ischemic event. Oxidative stresses associated with reperfusion may cause damage to the affected tissues or organs. Ischemia-reperfusion injury is characterized biochemically by a depletion of oxygen during an ischemic event followed by reoxygenation and the concomitant generation of reactive oxygen species during reperfusion. In some embodiments, the compositions provided herein are administered at the time of reperfusion. “At the time of reperfusion” can range, in some embodiments, from two hours before to 2 hours after reperfusion, as well as right at the same time of reperfusion. This implies that the compositions provided herein can be administered at the same time as, for example, a thrombolytic agent is administered, or at the time of performing a surgical intervention to eliminate the clot obstructing the blood flow.

There are provided, in some embodiments, methods of treating myocardial infarction. In some embodiments, the method comprises: administering a therapeutic composition to a subject before, during, and/or after reperfusion of ischemic cardiac tissue, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b). Myocardial infarction can be a cardiac ischemic event.

There are provided, in some embodiments, methods of inducing cardiomyocyte regeneration, cardiac repair, vasculogenesis and/or cardiomyocyte differentiation following a cardiac ischemic event. In some embodiments, the method comprises: administering a therapeutic composition to a subject before, during, or after reperfusion of ischemic cardiac tissue, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

There are provided, in some embodiments, methods of treating a disease or disorder associated with dysregulation of FHL1 and/or TNNT2. In some embodiments, the method comprises: administering a therapeutic composition to a subject in need thereof, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

There are provided, in some embodiments, methods of treating a kidney condition of a subject and/or protecting a kidney of a subject from injury. In some embodiments, the method comprises: administering a therapeutic composition to the subject, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

At least one of the one or more miR-99a antagonists can comprise an anti-miR-99a comprising a nucleotide sequence having at least 80%, 85%, 90%, 95%, 96%, 97, 98%, 99% or 100% identity to a sequence selected from the group consisting of SEQ ID NOs 47, 48, 50, 52, and 54. At least one of the one or more miR-100-5p antagonists can comprise an anti-miR-100-5p comprising a nucleotide sequence having at least 80%, 85%, 90%, 95%, 96%, 97, 98%, 99% or 100% identity to a sequence selected from the group consisting of SEQ ID NOs 46, 49, 51, 53, and 55. At least one of the one or more Let-7a-5p antagonists can comprise an anti-miR-Let-7a-5p comprising a nucleotide sequence having at least 80%, 85%, 90%, 95%, 96%, 97, 98%, 99% or 100% identity to a sequence selected from the group consisting of SEQ ID NOs: 37, 39, and 40-45. At least one of the one or more Let-7c-5p antagonists can comprise an anti-miR-Let-7c-5p comprising a nucleotide sequence having at least 80%, 85%, 90%, 95%, 96%, 97, 98%, 99% or 100% identity to a sequence selected from the group consisting of SEQ ID NOs: 36, 38, and 40-45.

At least one of the one or more miR-99a antagonists can comprise an anti-miR-99a comprising a nucleotide sequence having one or more mismatched nucleobases with respect to a sequence selected from the group consisting of SEQ ID NOs: 47, 48, 50, 52, and 54. At least one of the one or more miR-100-5p antagonists can comprise an anti-miR-100-5p comprising a nucleotide sequence having one or more mismatched nucleobases with respect to a sequence selected from the group consisting of SEQ ID NOs: 46, 49, 51, 53, and 55. At least one of the one or more Let-7a-5p antagonists can comprise an anti-miR-Let-7a-5p comprising a nucleotide sequence having one or more mismatched nucleobases with respect to a sequence selected from the group consisting of SEQ ID NOs: 37, 39, and 40-45. At least one of the one or more Let-7c-5p antagonists can comprise an anti-miR-Let-7c-5p comprising a nucleotide sequence having one or more mismatched nucleobases with respect to a sequence selected from the group consisting of SEQ ID NOs: 36, 38, and 40-45.

At least one of the anti-miRs can comprise one or more chemical modifications selected from the group consisting of a modified internucleoside linkage, a modified nucleotide, and a modified sugar moiety, and combinations thereof. The one or more chemical modifications can comprise a modified internucleoside linkage. The modified internucleoside linkage can be selected from the group consisting of a phosphorothioate, 2′-Omethoxyethyl (MOE), 2′-fluoro, alkylphosphonate, phosphorodithioate, alkylphosphonothioate, phosphoramidate, carbamate, carbonate, phosphate triester, acetamidate, carboxymethyl ester, and combinations thereof. The modified internucleoside linkage can comprise a phosphorothioate internucleoside linkage. At least one of the one or more chemical modifications can comprise a modified nucleotide. The modified nucleotide can comprise a locked nucleic acid (LNA). The locked nucleic acid (LNA) can be incorporated at one or both ends of the modified anti-miR. The modified nucleotide can comprise a locked nucleic acid (LNA) chemistry modification, a peptide nucleic acid (PNA), an arabino-nucleic acid (FANA), an analogue, a derivative, or a combination thereof. At least one of the one or more chemical modifications can comprise a modified sugar moiety. The modified sugar moiety can be a 2′-O-methoxyethyl modified sugar moiety, a 2′-methoxy modified sugar moiety, a 2′-O-alkyl modified sugar moiety, a bicyclic sugar moiety, or a combination thereof. The modified sugar moiety can comprise a 2′-O-methyl sugar moiety.

The cloning or expression vector disclosed herein can be a viral vector. The viral vector can be a lentiviral vector or an adeno-associated viral (AAV) vector. The cloning or expression vector can comprise: (a) a nucleotide sequence having at least 80%, 85%, 90%, 95%, 96%, 97, 98%, 99% or 100% identity to each of the nucleotide sequences set forth in SEQ ID NOs: 59-64; (b) a nucleotide sequence having at least 80%, 85%, 90%, 95%, 96%, 97, 98%, 99% or 100% identity to each of the nucleotide sequences set forth in SEQ ID NOs: 86-89; or (c) a nucleotide sequence having at least 80%, 85%, 90%, 95%, 96%, 97, 98%, 99% or 100% identity to each of the nucleotide sequences set forth in the SEQ ID NOs indicated in (a) and (b). The cloning or expression vector can comprise a nucleotide sequence having least 80%, 85%, 90%, 95%, 96%, 97, 98%, 99% or 100% identity to the nucleotide sequence of SEQ ID NO: 85. In some embodiments, the plurality of miR antagonists are encoded by the same expression cassette or vector. In some embodiments, the plurality of miR antagonists are encoded by different expression cassettes or vectors.

The cloning or expression vector comprises a nucleotide sequence having least 80%, 85%, 90%, 95%, 96%, 97, 98%, 99% or 100% identity to the nucleotide sequence of SEQ ID NO: 101. In some embodiments, the expression cassette comprises a tough decoy (TuD) cassette comprising a nucleotide sequence encoding one or more miR-99a antagonists. In some embodiments, the TuD cassette comprises one or more promoter sequences operably linked to the nucleotide sequence encoding one or more miR-99a antagonists, optionally the one or more promoter sequences comprise a H1 promoter and/or a U6 promoter. In some embodiments, the cloning or expression vector comprises two or more TuD cassettes. In some embodiments, an effective dose of a therapeutic composition comprising a cloning or expression vector comprising two or more TuD cassettes is at least about 1.1-fold less than an effective dose of a therapeutic composition comprising a cloning or expression vector comprising one TuD cassette. In some embodiments, the TuD cassette comprises a nucleotide sequence having least 80%, 85%, 90%, 95%, 96%, 97, 98%, 99% or 100% identity to the nucleotide sequence of SEQ ID NO: 98. In some embodiments, the cloning or expression vector comprises a nucleotide sequence having least 80%, 85%, 90%, 95%, 96%, 97, 98%, 99% or 100% identity to the nucleotide sequence of SEQ ID NO: 99. In some embodiments, the cloning or expression vector comprises a nucleotide sequence having least 80%, 85%, 90%, 95%, 96%, 97, 98%, 99% or 100% identity to the nucleotide sequence of SEQ ID NO: 100.

In some embodiments, administering the therapeutic composition occurs before the onset of the cardiac ischemic event. In some embodiments, administering the therapeutic composition occurs during the cardiac ischemic event. The therapeutic composition can be administered about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, about 72 hours, or about 96 hours prior to reperfusion of ischemic cardiac tissue. In some embodiments, administering the therapeutic composition occurs concurrent with reperfusion of ischemic cardiac tissue. In some embodiments, administering the therapeutic composition occurs after reperfusion of ischemic cardiac tissue. The therapeutic composition can be administered about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, about 72 hours, about 96 hours, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, or about 20 days, after reperfusion of ischemic cardiac tissue.

The therapeutic composition can comprise a plurality of microRNA (miR) antagonists, the administration can comprise subcutaneous administration, systemic administration, and/or intra-coronary administration, and the therapeutic composition can be administered at a dose of about 0.0001 mg/kg to 100 mg/kg (e.g., about 0.08 mg/kg, about 0.24 mg/kg, about 0.81 mg/kg, about 1.22 mg/kg, about 2.44 mg/kg, about 3.25 mg/kg, about 4.06 mg/kg, about 4.89 mg/kg, about 5.69 mg/kg, about 6.50 mg/kg, about 7.32 mg/kg, or about 8.13 mg/kg). The therapeutic composition can comprise a plurality of microRNA (miR) antagonists, the administration can comprise intra-ventricular administration and/or intra-myocardial administration, and the therapeutic composition can be administered at a dose of about 0.0001 mg/kg to 100 mg/kg (e.g., about 0.004 mg/kg, about 0.012 mg/kg, about 0.0405 mg/kg, about 0.061 mg/kg, about 0.122 mg/kg, about 0.1625 mg/kg, about 0.203 mg/kg, about 0.2445 mg/kg, about 0.2845 mg/kg, about 0.325 mg/kg, about 0.366 mg/kg, or about 0.4065 mg/kg). In some embodiments, subcutaneous administration of the therapeutic composition yields increased survival and reduced incidence of cardiac thrombus as compared to intravenous administration of the therapeutic composition.

The therapeutic composition can comprise a viral vector, and the administration can comprise intravenous systemic administration and/or intra-coronary administration at a dose of about 1.0×10⁵ vg/kg to 1.0×10¹⁹ vg/kg (e.g., about 2.5×10¹² vg (viral genome)/kg, about 2.5×10¹³ vg/kg, about 2.5×10¹⁴ vg/kg, or about 2.5×10¹⁵ vg/kg). The therapeutic composition can comprise a viral vector, the administration can comprise intra-ventricular administration and/or intra-myocardial administration, and the therapeutic composition can be administered at a dose of about 1.0×10⁵ vg/kg to 1.0×10¹⁹ vg/kg (e.g., about 0.125×10¹² vg/kg, about 0.125×10¹³ vg/kg, about 0.125×10¹⁴ vg/kg, or about 0.125×10¹⁵ vg/kg).

The therapeutic composition can be a pharmaceutical composition. The subject can be a mammal (e.g., a human). The dose can be administered in a single administration. The dose can be administered over multiple administrations. The method can comprise: repeated administration of the therapeutic composition to the subject. The repeated administration can comprise administration of one or more additional doses of the therapeutic composition to the subject. The number of additional doses can vary, and can range from 1 additional dose to 100 additional doses. The one or more additional doses can the same, larger, or smaller, than the initial administration. The one or more additional doses can administered in the same or manner as the initial administration, The repeated administration can comprise administration of one or more additional doses of the therapeutic composition to the subject about 1 minute to about 1000 days (e.g., about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, about 72 hours, about 96 hours, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, and/or about 20 days) after reperfusion of ischemic cardiac tissue.

The methods disclosed herein can comprise administrating an effective amount of at least one additional therapeutic agent or at least one additional therapy to the subject for a combination therapy. Each of the therapeutic composition and the at least one additional therapeutic agent or therapy can be administered in a separate formulation or can be administered together in a single formulation. In some embodiments, the therapeutic composition and the at least one additional therapeutic agent or therapy are administered sequentially, are administered concomitantly, and/or are administered in rotation. The at least one additional therapeutic agent or therapeutic therapy can be selected from the group consisting of Idebenone, Eplerenone, VECTTOR, AVI-4658, Ataluren/PTC124/Translarna, BMN044/PRO044, CAT-1004, microDystrophin AAV gene therapy (SGT-001), Galectin-1 therapy (SB-002), LTBB4 (SB-001), rAAV2.5-CMV-minidystrophin, glutamine, NFKB inhibitors, sarcoglycan, delta (35 kDa dystrophin-associated glycoprotein), insulin like growth factor-1 (IGF-1) expression, genome editing through the CRISPR/Cas9 system, any gene delivery therapy aimed at reintroducing a functional recombinant version of the dystrophin gene, Exon skipping therapeutics, read-through strategies for nonsense mutations, cell-based therapies, utrophin upregulation, myostatin inhibition, anti-inflammatories/anti-oxidants, mechanical support devices, a biologic drug, a gene therapy or therapeutic gene modulation agent, any standard therapy for muscular dystrophy, and combinations thereof. The at least one additional therapeutic agent or therapeutic therapy can be selected from the group comprising a percutaneous coronary intervention, coronary artery bypass grafting, thrombolytic therapy, anti-platelet therapy, heparin, warfarin, fibrinolytics, oxygen therapy, a vasodilator, pain medication, a beta blocker, an angiotensin-converting enzyme (ACE) inhibitor, an angiotensin receptor blocker (ARB), a glycoprotein antagonist, a statin, an aldosterone antagonist, an implantable cardiac defibrillator (ICD), or any combination thereof.

Reperfusion of ischemic cardiac tissue can comprise a percutaneous coronary intervention, coronary artery bypass grafting, thrombolytic therapy, anti-platelet therapy, heparin, warfarin, fibrinolytics, oxygen therapy, a vasodilator, pain medication, a beta blocker, an angiotensin-converting enzyme (ACE) inhibitor, an angiotensin receptor blocker (ARB), a glycoprotein antagonist, a statin, an aldosterone antagonist, an implantable cardiac defibrillator (ICD), or any combination thereof.

In some embodiments, the subject has or is suspected of having a cardiac disease. The cardiac disease can be myocardial infarction, ischemic heart disease, dilated cardiomyopathy, heart failure (e.g., congestive heart failure), ischemic cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis, aortic stenosis, aortic regurgitation, mitral stenosis, mitral regurgitation, mitral prolapse, pulmonary stenosis, pulmonary regurgitation, tricuspid stenosis, tricuspid regurgitation, congenital disorder, genetic disorder, or any combination thereof. The subject can be affected by a condition selected from the group comprising alcoholic cardiomyopathy, coronary artery disease, congenital heart disease, nutritional diseases affecting the heart, ischemic cardiomyopathy, hypertensive cardiomyopathy, valvular cardiomyopathy, inflammatory cardiomyopathy, cardiomyopathy secondary to a systemic metabolic disease, dilated cardiomyopathy (DCM), hypertrophic cardiomyopathy (HCM), arrhythmogenic right ventricular cardiomyopathy (ARVC), restrictive cardiomyopathy (RCM), noncompaction cardiomyopathy, supravalvular aortic stenosis (SVAS), vascular scarring, atherosclerosis, chronic progressive glomerular disease, glomerulosclerosis, progressive renal failure, vascular occlusion, hypertension, stenosis, diabetic retinopathy, or any combination thereof.

The cardiac ischemic reperfusion injury can comprise cardiac ischemic damage, cardiac reperfusion injury, or a combination thereof. In some embodiments, the administration reduces cardiac ischemic damage, cardiac reperfusion injury, or a combination thereof, as compared to a control subject. In some embodiments, the administration reduces creatine kinase levels as compared to a control subject. The cardiac ischemic reperfusion injury can comprise injuries caused by the cardiac ischemia event, reperfusion injuries, or a combination thereof.

The cardiac ischemic event can comprise one or more of myocardial infarction, coronary artery bypass grafting (CABG), cardiac bypass surgery, cardiac transplantation, and angioplasty. The cardiac ischemic event can comprise a vascular interventional procedure employing a stent, laser catheter, atherectomy catheter, angioscopy device, beta or gamma radiation catheter, rotational atherectomy device, coated stent, radioactive balloon, heatable wire, heatable balloon, biodegradable stent strut, a biodegradable sleeve, or any combination thereof.

In some embodiments, the administration results in one or more of (1) increased survival as compared to a control subject, (2) improved kidney function of the subject as compared to a control subject, (3) a decrease in blood urea nitrogen (BUN) levels as compared to a control subject, (4) a reduced scarring in the left ventricle of the subject and/or improved regional wall motion in the left ventricle of the subject as compared to a control subject, (5) a decrease in end diastolic volume and/or end systolic volume as compared to a control subject, (6) an increase in ejection fraction as compared to a control subject, (7) an increase in the number of cardiomyocytes and/or mRNAs encoding proteins that are involved in differentiated cardiomyocyte muscle structure and function as compared to a control subject, (8) an increase in the mRNA levels and/or protein levels of one or more of Ank2, Kdm6a, Grk6, K1h115, Adam22, Pfkp, Gorasp2, Ralgps1, Inppl1, Kdm3a, Kit, Sort1, Dv12, Sema6d, Tead1, B4galnt2, Ltbp4, Osbp19, Nfe2I1, Tnnt2, and Fhl1 as compared to a control subject, and (9) a decrease in the mRNA levels and/or protein levels of one or more of Asph, Map6, Zfp120, Ctnndl, Eya3, Tnnt2, Kdm3a, Myo18a, Ncoa6, Slc25a13, Rpe, Ralgps1, Gimap1, Myo5a, Zeb2, Arap1, Nt5c2, Phldb1, Ttn, Camta2, Mef2c, Slk, Uimc1, Mthfd1I, Mtus1, Ythdc1, and Eif2ak4 as compared to a control subject, and (10) an increase in one of more of cardiomyocyte formation, cardiomyocyte proliferation, cardiomyocyte cell cycle activation, mitotic index of cardiomyocytes, myofilament density, borderzone wall thickness, or any combination thereof, as compared to a control subject, by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) at a time point about 5 minutes to about 365 days after administration (e.g., about 5 minutes, about 10 minutes, about 15 minutes, about 20 minutes, about 25 minutes, about 30 minutes, about 40 minutes, about 50 minutes, or about 60 minutes, about 1 day, about 2 days, about 4 days, about 6 days, about 8 days, about 10 days, about 20 days, about 30 days, about 40 days, about 50 days, about 60 days, about 80 days, about 100 days, about 120 days, about 140 days, about 160 days, about 180 days, about 200 days, about 220 days, about 240 days, about 260 days, about 280 days, about 300 days, about 320 days, about 340 days, about 360 days, about 365 days, or a number or a range between any of these values). In some embodiments, the administration induces endogenous cardiomyocyte regeneration. In some embodiments, the administration enhances cardiac function in the subject as compared to a control subject. Enhancing cardiac function can comprise one or more of (i) improving left ventricular function, (ii) improving fractional shortening, (iii) improving ejection fraction, (iv) reducing end-diastolic volume, (v) decreasing left ventricular mass, and (v) normalizing of heart geometry, or (vi) a combination thereof. In some embodiments, the administration has no significant effect on body weight and/or heart weight. In some embodiments, the administration does not cause one or more of arrhythmia, after contractions (AC), and contraction failure (CF).

The compositions provided herein can also be used to inhibit an ischemia or ischemia-reperfusion injury to a cell, tissue or organ, ex vivo, prior to a therapeutic intervention (e.g., a tissue employed in a graft procedure, an organ employed in an organ transplant surgery). For example, prior to transplant of an organ into a host individual (e.g., during storage or transport of the organ in a sterile environment), the organ can be contacted with compositions provided herein (e.g., bathed in a solution comprising the compositions provided herein) to inhibit ischemia or ischemia-reperfusion injury.

The methods provided herein can treat a disease or disorder associated with one or more FHL1 mutations and/or one or more TNNT2 mutations. In some embodiments, the therapeutic composition increases the mRNA levels and/or protein levels of FHL1 and/or TNNT2. The disease or disorder can be a muscular dystrophy disorder or a muscular dystrophy-like muscle disorder. The muscular dystrophy disorder can be associated with Amyotrophic Lateral Sclerosis (ALS), Charcot-Marie-Tooth Disease (CMT), Congenital Muscular Dystrophy (CMD), Duchenne Muscular Dystrophy (DMD), Emery-Dreifuss Muscular Dystrophy (EDMD), Inherited and Endocrine Myopathies, Metabolic Diseases of Muscle, Mitochondrial Myopathies (MM), Myotonic Muscular Dystrophy (MMD), Spinal-Bulbar Muscular Atrophy (SBMA), or a combination thereof. The disease or disorder can be Limb girdle muscular dystrophy, X-linked myopathy with postural muscle atrophy (XMPMA), Reducing body myopathy (RBM), Scapuloperoneal (SP) syndrome, or any combination thereof. The disease or disorder can be hypertrophic cardiomyopathy (HCM), restrictive cardiomyopathy (RCM), dilated cardiomyopathy (DCM), or any combination thereof. The hypertrophic cardiomyopathy can be familial hypertrophic cardiomyopathy.

In some embodiments, the compositions disclosed herein exhibit a renal therapeutic effect. In some embodiments, the renal therapeutic effect comprises a renal protective effect or renal prophylactic effect. The methods provided herein can treat a kidney condition associated with a function of the subject's kidneys. The kidney condition can be selected from the group consisting of acute kidney diseases and disorders (AKD), acute kidney injury, acute and rapidly progressive glomerulonephritis, acute presentations of nephrotic syndrome, acute pyelonephritis, acute renal failure, idiopathic chronic glomerulonephritis, secondary chronic glomerulonephritis, chronic heart failure, chronic interstitial nephritis, chronic kidney disease (CKD), chronic liver disease, chronic pyelonephritis, diabetes, diabetic kidney disease, fibrosis, focal segmental glomerulosclerosis, Goodpasture's disease, diabetic nephropathy, hereditary nephropathy, interstitial nephropathy, hypertensive nephrosclerosis, IgG4-related renal disease, interstitial inflammation, lupus nephritis, nephritic syndrome, partial obstruction of the urinary tract, polycystic kidney disease, progressive renal disease, renal cell carcinoma, renal fibrosis, graft versus host disease after renal transplant, and vasculitis. The methods provided herein can protect a kidney of a subject from an injury associated with one or more of surgery, radiocontrast imaging, radiocontrast nephropathy, cardiovascular surgery, cardiopulmonary bypass, extracorporeal membrane oxygenation (ECMO), balloon angioplasty, induced cardiac or cerebral ischemic-reperfusion injury, organ transplantation, kidney transplantation, sepsis, shock, low blood pressure, high blood pressure, kidney hypoperfusion, chemotherapy, drug administration, nephrotoxic drug administration, blunt force trauma, puncture, poison, or smoking. The therapeutic composition can be administered in combination with a renal therapeutic agent, such as, for example, those selected from the group comprising dexamethasone, a steroid, budesonide, triamcinolone acetonide, an anti-inflammatory agent, an antioxidant, deferoxamine, feroxamine, a tin complex, a tin porphyrin complex, a metal chelator, ethylenediaminetetraacetic acid (EDTA), an EDTA-Fe complex, dimercapto succinic acid (DMSA), 2,3-dimercapto-1-propanesulfonic acid (DMPS), penicillamine, minocycline, prednisone, azathioprine, mycophenolate mofetil, mycophemolic acid, sirolimius, cyclorsporine, or tacrolimusan antibiotic, an iron chelator, a porphyrin, hemin, vitamin B 12, an Nrf2 pathway activator, bardoxolone, ACE inhibitors, enalapril, glycine polymers, antioxidants, glutathione, N acetyl cysteine, a chemotherapeutic, QPI-1002, QM56, SVT016426 (QM31), 16/86 (third generation ferrostatin), BASP siRNA, CCX140, BIIB023, CXA-10, alkaline phosphatase, Dnmtl inhibitor, THR-184, lithium, formoterol, IL-22, EPO, EPO derivative, agents that stimulate erthyropoietin, epoeitn alfa, darbepoietin alfa, PDGF inhibitor, CRMD-001, Atrasentan, Tolvaptan, RWJ-676070, Abatacept, Sotatercept, an anti-infective agent, an antibiotic, an anti-viral agent, an anti-fungal agent, an aminoglycoside, a nonsteroidal anti-inflammatory drug (NSAID), a diuretic drug, a statin, a senolytic, a corticosteroid, a glucocorticoid, a liposome, renin, angiotensin, ACE inhibitor, mediator of apoptosis, mediator of fibrosis, drug that targets p53, Apaf-1 inhibitor, RIPK1 inhibitor, RIPK3 inhibitor, inhibitor of IL17, inhibitor of IL6, inhibitor of IL23, inhibitor of CCR2, nitrated fatty acids, angiotensin blockers, agonists of the ALK3 receptor, and retinoic acid. The therapeutic composition can be administered in combination with a renal protective agent or a renal prophylactic agent, including, but not limited to, thiazide, bemetanide, ethacrynic acid, furosemidem torsemide, glucose, mannitol, amiloride, spironolactone, eplerenone, triamterene, potassium canrenoate, bendroflumethiazide, hydrochlorothiazide, vasopressin, amphotericin B, acetazolamide, tovaptan, conivaptan, dopamine, dorzolamide, bendrolumethiazide, hydrochlorothiazide, caffeine, theophylline, theobromine, a statin, a senolytic, navitoclax obatoclax, a corticosteroid, prednisone, betamethasone, fludrocortisone, deoxycorticosterone, aldosterone, cortisone, hydrocortisone, belcometasone, mometasone, fluticasone, prednisolone, methylprednisolone, triamcinolone acetonide, a glucocorticoid, dexamethasone, a steroid, budesonide, triamcinolone acetonide, an anti-inflammatory agent, an antioxidant, a nonsteroidal anti-inflammatory drug (NSAID), deferoxamine, iron, tin, a metal, a metal chelate, ethylenediaminetetraacetic acid (EDTA), dimercap to succinic acid (DMSA), 2,3-dimercapto-1-propanesulfonic acid (DMPS), penicillamine, an antibiotic, an aminoglycoside, an iron chelator, a porphyrin, an Nrf2 pathway activator, bardoxolone, ACE inhibitors, enalapril, glycine polymers, antioxidants, glutathione, N-acetyl cysteine, a PDGF inhibitor, lithium, ferroptosis inhibitors, vitamin B 12QPI-1002, QM56, SVT016426 (QM31), 16/86 (third generation ferrostatin), BASP siRNA, CCX140, BIIB023, CXA-10, alkaline phosphatase, Dnmtl inhibitor, THR-184, lithium, formoterol, IL-22, EPO, EPO derivative, agents that stimulate erthyropoietin, epoeitn alfa, darbepoietin alfa, PDGF inhibitor, CRMD-001, Atrasentan, Tolvaptan, RWJ-676070, Abatacept, Sotatercept, an anti-infective agent, an antibiotic, an anti-viral agent, an antifungal agent, an aminoglycoside, a nonsteroidal anti-inflammatory drug (NSAID), a diuretic drug, a statin, a senolytic, a corticosteroid, a glucocorticoid, a liposome, renin, angiotensin, ACE inhibitor, mediator of apoptosis, mediator of fibrosis, drug that targets p53, Apaf-1 inhibitor, RIPK1 inhibitor, RIPK3 inhibitor, inhibitor of IL17, inhibitor of IL6, inhibitor of IL23, inhibitor of CCR2, nitrated fatty acids, angiotensin blockers, agonists of the ALK3 receptor, SGLT2 modulator, and/or retinoic acid. The therapeutic composition can improve one or more markers of kidney function in the subject, such as, for example, those selected from the group comprising reduced blood urea nitrogen (BUN) in the subject, reduced creatinine in the blood of the subject, improved creatinine clearance in the subject, reduced proteinuria in the subject, reduced albumin:creatinine ratio in the subject, improved glomerular filtration rate in the subject, reduced NAG in the urine of the subject, reduced NGAL in the urine of the subject, reduced KIM-1 in the urine of the subject, reduced IL-18 in the urine of the subject, reduced MCP1 in the urine of the subject, reduced CTGF in the urine of the subject; reduced collagen IV fragments in the urine of the subject; reduced collagen III fragments in the urine of the subject; and reduced podocyte protein levels in the urine of the subject, wherein the podocyte protein is selected from nephrin and podocin, reduced cystatin C protein in the blood of a subject, reduced β-trace protein (BTP) in the blood of a subject, and reduced 2-microglobulin (B2M) in the blood of a subject.

### IV. Combination Therapies

In some embodiments, the therapeutic compositions and pharmaceutical formulations including the microRNA antagonists disclosed herein, such as those provided in the Sequence Listing, or those including a combination of the microRNA antagonists disclosed herein, or an expression cassette comprising a nucleotide sequence encoding one or more microRNA antagonists disclosed herein, or a vector comprising one or more of such expression cassettes, can be used in combination with one or more additional therapeutic agents. In some embodiments, the therapeutic compositions and pharmaceutical formulations including the microRNA antagonists disclosed herein, such as those provided in the Sequence Listing, or those including a combination of the microRNA antagonists disclosed herein, or an expression cassette comprising a nucleotide sequence encoding one or more microRNA antagonists disclosed herein, or a vector comprising one or more of such expression cassettes, can be used in combination with one or more therapeutic therapies.

Generally, any therapeutic approach pharmacological or non-pharmacological for muscular dystrophies can be suitably employed as additional therapeutic agents and therapies in the methods disclosed herein. Examples of additional therapeutic agents and therapies that can be used in combination with the microRNA antagonists disclosed herein, or a composition or formulation that include a combination of the microRNA antagonists disclosed herein, or an expression cassette comprising a nucleotide sequence encoding one or more microRNA antagonists disclosed herein, or a vector comprising one or more of such expression cassettes, include, but are not limited to, Idebenone, Eplerenone, VECTTOR, AVI-4658, Ataluren/PTC124/Translarna, BMN044/PRO044, CAT-1004, any gene therapy for MD including MicroDystrophin AAV gene therapy (SGT-001), Galectin-1 therapy (SB-002), LTBB4 (SB-001), rAAV2.5-CMV-minidystrophin, glutamine, NFKB inhibitors, sarcoglycan, delta (35 kDa dystrophin-associated glycoprotein), insulin like growth factor-1 (IGF-1) expression, genome editing through the CRISPR/Cas9 system, any gene delivery therapy aimed at reintroducing a functional recombinant version of the dystrophin gene, Exon skipping therapeutics, read-through strategies for nonsense mutations, cell-based therapies, utrophin upregulation, myostatin inhibition, anti-inflammatories/anti-oxidants, mechanical support devices, any standard therapy for muscular dystrophy, and combinations thereof.

Additional therapeutic agents useful for the methods of the present disclosure also include, but are not limited to, anti-platelet therapy, thrombolysis, primary angioplasty, Heparin, magnesium sulphate, Insulin, aspirin, cholesterol lowering drugs, angiotensin-receptor blockers (ARBs) and angiotensin-converting enzyme (ACE) inhibitors. In particular, ACE inhibitors have clear benefits when used to treat patients with chronic heart failure and high-risk acute myocardial infarction; this is possibly because they inhibit production of inflammatory cytokines by angiotensin II. A non-limiting listing of additional therapeutic agents and therapies includes ACE inhibitors, such as Captopril, Enalapril, Lisinopril, or Quinapril; Angiotensin II receptor blockers, such as Valsartan; Beta-blockers, such as Carvedilol, Metoprolol, and bisoprolol; Vasodilators (via NO), such as Hydralazine, Isosorbide dinitrate, and Isosorbide mononitrate; Statins, such as Simvastatin, Atrovastatin, Fluvastatin, Lovastatin, Rosuvastatin or pravastatin; Anticoagulation drugs, such as Aspirin, Warfarin, or Heparin; or Inotropic agents, such as Dobutamine, Dopamine, Milrinone, Amrinone, Nitroprusside, Nitroglycerin, or nesiritide; Cardiac Glycosides, such as Digoxin; Antiarrhythmic agents, such as Calcium channel blockers, for example, Verapamil and Diltiazem or Class III antiarrhythmic agents, for example, Amiodarone, Sotalol or, defetilide; Diuretics, such as Loop diuretics, for example, Furosemide, Bumetanide, or Torsemide, Thiazide diuretics, for example, hydrochlorothiazide, Aldosterone antagonists, for example, Spironolactone or eplerenone. Alternatively or in addition, other treatments of cardiac disease are also suitable, such as Pacemakers, Defibrillators, Mechanical circulatory support, such as Counterpulsation devices (intraaortic balloon pump or noninvasive counterpulsation), Cardiopulmonary assist devices, or Left ventricular assist devices; Surgery, such as cardiac transplantation, heart-lung transplantation, or heart-kidney transplantation; or immunosuppressive agents, such as Myocophnolate mofetil, Azathiorine, Cyclosporine, Sirolimus, Tacrolimus, Corticosteroids Antithymocyte globulin, for example, Thymoglobulin or ATGAM, OKT3, IL-2 receptor antibodies, for example, Basilliximab or Daclizumab are also suitable.

In some embodiments, at least one of the additional therapeutic agents or therapies includes a biologic drug. In some embodiments, the at least one additional therapeutic agent or therapy comprises a gene therapy or therapeutic gene modulation agent. As used herein, therapeutic gene modulation refers to the practice of altering the expression of a gene at one of various stages, with a view to alleviate some form of ailment. It differs from gene therapy in that gene modulation seeks to alter the expression of an endogenous gene, for example through the introduction of a gene encoding a novel modulatory protein, whereas gene therapy concerns the introduction of a gene whose product aids the recipient directly. Modulation of gene expression can be mediated at the level of transcription by DNA-binding agents, which can be for example, artificial transcription factors, small molecules, or synthetic oligonucleotides. Alternatively or in addition, it can also be mediated post-transcriptionally through RNA interference.

The therapeutic compositions, pharmaceutical formulations disclosed herein and the additional therapeutic agents or therapies can be further formulated into final pharmaceutical preparations suitable for specific intended uses. In some embodiments, the therapeutic composition and the additional therapeutic agent or therapy are administered in a single formulation. In some embodiments, each of the therapeutic composition and the additional therapeutic agent or therapy is administered in a separate formulation. In some embodiments of the methods disclosed herein, the therapeutic composition and/or the additional therapeutic agent or therapy is administered to the subject in a single dose. In some embodiments, the therapeutic composition and/or the additional therapeutic agent or therapy is administered to the subject in multiple dosages. In some embodiments, the dosages are equal to one another. In some embodiments, the dosages are different from one another. In some embodiments, the therapeutic composition and/or the additional therapeutic agent or therapy is administered to the subject in gradually increasing dosages over time. In some embodiments, the therapeutic composition and/or the additional therapeutic agent or therapy is administered in gradually decreasing dosages over time.

The order of the administration of the therapeutic compositions and pharmaceutical formulations, with one or more additional therapeutic agent or therapy, can vary. In some embodiments, a therapeutic composition or pharmaceutical formulation disclosed herein can be administered prior to the administration of all additional therapeutic agent or therapy. In some embodiments, a therapeutic composition or pharmaceutical formulation disclosed herein can be administered prior to at least one additional therapeutic agent or therapy. In some embodiment, a therapeutic composition or pharmaceutical formulation disclosed herein can be administered concomitantly with one or more additional therapeutic agent or therapy. In yet still other embodiments, a therapeutic composition or pharmaceutical formulation disclosed herein can be administered subsequent to the administration of at least one additional therapeutic agent or therapy. In some embodiments, a therapeutic composition or pharmaceutical formulation disclosed herein can be administered subsequent to the administration of all additional therapeutic agent or therapy. In yet some embodiments, a therapeutic composition or pharmaceutical formulation disclosed herein and at least one additional therapeutic agent or therapy are administered in rotation (e.g., cycling therapy). For examples, in some embodiments, a therapeutic composition or pharmaceutical formulation disclosed herein and at least one additional therapeutic agent or therapy are cyclically administered to a subject. Cycling therapy involves the administration of a first active agent or therapy for a period of time, followed by the administration of a second active agent or therapy for a period of time and repeating this sequential administration. Cycling therapy can reduce the development of resistance to one or more therapies, avoid or reduce the side effects of one or more therapies, and/or improve the efficacy of treatment.

In some embodiments, intermittent therapy is an alternative to continuous therapy. For example, intermittent therapy can be used for a period of 6 months on, followed by a period of 6 months off. In some embodiments, one or more therapeutic agents or therapies are provided for one month on, followed by one month off. In some embodiments, one or more therapeutic agents or therapies are provided for three months on, followed by three months off. Accordingly, one or more of the therapeutic compositions or pharmaceutical formulations disclosed herein can be provided before, during and/or after administering one or more additional therapeutic agents or therapies, as described above.

### EXAMPLES

Some aspects of the embodiments discussed above are disclosed in further detail in the following examples, which are not in any way intended to limit the scope of the present disclosure.

### Example 1

### Design of Inhibitory Oligonucleotides for Specific microRNAs

This Example demonstrates the design and composition of synthetic oligonucleotides that can be used as antagonists of miR-99a-5p, miR-100-5p, Let-7a-5p, and Let-7c-5p. Methods and compositions for ameliorating cardiac diseases and/or muscular dystrophy disorders with the microRNA antagonists have been previously disclosed, for example, in U.S. Pat. Pub. No. 2019/0249178, the content of which is hereby expressly incorporated by reference in its entirety.

Nucleotide sequences of the following human microRNAs were analyzed: miR-99a-5p, miR-100-5p, Let-7a-5p and Let-7c-5p. The sequences of these microRNAs and the sequences of the complementary antagonists are shown in Table 1 below. The bases highlighted in bold font correspond to base differences between let-7a-5p and let-7c-5p, or between miR-99a-5p and miR-100-5p. The seed sequence of all microRNAs is generally considered to be bases 2-8 starting from the 5′ end. Without wishing to be bound by any particular theory, the nucleobases within the seed sequence of a microRNA are believed to be the bases that make the biggest contribution to deciding which mRNAs will be targeted by the microRNA. In the sequences listed in Table 1 below, the seed sequences are underlined.

TABLE 1
Nucleotide sequences of human miR-99-5p, miR-100-5p, Let-7a-
5p, and Let-7c-5p and the complementary inhibitory sequences
that can be incorporated into any suitable vectors such as,
for example, viral vector for cardiac muscle generation.

| | |
|---|---|
| >hsa-let-7a-5p MIMAT0000062 | |
| 5′-UGA GGU AGU AGG UUG UAU AGUU-3′ Sense | (SEQ ID NO: 1) |
| 3′-ACU CCA UCA UCC AAC AUA UCAA-5′ Anti-sense | (SEQ ID NO: 2) |
| >hsa-let-7c-5p MIMAT0000064 | |
| 5′-UGA GGU AGU AGG UUG UAU GGUU-3′ Sense | (SEQ ID NO: 3) |
| 3′-ACU CCA UCA UCC AAC AUA CCAA-5′ Anti-sense | (SEQ ID NO: 4) |
| >hsa-miR-99a-5p MIMAT0000097 | |
| 5′-AAC CCG UAG AUC CGA UCU UGUG-3′ Sense | (SEQ ID NO: 5) |
| 3′-UUG GGC AUC UAG GCU AGA ACAC-5 Anti-sense | (SEQ ID NO: 6) |
| >hsa-miR-100-5p MIMAT0000098 | |
| 5′-AAC CCG UAG AUC CGA ACU UGUG-3′ Sense | (SEQ ID NO: 7) |
| 3′-UUG GGC AUC UAG GCU UGA ACAC-5′ Anti-sense | (SEQ ID NO: 8) |

To further assess the sequence conservation of the corresponding microRNA homologs from different mammalian species were also examined. As shown in Table 2 below, the nucleotide sequences of miR-99a-5p, miR-100-5p, Let-7a-5p and Let-7c-5p from different mammalian species were observed to exhibit high degrees of sequence homology. The nucleotide sequences of Let-7a-5p are 100% homologous across all species analyzed. The nucleotide sequences of Let-7c-5p are also 100% homologous across all species analyzed. The sequence of miR-99a-5p from dog lacks nucleobase #, otherwise all other sequences are homologous. Dog is missing miR-100 miRNA, otherwise all other sequences are homologous.

TABLE 2
Sequence homology of miR-99a-5p, miR-100-5p, Let-7a-5p and
Let-7c-5p homologs. Dre: Danio rerio (zebrafish),
Hsa: Homo sapiens (human), Ptr : Pan troglodytes
(chimpanzee), Cfa: Canis familiaris (dog), Ssc: Sus scrofa
(minipig), Rno: Rattus norvegicus (rat), Mmu: Mus musculus
(mouse).

| | | | |
|---|---|---|---|
| dre-let-7a-5p | UGAGGUAGUAGGUUGUAUAGUU | | (SEQ ID NO: 9) |
| mmu-let-7a-5p | UGAGGUAGUAGGUUGUAUAGUU | | (SEQ ID NO: 10) |
| rno-let-7a-5p | UGAGGUAGUAGGUUGUAUAGUU | | (SEQ ID NO: 11) |
| ssc-let-7a-5p | UGAGGUAGUAGGUUGUAUAGUU | | (SEQ ID NO: 12) |
| ptr-let-7a-5p | UGAGGUAGUAGGUUGUAUAGUU | | (SEQ ID NO: 13) |
| hsa-let-7a-5p | UGAGGUAGUAGGUUGUAUAGUU | | (SEQ ID NO: 14) |
| cfa-let-7a-5p | UGAGGUAGUAGGUUGUAUAGUU | | (SEQ ID NO: 15) |
| dre-let-7c-5p | UGAGGUAGUAGGUUGUAUGGUU | | (SEQ ID NO: 16) |
| mmu-let-7c-5p | UGAGGUAGUAGGUUGUAUGGUU | | (SEQ ID NO: 17) |
| rno-let-7c-5p | UGAGGUAGUAGGUUGUAUGGUU | | (SEQ ID NO: 18) |
| ssc-let-7c-5p | UGAGGUAGUAGGUUGUAUGGUU | | (SEQ ID NO: 19) |
| ptr-let-7c-5p | UGAGGUAGUAGGUUGUAUGGUU | | (SEQ ID NO: 20) |
| hsa-let-7c-5p | UGAGGUAGUAGGUUGUAUGGUU | | (SEQ ID NO: 21) |
| cfa-let-7c-5p | UGAGGUAGUAGGUUGUAUGGUU | | (SEQ ID NO: 22) |
| dre-miR-99a-5p | AACCCGUAGAUCCGAUCUUGUG | 22 | (SEQ ID NO: 23) |
| mmu-miR-99a-5p | AACCCGUAGAUCCGAUCUUGUG | 22 | (SEQ ID NO: 24) |
| rno-miR-99a-5p | AACCCGUAGAUCCGAUCUUGUG | 22 | (SEQ ID NO: 25) |
| cfa-miR-99a | AACCCGUAGAUCCGAUCUUGU | 21 | (SEQ ID NO: 26) |
| ssc-miR-99a | AACCCGUAGAUCCGAUCUUGUG | 22 | (SEQ ID NO: 27) |
| ptr-miR-99a | AACCCGUAGAUCCGAUCUUGUG | 22 | (SEQ ID NO: 28) |
| hsa-miR-99a-5p | AACCCGUAGAUCCGAUCUUGUG | 22 | (SEQ ID NO: 29) |
| dre-miR-100-5p | AACCCGUAGAUCCGAACUUGUG | | (SEQ ID NO: 30) |
| mmu-miR-100-5p | AACCCGUAGAUCCGAACUUGUG | | (SEQ ID NO: 31) |
| rno-miR-100-5p | AACCCGUAGAUCCGAACUUGUG | | (SEQ ID NO: 32) |
| ssc-miR-100 | AACCCGUAGAUCCGAACUUGUG | | (SEQ ID NO: 33) |
| ptr-miR-100 | AACCCGUAGAUCCGAACUUGUG | | (SEQ ID NO: 34) |
| hsa-miR-100-5p | AACCCGUAGAUCCGAACUUGUG | | (SEQ ID NO: 35) |

A total of twenty (20) anti-miR oligonucleotide compounds were designed, including ten for the let-7a-5p/let-7c-5p family and ten for the miR-99a-5p/miR-100-5p family. Two anti-miR designs targeting Let-7c-5p are JRX0100, JRX0102 and could be used to inhibit Let-7a-5p. Two anti-miR designs targeting Let-7a-5p are JRX0101 and JRX0103 and could be used to inhibit Let-7a-5p. Six anti-miR designs targeting both let-7a-5p and Let-7c-5p are JRX0104, JRX0105, JRX0106, JRX0107, JRX0108, and JRX0109. Five anti-miR designs targeting miR-100a are JRX0110, JRX0113, JRX0115, JRX0117, and JRX0119. Five anti-miR designs targeting miR-99a are JRX0111, JRX0112, JRX0114, JRX0116, and JRX0118. In this experiment, the designs used locked nucleic acid (LNA) chemistry modifications (+), in which the 2′-O-oxygen is bridged to the 4′ position via a methylene linker to form a rigid bicycle, locked into a C3′-endo (RNA) sugar conformation allowing for resistance to nuclease degradation and extremely high affinity for its complementary RNA base. These modifications were particularly incorporated at each end of the molecules as designated by (+) in the sequences in Table 3 for stability, by e.g. enhancing resistance to exonucleases, and in the region complementary to the seed to increase affinity for their targeted miR and thus increased potency as a microRNA inhibitor. The backbone of the anti-miRs is phosphorothioate (indicated by * in Table 3 below) to enable a broad distribution in animals. This type of backbone functions by steric blockade of a specific microRNA in the RISC complex. The anti-miR oligonucleotide compounds were carefully kept relatively short, to avoid the possible of forming heteroduplexes, but long enough to bind plasma proteins efficiently and keep them from being filtered out of circulation in the kidneys and thus improve their biodistribution properties. A summary of 20 anti-miR designs and their respective target microRNAs is shown in Table 3 below.

As indicated in Table 3, some of the miR-7 family anti-miRs are 100% homologous to both let-7c-5p and c isoforms of interest and will inhibit both members. In contrast, the miR-99a-5p and miR-100 family anti-miRs are each only 100% homologous to one of the family members due to the position of the one base that is different in these miRs. However, in reality all of the anti-miRs designed for each of the two families can inhibit both members of the family of interest because, similarly to target recognition, the seed region (bases 2-8) is the most important region for determining anti-miR activity.

TABLE 3
Summary of twenty anti-miR designs disclosed herein

| | | | | | No. | | | |
|---|---|---|---|---|---|---|---|---|
| | | | No. | | LNAs | | Nomenclature/ | SEQ |
| | | | LNAs | Stretch | in Seed | Nomenclature/Sequence/ | Sequence/ | ID |
| Name | Target | Length | PLUS | of DNA | * | Structure | Structure | NO |
| JRX0100 | let-7c | 19 | 9 | 3 | 5 | +C*+C*A*T*+A*C*A*A*+C*C*T | CCATACAACCTA | 36 |
| | | | | | | *A*+C*T*+A*C*+C*+T*+C | CTACCTC | |
| JRX0101 | let-7a | 19 | 9 | 3 | 5 | +C*+T*A*T*A*+C*A*A*C*+C*T | CTATACAACCTA | 37 |
| | | | | | | *A*+C*+T*A*C*+C*+T*+C | CTACCTC | |
| JRX0102 | let-7c | 18 | 9 | 3 | 5 | +C*+A*T*A*C*A*+A*C*C*T*A* | CATACAACCTAC | 38 |
| | | | | | | +C*T*+A*+C*C*+T*+C | TACCTC | |
| JRX0103 | let-7a | 18 | 9 | 3 | 5 | +T*+A*T*A*C*+A*A*C*+C*T*A | TATACAACCTACT | 39 |
| | | | | | | *C*+T*+A*+C*C*+T*+C | ACCTC | |
| JRX0104 | let-7a/c | 17 | 9 | 3 | 5 | +A*T*A*C*A*+A*C*C*+T*A*+C | ATACAACCTACT | 40 |
| | | | | | | *T*+A*+C*C*+T*+C | ACCTC | |
| JRX0105 | let-7a/c | 17 | 9 | 3 | 5 | +A*+T*A*C*A*+A*C*+C*T*A*+ | ATACAACCTACT | 41 |
| | | | | | | C*T*+A*C*+C*+T*+C | ACCTC | |
| JRX0106 | let-7a/c | 16 | 8 | 3 | 5 | +T*+A*C*A*A*+C*C*T*A*+C*T | TACAACCTACTA | 42 |
| | | | | | | *+A*+C*C*+T*+C | CCTC | |
| JRX0107 | let-7a/c | 16 | 8 | 3 | 5 | +T*+A*C*A*A*+C*C*T*A*+C*T | TACAACCTACTA | 43 |
| | | | | | | *+A*C*+C*+T*+C | CCTC | |
| JRX0108 | let-7a/c | 15 | 8 | 3 | 5 | +A*+C*A*A*C*+C*T*A*+C*T*+ | ACAACCTACTAC | 44 |
| | | | | | | A*+C*C*+T*+C | CTC | |
| JRX0109 | let-7a/c | 15 | 9 | 3 | 6 | +A*+C*A*A*+C*C*T*A*+C*+T* | ACAACCTACTAC | 45 |
| | | | | | | +A*C*+C*+T*+C | CTC | |
| JRX0110 | miR- | 19 | 9 | 3 | 5 | +C*+A*A*G*+T*T*C*G*+G*A*T | CAAGTTCGGATC | 46 |
| | 100 | | | | | *C*+T*A*+C*G*+G*+G*+T | TACGGGT | |
| JRX0111 | miR-99 | 19 | 9 | 3 | 5 | +C*+A*A*G*A*+T*C*G*G*+A*T | CAAGATCGGATC | 47 |
| | | | | | | *C*+T*+A*C*G*+G*+G*+T | TACGGGT | |
| JRX0112 | miR-99 | 18 | 9 | 3 | 5 | +A*+A*G*+A*T*C*G*+G*A*T*C | AAGATCGGATCT | 48 |
| | | | | | | *+T*A*+C*+G*G*+G*+T | ACGGGT | |
| JRX0113 | miR- | 18 | 9 | 3 | 5 | +A*+A*G*T*T*+C*G*G*+A*T*C | AAGTTCGGATCT | 49 |
| | 100 | | | | | *T*+A*+C*+G*G*+G*+T | ACGGGT | |
| JRX0114 | miR-99 | 17 | 9 | 3 | 5 | +A*+G*A*T*C*+G*G*A*+T*C*+ | AGATCGGATCTA | 50 |
| | | | | | | T*A*+C*+G*G*+G*+T | CGGGT | |
| JRX0115 | miR- | 17 | 9 | 3 | 5 | +A*+G*T*T*C*+G*G*+A*T*C*+ | AGTTCGGATCTA | 51 |
| | 100 | | | | | T*A*+C*G*+G*+G*+T | CGGGT | |
| JRX0116 | miR-99 | 16 | 8 | 3 | 5 | +G*+A*T*C*G*+G*A*T*C*+T*A | GATCGGATCTAC | 52 |
| | | | | | | *+C*+G*G*+G*+T | GGGT | |
| JRX0117 | miR- | 16 | 8 | 3 | 5 | +G*+T*T*C*G*+G*A*T*C*+T*A | GTTCGGATCTAC | 53 |
| | 100 | | | | | *+C*G*+G*+G*+T | GGGT | |
| JRX0118 | miR-99 | 15 | 8 | 3 | 5 | +A*+T*C*G*G*+A*T*C*+T*A*+ | ATCGGATCTACG | 54 |
| | | | | | | C*+G*G*+G*+T | GGT | |
| JRX0119 | miR- | 15 | 9 | 3 | 6 | +T*+T*C*G*+G*A*T*C*+T*+A* | TTCGGATCTACG | 55 |
| | 100 | | | | | +C*G*+G*+G*+T | GGT | |

As described in further detail below, the inhibitory activity of these synthetic anti-miRs can be subsequently assessed by using a commercially reporter vector system, pMIR-REPORT™ miRNA Expression Reporter Vector System, made available by Applied Biosystems® (Part Number AM5795, Applied Biosystems). In this system, microRNA binding sites of interest are inserted the multiple cloning sites located downstream of the coding sequence of the reporter luciferase.

### Example 2

### Design of Adeno-Viral Vector JBT-miR1

This Example summarizes experimental results illustrating the design of a modified hairpin Zip construct and vector expressing inhibitory sequences of the microRNAs miR-99a, miR-100-5p, miR-Let-7a-5p, and miR-Let-7c-5p using RNAi technology. In this experiment, RNAi technology was implemented within a target cell in the form of a base-pair short hairpin (sh) RNA (shRNA), which is processed into an approximately 20 base pair small interfering RNA through the endogenous miR pathway. A small hairpin RNA or short hairpin RNA (shRNA) is typically defined as an artificial RNA molecule with a tight hairpin turn that can be used to silence target gene expression via RNA interference (RNAi). To evaluate the potential therapeutic use of anti-miR-99/100 and anti-Let-7a/c to regenerate cardiac muscle in the murine heart, two recombinant viruses expressing complementary inhibitory sequences to Let-7a/c and miR-99/100 were made by AAV2 Inverted Terminal Repeat (ITR) sequences cross packaged into AAV9 capsids (AAV2/9). The AAV2/9 serotype has clear cardiac tropism. Viral delivery of complementary sequences to miRs is a common approach. In this experiment, AAV vectors were selected as being optimal in cardiovascular gene therapy since they a) contain no viral protein-coding sequences to stimulate an immune response, b) do not require active cell division for expression to occur and c) have a significant advantage over adenovirus vectors because of their stable, long-term expression of recombinant genes in cardiomyocytes in vivo.

In this experiment, a modified hairpin Zip construct expressing (1) the Let-7a-5p and miR-99a-5p inhibitory sequences under the H1 promoter and U6 promoter, respectively; and (2) Let-7c-5p and miR-100-5p inhibitory sequences under the regulation of the H1 promoter and U6 promoter, respectively. A summary of the nucleotide sequences of anti-miR antagonists and loop sequence inserted into the pAV-4 in1shRNA-GFP vector to generate the viral vector JBT-miR1 is provided in Table 4 below. In this experiment, the nucleotide sequences encoding the foregoing antagonists were cloned in the pAV-4in1shRNA-GFP vector. The nucleotide sequences corresponding to the four miR inhibitory sequences were inserted into the pAV-4inlshRNA-GFP vector between the ITR sites of the vector and specifically within the BamH1 and HindIII cloning site, and were separated by a loop sequence, TGTGCTT (SEQ ID NO: 56). In the resulting vector, expression of each inhibitory sequence was regulated by alternate human U6 promoter or the H1 promoter driving the expression of a short hairpin RNA (shRNA) against miR-99a-5p, 100, Let-7a-5p and Let-7c.

Also inserted into the vector was a CMV promoter driving the expression of a Green Fluorescent Protein (GFP) reporter, which in turn allows for detection in various tissues for preclinical studies, followed by a Simian virus 40 (SV40) sequence which is a polyomavirus binding site that initiates DNA replication at the origin of replication allowing for replication of in mammalian cells expressing SV40 large T. It is contemplated however that, these sequences can also be suitably removed from vectors designed for use in human drugs.

Vector genomes with AAV2 ITR sequences were cross-packaged into AAV9 capsids via triple transfection of AAV-293 cells (J. Fraser Wright, Human Gene Therapy, 20:698-706, July 2009), and then purified by iodixanol gradient centrifugation. Titers of the AAV vectors, which is defined as viral genomes (vg)/ml, were then determined by a qPCR-based assay. In this experiments, the following primers were used for amplifying the mouse U6 promoter: 5′-TCGCACAGACTTGTGGGAGAA-3′ (SEQ ID NO: 57) (forward) and 5′ CGCACATTAAGCCTCTATAGTTACTAGG-3′ (SEQ ID NO: 58) (reverse).

Known copy numbers of plasmids carrying the corresponding expression cassettes were used to construct standard curves for quantification. The virus was manufactured and sequenced by Vigene Biosciences Inc. (Rockville, Md.) using manufacturer's recommended safety precautions and procedures.

TABLE 4
Summary of the nucleotide sequences of anti-miR antagonists and
loop sequence inserted into the BamH1 and HindIII cloning site
of the pAV-4in1shRNA-GFP vector to generate the viral vector
JBT-miR1.

| Target | Hairpin | SEQ ID NO |
|---|---|---|
| let-7a-5p | GTGAGGTAGTAGGTTGTATAGTTTCAAGAGAAC | 59 |
| | TATACAACCTACTACCTCATTTTT | |
| miR-99a-5p | GAACCCGTAGATCCGATCTTGTGTCAAGAGCAC | 60 |
| | AAGATCGGATCTACGGGTTTTTTT | |
| (H1-)let-7a-5p & | GTGAGGTAGTAGGTTGTATAGTTTCAAGAGAAC | 61 |
| (U6)-miR-99a- | TATACAACCTACTACCTCATTTTTGAGCTCAAAA | |
| 5p | AAACCCGTAGATCCGATCTTGTGCTCTTGACACA | |
| | AGATCGGATCTACGGGTTC | |
| let-7c-5p | GTGAGGTAGTAGGTTGTATGGTTTCAAGAGAAC | 62 |
| | CATACAACCTACTACCTCATTTTT | |
| miR-100-5p | GAACCCGTAGATCCGAACTTGTGTCAAGAGCAC | 63 |
| | AAGTTCGGATCTACGGGTTTTTTT | |
| (H1-)let-7C-5p | GTGAGGTAGTAGGTTGTATGGTTTCAAGAGAAC | 64 |
| & (U6)-miR- | CATACAACCTACTACCTCATTTTTGAGCTCAAAA | |
| 100-5p | AAACCCGTAGATCCGAACTTGTGCTCTTGACAC | |
| | AAGTTCGGATCTACGGGTTC | |

The nucleotide sequence of the JBT-miR1 viral vector design is set forth at SEQ ID NO: 85 in the Sequence Listing.

As described in Example 1 above, a total of twenty (20) anti-miR oligonucleotide compounds were designed. The sequences of these anti-miR oligonucleotide compounds are shown in Table 5 below. Any combination of the sequences of anti-miR oligonucleotide compounds disclosed in Table 5 below can be inserted into the BamH1 and HindIII cloning site of the pAV-4in1shRNA-GFP vector to generate other viral delivery systems for miR-99a, miR-100-5p, Let-7a-5p and Let-7c-5p inhibition.

TABLE 5

| | | No. | | No. | | SEQ |
|---|---|---|---|---|---|---|
| | | LNAs | Stretch | LNAs in | | ID |
| Target | Length | PLUS | of DNA | Seed * | Sequence | NO |
| let-7c | 19 | 9 | 3 | 5 | CCATACAACCTACTACCTC | 65 |
| let-7a | 19 | 9 | 3 | 5 | CTATACAACCTACTACCTC | 66 |
| let-7c | 18 | 9 | 3 | 5 | CATACAACCTACTACCTC | 67 |
| let-7a | 18 | 9 | 3 | 5 | TATACAACCTACTACCTC | 68 |
| let-7a/c | 17 | 9 | 3 | 5 | ATACAACCTACTACCTC | 69 |
| let-7a/c | 17 | 9 | 3 | 5 | ATACAACCTACTACCTC | 70 |
| let-7a/c | 16 | 8 | 3 | 5 | TACAACCTACTACCTC | 71 |
| let-7a/c | 16 | 8 | 3 | 5 | TACAACCTACTACCTC | 72 |
| let-7a/c | 15 | 8 | 3 | 5 | ACAACCTACTACCTC | 73 |
| let-7a/c | 15 | 9 | 3 | 6 | ACAACCTACTACCTC | 74 |
| miR-100 | 19 | 9 | 3 | 5 | CAAGTTCGGATCTACGGGT | 75 |
| miR-99 | 19 | 9 | 3 | 5 | CAAGATCGGATCTACGGGT | 76 |
| miR-99 | 18 | 9 | 3 | 5 | AAGATCGGATCTACGGGT | 77 |
| miR-100 | 18 | 9 | 3 | 5 | AAGTTCGGATCTACGGGT | 78 |
| miR-99 | 17 | 9 | 3 | 5 | AGATCGGATCTACGGGT | 79 |
| miR-100 | 17 | 9 | 3 | 5 | AGTTCGGATCTACGGGT | 80 |
| miR-99 | 16 | 8 | 3 | 5 | GATCGGATCTACGGGT | 81 |
| miR-100 | 16 | 8 | 3 | 5 | GTTCGGATCTACGGGT | 82 |
| miR-99 | 15 | 8 | 3 | 5 | ATCGGATCTACGGGT | 83 |
| miR-100 | 15 | 9 | 3 | 6 | TTCGGATCTACGGGT | 84 |

The nucleotide sequence of the JBT-miR1 viral vector design is set forth at SEQ ID NO: 85 in the Sequence Listing.

### Example 3

### Design of Viral Vector JBT-miR2

This Example summarizes experimental results illustrating the design of another viral vector, named JBT-miR2, which expresses tough decoys (also known as TuDs) that can be superior to zips (JBT-miR1) (Takeshi et al. 2009). In brief, four 120-based oligonucleotide sequences were inserted into between the ITR sites of the vector and in the BamH1 and HindIII cloning site to generate the TuDs that can inhibit the let-7 and miR-99a-5p families when inserted into a viral delivery system. In the nucleotide sequences of the foregoing oligonucleotides shown below, bold characters correspond to the respective miR binding sites.

let-7a-5p
(SEQ ID NO: 86)
GACGGCGCTAGGATCATCAACAACTATACAACCAATGTACTACCTCACAA
GTATTCTGGTCACAGAATACAACAACTATACAACCAATGTACTACCTCAC
AAGATGATCCTAGCGCCGTC.
let-7a-5p Reverse Complement
(SEQ ID NO: 87)
GACGGCGCTAGGATCATCTTGTGAGGTAGTACATTGGTTGTATAGTTGTT
GTATTCTGTGACCAGAATACTTGTGAGGTAGTACATTGGTTGTATAGTTG
TTGATGATCCTAGCGCCGTC
miR-99a-5p
(SEQ ID NO: 88)
GACGGCGCTAGGATCATCAACCACAAGATCGGAAATGTCTACGGGTACAA
GTATTCTGGTCACAGAATACAACCACAAGATCGGAAATGTCTACGGGTAC
AAGATGATCCTAGCGCCGTC
miR-99a-5p Reverse Complement
(SEQ ID NO: 89)
GACGGCGCTAGGATCATCTTGTACCCGTAGACATTTCCGATCTTGTGGTT
GTATTCTGTGACCAGAATACTTGTACCCGTAGACATTTCCGATCTTGTGG
TTGATGATCCTAGCGCCGTC.

In some experiments, restriction sites were added to the oligonucleotides which in turn facilitate their subcloning into the appropriate vectors. The 5′ end of these sequences were cloned adjacent to the promoter sequence (e.g., the U6 promoter) and the 3′ end was cloned against a PolII termination sequence (e.g., TTTTT).

### Example 4

### Use of MiRNA Inhibitors to Mitigate Cardiac Ischemic Reperfusion Injury

This Example summarizes experimental results illustrating methods using the compositions provided herein for the mitigation of cardiac ischemic reperfusion injury.

In the developed world Ischemic heart disease (IHD) is the main cause of death for both males and females. IHD can be caused by an acute myocardial infarction (MI) which limits sufficient blood flow to the heart leading to irreversible death of cardiomyocytes. Scar formation following permanent cardiomyocyte death is associated with progressive heart function deterioration in most patients along with a significant increased probability of subsequent cardiovascular events and mortality. Despite a broad therapeutic arsenal to treat an MI, most importantly timely coronary interventions in association with thrombolytic and antiplatelet therapy that restore arterial perfusion, many patients fail to recover full cardiac function because current treatments do not promote regeneration of heart muscle and prevent the transition to heart failure (HF). Investigative treatments, such as exogenous stem cell therapy approaches have been extensively explored, but have not led to clinical translation for a multitude of reasons, including the inability of exogenous stem cells to integrate with the damaged myocardium, rejection of cells, inconsistencies in stem cell manufacture, clinical study design and execution, complications and inadequate, unbiased preclinical, unbiased testing prior to clinical translation. Hence alternative strategies that induce endogenous cardiac muscle regeneration are being investigated as novel therapeutic approaches to MI Injury.

Amongst these new approaches are therapeutics targeting miRNAs which are key regulators in almost all biological processes. MicroRNAs (miRNAs) consist of non-coding RNA molecules that are 21 to 24 nucleotides in length and function in RNA silencing and post transcriptional regulation of gene expression and act via base-pairing with complementary sequences within mRNA molecules, silencing the mRNA, and modulating target protein expression and downstream signaling pathways.

Studies have shown that adult mice can regenerate their heart muscle by a process which occurs by dedifferentiation of mature cardiomyocytes followed by proliferation and re-differentiation by the targeting of specific miRNAs. This process illustrates two important facts: 1) endogenous cells within the mammalian heart represent a larger and more efficient pool of regenerative precursors than exogenous stem cells and 2) regeneration is an innate property of mammalian hearts and can lead to functional recovery, albeit inefficiently, in adults. A number of miRNAs have been shown to regulate endogenous regeneration of cardiomyocytes. The miRNAs hsa-miR-199a-3p and hsa-miR-590-3p were shown to stimulate cardiomyocyte proliferation and improve cardiac function in response to MI. In another study, the function of miRNAs in regulating cardiomyocyte proliferation and heart regeneration was linked to the Hippo/Yap pathway, in which members of the miR302-367 cluster directly target key components of the Hippo/Yap pathway. Both miR-34a and the miR-17-92 cluster of miRNAs have been shown to regenerate cardiomyocytes in mice after an MI. Others have shown that loss of miR-128 promotes cardiac muscle regeneration in the mouse and loss of miR-15 protects against MI injury in both mice and pigs. To note miR-15 is no longer being developed as a treatment for heart muscle regeneration and suggests that targeting a single miRNA may not have a therapeutic effect on heart and is supported by the fact that over 60 miRNAs have significant expression changes in lower vertebrate heart regeneration.

One detailed study by Aitor et al. identified that four miRs: miR-99, miR-100, let-7a and let-7c which are clustered in two well-defined genomic locations are strongly down regulated in expression during Zebrafish heart regeneration. Unlike others, this paper highlighted that a combination of inhibition of four miRNAs is necessary for mammalian heart muscle regeneration after an MI. This finding is supported by a common role for the miR-99a/let-7c cluster in regulating vertebrate cardiomyogenesis with epigenetic remodeling and cell cycle control the two key steps controlling this regenerative process. MIRANDA-based miR-UTR binding predictions showed a strong interaction for miR-99/100 with zebrafish FNTβ (beta subunit of farnesyl-transferase) and SMARCA5 (SWI/SNF-related matrix associated actin-dependent regulator of chromatin subfamily a, member 5), linking the miR-let-7a/c, miR-99/100 families to cell cycle and epigenetic control in cardiomyocytes. Interestingly miR-99/100 and let-7a/c levels are low during early mammalian heart development and promote quick cardiac mass growth, but increase exponentially during late development, with a corresponding decrease in FNTβ and SMARCA5 protein levels to block further cardiomyocyte proliferation. Postmortem analysis of injured human heart tissue, suggests that these miRs constitute a conserved roadblock to cardiac regeneration in adults. Modified zip construct inhibitors to miR-99/100 and let-7a/c were cloned into two adeno associated viruses (AAV2/9), and injected Intra-myocardial at a dose of 1×10¹¹ viral genomes (vg) to adult mice with a permanent MI. The viral delivered inhibitors permitted dedifferentiation and proliferation and re-differentiation cycling of Cardiomyocytes leading to increased fractional shortening (FS), ejection fraction (EF) and Left ventricular anterior wall (LVAW) thickness 90 days post-MI with a reduction in scarring.

By leveraging the regenerative capabilities of cardiomyocytes, as a translational, therapeutic approach, an optimized single virus (JBT-miR2) was developed that delivers two transcribed miR-binding RNAs for let-7a/c and miR-99/100, known as tough decoys (TuDs) to the heart to permit cardiomyocyte regeneration. TuDs consist of artificial single strands of RNA with one antisense miR binding domain (Decoy) or a stabilized stem-loop with two miR binding domains that sequester the miRNA into stable complexes through complementary base pairing. This disables a particular RNA interference pathway, acting in part, by targeting miRs for destruction by recruiting the tailing and trimming pathway to decrease target miR steady-state abundance. In addition to a viral delivered therapeutic strategy, this example describes the design and testing of two synthetic oligonucleotide antagomiRs to miR-99/100 and let-7a/c, known as JN-101. JN-101 oligonucleotides possess a Lock Nucleic Acid (LNA) configuration and are stable in the blood stream, resistant to degradation and inhibit the miRNA via the RISC complex. JN-101 improves wall motion and heart function in mice with IR injury. In mice with a transient IR injury, this example demonstrates with comprehensive functional global and regional cardiac imaging together with histological and biomarker data that combined inhibition of miR-99/miR-100 and let-7a/c mitigates cardiac muscle injury in mice with IR injury leading to increased heart function. Thus, the methods and compositions provided herein can be translational therapeutics that can be administered with standard of care following an MI.

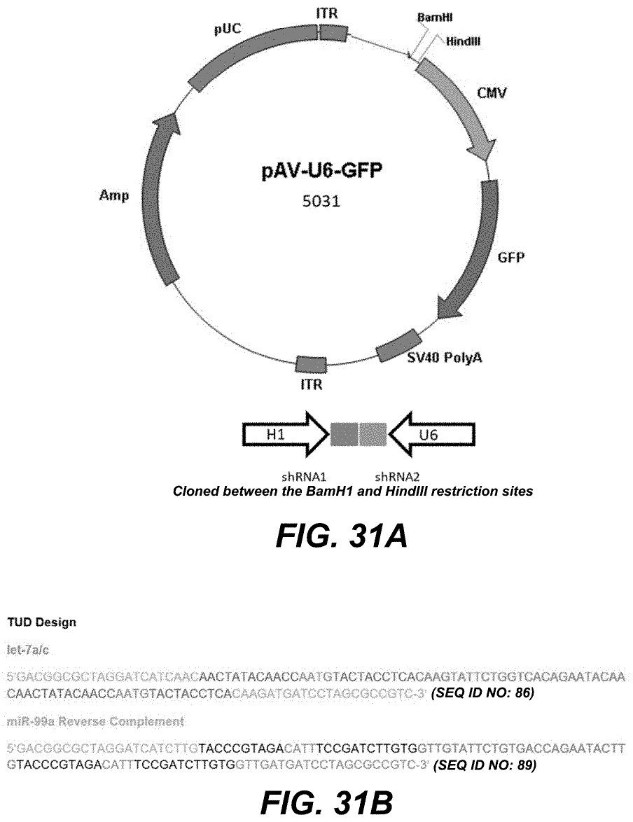

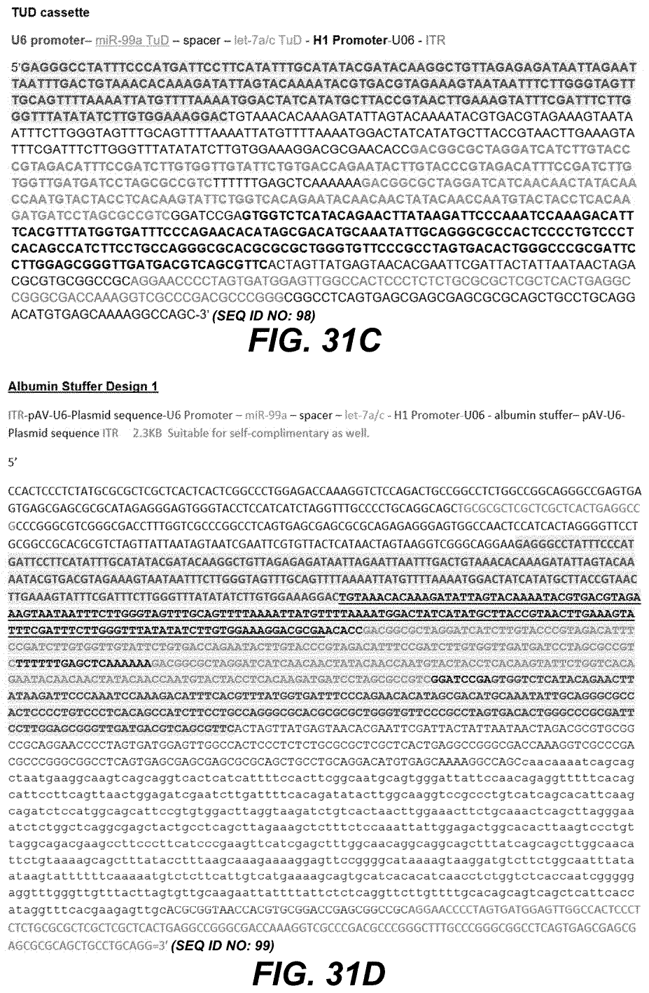

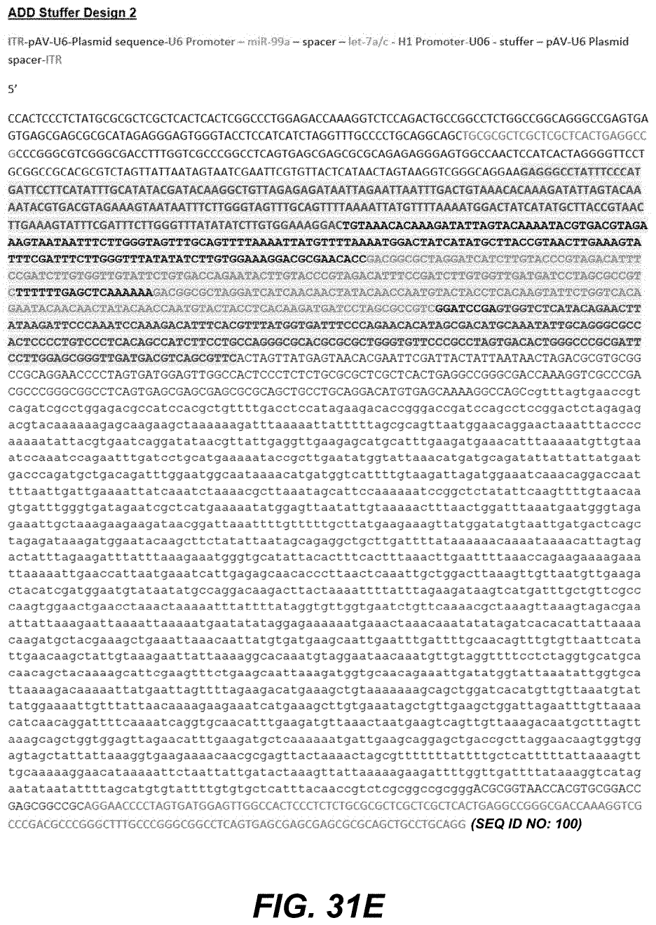

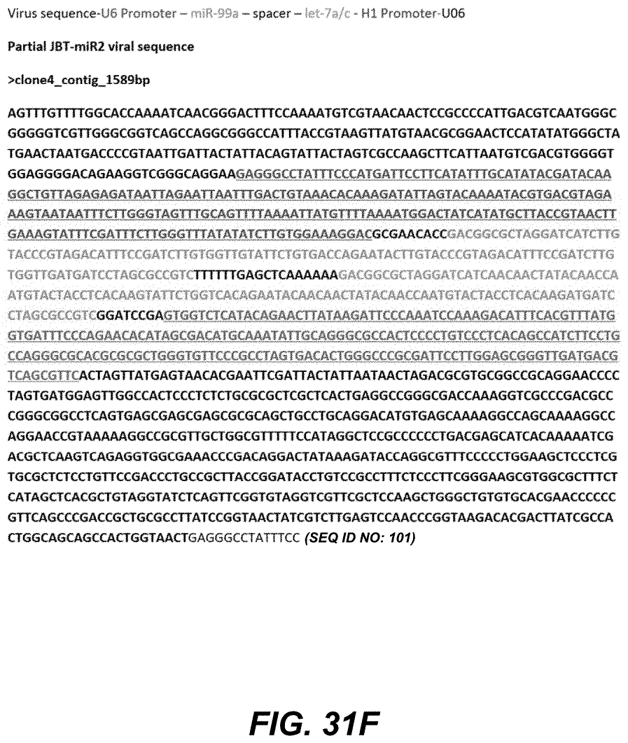

In some embodiments of the compositions provided herein, the pAV-U6-GFP vector was used as the basic cloning vector. In some embodiments, inserted into the pAV-U6-GFP vector in the BamH1 and HindIII cloning site are the two TuD inhibitor sequences separated by a loop sequence, TGTGCTT. Each inhibitor can be regulated by alternate human U6 or the H1 promoters that drive the expression of, for example, the miR-99/100 and Let-lac TuDs, cloned between the two AAV2 ITRs. Vector genomes with AAV2 ITR sequences were crosspackaged into AAV9 capsids via triple transfection of AAV-293 cells, then purified by ammonium sulfate fractionation and iodixanol gradient centrifugation. Titers of the AAV vectors [viral genomes (vg)/ml] were determined by qPCR to the two ITR sequences. Known copy numbers of plasmids carrying the corresponding expression cassettes were used to construct standard curves for quantification. FIGS. 31A-31F depict non-limiting exemplary schematics regarding the design of compositions provided herein. FIG. 31A depicts the pAV-U6-GFP vector and insert employed in some of the compositions provided herein (e.g., JBT-miR2). FIG. 31B depicts non-limiting exemplary sequences employed in the design of TuDs provided herein (SEQ ID NOS: 86 and 89). FIG. 31C depicts a non-limiting exemplary TuD cassette that was inserted into pAV-U6 GFP (SEQ ID NO: 98). One or more of the TUD cassettes can be inserted into a cloning or expression vector described herein (e.g., cloned between the two ITR sequences). More than one TuD cassette can be inserted between the ITRs and, in some embodiments, can decrease the amount of virus that needs to be administered to subjects described herein. In some embodiments of the compositions provided herein (e.g., preclinical and clinical translation), the CMV promoter driving GFP and the SV40 termination sequence will be removed and inserted with “Stuffer DNA” (e.g., sequences shown in FIGS. 31D-31E). FIG. 31D depicts Albumin Stuffer Design 1 (SEQ ID NO: 99) and FIG. 31E depicts ADD Stuffer Design 2 (SEQ ID NO: 100). FIG. 31F depicts a portion of the nucleotide sequence of JBT-miR2 (SEQ ID NO: 101).

### Methods

### Viral and Inhibitor Design and In Vitro Infection Experiments

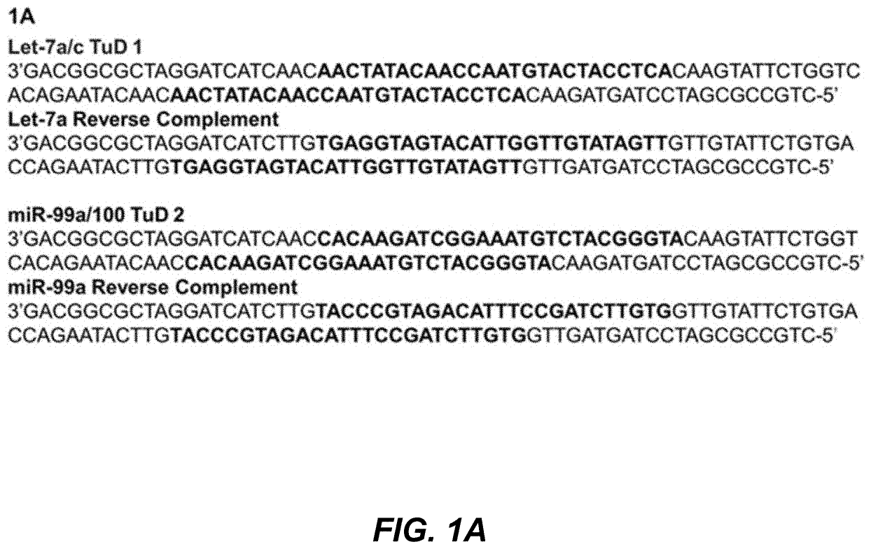

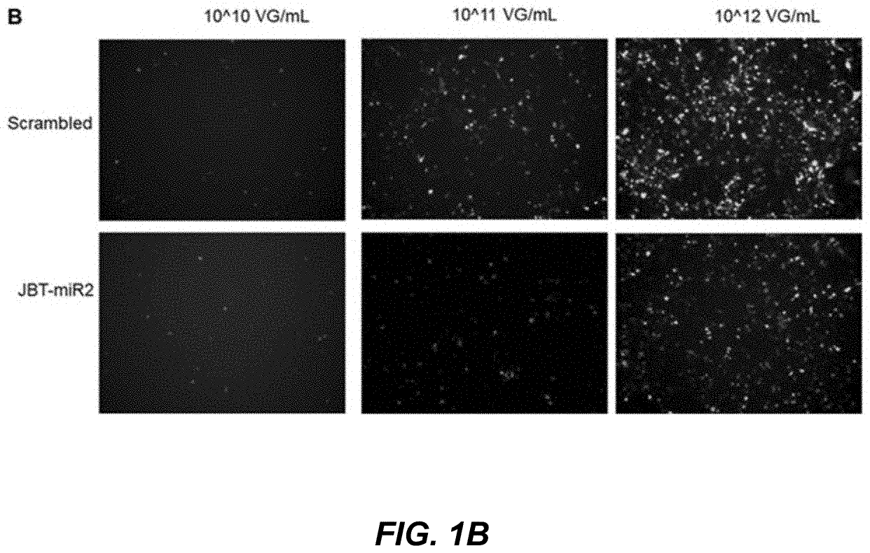

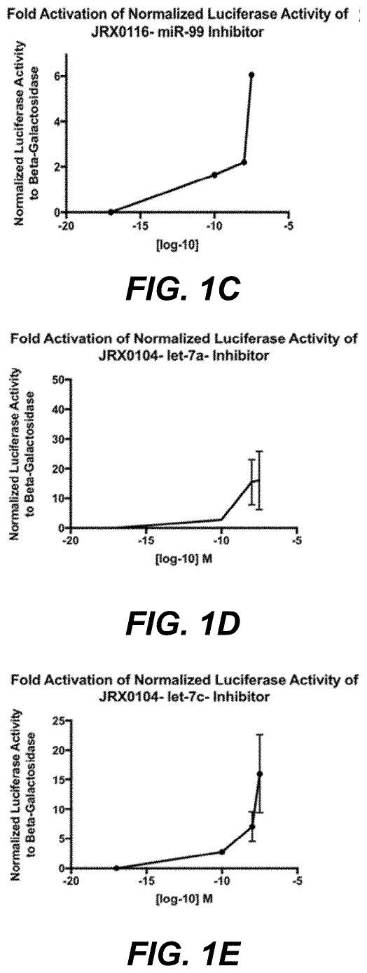

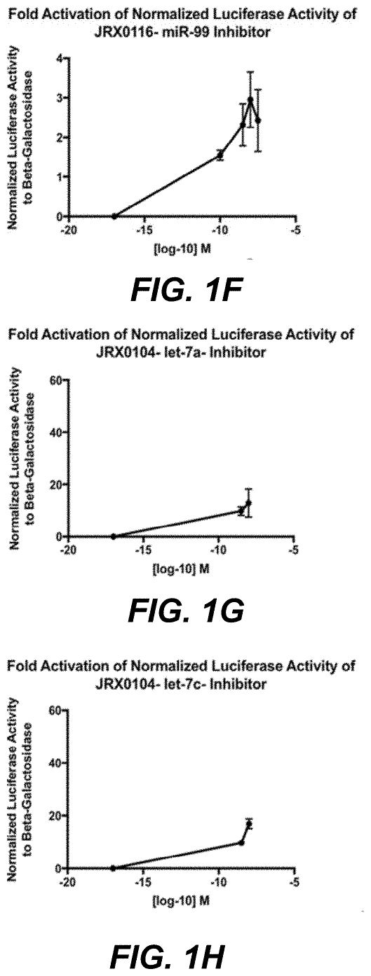

TuDs can comprise artificial single strands of RNA with one antisense miR binding domain (Decoy) or a stabilized stem-loop with two miR binding domains that sequester the miR into stable complexes through complementary base pairing. This can disable a particular RNA interference pathway, acting in part, in some embodiments, by targeting miRs for destruction by recruiting the tailing and trimming pathway to decrease target miR steady-state abundance. RNAi technology is an intense area of research for the development of new therapies, with several studies demonstrating the use of AAV for delivering small signaling oligonucleotides in vivo. Viral delivery of TuDs is accepted in the field as strong miR inhibitors with a single virus delivery, addressing a foreseeable problem of manufacturing multiple drug delivery systems. Inserted into the pAV-U6-GFP vector are the two TuDs cloned at the 5′ ends next to the human U6 or H1 promoters that drive their expression (FIG. 1A). The virus, designed by Jaan Biotherapeutics was made by Vigene Biosciences, 9430 Key West Ave, Suite 105, Rockville, Md. 20850 at in vivo grade quality with multiple batches of virus used in the study. Certificates of analysis were provided for all batches of virus manufactured. Virus was stored at −80° F. and thawed once at the time of use. 293T cells were plated at a density of 8×10\hat{ }4 cells per well in 24 well tissue culture plates. For 10\hat{ }10 vg/mL the viral concentration is approximately 10\hat{ }5/cell. Increasing concentrations of 10\hat{ }11 vg/mL and 10\hat{ }12 vg/mL for the same number of cells were also tested. Polybrene was used to infect the cells as per manufactures guidance (https://www.addgene.org/protocols/generating-stable-cell-lines) which can enhance virus-cell contact. Cells were infected in serum free media for 6 h. Subsequently the serum free media was supplemented with Fetal Bovine Serum (FBS) so the final concentration was 5% (v/v). Media was not changed and the cells were imaged at Day 7 post-infection (FIG. 1B).

### JN-101 AntagomiR Selection and Design

Twenty antagomiRs for miR-99/100 and let-7a/c were screened for bioactivity using the pMIR-REPORT™ miRNA Expression Reporter Vector System (Part Number AM5795, Applied Biosystems). The antagomiRs can act via steric blockade of a specific miRNA in the RNA-induced silencing complex, or RISC complex. The backbone of the antagomiRs is phosphorothioate, and they were designed as such so that they are less than 19 nucleotides in length with no large DNA gaps. The pMIR-REPORT™ miRNA Expression Reporter Vector System comprises an experimental firefly luciferase reporter vector where the 3′ UTR of the luciferase gene contains a multiple cloning site for insertion of predicted miRNA binding targets. The specific miRNA target sequences were cloned into the multiple cloning site in the pMIR-REPORT and the luciferase reporter is subjected to regulation that mimics the miRNA target which should induce a dose-dependent increase in luciferase activity when cells transfected with the reporter constructs are incubated with increasing concentrations of the relevant antagomiRs.

Two cells types were used to test the efficiency of the antagomiRs, HeLa cells and primary neonatal rat ventricular cardiomyocytes. HeLa cells were cultured in Minimum Essential Media with Earle's Balanced Salt Solution (Hyclone) supplemented with 2 mM L-Glutamine, 1 mM Sodium Pyruvate, 1 nM non-essential Amino Acids, and 10% FBS (PAA) and penicillin streptomycin. The cells were plated in serum containing media without antibiotics in 96-well plates (1×10⁴ cells/well) 24 hours prior to transfection and were at a confluency of between 30-70% at the time of transfection. Cells were transfected with 50 ng/well of the LUC reporter plasmid and 10 ng/well of the β-gal reporter plasmid for 2 hours with 0.1, 1, 10 or 50 nanomol/L (nM) using Lipofectamine 2000 (Life Technologies, Cat #11668-019) according to the manufacturer's instructions using Opti-MEM® Medium and normal growth medium in a final volume of 200 μl/well. Reporter plasmids (pMIR-REPORT™ or LUC plasmid) were transfected alone. β-gal expression from this control plasmid was used to normalize variability due to differences in cell viability and transfection efficiency. Rat neonatal rat cardiomyocytes were isolated and plated on Primaria coated plates at density of 80,000 cells per well (24 well). Twenty-four hours after plating the cells were transfected with 500 ng/well of the LUC reporter plasmid and 100 ng/well of the β-gal reporter plasmid for 5 hours with 0.1, 1, 3, 10 or 50 nanomol/L (nM) using Lipofectamine 2000 (Life Technologies, Cat #11668-019) according to the manufacturer's instructions using Opti-MEM® Medium and normal growth medium in a final volume of 600 μl/well. Reporter plasmids (pMIR-REPORT™ or LUC plasmid) were transfected alone. The data demonstrated that the majority of antagomiRs were effective at inhibition of the specific miRNAs as determined by an increase the activity of the corresponding luciferase reporters in a dose-dependent manner in both Hela cells and neonatal rat ventricular cardiac myocytes, suggesting that the antagomiRs specifically bind to the corresponding endogenous miRNAs and inhibit their activity. It is important that the antagomiRs are tested using different cell types and that repeatable, consistent data are observed between experiments. The experiments led to the selection of two antagomiRs for in vivo experiments, JRX0104, let-7a/c antagomiR 5′-ATACAACCTACTACCTC-3′ and JRX0116 miR-99/100 antagomiR 5′-GATCGGATCTACGGGT-3′, that would be administered together for experiments, collectively known as JN-101. FIGS. 1C-1H shows the efficacy of JRX0116 and JRX0104 on miRNA-99/100 and Let-7a/c pMIR-REPORT™ constructs in both cell types.

### Summary of In Vivo Study Design

JBT-miR2 or Control Virus:

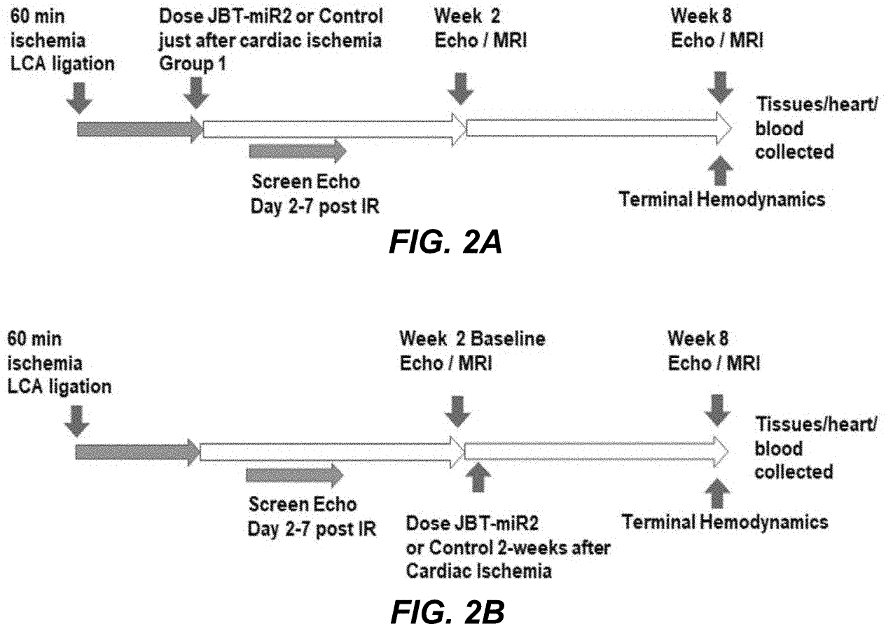

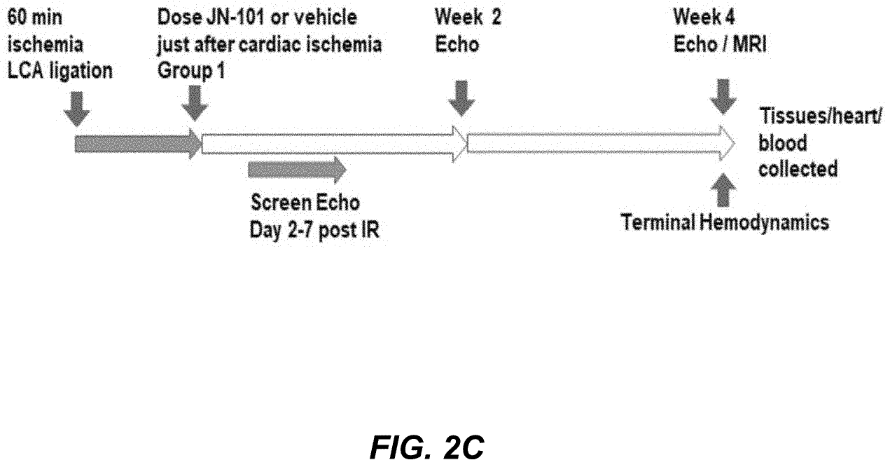

In this double-blind randomized and placebo controlled study, sixty-six (N=66) male CD1 mice were subjected to a 60 min ligation of the left coronary artery (LCA). JBT-miR2 (17 mice) or Scrambled Control virus (Control, N=19 mice) were administered at the time of reperfusion at a dose of 1×10¹¹ vg/mouse diluted in 100 μl of sterile saline for injection, IV via the retro-orbital vein (Group 1, FIG. 2A). The mice were subjected to a screen 2D-Echocardiography (ECHO) between Days 2-7 to confirm consistent surgical injury. At weeks 2 and week 8 post IR, follow-up ECHO and MRI imaging were performed, followed by terminal hemodynamics (HEMO) and tissue and blood collection at week 8. A second group of mice (Group 2, FIG. 2B), were administered with JBT-miR2 (N=14 mice) or Control (N=16 mice) 2-weeks after IR following a week-2 Baseline ECHO and MRI. The mice were subjected to the same procedures as Group 1, with an 8-week follow-up from the time of reperfusion.

JN-101 or Vehicle:

Twenty-four (24) mice were subjected to 60 minutes of IR surgery and were administered with 10 mg/kg of JN-101 in 400 μl (200 μl of JRX0104, 2000 JRX0116) as a sterile SC injection a few minutes after reperfusion (N=12) or Vehicle which consists of sterile injectable saline (N=12) (FIG. 2C). The mice were subjected to a screen 2D-ECHO on Day 2-7 and a follow-up 2D-ECHO at Week-2 and Week-4, followed by terminal hemodynamics and tissue and blood collection. Mice were subjected to an MRI at 4-weeks.

### Ischemic Reperfusion (IR) Surgical Procedures

In vivo procedures were conducted according to established guidelines, and with the approval of the University of California San Diego (UCSD) Institutional Animal Care and Use Committee (IACUC). Male, CD1 mice (8-12 weeks of age) weighing ˜30-40 grams were purchased from Charles River, Mass. 01867. CD1 mice were anesthetized with Ketamine (50 mg/kg) and Xylazine (5 mg/kg) by i.p. injection for initial induction and then maintained on isoflurane (0.75-1.5%) for complete induction of anesthesia throughout the procedure. The mice were intubated with a pressure ventilator (Kent Scientific, PhysioSuite. Peak inspiration pressure around 13 cm H2O, inspiration rate 100-110/min). When the mice were considered stable, and anesthesia was confirmed, a skin incision was made from the midsternal line toward the left armpit, and the chest wall was opened with a 1 cm lateral cut along the left side of the sternum, cutting between the 3rd and 4th ribs to expose the left ventricle (LV) of the heart. The ascending aorta and main pulmonary artery were identified; the left anterior descending coronary artery (LCA) was then located as it traverses the anterior wall of the heart, between the left and right ventricles. LCA occlusion was performed by tying an 8-0 prolene suture ligature on a piece of 2-0 silk suture. Occlusion of the artery was assessed by blanching of the territory of perfusion of the LCA, along with acute ST segment elevation on limb-lead electrocardiographic leads. When the mouse was stable for at least 5 minutes, the mouse can be moved to a second ventilator without isoflurane and the body temperature of the mouse was maintained with a water circulated warming pad for the remaining ischemic period. An additional mixture of Ketamine (50 mg/kg) and Xylazine (5 mg/kg) (i.p.) and 200-300 μl normal saline (i.p.) according to the body weight of the mice in grams was given at this time. The mice were monitored closely by the operator for the whole procedure and recovery. The mouse heart were under ischemia for 60 minutes according to the study requirement. After 60 mins, the 2-0 suture was removed and the heart was reperfused.

Reperfusion was confirmed by observing return of blood flow in the epicardial coronary arteries. JBT-miR2 or scrambled control virus (Control) were administered at 1×10¹¹ vg/mouse diluted in 100 μl of saline via the retro-orbital vein at the time of reperfusion using a 27 gauge needle (305109, 27 Gauge-Regular Bevel ½ Inch-Gray). Alternatively, 200 μl of JRX0104 (10 mg/kg diluted in sterile saline for injection) and 200 μl of JRX0116 (10 mg/kg diluted in sterile saline for injection) (400 μl of JN-101) or 400 μl of sterile saline were administered as a subcutaneous bolus injection into the loose skin over the back if the neck.

The suture was then removed from around the LCA; once the mouse was hemodynamically stable, the chest was closed. An additional 100-200 μl of normal saline (i.p.) was given according to the body weight of the mouse. The chest was then closed with one layer of suture of 5-0 braided black silk through the chest wall and the skin was closed with NEXABAND® Liquid topical tissue adhesive. Air was evacuated from the chest at the time of incision closure. Postoperatively, animals were observed closely for signs of discomfort or shortness of breath. Buprenorphine (0.1 mg/kg) (100 μl) was given 15-30 min prior to anticipated recovery or Buprenorphine HCl extended-release injectable suspension (3.25 mg/kg) were given subcutaneously, 15-30 min prior to the anticipated recovery time of animals. No animals were euthanized because of acute distress after the surgical procedure or in aftercare. The animals were observed daily for 5 days post-surgery and weighed before and after any study procedure.

### Cardiac MRI

With any cardiac regenerative therapy improvement in heart function, reduction in volumes and scar size should be sought. ECHO is commonly used in experimental animals and man because of its ease of use and accuracy in defining cardiac dimensions, but is limited by planographic views that do not reflect the full extent of myocardial damage. In contrast, three dimensional (3D) cardiac MRI (3D-MRI) reconstruction with finite element analysis of endocardial area provides predictive, quantitative measures of LV and MI size, global LV function, regional endocardial wall motion abnormality (nodal displacement, surface element % change) at week 2 and 8 post IR for virus treated mice or at week-4 for JN-101 treated mice.

Randomized N=6-7 mice/Grp were weighed and anesthetized with isoflurane 1.5-2.5% with 100% 02. MRI was performed using a horizontal Bruker Biospec 7T/20 MRI system for small animals (Bruker, Germany). For FLASH-cine image acquisition, EKG and respiratory signals were sent to a gating system (SA Instruments). Images were acquired with the parameters: FOV: 1.5×2.0 cm, matrix size: 128×128, slice thickness: 1 mm, inter-slice distance: 0 mm, echo time: 2.1 msec, repetition time: 6 msec, 6 average, flip angle: 40°, and 20 frames per cardiac cycle. A single slice acquisition time was 110 secs and was synchronized with QRS complex peak. The size of myocardial gray zone (a mixture of normal and infarcted tissue) identified from late gadolinium enhanced (LGE) MRI was an independent predictor of adverse cardiac events post-infarction. For LGE imaging, 30 μL of 0.5 mmol/kg Gd-DTPA (Magnevist®, Schering Healthcare, UK) was given i.p. 20-50 mins prior to the MR scan. Short axis planes were set to be perpendicular to the coronary plane and the long axis. Nine contiguous short axis slices were required to cover the entire LV.

LV Size, Global Function and EF:

For ED and ES images, volumetric data were determined from the product of compartment area and slice thickness (1 mm). EDV and ESV were calculated from the summation of all slices and the EF derived.

MI Size:

ED images of each slice were selected for scar delineation.

3D Reconstruction and Wall Motion Analysis:

The borders derived from the images were imported into Continuity (6.4b revision 6734, National Biomedical Computation Resource).

Quantification and Normalization:

The simplest normalization for ventricular size was to calculate the ratio of displacement at each node to the calculated end-diastolic endocardial surface area (EDSA). An alternative normalization was to calculate a “Z” score for each parameter, based upon the SD derived from the distribution at each node measured in a control group of mice (N=8) of the same age and species. For 3D endocardial wall motion, a node that moves <2.0 SDs from the mean of control hearts was classified as a hypokinetic/akinetic abnormal node. If a surface element has >3 abnormal nodes on its endocardial surface, the element was classified as an abnormal element. The amount of abnormal myocardium was calculated by summing the number of nodes with Z scores <2.0 SDs. Matlab® (MathWorks, MA) and Continuity Software were used for all analyses.

### 2D-Echocardiography

2D-ECHO was conducted on all mice. A screen ECHO was done between day's 2-7 post IR to confirm comparable area of risk (AOR) injury, with follow-up at weeks 2 and 8 for JBT-miR2 or scrambled control virus treated animals and week 2 and week 4 post IR surgery for JN-101 or control treated mice. The week 2 ECHO was considered baseline for mice randomized to JBT-miR2 or Control virus groups in Group 2, before any viral treatment. Mice were anesthetized with isoflurane 1.5-2.5% with 100% O₂ and imaged using a Visual Sonics 2100 machine and a 30-MHz transducer.

### Hemodynamics

Surviving mice at week 8 post IR (JBT-miR2 or Control virus) or at 4-weeks post IR (JN-101 or sterile saline for injection control) were anesthetized with an i.p. injection of KX, intubated, ventilated and the right carotid artery exposed. A 1.4 French high-fidelity catheter-tip micro manometer (Millar Instruments, TX) was inserted retrograde into the aorta via the left carotid artery and advanced into the LV. After bilateral vagotomy, baseline pressures were recorded until stable and dobutamine was given through the femoral vein at sequentially increasing doses of 0.75, 2, 4, 6 and 8 μg/kg/min. Pressure data was recorded with the Chart acquisition system (AD Instruments) and analyzed by a custom-made program. Parameters include (mmHg): Aortic mean pressure, Right atrial pressure, Max Pressure, End Diastolic Pressure, Cardiac output (ml/min), Systemic vascular resistance (mmHg/ml). Max and Min Peak (—/+) dP/dt (mmHg/s) were also measured.

### Necropsy, Histology and Terminal Procedures

For JBT-miR2 or Control virus treated hearts, after hemodynamics, hearts were removed and cannulated via the aorta and perfused with relaxing buffer; 77 mM Nacl, 4.3 mM Na2HPO4.7H2O, 1.47 mM KHPO4, 62.7 nM KCL under gravity. The hearts were fixed in 10% Neutral Buffered Formalin. Other tissues (lung, spleen, liver, kidney, skin, skeletal muscle, brain) were removed and stored in 10% Neutral Buffered Formalin. Tissues were embedded in paraffin blocks for histology. Samples of tissue, including heart muscle were retained for Next Generation Sequencing (NGS), RNAseq and quantitative PCR.

### Quantitative Histology Group 1 Virus

For quantification of scar size, eight hearts (N=3 Control and N=5 JBT-miR2, Group 1) were trimmed above the ligation for cross section orientation, processed in its own cassette and embedded cut side down. Each FFPP block was cut at 4 μm onto positively charged slides with six serial sections collected at three different levels. IF staining was performed on FFPE mouse hearts using a Leica Bond automated immunostainer with previously validated conditions. Heat induced antigen retrieval was performed using Leica Bond Epitope Retrieval Buffer 2 (EDTA solution, pH 9.0) for 20 minutes (ER2 (20)). Non-specific antibody binding was blocked using Novolink Protein Block (Leica, cat #RE7280-CE, lot #6064062) for 20 minutes. Endogenous peroxidase was blocked using Novolink Peroxide Block (Leica, cat #RE7280-CE, lot #6056766) for 10 minutes. Non-specific antibody binding was blocked using Novolink Protein Block (Leica, cat #RE7280-CE, lot #6064062) for 20 minutes. Anti-MHC antibody was applied manually with an overnight incubation. The following day, Goat anti-Mouse Alexa Fluor Plus® 488 was applied for 60 minutes. Slides were mounted with DAPI in Fluor gel II for nuclear visualization.

### Immunohistochemistry Optimized Staining Conditions

Whole slide images were generated in brightfield and fluorescence using a Pannoramic SCAN (30 Histech) and were provided on a USB data drive together with image viewing software (Case Viewer). Quality Control: Images were assessed for quality by an image specialist and any images not meeting the quality criteria were rescanned. Representative snapshot images are included in this Example. The scale bar represents 50 μm. Image analysis data were generated by automated analysis of whole slide images using an integrated whole slide image management and automated image analysis workflow called ImageDx™. Each image was first assessed for quality using a precise focus measurement followed by an accuracy check. All tissue and staining artifacts were digitally excluded from the reported quantification. The analysis process includes automated identification of tissue, followed by segmentation of regions of interest and then classification of cells positive for specific marker immunoreactivity. These identified regions were then quantified for precise positivity. For Masson's Trichrome samples, collagen positive image data was isolated from the total tissue image data. The algorithm identified tissue with respect to location on the slide and then the yellow regions corresponding to the collagen for Masson's Trichrome staining. These identified regions were then quantified for positivity. Quantitative data points were generated to measure positive cells per tissue area.

### Cell Size Analysis

Cell size determination was created by the nuclear segmentation based on the nuclear counter-stain (DAPI in fluorescence). The size of a given nuclei was derived from the area of the counter-stain and was converted to microns. Density graphs show the distribution of heart muscle cell nuclei size, and the approximate cytoplasm, together to create a measurement of cell size.

### Qualitative Histology Group 1 and 2 Hearts and Tissues

Hearts from both Group 1 (N=3 JBT-miR2, N=4 Control) and Group 2 (N=4 JBT-miR2 and Control) and tissues received in formalin were examined grossly, dissected for placement into cassettes and processed into paraffin. Histologic sections were stained with H&E and assessed qualitatively as follows: Normal=0, 1 (+) to 3 (+++) Minimal to Maximal fibrosis and thinning. Non-cardiac tissue (brain, lungs, liver, spleen, kidney, skin and skeletal muscle) were stained with H&E for toxicity. Pathology assessment of toxicological changes were determined using the General Approach to Histopathologic Evaluation. Coded H&E stained histologic sections of liver, kidney, and spleen were evaluated in a first pass, blind fashion. Results were then reviewed and evaluated after breaking the code so as to incorporate organ weights and associated functional and biochemical results. Specific histologic features evaluated are as follows: Liver: micro and macro steatosis, vacuolar or hydropic cytoplasmic degeneration, apoptosis, necrotic foci, hemorrhage, infiltration of inflammatory cells, and bi-nucleated cells. Kidney: diminution and distortion of glomeruli, acute tubular necrosis, dilation of tubules, renal tubular epithelial cell vacuolation, hyaline droplets, thrombotic microangiopathy, mesangiolysis, edema, necrosis, infiltration of inflammatory cells. Spleen: distorted lymphoid architecture, minimized lymphoid follicles, the presence of granular leukocytes and giant macrophages. Heart: cytoplasmic vacuolization, myocyte necrosis, contraction band necrosis, infiltration of macrophages and neutrophils, myocardial fibrosis Abnormalities in the tissue sections, if present, were semi quantitatively graded from 0 (normal structure) to 3 (severe pathological changes).

### Metabolic Blood Function Tests (MFTs)

After a lethal injection of KX, blood was collected through the abdominal into BD SST Microtainer REF 365967 tubes and centrifuged at 5000×g, for 10 mins at 4° C. Serum samples were frozen at −80° C. until testing for M. Blood test results were designated normal or abnormal by the testing laboratory and the relative concentrations of each analyte were expressed as per/unit (analyzed by Rabbit and Rodent Diagnostic Services (RADA), San Diego, 92121).

### PCR Detection of JBT-miR2 in Tissues

Total RNA was isolated from ˜20 mg of total wet tissue (liver, skeletal muscle, brain, lung, spleen, heart, kidney) for two mice treated with either JBT-miR2 or Control virus using the mirVana miRNA Isolation Kit (ThermoFisher, cat #AM1560) according to the manufacturer's instructions. RNA samples were diluted to 2 ng/μl. Total RNA was reverse transcribed to cDNA using the High-Capacity RNA-to-cDNA Kit (ThermoFisher, cat #4387406) according to the manufacturer's instructions in a total volume of 20 μl using a thermal cycler ABI9700, with reactions at 37° C. for 60 min, 95° C. for 5 min, and 4° C. hold. The following qPCR procedure shown in Table 6 were conducted using a 96-well plate using the following Taqman primers according to the manufacturer's instructions as stated below.

TABLE 6
Qper Analysis

| Mouse | | | |
|---|---|---|---|
| microRNA | | Link and sequences | |
| Sequence | Vendor/cat # | Life Technologies | PCR Reactions |
| let-7a | MI0000060 | UGAGGUAGUAGGUUGU | Taqman small RNA assay (20x)-1 μl |
| | hsa-let-7a, | AUAGUU (SEQ ID NO: 90) | RT-products-1 μl |
| | Catalog # 4427975 | | Taqman master mix/primers-10 μl |
| | | | ddhH₂O-8 μl |
| | | | 95° C. 10 min, (95° C. 15 secs, 60° C. |
| | | | 1 min) × 30, 4° C. Hold |
| let-7c | MI0000064 | UGAGGUAGUAGGUUGU | Taqman small RNA assay (20x)-1 μl |
| | hsa-let-7c | AUGGUU (SEQ ID NO: 91) | RT-products- 1 μl |
| | Catalog # 4427975 | | Taqman master mix/primers-10 μl |
| | | | ddhH₂O-8 μl |
| | | | 95° C. 10 min, (95° C. 15 secs, 60° C. |
| | | | 1 min) × 30, 4° C. Hold |
| miR-100 | MI0000102 | AACCCGUAGAUCCGAAC | Taqman small RNA assay (20x)-1 μl |
| | hsa-miR-100 | UUGUG (SEQ ID NO: 92) | RT-products- 1 μl |
| | Catalog # 4427975 | | Taqman master mix/primers-10 μl |
| | | | ddhH₂O-8 μl |
| | | | 95° C. 10 min, (95° C. 15 secs, 60° C. |
| | | | 1 min) × 30, 4° C. Hold |
| miR-99a | MI0000101 | AACCCGUAGAUCCGAUC | Taqman small RNA assay (20x)-1 μl |
| | hsa-miR-99a | UUGUG (SEQ ID NO: 93) | RT-products-1 μl |
| | Catalog #4427975 | | Taqman master mix/primers-10 μl |
| | | | ddhH₂O-8 μl |
| | | | 95° C. 10 min, (95° C. 15 secs, 60° C. |
| | | | 1 min) × 30, 4° C. Hold |
| GAPDH | Mm99999915_g1 | | Taqman small RNA assay (20x)-1 μl |
| (reference) | Gapdh | | RT-products-1 μl |
| | Catalog #4453320 | | Taqman master mix/primers-10 μl |
| | | | ddhH₂O-8 μl |
| | | | 95° C. 10 min, (95° C. 15 secs, 60° C. |
| | | | 1 min) × 30, 4° C. Hold |
| Human 6 | Two sets of primers | >HsU6R | Taqman small RNA assay (20x)-1 μl |
| promoter | were used | GCTAATCTTCTCTGTATC | RT-products-1 μl |
| | | GTTCCA (SEQ ID NO: 94) | Taqman master mix/primers-10 μl |
| | | >HsU6F | ddhH₂O-8 μl |
| | | GGATCAGCGTTTGAGTA | 95° C. 2 min, (95° C. 30 secs, 50° C. |
| | | AGAG (SEQ ID NO: 95) | 30 secs, 72° C. 1 min) × 25, 72° C. |
| | | >Human-U6-52 | 2 min 10° C. Hold |
| | | GCCTATTTCCCATGATTC | |
| | | CTTC (SEQ ID NO: 96) | |
| | | >Human-U6-32 | |
| | | GGTGTTTCGTCCTTTCCA | |
| | | C (SEQ ID NO: 97) | |

### Next Generation Sequencing

Samples were uniformly cut encompassing 2 mm of tissue at the apex of the LV from the hearts of 2 mice treated with JBT-miR2 (hearts from mice 309 and 310) and Control virus (Heart 315 and 316). The tissues were snap frozen under liquid nitrogen and stored at −80° C. until analysis. The samples of tissue were from Group 2 mice treated with virus at 8-weeks post IR. RNA was isolated using Trizol reagent as per the manufacturers' instructions. RNA degradation and contamination was monitored on 1% agarose gels. RNA purity was checked using the NanoPhotometer® spectrophotometer (IMPLEN, CA, USA). RNA concentration was measured using Qubit® RNA Assay Kit in Qubit® 2.0. Fluorimeter (Life Technologies, CA, USA). RNA integrity was assessed using the RNA Nano 6000 Assay Kit of the Bio analyzer 2100 system (Agilent Technologies, CA, USA).

Library Preparation for lncRNA Sequencing

A total amount of 3 μg RNA per sample was used as input material for the RNA sample preparations. Firstly, ribosomal RNA was removed by Epicentre Ribo-zero™ rRNA Removal Kit (Epicentre, USA), and rRNA free residue was cleaned up by ethanol precipitation. Subsequently, sequencing libraries were generated using the rRNA-depleted RNA by NEBNext® Ultra™ Directional RNA Library Prep Kit for Illumina® (NEB, USA) following manufacturer's recommendations. Briefly, fragmentation was carried out using divalent cations under elevated temperature in NEBNext First Strand Synthesis Reaction Buffer (5×). First strand cDNA was synthesized using random hexamer primer and M-MuLV Reverse Transcriptase (RNaseH-). Second strand cDNA synthesis was subsequently performed using DNA polymerase I and RNase H. In the reaction buffer, dNTPs with dTTP were replaced by dUTP. Remaining overhangs were converted into blunt ends via exonuclease/polymerase activities. After adenylating of 3′ ends of DNA fragments, NEB Next Adaptor with hairpin loop structure were ligated to prepare for hybridization. In order to select cDNA fragments of preferentially 150-200 bp in length, the library fragments were purified with AMPure XP system (Beckman Coulter, Beverly, USA). Then 3 μl USER Enzyme (NEB, USA) was used with size-selected, adaptor-ligated cDNA at 37° C. for 15 min followed by 5 min at 95° C. before PCR. Then PCR was performed with Phusion High-Fidelity DNA polymerase, Universal PCR primers and Index (X) Primer. At last, products were purified (AMPure XP system) and library quality was assessed on the Agilent Bio analyzer 2100 system.

### Data Analysis

Quality Control:

Raw data (raw reads) of fastq format were firstly processed through in-house perl scripts. In this step, clean data (clean reads) were obtained by removing reads containing adapter, reads on containing ploy-N and low quality reads from raw data. At the same time, Q20, Q30 and GC content of the clean data were calculated. All the downstream analyses were based on the clean data with high quality.

Mapping to the Reference Genome:

Reference genome and gene model annotation files were downloaded from genome website directly. Index of the reference genome was built using bowtie2 v2.2.8 and paired-end clean reads were aligned to the reference genome using HISAT2 v2.0.4. HISAT2 was run with ‘-rna-strandness RF’, other parameters were set as default.

Transcriptome Assembly:

The mapped reads of each sample were assembled by StringTie (v1.3.1) in a reference-based approach. StringTie uses a novel network flow algorithm as well as an optional de novo assembly step to assemble and quantitate full-length transcripts representing multiple splice variants for each gene locus.

Coding Potential Analysis:

Picard-tools v1.41 and samtools v0.1.18 were used to sort, remove duplicated reads and merge the bam alignment results of each sample. GATK3 software was used to perform SNP calling. Raw vcf files were filtered with GATK standard filter method and other parameters (cluster: 3; WindowSize: 35; QD<2.0 or FS>60.0 or MQ<40.0 or SOR>4.0 or MQRankSum <−12.5 or ReadPosRankSumØ-8.0 or DP<10). CNCI (Coding-Non-Coding-Index) (v2) profiles adjoining nucleotide triplets to effectively distinguish protein-coding and non-coding sequences independent of known annotations (Sun et al. 2013). CPC (Coding Potential Calculator) (0.9-r2) mainly through assess the extent and quality of the ORF in a transcript and search the sequences with known protein sequence database to clarify the coding and non-coding transcripts. The NCBI eukaryotes' protein database was used and set the e-value ‘le-10’ in the analysis.

Pfam-Scan:

Each transcript was translated in all three possible frames and Pfam Scan (v1.3) was used to identify occurrence of any of the known protein family domains documented in the Pfam database (release 27; used both Pfam A and Pfam B). Any transcript with a Pfam hit would be excluded in following steps. Pfam searches use default parameters of −E 0.001-domE 0.001.

phyloCSF:

PhyloCSF (phylogenetic codon substitution frequency) (v20121028) examines evolutionary signatures characteristic to alignments of conserved coding regions, such as the high frequencies of synonymous codon substitutions and conservative amino acid substitutions, and the low frequencies of other missense and non-sense substitutions to distinguish protein-coding and non-coding transcripts. Multi-species genome sequence alignments were built and phyloCSF was run with default parameters. Transcripts predicted with coding potential by either/all of the four tools above were filtered out, and those without coding potential were the candidate set of lncRNAs.

Conservative Analysis:

Phast (v1.3) is a software package containing statistical programs, most used in phylogenetic analysis, and phastCons is a conservation scoring and identification program of conserved elements. The program phyloFit was used to compute phylogenetic models for conserved and non-conserved regions among species and then gave the model and HMM transition parameters to phastCons to compute a set of conservation scores of lncRNA and coding genes.

Target Gene Prediction:

Cis Role of Target Gene Prediction

Cis role is lncRNA acting on neighboring target genes. Coding genes were searched 10 k/100 k upstream and downstream of lncRNA and then analyzed their function next.

Trans Role of Target Gene Prediction

Trans role is lncRNA to identify each other by the expression level. The expressed correlation between lncRNAs and coding genes with custom scripts was calculated; otherwise, the genes from different samples were clustered with WGCNA57 to search common expression modules and then analyzed their function through functional enrichment analysis.

Quantification of Gene Expression Level:

Cuffdiff (v2.1.1) was used to calculate FPKMs of both lncRNAs and coding genes in each sample. Gene FPKMs were computed by summing the FPKMs of transcripts in each gene group. FPKM means fragments per kilo-base of exon per million fragments mapped, calculated based on the length of the fragments and reads count mapped to this fragment.

Differential Expression Analysis:

The Ball gown suite includes functions for interactive exploration of the transcriptome assembly, visualization of transcript structures and feature-specific abundances for each locus, and post-hoc annotation of assembled features to annotated features. Transcripts with a P-adjust <0.05 were assigned as differentially expressed.

Cuffdiff provides statistical routines for determining differential expression in digital transcript or gene expression data using a model based on the negative binomial distribution. Transcripts with a P-adjust <0.05 were assigned as differentially expressed.

GO and KEGG Enrichment Analysis:

Gene Ontology (GO) enrichment analysis of differentially expressed genes or lncRNA target genes were implemented by the GOseq R package, in which gene length bias was corrected. GO terms with corrected P Value less than 0.05 were considered significantly enriched by differential expressed genes. KEGG is a database resource for understanding high-level functions and utilities of the biological system, such as the cell, the organism and the ecosystem, from molecular-level information, especially large-scale molecular datasets generated by genome sequencing and other high-throughput experimental technologies (http://www.genome.jp/kegg/). KOBAS software was used to test the statistical enrichment of differential expression genes or lncRNA target genes in KEGG pathways.

PPI (Protein Protein Interaction):

PPI analysis of differentially expressed genes was based on the STRING database, which known and predicted Protein-Protein Interactions. For the species existing in the database, the networks were constructed by extract the target gene list from the database; Otherwise, Blastx (v2.2.28) was used to align the target gene sequences to the selected reference protein sequences, and then the networks were built according to the known interaction of selected reference species.

Alternative Splicing Analysis:

Alternative splicing events were classified to 12 basic types by the software Asprofile v1.0. The number of AS events in each sample was estimated, separately.

Snp Analysis:

Picard-tools v1.96 and samtools v0.1.18 were used to sort, mark duplicated reads and reorder the bam alignment results of each sample. GATK2 software was used to perform SNP calling.

### Statistical Analysis

Statistical analysis was done by individuals who were unaware of treatment allocation. All quantitative data are expressed as Mean±SD. Efficacy (ECHO, MRI, HEMO parameters and scar size) and safety (MFTs, Pathology, and survival) of JBT-miR2 compared with Control at 2-weeks and 8-weeks post IR when appropriate. JN-101 parameters were compared at 2-weeks and 4-weeks post-IR when appropriate. For serial data the main effects of treatment were tested using two-factor ANOVA for repeated measures, and differences between the treatments at specific time points and doses with post hoc comparisons by the Student-Newman-Keuls test. P<0.05 was considered statistically significant. Systat 13.0 was used for MRI linear modeling, nesting of wall motion agreement between parameters. Survival was determined using Kaplan-Meier analysis of the number of mice remaining infected each day. Histology: Quantification of scar size and increased numbers of MHC stained CMs/area confirmed CM proliferation using Image^{DX}.

### Pathology in Off-Target Tissues Normal Mice

Batch 1 Formal Pathology Report

Overview: The study was of antimiRs vs controls injected with saline. Wet tissues received in formalin were examined grossly, dissected for placement into cassettes and processed into paraffin. Histologic sections were prepared and stained with H&E. Paraffin blocks of tissue remain available for any further analysis, such as immunohistochemical studies, that might be requested.

Analysis: Tissues that were available for each specimen ID (5-7; 29; 34-40; 45-48; 77-80. All specimens had liver and kidney available, and with two exceptions (29; 34) all had lung and heart as well.

There were no specific histologic abnormalities seen in lung, heart and kidneys. Similarly, skeletal muscle and skin showed no histologic abnormalities. There were vacuolar changes in hepatocytes as shown in column C of the attached spread sheet. These changes do not correlate with experimental variables, and are attributed to fixation artifact and/or post-mortem interval.

Conclusion: There are no histologic changes that correlate with experimental variables.

Batch 2—Formal Pathology Report

Overview: Wet tissues received in formalin were examined grossly, dissected for placement into cassettes and processed into paraffin. Histologic sections were prepared and stained with H&E. Paraffin blocks of tissue remain available for any further analysis, such as immunohistochemical studies, that might be requested.

Analysis: Tissues that were available for each specimen ID (85-88; 113-116, 125-128; 152-155). All specimens had liver and kidney available.

One lung specimen showed congestion (858). There were no additional specific histologic abnormalities seen in lung, heart and kidneys. Similarly, skeletal muscle and skin showed no histologic abnormalities.

Conclusion: There are no histologic changes indicative of toxicologic changes amongst the four groups.

### Results

### In Vitro Expression

Hek-293 cells were transfected with increasing concentrations of Control or JBT-miR2 virus. Green Fluorescent protein expression was detected at all concentrations but with ˜70% infection observed with the highest concentration of 10\hat{ }12vg/mL media (FIG. 1B). FIGS. 1C-1H show the efficacy of JRX0116 and JRX0104 on miRNA-99 and Let-7a/c pMIR-REPORT™ constructs in both HeLa cells and neonatal rat ventricular cardiomyocytes.

### Ischemia Reperfusion Experiments

### Survival JBT-miR2

Although not powered for a survival study, and compounded by the lethality of surgically induced IR injury, in both Group 1 and Group 2, JBT-miR2 increased survival by ˜20% compared to Control. Group 1: 53% of JBT-miR2 treated mice survived (9/17 mice), compared with 42% of Control (8/19 mice) to 8-weeks post IR. Group 2: Survival in JBT-miR2 treated mice was 78% (11/14 mice, including a mouse that died during the 8-week ECHO), compared with 56% in Control (9/16) virus treated animals, 8-weeks post-IR (Table 7).

TABLE 7
Final deposition of mice at 8-weeks post Ischemia Reperfusion

| | | Animals | | | Animals |
|---|---|---|---|---|---|
| | No. of | Survived to | | No. of | Survived to |
| Treatment | Operated | Week 8 | Treatment | Operated | week 8 |
| Group 1 | mice | N (%) | Group 2 | mice | N (%) |
| JBT-miR2 | 17 | 9 (53) | JBT-miR2 | 14 | 11 (78)* |
| Control Virus | 19 | 8 (42) | Control Virus | 16 | 9 (56) |

*Includes one mouse that died after 8-week Echocardiography

### Survival JN-101:

SC administration of 10 mg/kg JN-101 at the time of reperfusion after ischemia, showed a survival of 83% of mice (n=10/n=12) compared to 67% of mice surviving to 4-weeks post IR when administered with Vehicle at reperfusion (n=8/n=12) (Table 8).

TABLE 8
Survival of mice in each group

| | No. of | Animals Survived |
|---|---|---|
| Treatment | Operated mice | to Week 4 N (%) |
| JN-101 | 12 | 10 (83) |
| Vehicle | 12 | 8 (67) |

### Dose of JN-101 Administered to Normal Mice

JN-101 was administered to 152 uninjured mice at increasing doses (Table 9). No adverse pathological effects were observed at doses up to 15 mg/kg when the study drug was administered by subcutaneous injection. These data showed that SC administration as a single dose of 15 mg/kg was safe in mice up to 15 days following a single injection. SC administration as a single dose of 10 mg/kg was safe in mice up to 25 days following a single injection. Decreases in Left ventricular Inner Diastolic Diameter in diastole (LVIDd) and Left ventricular Inner Diastolic Diameter in Systole (LVIDs) were observed in JN-101 treated mice. Increases in Left Ventricular Posterior wall diameter (LVPWd) were observed in JN-101 treated mice. There were no changes in heart rate (HR), Ejection Fraction (EF) in uninjured mice. A dose dependent increase in heart weight (HW) to body weight (BW) ratio were observed with all mice treated with JN-101, but no changes in BW were observed. There were no study drug related deaths in the 25 day period. All metabolic blood function tests were within the normal range in normal mice. There were no abnormalities found in off-target tissues including the liver, skin, skeletal muscle, kidney and lung. An optimal dose of 10 mg/kg JN-101 was selected to move forward for testing in this Example in mice with a transient IR Injury.

All mice will had a 2D-Echo on day of sacrifice. Hearts were collected from all mice and analyzed for miR knockdown using RT-PCR (n=4). Histological analysis was conducted on tissues from saline treated mice and mice administered with 15 mg/kg of anti-miRs (high dose) (n=4/group). Blood was collected from all mice and stored. Comprehensive metabolic panel analysis was conducted on plasma from saline treated mice, and mice treated with 15 mg/kg of anti-miRs (high dose) (n=4/group) at different time points. (Table 9).

TABLE 9
Effects of Subcutaneous Administration of JN-101 in Normal Mice
Schematic representation of Aim 1 A/B/C

| | Saline | 0.033 | | | 1 | | | 15 | |
|---|---|---|---|---|---|---|---|---|---|
| Mg/kg | Control | (Low) | 0.1 | 0.3 | (Mid) | 3.3 | 10 | (High) | Sacrifice Day |
| Group 1 | 8 | 4 | 4 | 4 | 4 | 4 | 4 | 8 | Day 2 |
| Group 2 | 8 | 4 | 4 | 4 | 4 | 4 | 4 | 8 | Day 8 |
| Group 3 | 8 | 4 | 4 | 4 | 4 | 4 | 4 | 8 | Day 15 |
| Group 4 | 8 | 4 | 4 | 4 | 4 | 4 | 4 | 8 | Day 28 |

### Optimal Route of Administration of JN-101 is SC

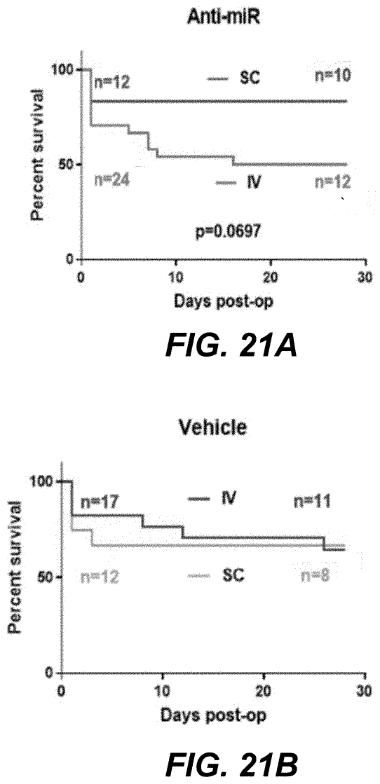

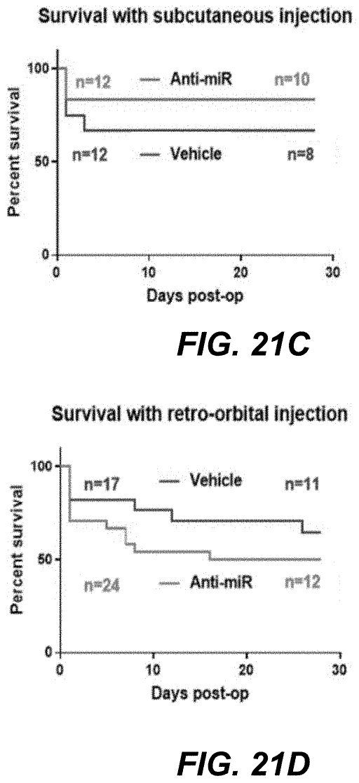

An initial cohort of forty-one (41) animals were subjected to a 60 minute ligation of the LCA and JN-101 was administered IV via the femoral vein in a volume of 200 μl. The study was halted after nine surviving mice who were treated with JN-101 experience a cardiac thrombus (9/17, 52.9%) which was detected by 2D-Echocardiography. No animals treated with Vehicle experienced a thrombus. Cardiac thrombi have been seen with other study drugs of high viscosity including those administered with Bovine Serum Albumin by the testing laboratory. An increase in mortality was seen in mice treated with JN-101 by IV infusion. As a result of these initial findings study drug administration was changed to the SC route (Tables 10A-10D; FIGS. 21A-21D).

TABLE 10A
Optimal Route of Administration of
JN-101 Post IR Survival Statistics

| | | Animals | | |
|---|---|---|---|---|
| | Treatment | Operated | Total Survival | |
| Subcutaneous Injection | Anti-miR | 12 | 10 | 83% |
| | Vehicle | 12 | 8 | 67% |

TABLE 10B
Optimal Route of Administration of
JN-101 Post IR Survival Statistics

| | | Animals | | |
|---|---|---|---|---|
| | Treatment | Operated | Total Survival | |
| Retro-orbital or femoral | Anti-miR | 24 | 12 | 50% |
| (intravenous) Injection | Vehicle | 17 | 11 | 65% |

TABLE 10C
Optimal Route of Administration of JN-
101 - Post IR Thrombus Statistics

| | |
|---|---|
| Subcutaneous Injection | No Thrombus Observed |

TABLE 10D
Optimal Route of Administration of JN-
101 - Post IR Thrombus Statistics

| | Treatment (animals | Screen | 2 | 4 |
|---|---|---|---|---|
| | screened) | 3-5 days | weeks | weeks |
| Intravenous | Anti-miR (n = 17) | 9* | 0 | 0 |
| Injection | Vehicle (n = 15) | 0 | 0 | 0 |

*only 3 animals with thrombus survived for 2 and 4 weeks echoes

### Global Heart Function JBT-miR2

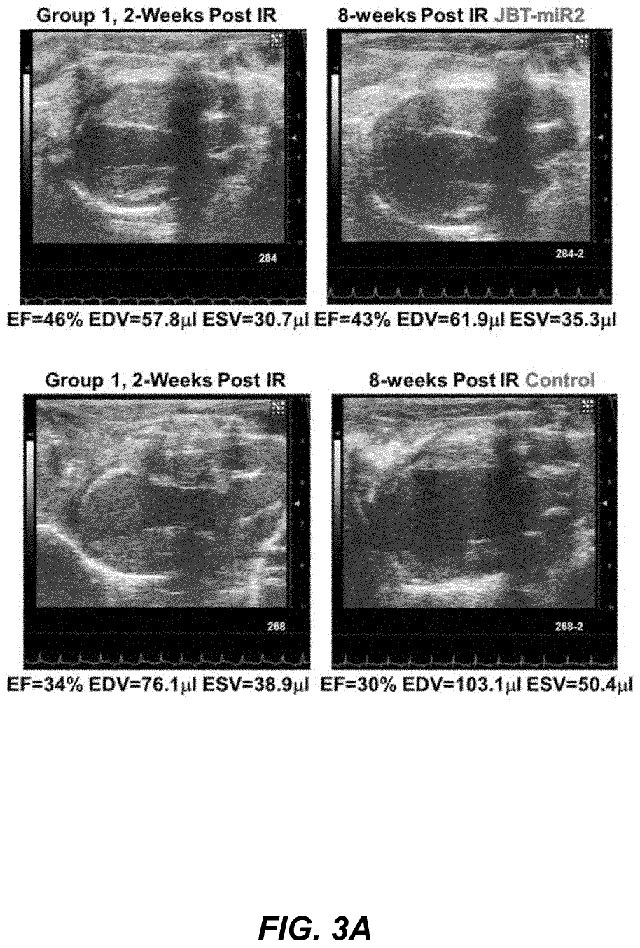

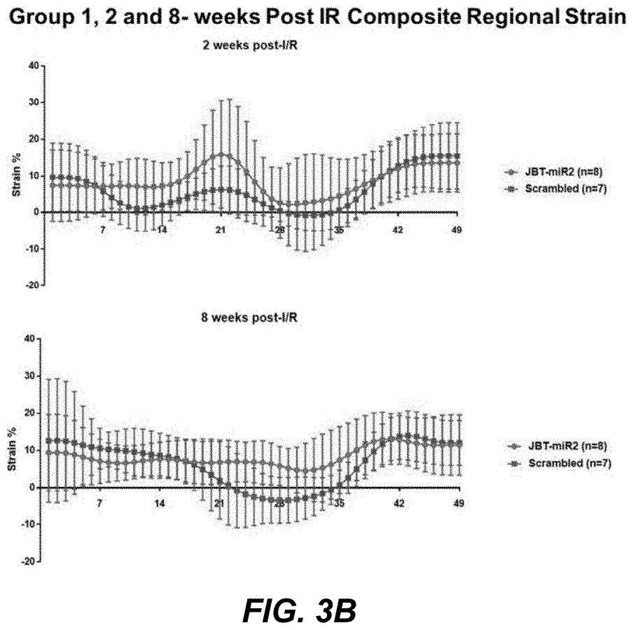

In Group 1 mice, both MRI and ECHO showed consistent value changes of LV global indices, increasing confidence in JBT-miR2 efficacy (Tables 11A-11C). 2-weeks post virus administration, EF increased by ˜23% in JBT-miR2 treated mice compared to Control (P=0.093, ECHO). Similarly, End Diastolic Volume (EDV) and End Systolic Volume (ESV) were reduced by 23% (P=0.05, ECHO) and 32-34% (P=0.03, ECHO), respectively. The weaning effect at week 8 may be due to the systemic IV route of administration which dilutes the levels of virus that reach the heart at an effective concentration (FIG. 3A). For Group 2 mice, the 8-week ECHO values were subtracted from the 2-week baseline values, before treatment. ECHO demonstrated an 8 μl decrease in EDV and ESV at 8-weeks post IR, 6-weeks post treatment compared to mice treated with Control Virus. In addition there was a 3% increase in EF in JBT-miR2 treated mice compared to mice treated with Control virus. This data suggests that in some embodiments a higher or cardiac localized injection of JBT-miR2 may be beneficial in the established heart failure setting.

TABLE 11A
Global ECHO and MRI LV Indices JBT-
miR2 or Control Virus Treated Mice

| | | 2-weeks | 8-weeks |
|---|---|---|---|
| | | EDV μl | EDV μl |
| Group 1 | | | |
| ECHO | | | |
| N = 8* | JBT | 63.7 ± 15.5 | 67.9 ± 22.4 |
| N = 7* | Cont. | 82.9 ± 16.3 | 88.4 ± 27 |
| | P-Value | 0.051 | 0.166 |
| | % Change from Cont. | −23.2 | −23.3 |
| MRI | | | |
| N = 6 | JBT | 101.2 ± 14.7 | 133.3 ± 21.5 |
| N = 5* | Cont. | 131.5 ± 30.5 | 154.2 ± 36.8 |
| | P-Value | 0.12 | 0.35 |
| | % Change from Cont. | −23.1 | −13.6 |
| Group 2 | | | |
| ECHO | | | |
| N = 10 | JBT | 71.3 ± 17.1 | 66.9 ± 14 |
| N = 9 | Control | 72.8 ± 28.2 | 76.5 ± 38 |
| | P-Value | 0.88 | 0.52 |
| | Δ 8- vs- 2-weeks JBT | | −4 ± 14 |
| | Δ 8- vs- 2-weeks Cont. | | +4 ± 17 |
| | Δ value P Value | | 0.31 |

*Mice considered “normal’ were removed from the analysis following the screen 2D-Echocardiograph (JBT-miR2 treated mouse 281 and Control treated mouse 272).
JBT = JBT-miR2, Scrambled Control virus = Cont. EDV = End Diastolic Volume (μl). ESV = End Systolic Volume (μl)

TABLE 11B
Global ECHO and MRI LV Indices JBT-
miR2 or Control Virus Treated Mice

| | | 2-weeks | 8-weeks |
|---|---|---|---|
| | | ESV μl | ESV μl |
| Group 1 | | | |
| ECHO | | | |
| N = 8* | JBT | 39.2 ± 12.9 | 43 ± 15.5 |
| N = 7* | Cont. | 57.4 ± 14.9 | 61.4 ± 20.4 |
| | P-Value | 0.037 | 0.099 |
| | % Change from Cont. | −31.8 | −29.9 |
| MRI | | | |
| N = 6 | JBT | 56.9 ± 12.5 | 82.5 ± 23.5 |
| N = 5* | Cont. | 86.3 ± 27.4 | 100.3 ± 36.6 |
| | P-Value | 0.10 | 0.43 |
| | % Change from Cont. | −34 | −17.7 |
| Group 2 | | Baseline | Follow-up |
| ECHO | | ESV μl | ESV μl |
| N = 10 | JBT | 44.8 ± 19.9 | 41.4 ± 16.4 |
| N = 9 | Control | 44.6 ± 27.8 | 49.8 ± 40.3 |
| | P-Value | 0.99 | 0.6 |
| | Δ 8- vs- 2-weeks JBT | | −3 ± 12 |
| | Δ 8- vs- 2-weeks Cont. | | +5 ± 19 |
| | Δ value P Value | | 0.29 |

TABLE 11C
Global ECHO and MRI LV Indices JBT-
miR2 or Control Virus Treated Mice

| | | 2-weeks | 8-weeks |
|---|---|---|---|
| | | EF % | EF % |
| Group 1 | | | |
| ECHO | | | |
| N = 8* | JBT | 38 ± 8 | 36 ± 6 |
| N = 7* | Cont. | 31 ± 6 | 31 ± 6 |
| | P-Value | 0.093 | 0.175 |
| | % Change from Cont. | +22.7 | +8.3 |
| MRI | | | |
| N = 6 | JBT | 44.3 ± 6.5 | 39.4 ± 8.1 |
| N = 5* | Cont. | 35.9 ± 7.5 | 37.1 ± 9.7 |
| | P-Value | 0.11 | 0.72 |
| | % Change from Cont. | +23.5 | +6.0 |
| Group 2 | | | |
| ECHO | | | |
| N = 10 | JBT | 39 ± 14 | 39 ± 14 |
| N = 9 | Control | 42 ± 14 | 40 ± 13 |
| | P-Value | 0.69 | 0.93 |
| | Δ 8- vs- 2-weeks JBT | | 0 ± 9 |
| | Δ 8- vs- 2-weeks Cont. | | −3 ± 10 |
| | Δ value P Value | | 0.83 |

### Global Heart Function JN-101

When JN-101 (10 mg/kg) or Vehicle was administered at the time of reperfusion, similar reductions in cardiac volumes were observed as for JBt-miR2 treatment (Tables 12A-12B). End Diastolic Volume (EDV) and End Systolic Volume (ESV) were reduced by 32.4% (P=0.027, ECHO) and 36.8% (P=0.097, ECHO), respectively at 2 weeks post IR and JN-101 administration at reperfusion compared to vehicle. At 4-weeks post JN-101 administration, EDV was reduced by 19.1% as determined by ECHO and 17.8% as measured by MRI compared with vehicle. At 4-weeks post JN-101 administration, ESV was reduced by 21.36% as determined by ECHO and 26.36% as measured by MRI compared with vehicle showing consistency in relative cardiac volume changes with both modalities. At 4-weeks post IR, EF increased by 8.6% (ECHO) and 13.14% (MRI) relative to vehicle.

TABLE 12A
Global ECHO and MRI LV Indices JN-101 or Vehicle Treated Mice at Reperfusion

| | | EDV μl | ESV μl | EF % | Max dV/dt |
|---|---|---|---|---|---|
| Week 2 | | | | | |
| ECHO | | | | | |
| N = 8* | JN-101 | 58.5 ± 8.4 | 34.2 ± 5.8 | 41 ± 6 | 0.75 ± 0.22 |
| N = 8 | vehicle | 86.5 ± 31.1 | 54.1 ± 30.1 | 40 ± 13 | 0.99 ± 0.23 |
| | P-Value | 0.027 | 0.087 | 0.92 | 0.05 |
| | % Change from | −32.4 | −36.8 | +1.2 | −23.9 |
| | Vehicle. | | | | |
| Week 4 | | | | | |
| N = 8* | JN-101 | 61.7 ± 17.2 | 36.9 ± 10.3 | 39 ± 9 | 0.67 ± 0.36 |
| N = 8 | Vehicle | 76.3 ± 36.7 | 46.9 ± 34.7 | 42 ± 11 | 1.07 ± 0.34 |
| | P-Value | 0.33 | 0.45 | 0.48 | 0.04 |
| | % Change from | −19.1 | −21.36 | +8.6 | −37.05 |
| | Vehicle. | | | | |
| Week 4 | | | | | |
| MRI | | EDV μl | ESV μl | EF % | ED-SA |
| N = 10 | JN-101 | 102.4 ± 17.44 | 56.7 ± 19.95 | 45.8 ± 10.43 | 98.76 ± 10.65 |
| N = 7 | Vehicle | 124.57 ± 43.2 | 77.05 ± 41.44 | 40.5 ± 9.78 | 112.4 ± 24.8 |
| | P-Value | 0.24 | 0.26 | 0.30 | 0.21 |
| | % Change from | −17.8 | −26.36 | +13.14 | −12.11 |
| | Vehicle. | | | | |

*211 excluded due to poor image quality in JN-101 treated animals;
*215 from the JN-101 treated animals was removed due to extracardiac mass

TABLE 12B
Global ECHO and MRI LV Indices JN-101
or Vehicle Treated Mice at Reperfusion

| | | EDLVM | dσ*/dtmax | HR (BPM) |
|---|---|---|---|---|
| Week 2 | | | | |
| ECHO | | | | |
| N = 8* | JN-101 | 82 ± 14 | 0.014 ± 0.005 | 610 ± 72 |
| N = 8 | vehicle | 85 ± 9 | 0.018 ± 0.005 | 535 ± 69 |
| | P-Value | 0.58 | 0.16 | 0.05 |
| | % Change from Vehicle. | −4.0 | −20.6 | +14.2 |
| Week 4 | | | | |
| N = 8* | JN-101 | 81 ± 11 | 0.013 ± 0.008 | 579 ± 66 |
| N = 8 | Vehicle | 85 ± 11 | 0.019 ± 0.005 | 594 ± 33 |
| | P-Value | 0.56 | 0.08 | 0.59 |
| | % Change from Vehicle. | −3.84 | −32.76 | −2.45 |

| Week 4 | | | |
|---|---|---|---|
| MRI | | ES-SA | SA-% Chg |
| N = 10 | JN-101 | 70.43 ± 14.64 | 29.24 ± 7.8 |
| N = 7 | Vehicle | 84.98 ± 27.6 | 25.5 ± 7.43 |
| | P-Value | 0.24 | 0.33 |
| | % Change from Vehicle. | −17.12 | +14.87 |

*211 excluded due to poor image quality in JN-101 treated animals;
*215 from the JN-101 treated animals was removed due to extracardiac mass

### Consistency of Ischemic Reperfusion Injury Between Randomized Groups Before Treatment

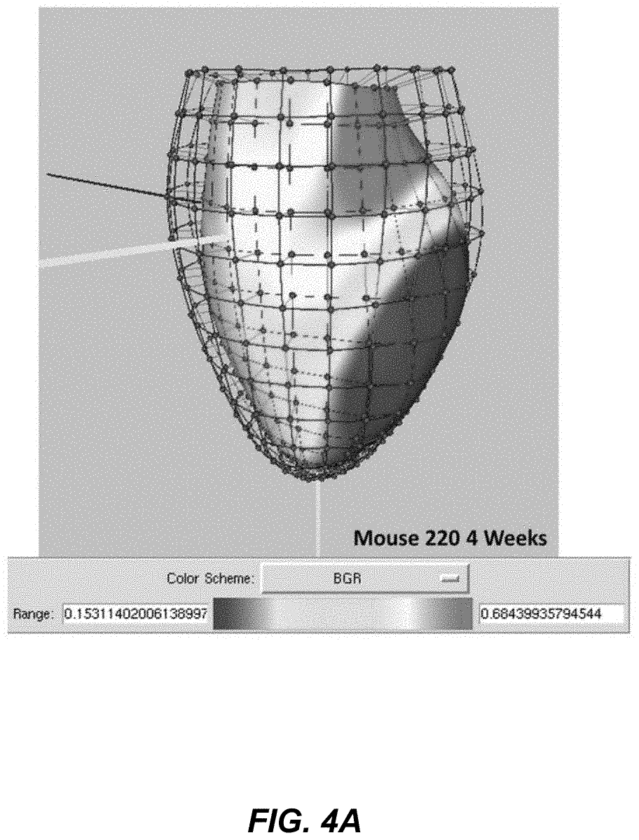

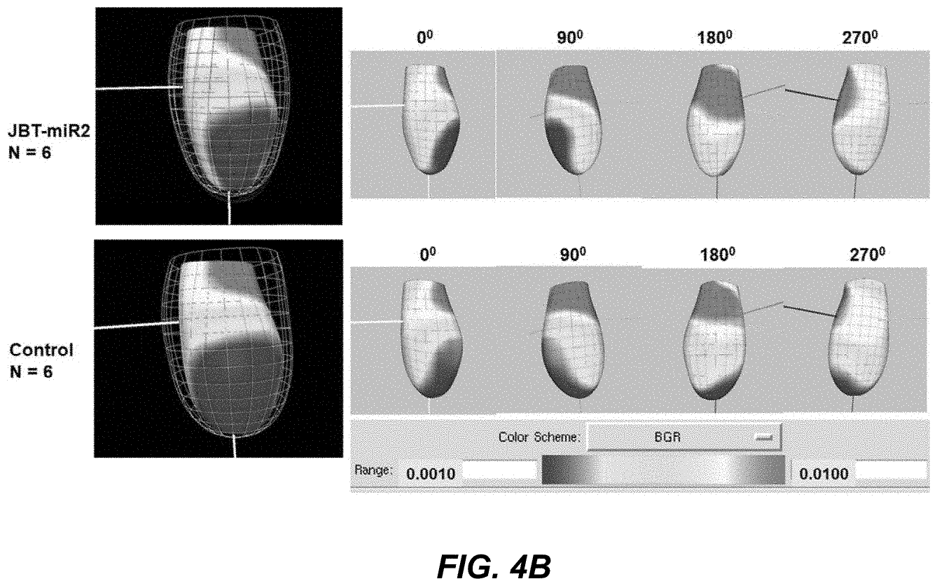

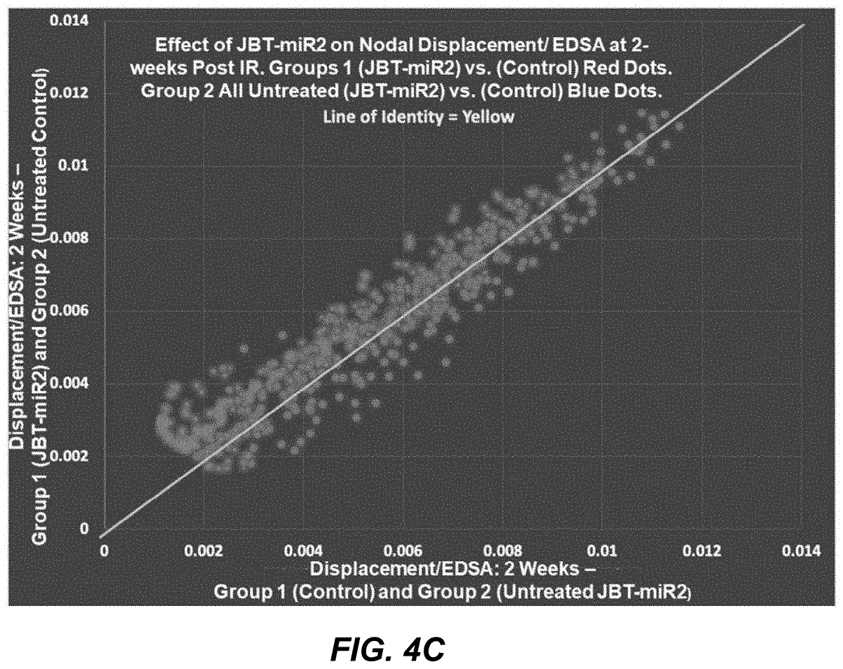

Confidence in the consistency of surgical injury and area of risk (AOR) between JBT-miR2 and Control Groups is reflected by comparable 2-week baseline ECHO measures of LV indices for Group 2 mice (Tables 11A-11C) and that nodal displacement/EDSA is not shifted from the line of identity in untreated Groups at 2-weeks (blue dots) (FIG. 4C).

### Regional Heart Function JBT-miR2

For Group 1 mice, regional wall motion improvement was demonstrated by both ECHO and MRI modalities. Strain increased by 10% in the infarcted area (nodes 23-37) (a 50-node perimeter ECHO analysis of the left ventricle (LV)) at week 2 and week 8 post JBT-miR2 administration compared with Control (FIG. 3B).

Using MRI, the LV endocardial shape was reconstructed from 9 separate, stacked slices, taken at a spatial resolution of 0.5 mm, from base to apex (FIG. 4A). The shape at end-diastole (ED) and end-systole (ES) were both fitted, by a least squares routine, to a prolate spheroid, with 300 equidistant nodes on its surface. Displacement in space of each node is then calculated between ED and ES, and then normalized for overall LV size by the end-diastolic surface area (EDSA) providing a finite element measure of myocardial shortening (contraction). Composite images were obtained by averaging data at each of the corresponding nodes defined by the prolate spheroid fit. The LV ES shape is color-coded topographically to reflect low to high degrees of nodal displacement. Note the low displacement values (less dark blue) in the area of the antero-apical infarction of mice 2-weeks after a single administration of JBT-miR2 (FIG. 4B). This data is represented graphically in FIG. 4C with red nodal displacement normalized to ED surface area (EDSA) shifted above the line of identity with JBT-miR2 treatment. Effects were not as prominent for Group 2 (data not shown).

### Regional Heart Function JN-101

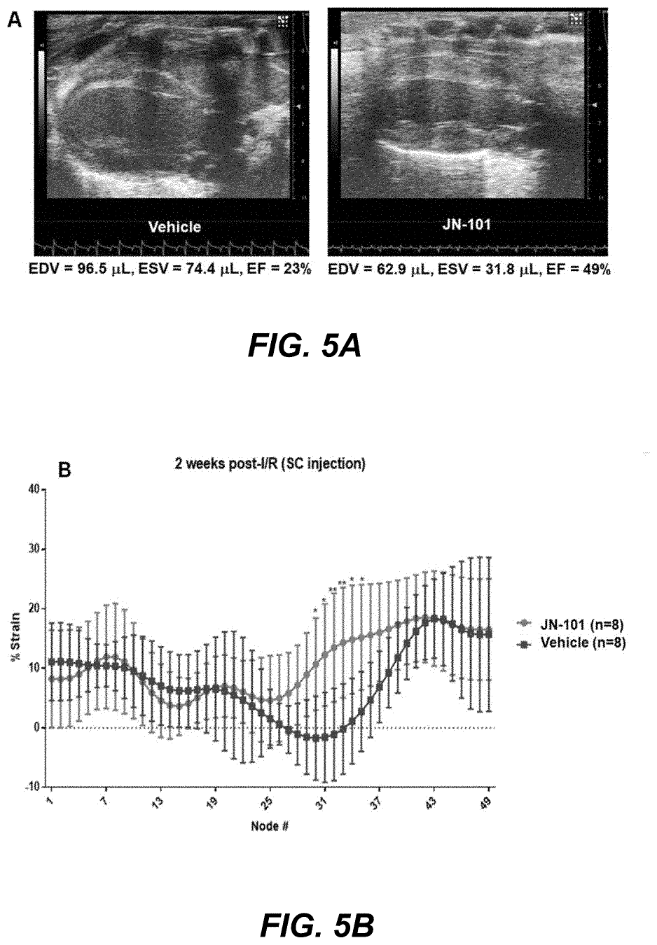

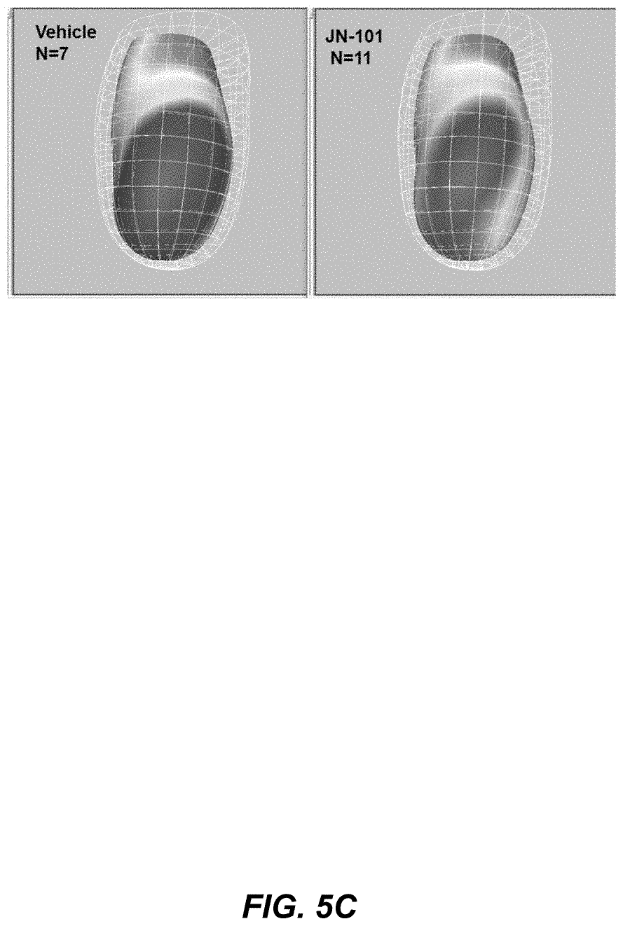

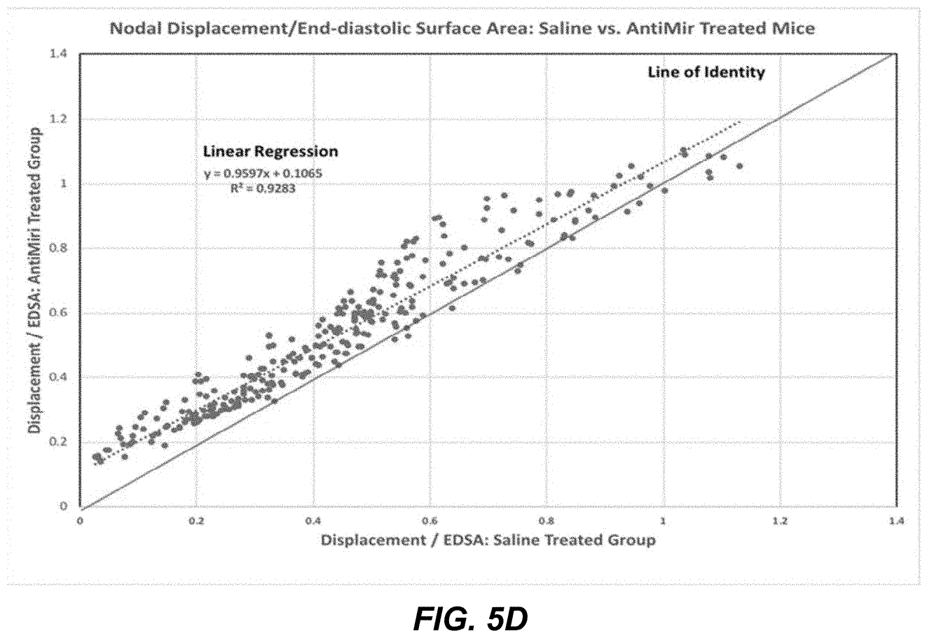

Consistent with the cardiac volume decreases at 2-weeks post JN-101 administration, % strain significantly increased in the infarcted area of the left ventricle at nodes 30 to 36 (p<0.05 node 30, 31, 34 36 and P<0.01 nodes 32 and 33) (FIGS. 5A and B). Composite 3D images of the LV of JN-101 treated mice showed a strong and statistically significant indication that the JN-101 is causing a significant enhancement of basal myocardial shortening in the weeks following IR injury compared to Vehicle 4-weeks following treatment (FIGS. 5C and D).

### Terminal Hemodynamics JBT-miR2

Group 1:

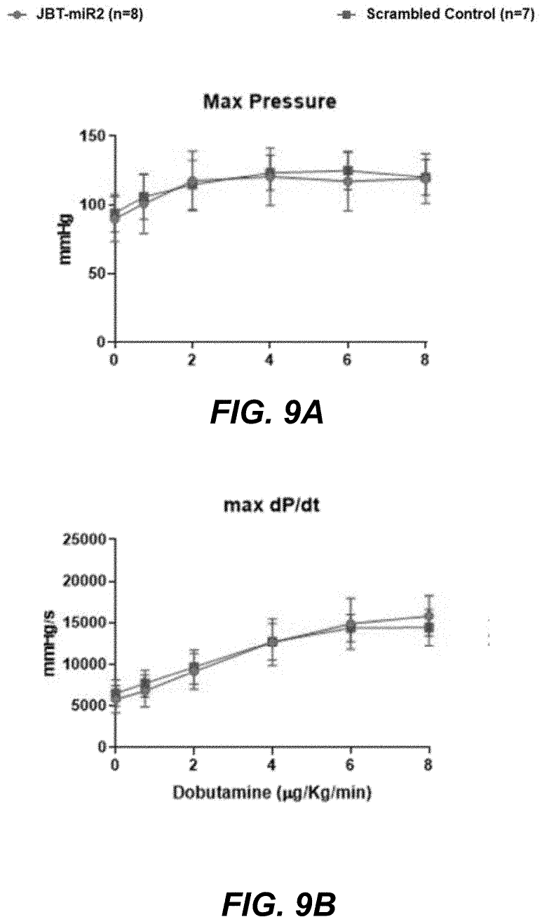

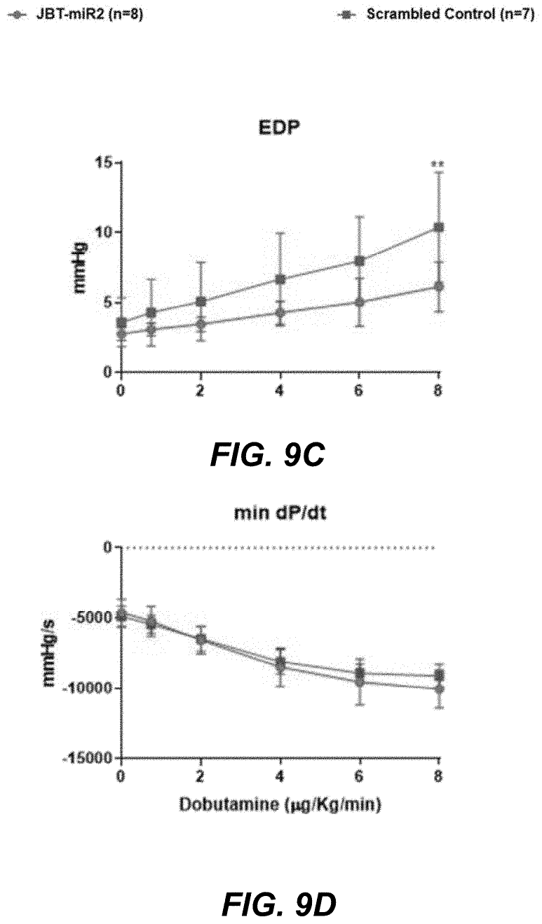

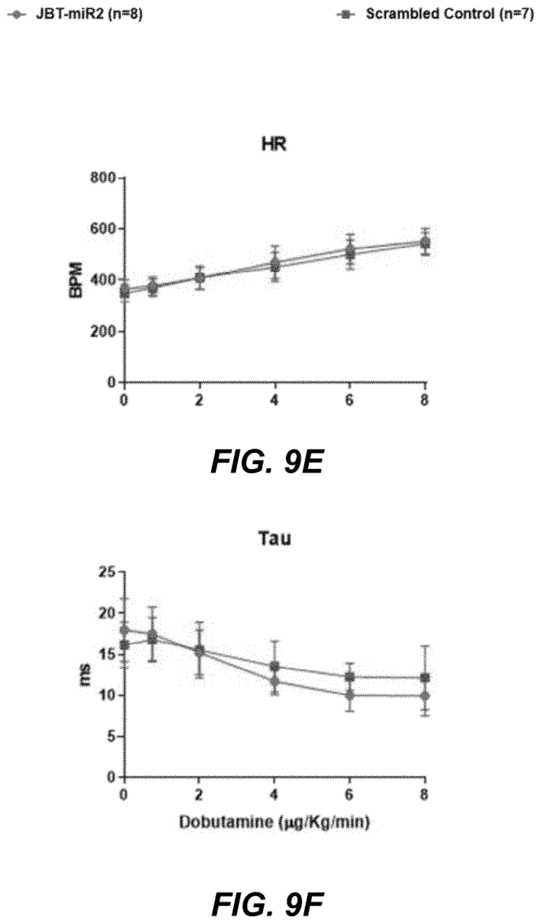

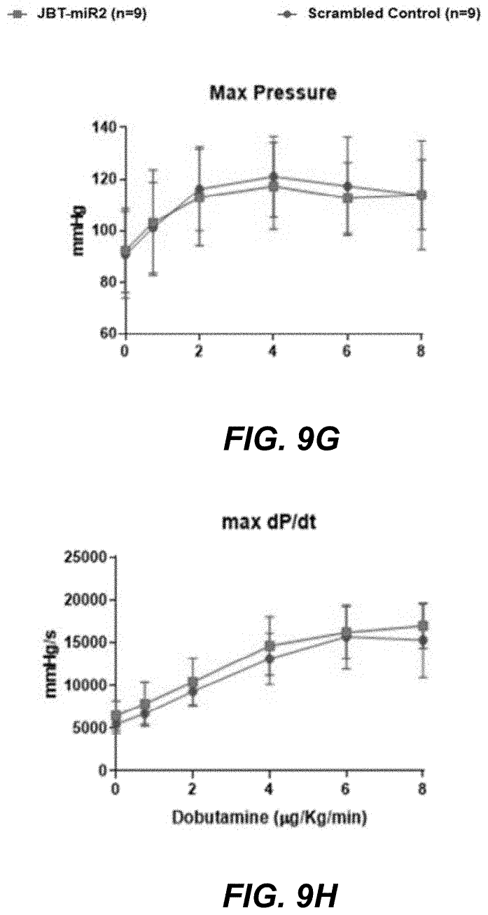

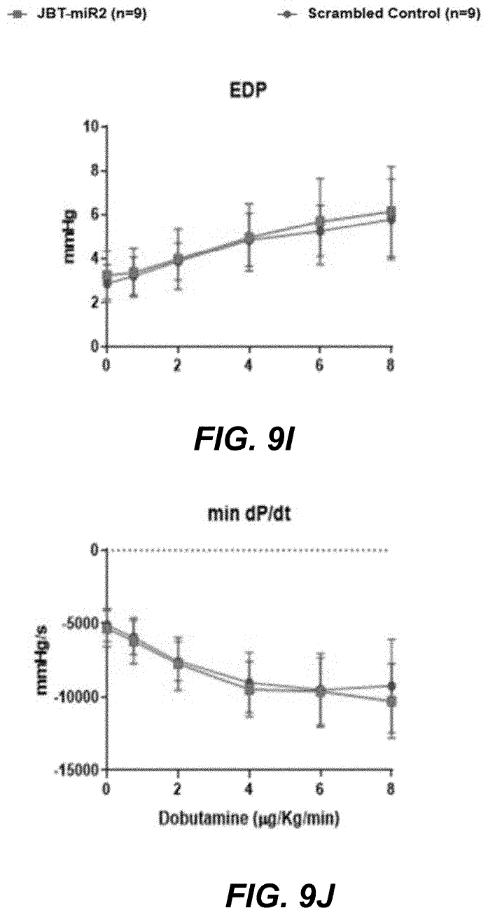

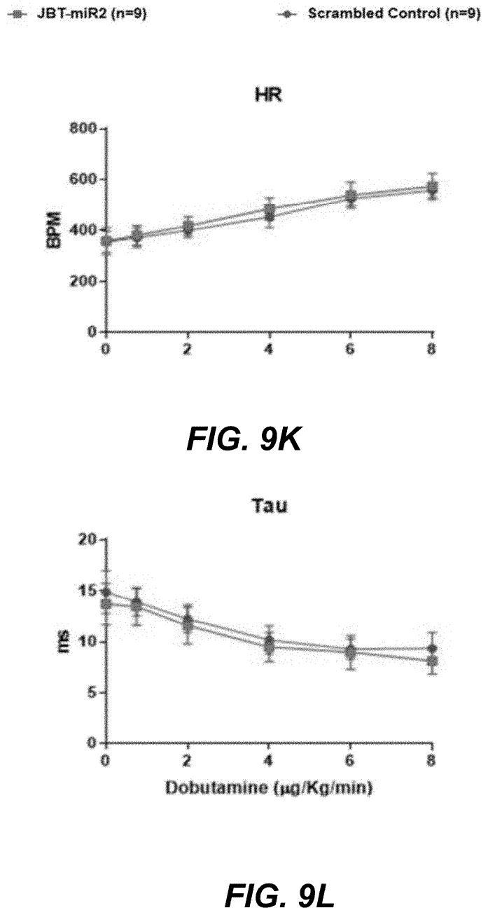

When JBT-miR2 was administered to mice at the time of reperfusion following Ischemia, no difference in maximum pressure between the JBT-miR2 treated group and the Control group were found indicating that the drug does not hinder the heart's ability to dilate. There were no differences in heart rate at baseline and with dobutamine stimulation between the treatment groups. Hearts treated with JBT-miR2 experienced nearly significantly to significantly lower end diastolic pressure (EDP) when stimulated with 6 μg of dobutamine, (5.1 mmHg JBT-miR2 Vs. 8.0 mmHg Control P=0.056). With 8 μg of dobutamine stimulation EDP was 6.1 mmHg in JBT-miR2 treated mice Vs. 10.4 mmHg in Control virus treated mice, P=0.029, indicating ease of dilation. For mice administered with JBT-miR2, the maximum rate of pressure change (Max dP/dt) was 1392.1 mmHg/s higher when stimulated with 8 μg dobutamine (15820.7 mmHg/s JBT-miR2 vs. 14428.6 mmHg/s Control P=0.262) compared to mice treated with control virus, indicating an increase in contractility with JBT-miR2 treatment. The absolute value of the minimum rate of pressure change (Min dP/dt) was nearly significantly higher with 8 μg dobutamine stimulation (−10050.7 mmHg/s JBT-miR2 Vs. −9121.2 mmHg/s Control P=0.128) for mice treated with JBT-miR2 compared to mice administered with control virus, indicating an increase in diastolic function with JBT-miR2 administration. In hearts treated with JBT-miR2, exponential tau was significantly lower with 6 μg administration of dobutamine (10 ms JBT-miR2 vs 12.3 ms Control P=0.027) compared to mice administered with Control virus, further indicating the impact of the drug on increasing diastolic function (FIGS. 9A-9L).

Group 2:

When JBT-miR2 was administered to mice two weeks after reperfusion following Ischemia, there were no differences in maximum pressure between the JBT-miR2 treated group and the Control group at baseline or with dobutamine stimulation. There were no differences in heart rate at baseline or with dobutamine stimulation between the treatment groups. There were no significant differences in EDP, Min dP/dt, and Max dP/dt between hearts treated with JBT-miR2 and Control virus. To note, for both Min dP/dt and Max dP/dt, the average difference between JBT-miR2 and Control for both measurements was approximately 1000 mmHg/s higher for Max dP/dt and 1000 mmHg/s lower for Min dP/dt respectively. Exponential tau in hearts treated with JBT-miR2 was nearly significantly lower (1.2 ms difference) compared to Control virus when stimulated with 8 μg (8.1 ms JBTmiR2 Vs. 9.3 ms Control P=0.092), indicating an increased ability to relax consistent with when JBT-miR2 is given at the time of reperfusion in Group 1 mice (FIGS. 9A-9L).

### Terminal Hemodynamics JN-101

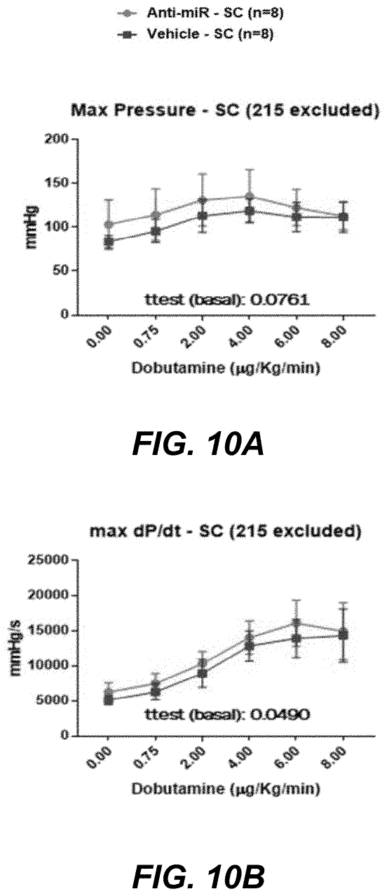

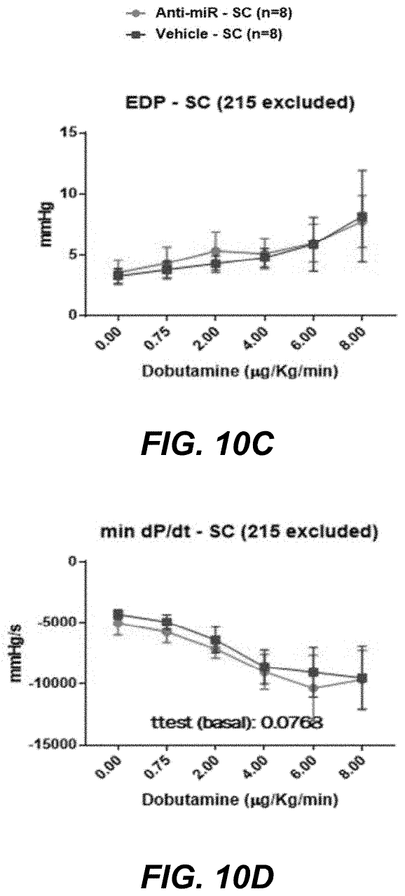

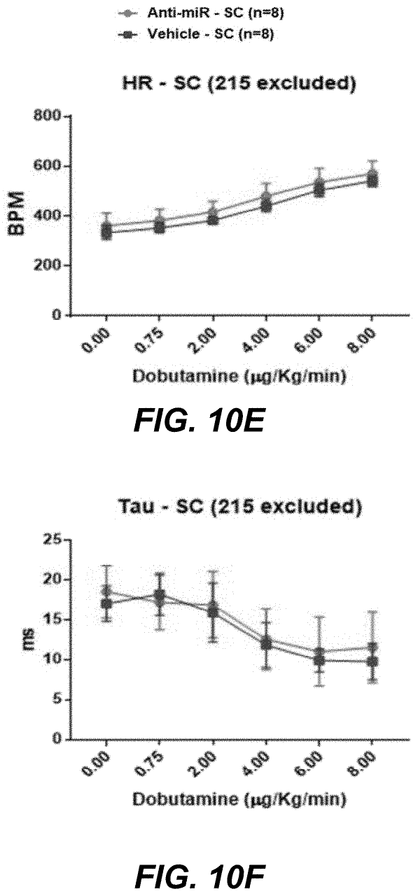

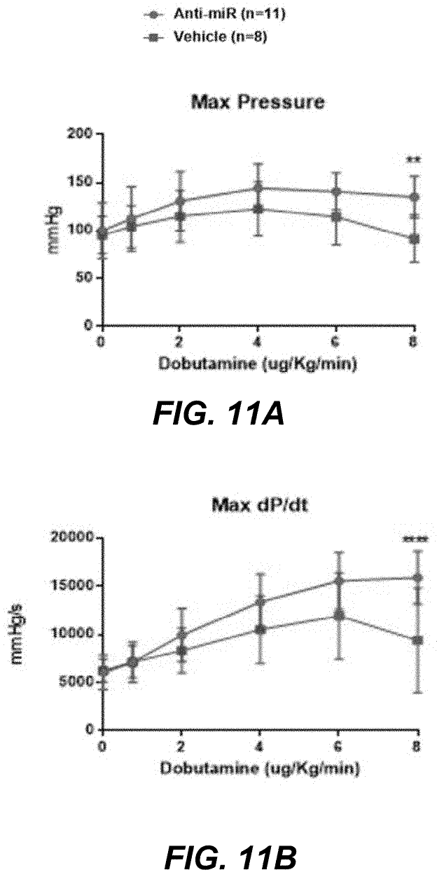

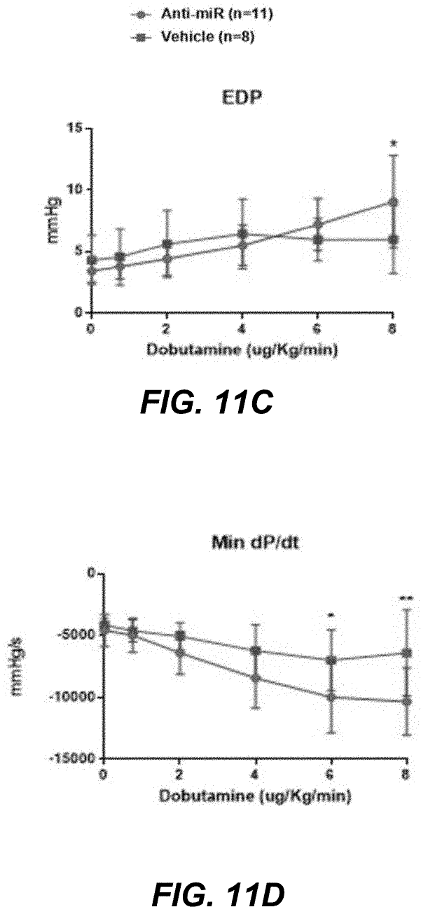

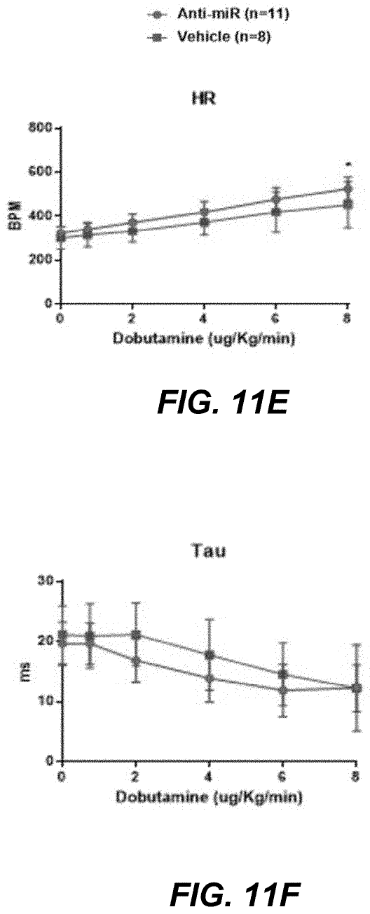

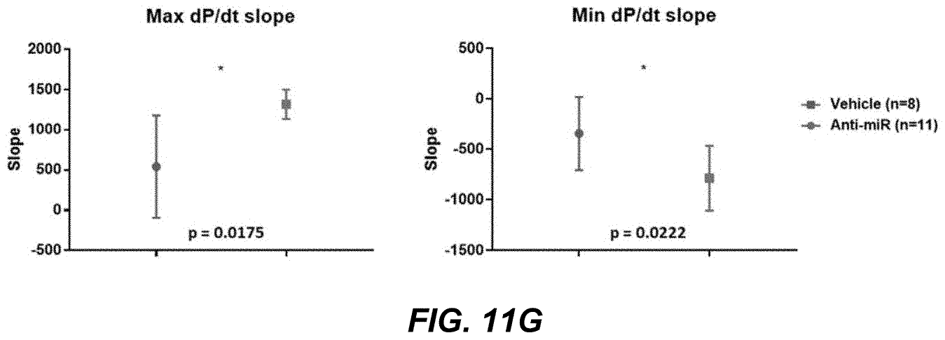

N-101 treated mice had a significant increased basal dP/dt max (6182.68+/−1415 mmHg/s JN-101 Vs. 5139.45+/−668.8 mmHg/s Vehicle P=0.049) 4-weeks after administration as measured by terminal hemodynamics, suggesting the formation of functionally active, mature cardiac myocytes with JN-101 treatment post cardiac ischemia (FIGS. 10A-10F). In a separate group of mice where JN-101 was administered 2-weeks after IR injury terminal hemodynamics showed significant differences in stimulation measured for Max Pressure mmHg with 8 μg dobutamine (P=0.0024) in JN-101 treated mice compared to Vehicle treated mice. Similarly JN-101 treated mice had elevated max dP/dt (mmHgs/s) with 8 μg dobutamine (P=0.0185) and increased relaxation Min dP/dt mmHg/s with 6 μg and 8 μg dobutamine (P=0.036 and P=0.0275). There were significant differences in the slope for Min and max dP/dt. These data suggest that JN-101 can be administered to mice with IR Injury, 2-weeks after the injury and suggests the formation of functionally active cardiac myocytes that can respond to dobutamine stimulation after a significant time lapse from the time of Ischemic injury (FIGS. 11A-11G).

### Histology

Scar Size JBT-miR2:

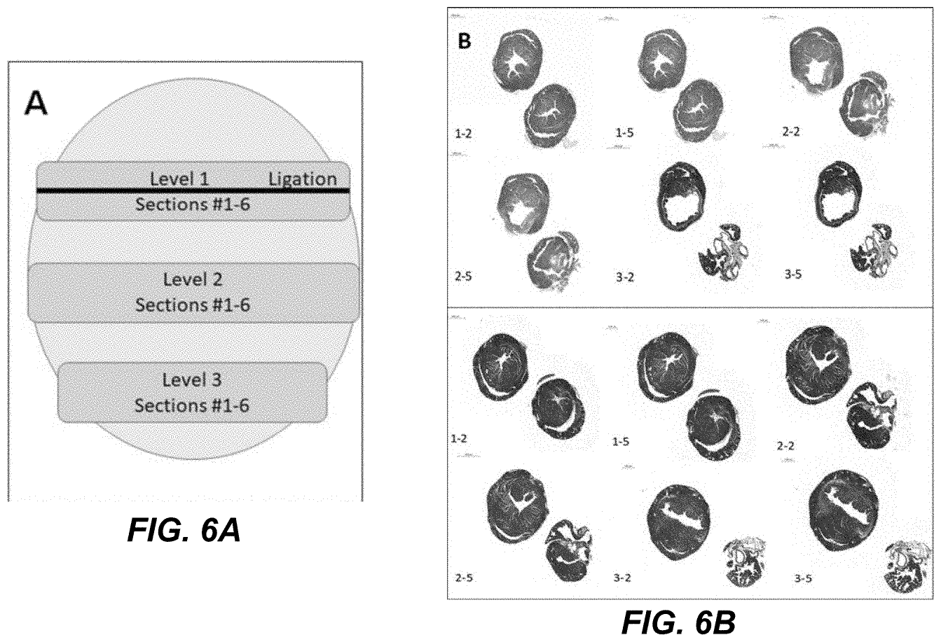

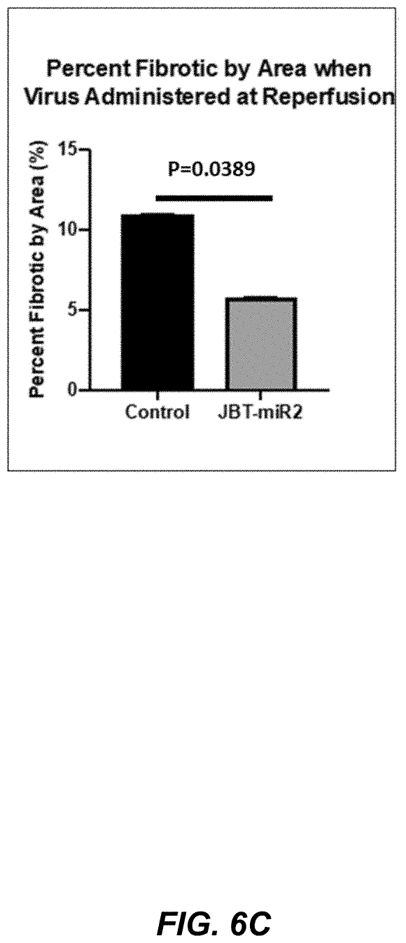

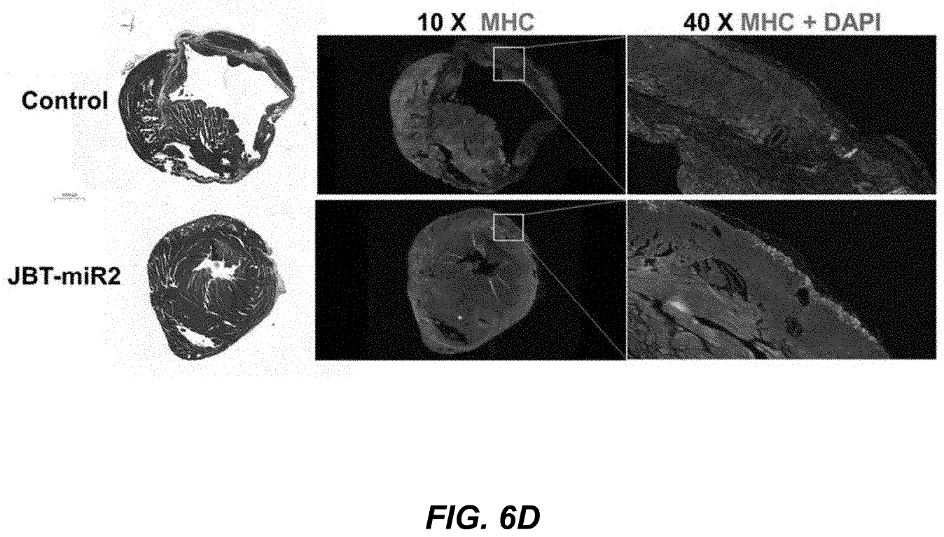

Hearts from select surviving mice were serially sectioned hearts from 1 mm above the LCA ligation to the apex a 4 μM intervals at three levels were stained with Trichrome Masson (TM) (FIG. 6A). Scar was quantified per using Image^{DX} software per mm\hat{ }² of total tissue of two serial sections per level, averaged and expressed as a percentage of total tissue area. In Group 1, JBT-miR2 treated hearts had a significant, 47.7% reduction in fibrotic tissue (5.73%, N=5) compared to hearts treated with Control (10.96%, N=3) (P=0.039) (FIG. 6C). Representative sections from a Control and JBT-miR2 treated mouse are shown in FIG. 6B.

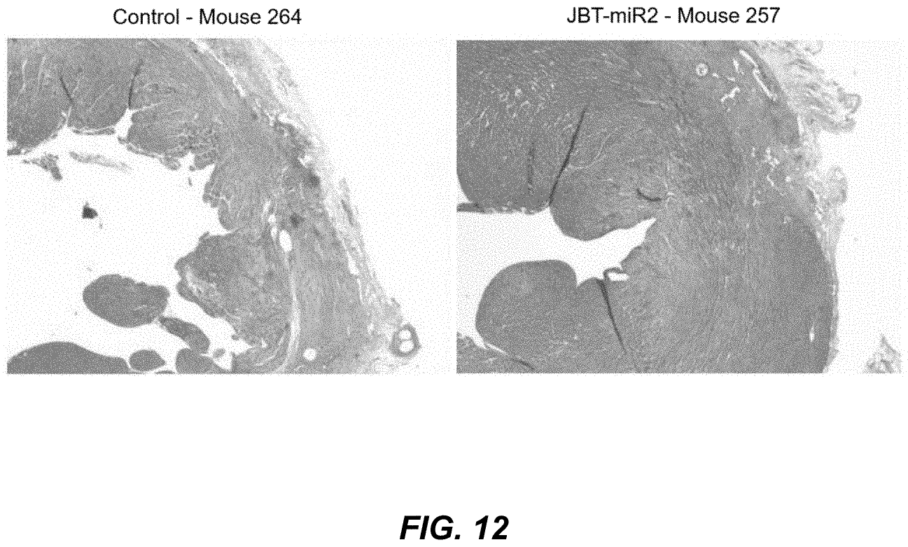

Qualitative pathology conducted by a board certified pathologist analyzed the remaining mice hearts from Group 1 (N=3 JBT-miR2, N=4 Control). Tissues received in formalin were examined grossly, dissected for placement into cassettes and processed into paraffin. Histologic sections were stained with H&E and assessed qualitatively as follows: Normal=0, 1 (+) to 3 (+++) Minimal to Maximal fibrosis and thinning. The official pathology findings were that: IBT-miR2 moderates ischemic injury when given at the time of reperfusion (Group 1, FIG. 12). For select Group 2 mice samples, “The hearts of 3 ischemic mice were histologically normalized when JBT-miR2 was given 2-weeks after reperfusion”. The pathology report concluded that JBT-miR2 moderates ischemic injury when given at the time of reperfusion and 2 weeks post-ischemia. The hearts of 3 ischemic mice in group C were histologically normalized when JBT-miR2 was given 2 weeks after ischemia. There were no histological changes indicative of toxicity observed in any tissues examined including the liver in both Group 1 and Group 2 mice.

Histology JBT-miR2:

The number of Myosin Heavy Chain (MHC) positive green Cardiomyocytes per section area was 14% greater in JBT-miR2 treated mice indicative of re-differentiation following proliferation [10,595±1036 Control N=3 vs 12,092±790 JBT-miR2 N=5, P=0.06, average of 6 sections/heart/mouse] (FIG. 6D). In support of CM specificity, miR downregulation did not affect proliferation of human fibroblasts or vascular cells.

### Distribution JBT-miR2:

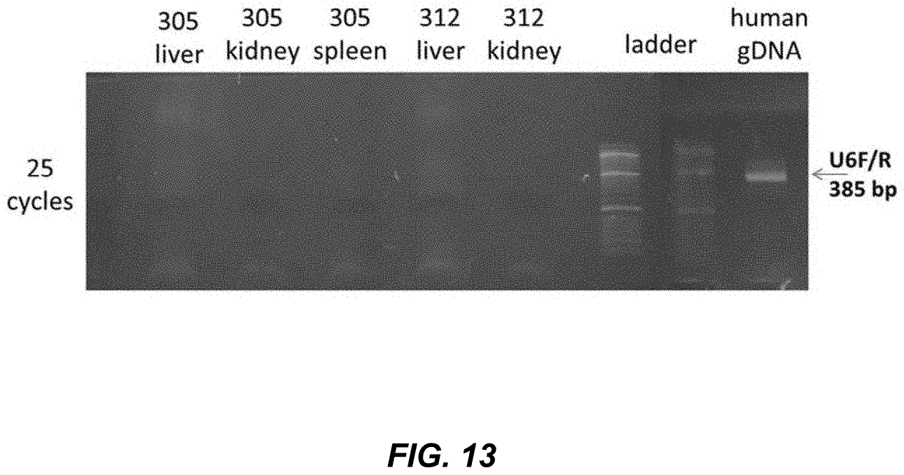

Virus and miR knock down was not detected in tissues as determined by qPCR using primers for the human U6 promoter 6-weeks after administration and is consistent with the non-integrative nature of the AAV2/9 (FIG. 13).

### Off-Target Histopathological and Pleiotropic Effects JBT-miR2:

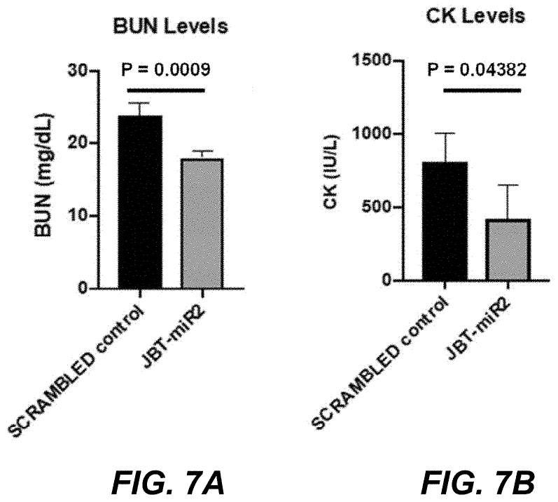

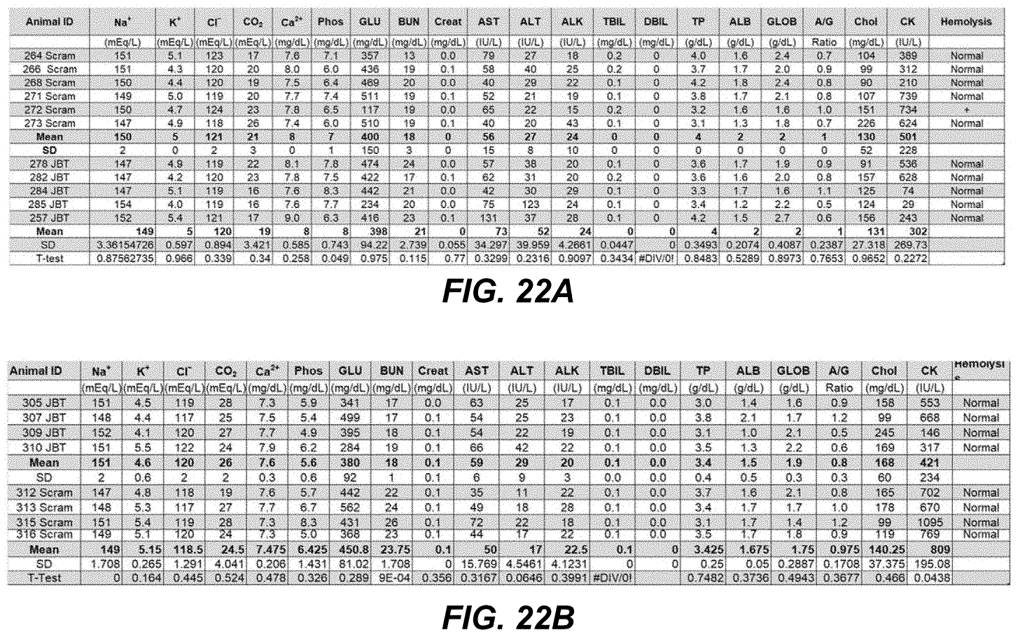

Metabolic Blood Function Tests (MFTs): MFTs on terminal blood collected at 8-weeks were analyzed by Rabbit and Rodent Diagnostic Services (RADA), San Diego, 92121. Group 1: No differences were measured between JBT-miR2 vs. Control treated mice for sodium, potassium, chloride, carbon dioxide, calcium, glucose, blood urea nitrogen, creatine, aspartate aminotransferase (AST), Alanine transaminase (ALT), Alkaline phosphatase, bilirubin, protein, albumin, globulin, cholesterol or creatine kinase (CK). However, in mice treated with JBT-miR2, CK levels decreased by ˜40% (JBT-miR2 (N=5) 302±269.7 vs Control (N=6) 501±228 IUL, P=0.22). Group 2: JBT-miR2 treated mice had significantly lower BUN levels than those treated with Control, possibly indicating that the drug aids kidneys in removing urea from the blood more efficiently (P=0.001). Mice treated with JBT-miR2 also had 50% lower CK levels (P=0.04) (FIG. 7, FIGS. 22A-22B). Pathology: General Approach to Histopathologic Evaluation of toxicity showed no indications of toxicological changes in the heart, spleen, lung, kidney, liver, skin, skeletal muscle and brain (Troyer). Body weight and heart rates were similar between Groups (FIG. 12).

### NGS JBT-miR2:

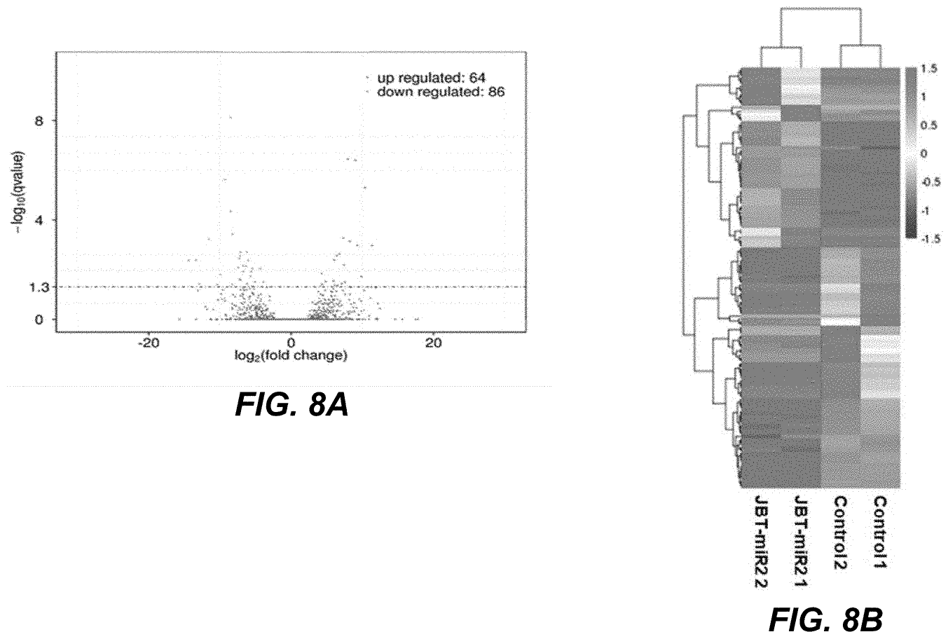

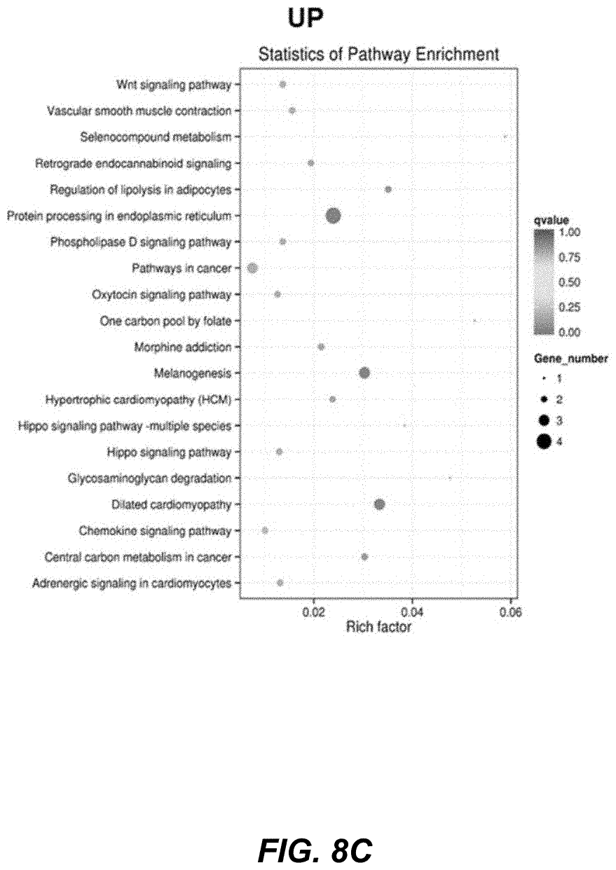

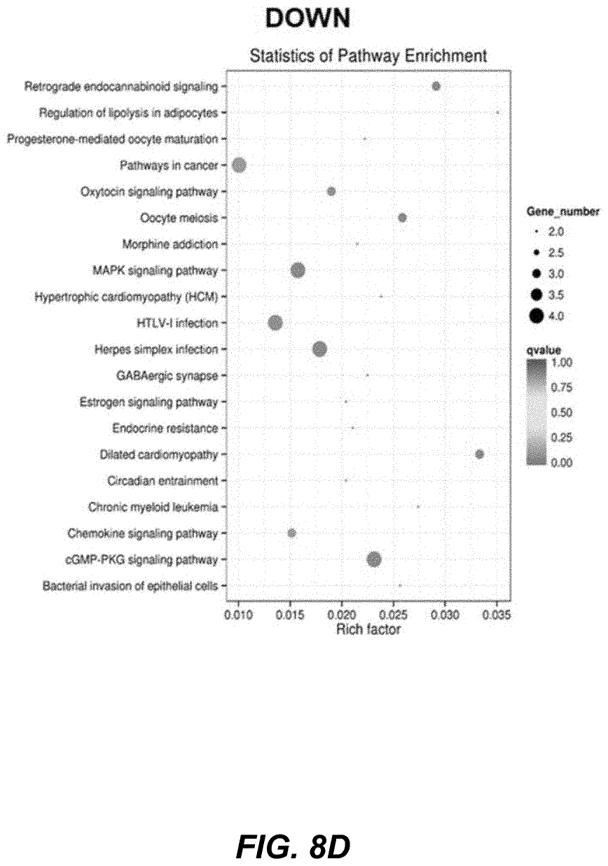

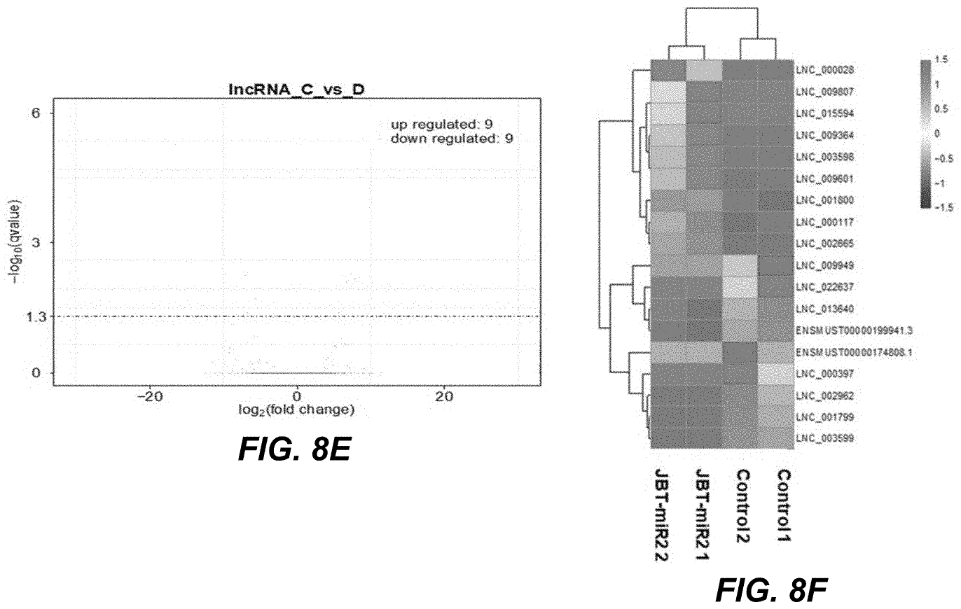

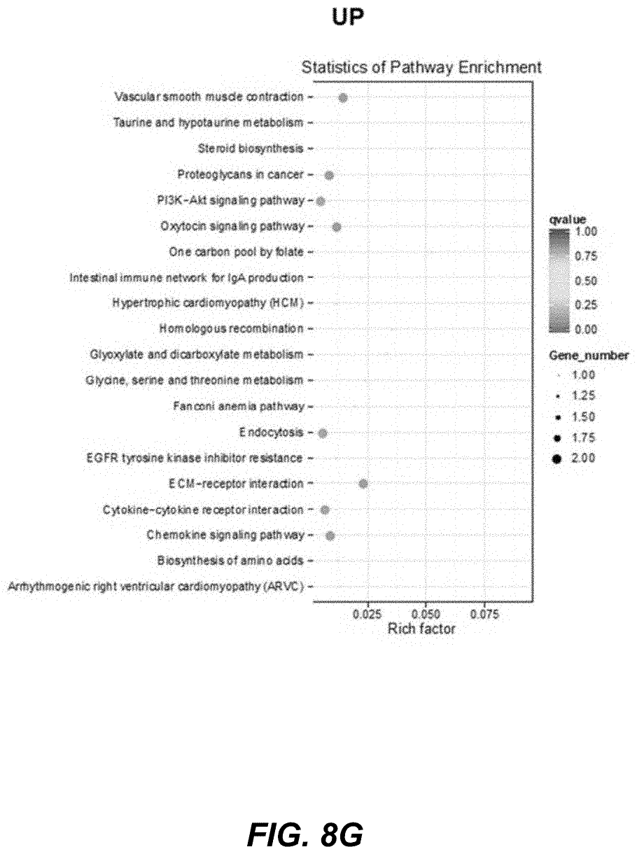

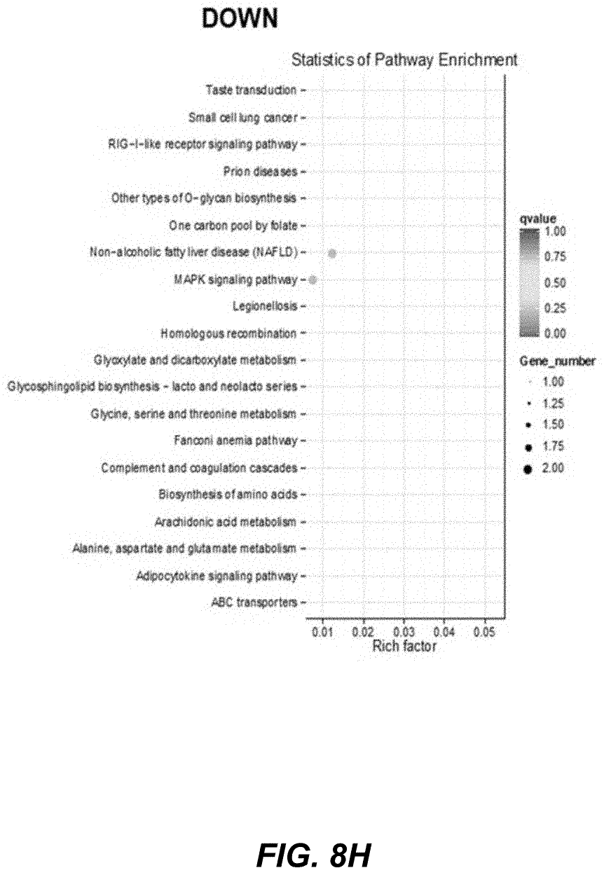

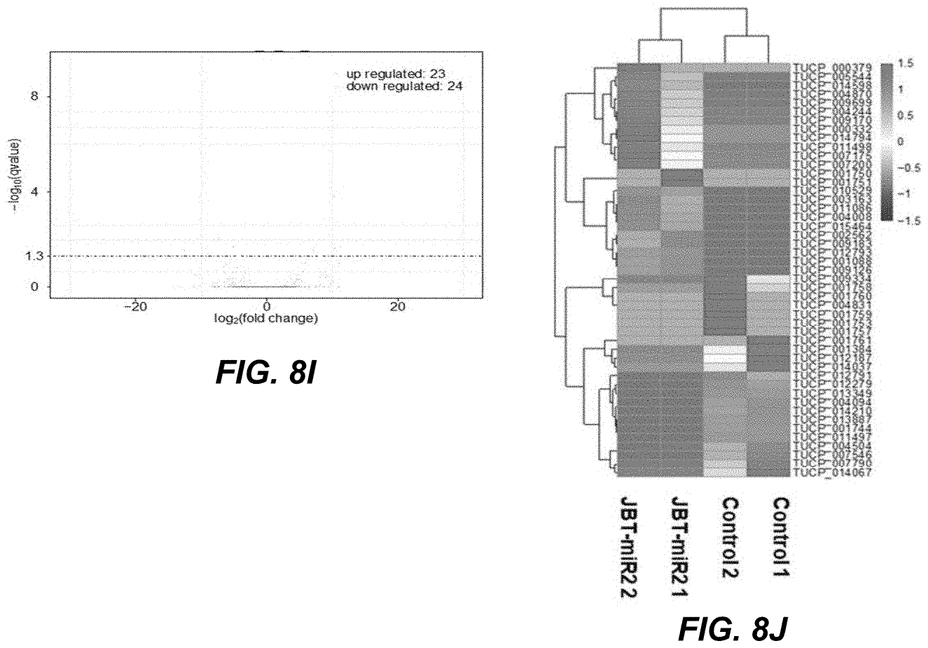

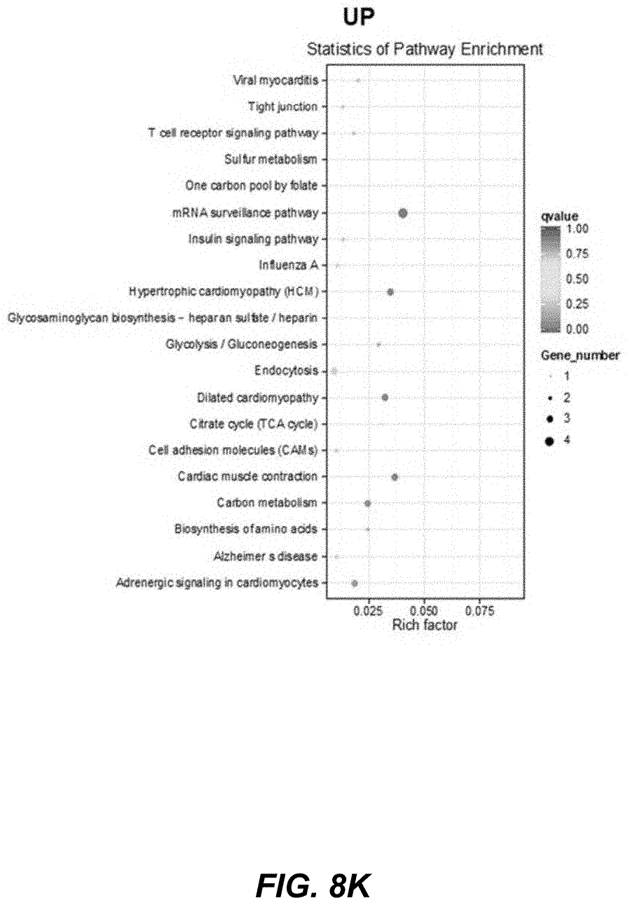

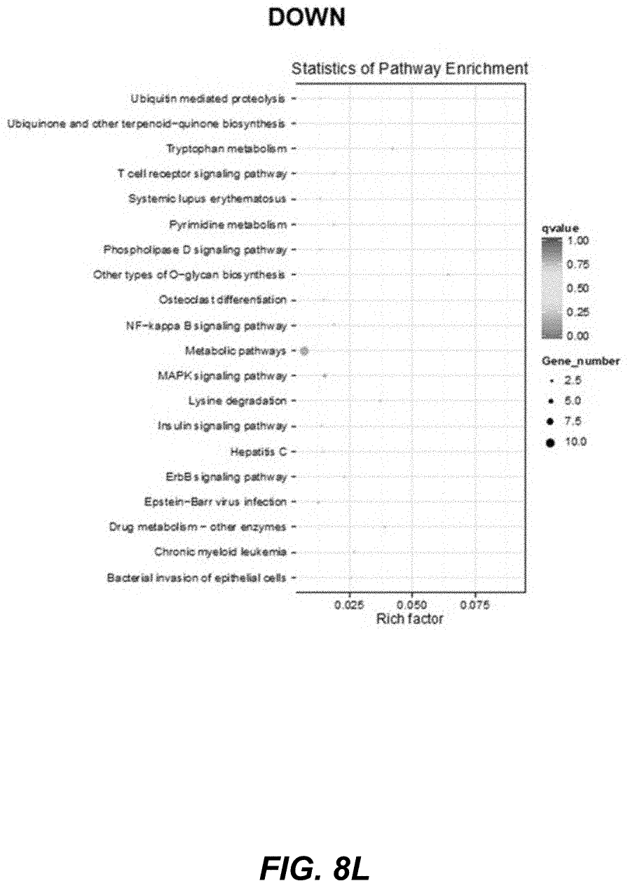

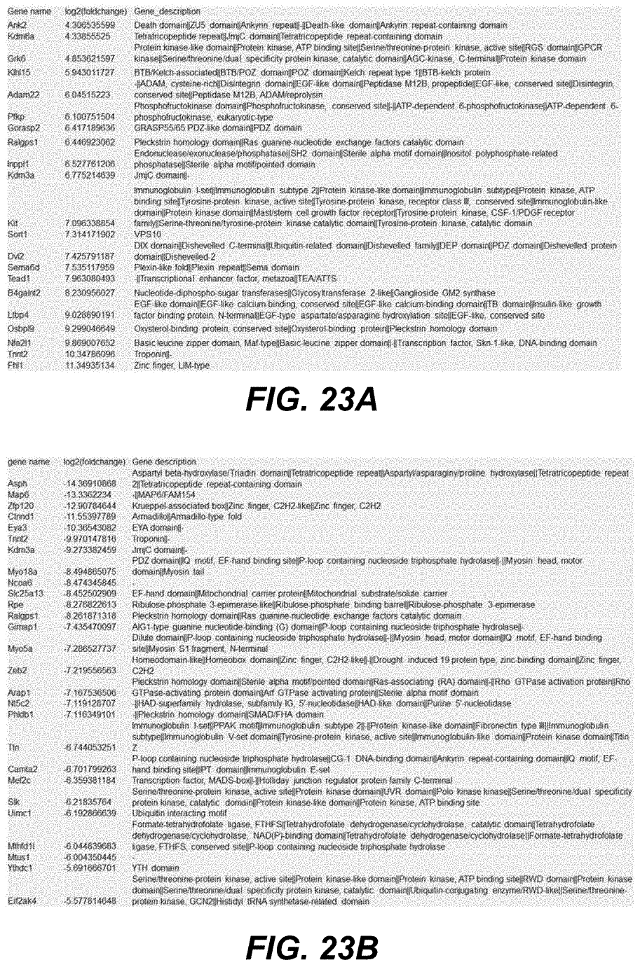

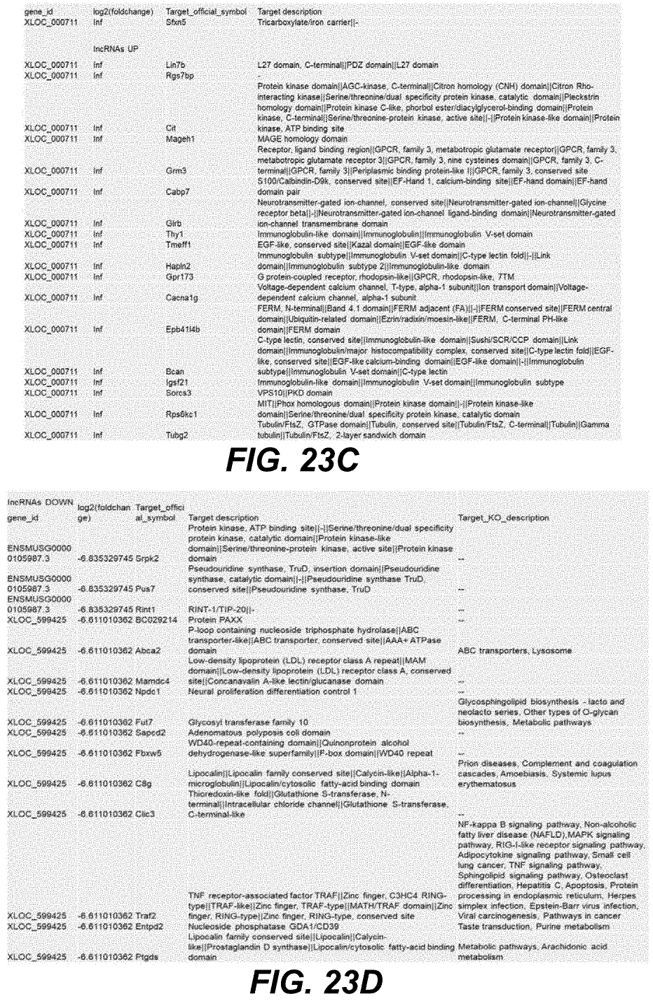

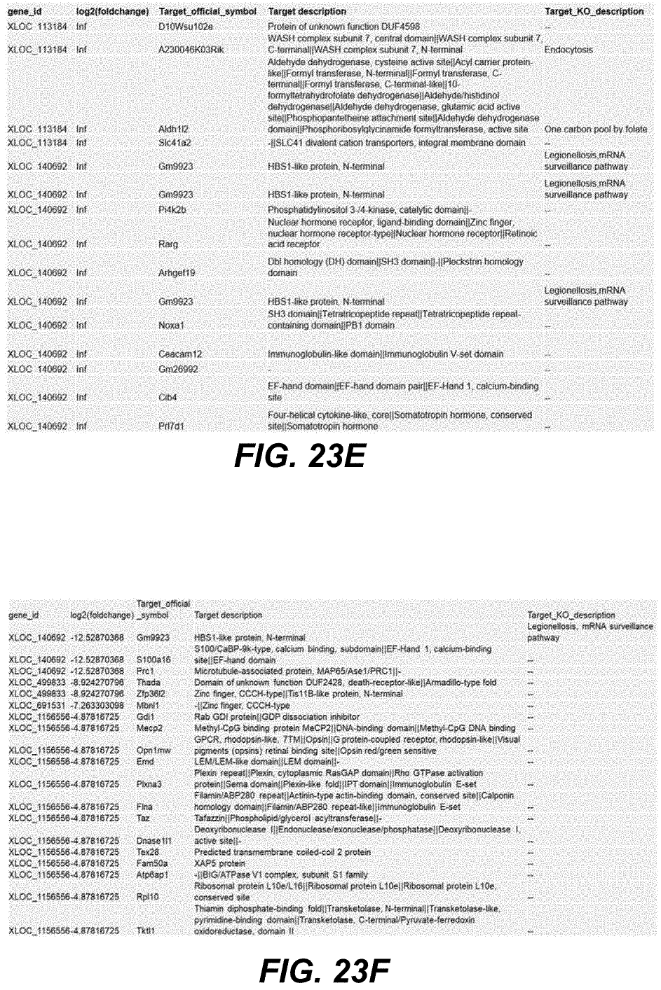

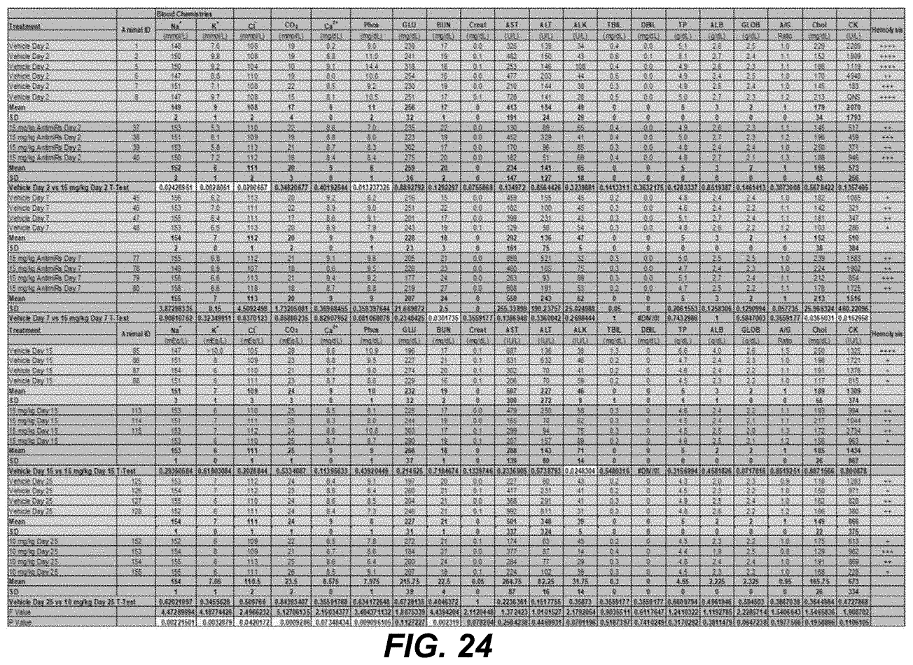

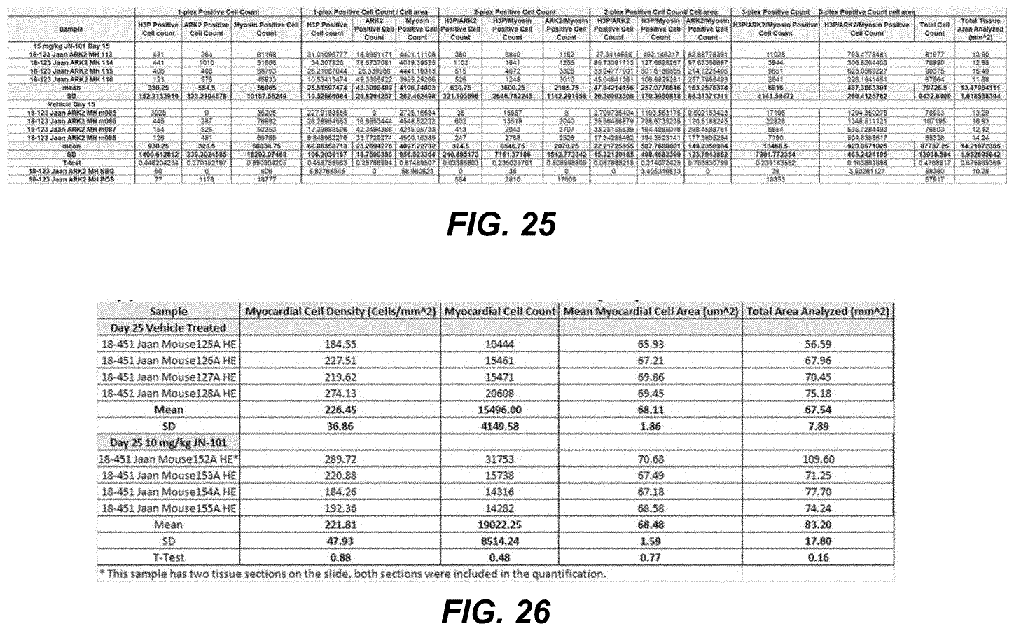

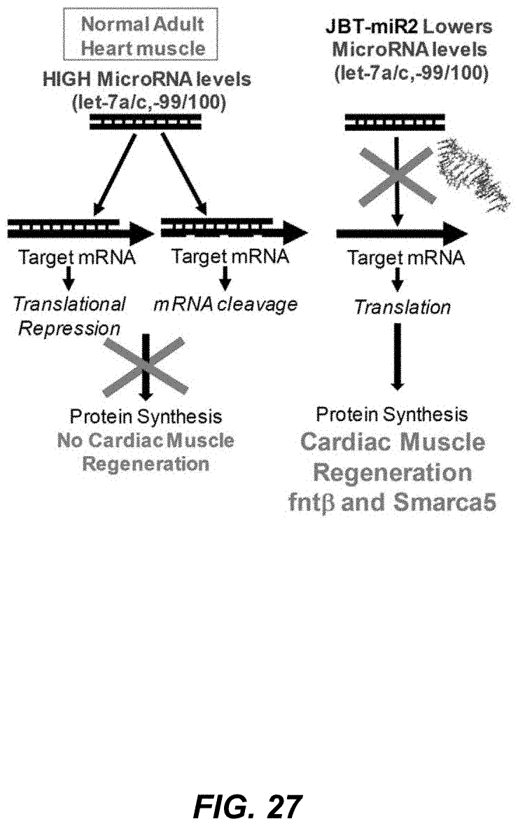

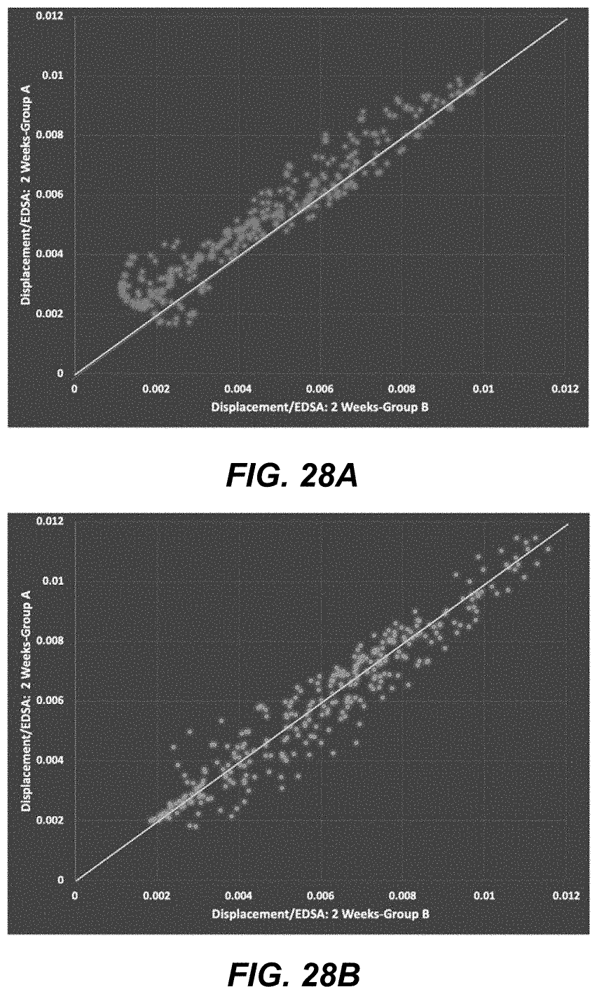

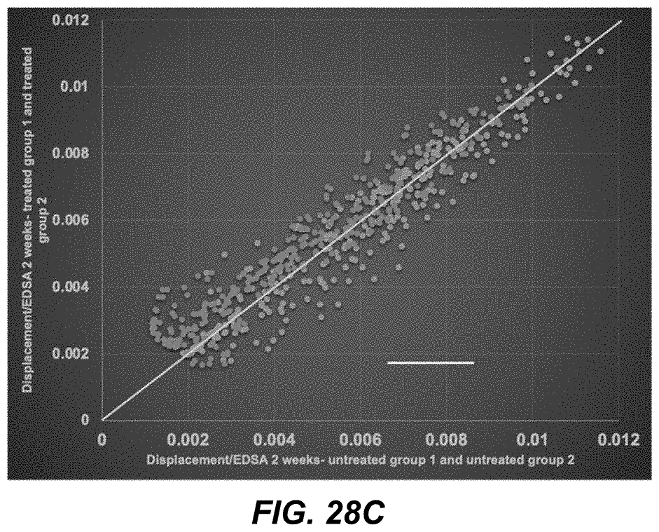

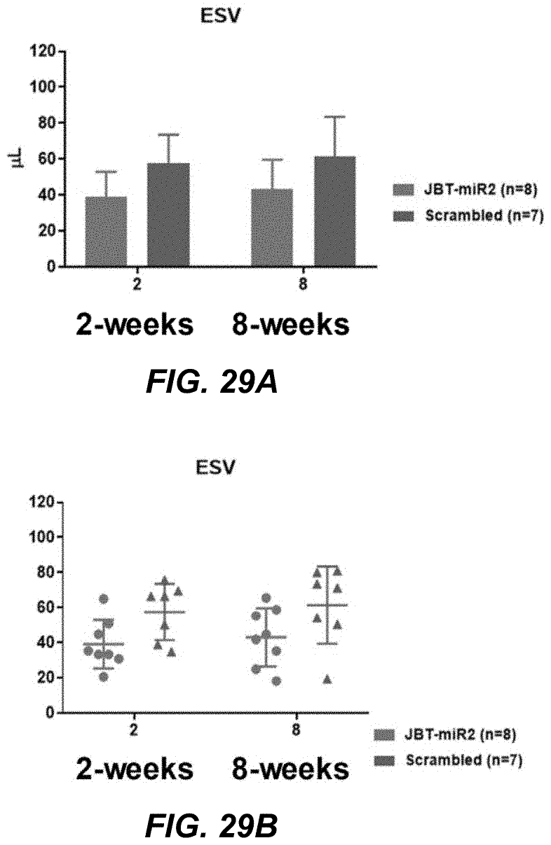

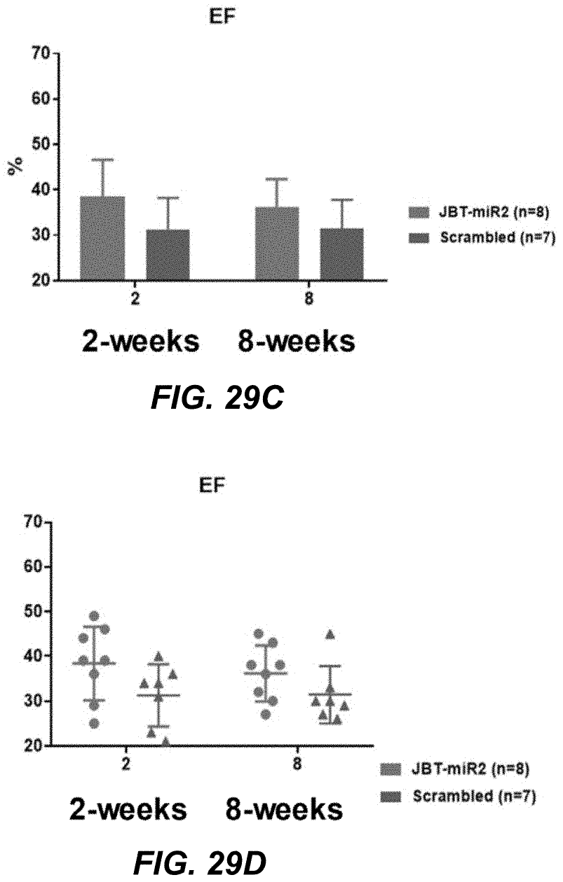

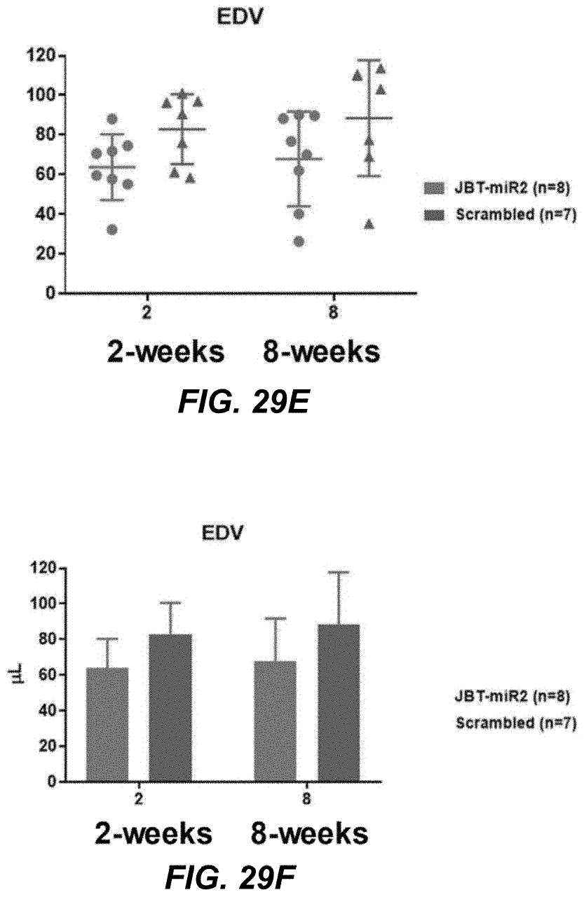

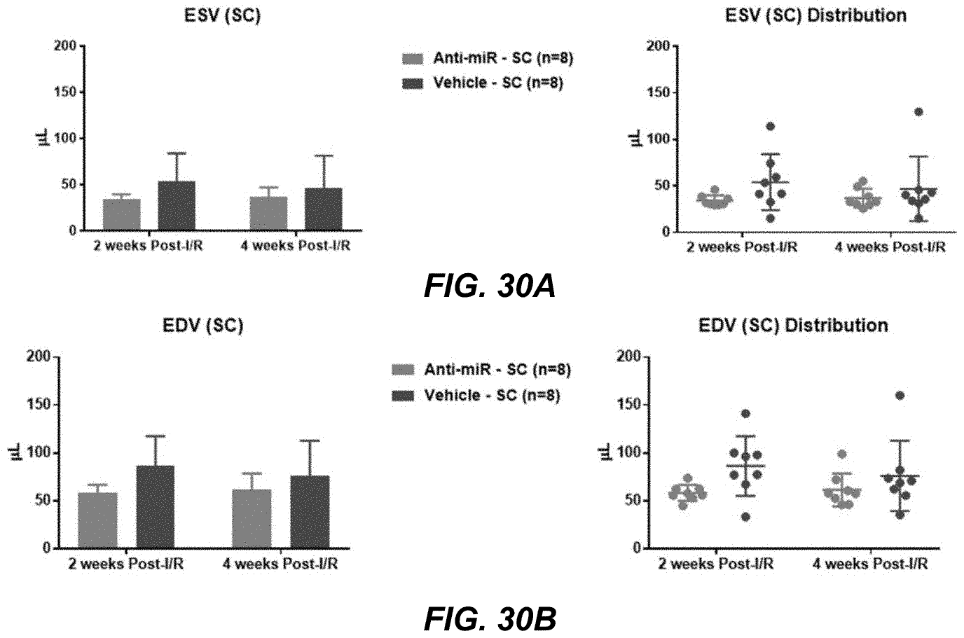

In sub-analysis of heart only, JBT-miR2 increased the expression of 64 mRNAs and decreased the expression of 86 mRNAs. KEGG analysis showed pathways involved in protein synthesis, intracellular signaling and cardiac muscle structure and function and myofibillar organization were upregulated. Four and a half LIM domains protein 1 is a protein that in humans is encoded by the FHL1 gene mRNA was upregulated >log 2 (fold change) of 11.34. Studies suggest that interactions between FHL1A and other proteins play a critical role in the assembly of sarcomeres, which are structures within muscle cells that are necessary for muscle tensing (contraction). These interactions also appear to be involved in chemical signaling within muscle cells, maintaining the structure of these cells, and influencing muscle growth and size. The second most highly expressed mRNA in the heart in response to JN-101 treatment was Troponin T2, (log 2 fold change of 10.35, P=1.28E-09) that is a critical regulator in cardiomyocyte contraction. Pathways involving cancer, hypertrophic cardiomyopathy were down regulated (FIGS. 8A-8D, FIGS. 23A-23B). Long non-coding RNAs (lncRNA) levels increased (9) and decreased (9) in heart following JBT-miR2 treatment (FIGS. 8E-8H) and are listed in FIGS. 23C-23D). Transcripts of Unknown coding protein (TUCP) both increased (23) and decreased (24) in heart tissue with JBT-miR2 treatment (FIGS. 8I-8L) and are listed in FIGS. 23E-23F. Multiple pathways are modulated with JBT-miR2 treatment and ongoing analysis will help us decipher those key pathways that are involved in cardiomyocyte regeneration.

### Body Weight and Arrhythmia:

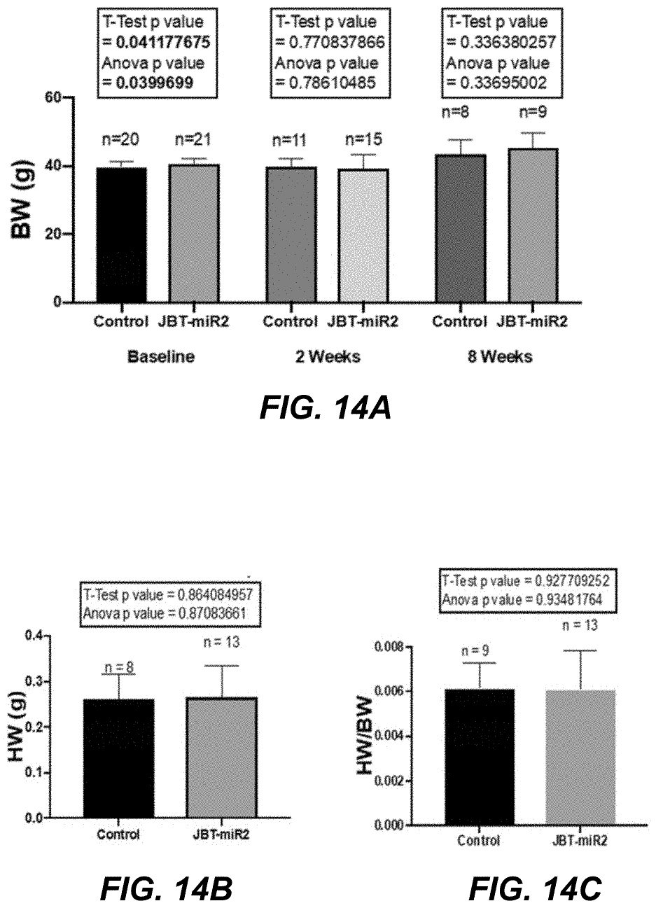

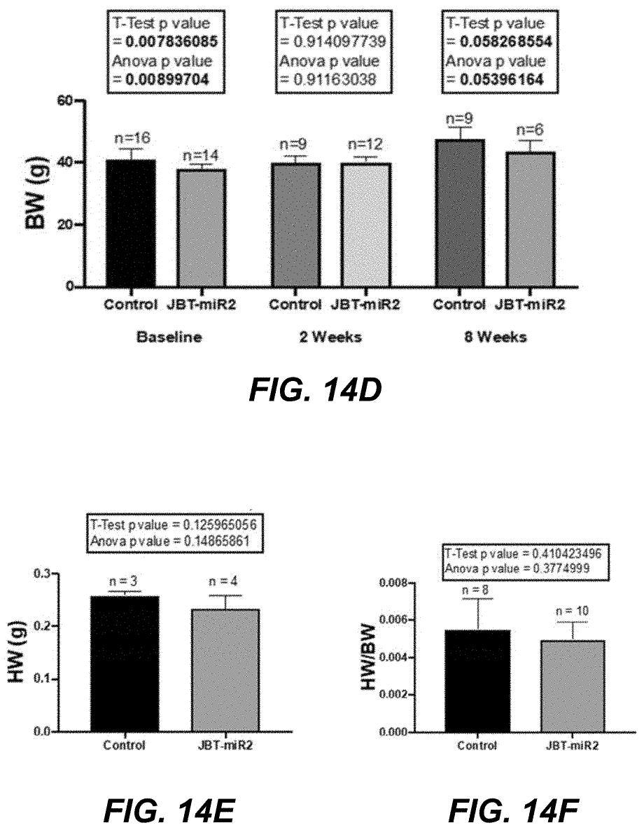

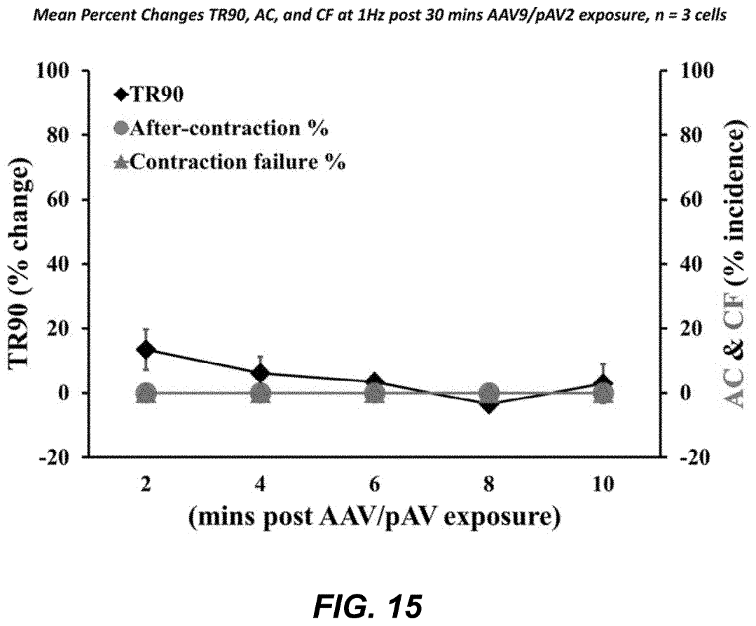

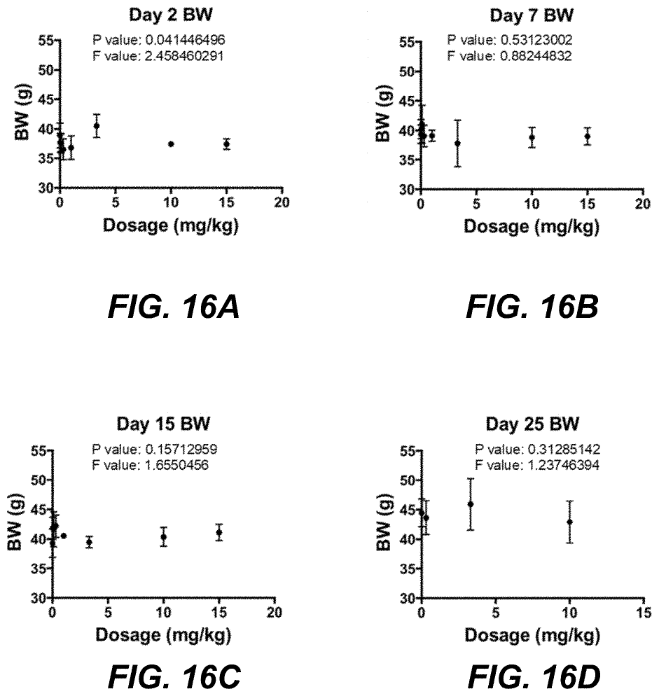

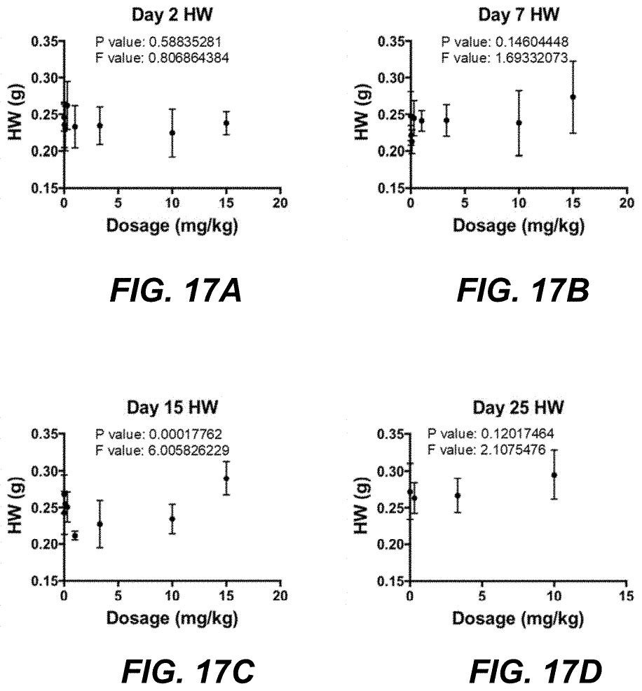

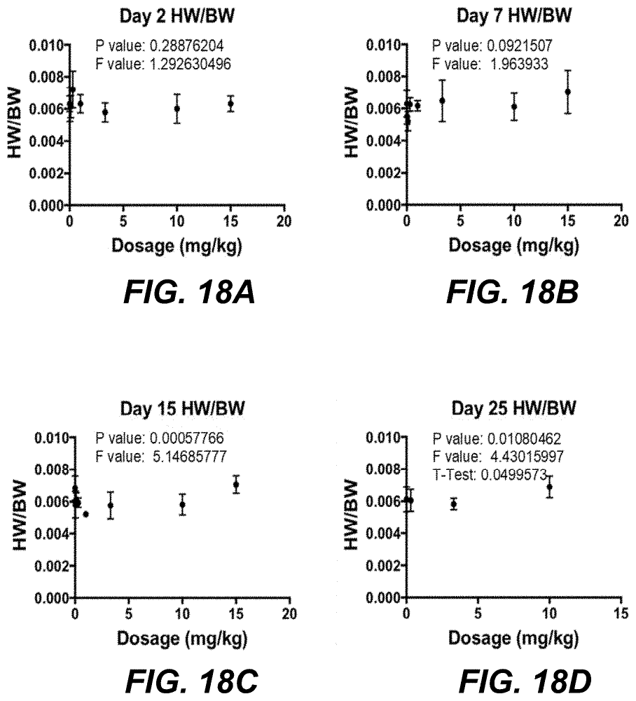

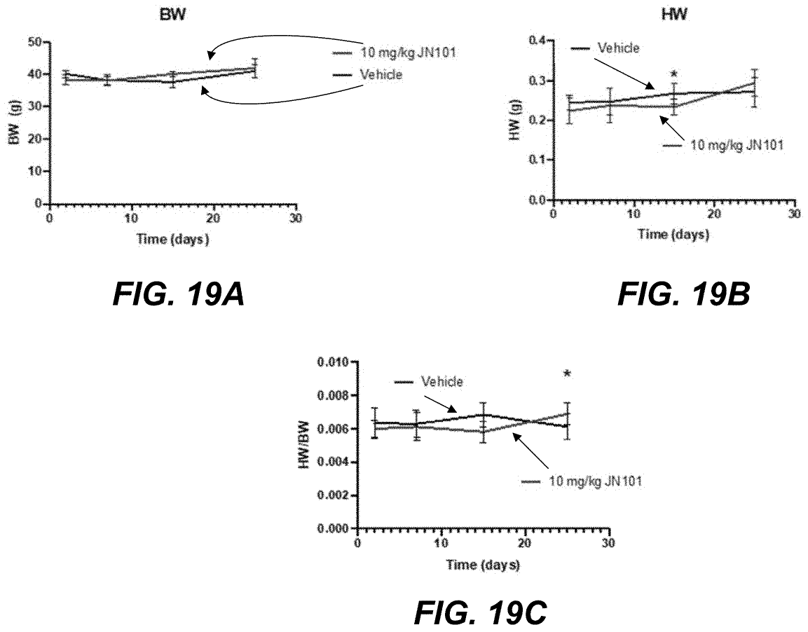

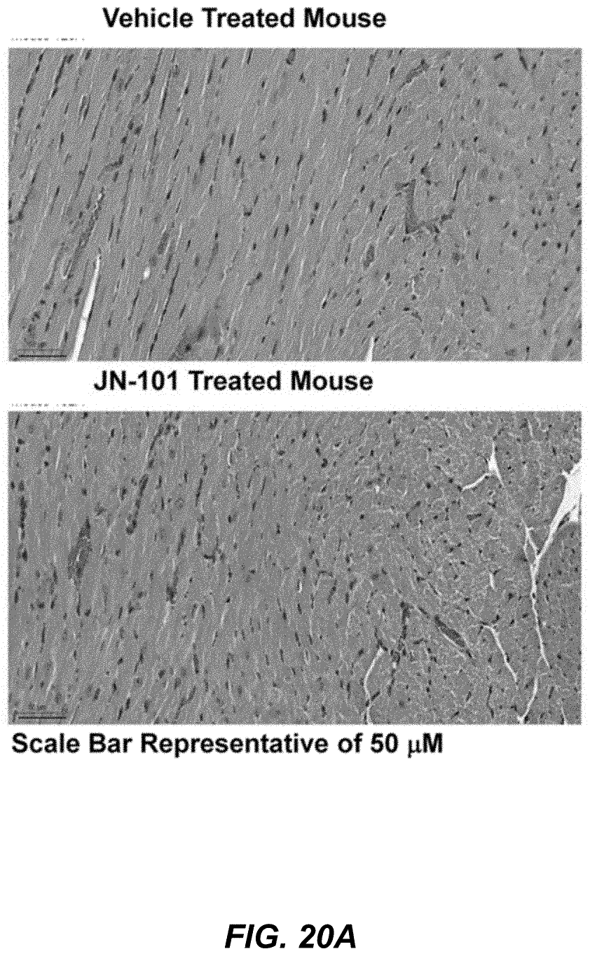

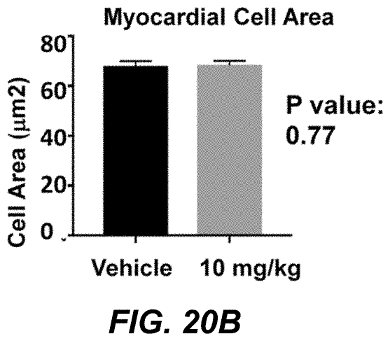

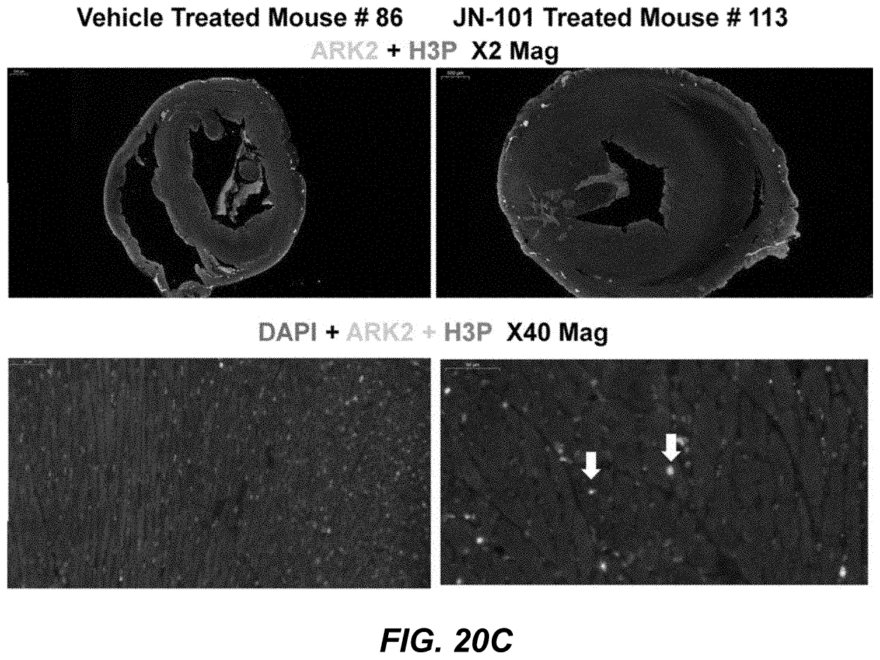

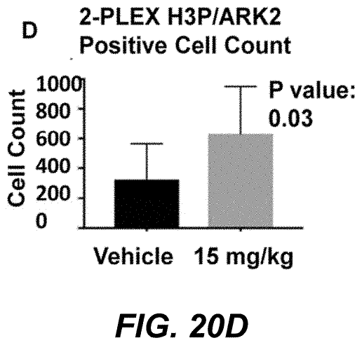

JBT-miR2 (FIGS. 14A-14F) or JN-101 (data not shown) had no effect on body weight or heart weight. No arrhythmia was seen in any mice. Primary human ventricular Cardiomyocytes isolated from a failed human heart transplant organ were incubated for 30 min with JBT-miR2 at 1×10″ vg/mL. No incidences of after contractions (AC) or contraction failure (CF) were found for 10 mins after incubation (FIG. 15).

### Analysis of BW and HW

JN-101 had no effect on uninjured mice BW up to doses of 15 mg/kg, measured at Day 15 post single administration and a dose of 10 mg/kg, measured at Day 25 post single administration compared to Vehicle. Dose dependent increases in HW were seen at Day 15 with effects seen at Day 7, but were not significant at Day 25. Interestingly the wet HW of JN-101 treated mice was less than those mice treated with Vehicle at Day 15. Significant dose-dependent increases in HW to BW ratio were measured at Day 25 post-study drug administration (P value: 0.01, F-Value=4.43, T-test P value P=0.05 Vehicle vs. 10 mg/kg JN-101). The increase in HW to BW ratio at Day 25 with 10 mg/kg JN-101 treatment may be related to regeneration of cardiac myocytes.

### Metabolic Blood Function Tests

SC injection of JN-101 (400 μl), resulted in slight elevations of sodium, potassium, chloride ions and phosphorous at Day 2 compared to Vehicle (50% Saline: 50% 10 mM Tris-HCL, 0.1 mM EDTA, pH 8.0), however the levels returned to normal and were not different from Vehicle on Day 7, 15 or 25 post-injection. Since the anti-miRs were dissolved in 10 mM Tris-HCL, 0.1 mM EDTA, pH 8.0 this could account for the higher levels of these ions at Day 2. CK and BUN and Cholesterol levels were elevated at Day 7 in the JN-101 treated mice compared to Vehicle, but were not different at any other time point. ALK levels were higher at Day 15 in JN-101 treated animals but normal at all other time points. All values were in the normal range for male CD-1 mice. No toxicity was observed (Stanley Roberts DABT).

### Cardiac Myocyte Proliferation and Cell Area

An additional experiment was conducted to confirm whether 1) cell division and 2) not hypertrophy were the causes of increase in wet HW:BW ratio at Day 25 post SC injection of 10 mg/kg JN-101. Hearts from 4 mice treated with 15 mg/kg of JN-101 Day 15 post SC Injection (Mice #113, 114, 115, 116) or Vehicle (Mice #85, 86, 87, 88) were sectioned, tripled stained with ARK-2, MHC, and H3P anti-bodies and quantified using Image^{DX}. 2-Plex cell count revealed an increase in H3P/ARK-2 positive cells, indicative of cytokinesis.

Hearts from 4 mice treated with 10 mg/kg JN-101, and sacrificed at Day 25 (Mice #152, 153, 154 and 155) or Vehicle (Mice #125, 126, 127, 128) were sectioned, and stained with trichrome Masson. Cell size was quantified using Image^{DX}. There were no differences in cell size or indication of cardiac myocyte hypertrophy (FIG. 26).

### Discussion

miRNAs are being intensively studied as therapeutic targets for a number of diseases including heart disease. By leveraging the regenerative capabilities of adult cardiomyocytes, as a translational, therapeutic approach, we demonstrate that an optimized virus, JBT-miR2, that delivers two transcribed miR-binding RNAs for let-7a/c and miR-99/100, significantly reduces cardiac muscle scarring, decreases cardiac volumes and increases heart function after a single administration at the time of reperfusion following transient cardiac IR injury in mice.

Similarly antagomiRs to the four miRs had similar effects in vivo, improving global and regional wall motion when administered at reperfusion following ischemia. The methods disclosed herein can be distinguished from the findings of Aguirre et al., who demonstrated that two virus's expressing modified zip construct inhibitors to these miRs mitigated ischemic injury in mice with permanent LCA ligation. The murine IR model with transient ligation of the LCA, used herein, is a more relevant model to human clinical use, with the virus, in some embodiments, to be administered shortly after a heart attack in the cardiac catheterization laboratory after perfusion has been restored to the heart muscle.

### Timing of Effectiveness

JBT-miR2 can be more effective when administered as close as possible to the time of reperfusion following ischemia as demonstrated in Group 1 mice, supported by the global ECHO and MRI data provided herein (Table 7). The reductions in cardiac volumes were evident at 2-weeks post administration but not significant at 8 weeks post IR. In other embodiments, a higher dose of virus and/or local cardiac administration may have more pronounced long-term positive effects on increasing cardiac function and reducing cardiac volumes in both Group 1 and Group 2 mice.

Group 2 mice treated with JBT-miR2 2-weeks after IR did show reductions in cardiac volume at 8-weeks post IR compared to Control virus as determined by ECHO, however the effects were not different between the JBT-miR2 and Control virus groups. In some embodiments, a higher dose of virus and/or local cardiac administration may have more pronounced long-term positive effects on increasing cardiac function and reducing cardiac volumes in mice with established heart failure.

### Regional Wall Motion

Unique to this study is the application of cardiac MRI 3D-regional wall motion measures of contraction with the ability to translate this imaging approach to large animal and human studies (FIG. 4). 2D-ECHO is limited by planographic views, permitting a single plane view of the left ventricle. JBT-miR2 significantly increased cardiac wall motion at 2-weeks post IR administration compared with Control virus (Group 1). These effects on wall motion were not evident when JBT-miR2 was administered 2-weeks after IR injury (Group 2), suggesting that, in some embodiments, higher doses (1×10¹²⁻¹⁴ vg/mouse) of virus and/or local cardiac administration may have more pronounced long-term positive effects on increasing cardiac function and reducing cardiac volumes in mice with established heart failure.

### Scar Size Reduction

Scar size reduction was assessed by two independent blinded parties' 8-weeks after IR using either Trichrome Masson's staining or Hematoxylin and eosin staining. Findings from both groups showed significant reductions in fibrotic tissue when JBT-miR2 was administered at reperfusion (Group 1) and 2-weeks after reperfusion (Group 2) (FIG. 6). CK levels, a marker of muscle injury, were significantly reduced (P=0.048) in Group 2 mice (FIG. 7). Since increased circulating levels of CK indicate muscle damage and were chronically elevated in patients with IHD, this suggests that JBT-miR2 decreases muscle damage, consistent with cardiac histology. High Blood Urea Nitrogen (BUN) levels may also be the consequence of a lack of blood flow to the kidneys, due to heart failure. Consistent with the increase in heart function, decrease in fibrosis and scar tissue and CK levels, BUN levels were significantly decreased in Group 2 mice (P<0.001). CK levels were reduced by 40% in JBT-miR2 treated Group 1 mice, however there were no changes in BUN levels. This could be due to circulating levels of CK and BUN are measured at 8-weeks post treatment in Group 1 vs. 6-weeks post treatment in Group 2 and that the effective period of the virus may, in some embodiments of the methods provided herein, be between 2 and 6 weeks after administration of JBT-miR2.

### Mechanism of Action

The mechanism of action of heart regeneration in zebrafish and neonatal mice, is thought to be a cardiomyocyte-mediated process that occurs by dedifferentiation of mature cardiomyocytes followed by proliferation and further redifferentiation. The current study design could not confirm the involvement of FNTβ and SMARCA5 in mouse cardiac muscle regeneration, since cardiac tissue was collected at necropsy for transcriptional RNA analysis following terminal hemodynamics at 8-weeks when the levels of both FNTβ and SMARCA5 are predicted to be significantly reduced. In normal mice treated with 15 mg/kg JN-101, an increase in ARK2 positive cells in the hearts was seen compared to vehicle (p=0.03). However in mice with ischemic injury significant differences in mRNA, TUCP and lncRNA levels at week 8 post IR were shown particularly mRNAs related to cardiac muscle structure and function (FIGS. 23A-23F). Of importance were increased levels in cardiac troponin T (cTnT), encoded by the gene TNNT2 in JBT-miR2 treated hearts and is a component of the troponin complex which allows actomyosin interaction and contraction to occur in response to Ca 2+ and mRNA levels of FHL1A. In combination with increased MHC positive cells (FIG. 14A-14F), this suggests that JBT-miR2 is increasing the numbers of differentiated cardiomyocytes post-IR injury.

### Safety

No safety concerns were evident with a single IV administration of JBT-miR2 in mice or SC administration of JN-101. This is partly because the virus and JN-101 are transiently present in the cardiac myocytes and are non-integrative. In addition there were no abnormal pathology changes in off-target tissues or increase in death related to expression of the Tuds or JN-101. On the contrary, there was a non-significant survival benefit in JBT-miR2 treated mice compared with those treated with control virus. MFTs showed no indication of liver toxicity which has been an issue with viral delivered therapies. The General Approach to Histopathologic Evaluation of toxicity showed no indications of toxicological changes in the heart, spleen, lung, kidney, liver, skin, skeletal muscle and brain (Troyer). Body weight and heart rates were similar between groups. Primary human ventricular Cardiomyocytes isolated from a failed human heart transplant organ were incubated for 30 min with JBT-miR2 at 1×10¹¹ vg/mL. No incidences of after contractions (AC) or contraction failure (CF) were found for 10 mins after incubation.

### Viral and AntagomiR Design and Translation

Our single virus expressing Tuds to miR-99/100 is highly innovative. Firstly, there are no commercial treatments that regenerate heart muscle. JBT-miR2 represents a first-in-class, novel, (and, in some embodiments, adjunct) therapy for cardiac IR injury by enhancing endogenous CM regeneration by targeting validated miR targets. Not only does JBT-miR2 remove the possibility of rejection of exogenous cells by promoting proliferation of endogenous CMs, it can simplify agent production over autologous strategies since cardiac progenitors were produced without the need to collect, culture and transplant stem cells. This situation has never been attained and represents a major conceptual advance. Secondly, JBT-miR2 is a refined single cardiotropic AAV2/9 construct that constitutes the next “stepping stone” in providing efficient and safe delivery of specific multi-RNAi regenerative therapy into the myocardium. Among the advantages of the compositions and methods provided herein include: (a) AAV vectors can be optimal in CV gene therapy to deliver TuDs since they contain no viral protein-coding sequences to stimulate an immune response, do not require active cell division for expression to occur and, have a significant advantage over adenovirus vectors because of their stable, long-term expression of genes in Cardiomyocytes in vivo; (b) in extensive studies, JBT-miR2 demonstrated efficacy in mice with IR injury, when administered IV at reperfusion, at a single low tested dose with no evidence of oncogenesis or organ toxicity; (c) the AAV2/9 virus is non-integrative which reduces the possibility of off-target effects and long-term toxicity; (d) JBT-miR2 can be administered by intra-myocardial or by intra-coronary injection, thereby reducing safety concerns; (e) unlike other currently available compositions and methods that were targeting single miRs, it is known that multiple miRs (>60) were involved in zebrafish heart regeneration, suggesting that targeting a single miR will not be effective in regenerating human heart; and (f) TuDs were superior to previously described modified Zip constructs as miR inhibitors in their design, potency, and specificity and efficacy.

JN-101 oligonucleotides act by inhibiting the endogenous microRNA within the RISC complex and act by a completely different mechanism to the Tuds expressed in virus.

### Human Equivalent Doses and Dosing Strategy

Based on the data that we have generated we can predict the pharmacologically active dose range of both JBT-miR2 and JN-101 in Humans using the Food and Drug Guidance for Industry Estimating the Maximum Safe Starting Dose in Initial Clinical Trials for Therapeutics in Adult Healthy Volunteers at the time of reperfusion and also in established heart failure.

In mice JN-101 therapeutic dose range without toxicity is 10 mg/kg/mouse administered in a volume of 400 μl. From this we can estimate that the effective and safe dose is between 1-100 mg/kg in mice having tested doses up to 15 mg/kg in normal mice without observing toxicity. The Human Equivalent Doses are therefore as follows administered subcutaneously or locally within the heart in patients based on body surface area. For mice the effective dose is divided by a factor of 12.3. The effective dose of JBT-miR2 in mice was 1×10¹¹ vg/mouse. In some embodiments, higher doses can be given based on the preclinical data presented herein and possibly local cardiac administration that will limit off-target side effects. Tables 13 and 14 provide estimates of human equivalent doses of compositions provided herein based on the murine studies.

TABLE 13
Conversion of Animal Doses to Human for JN-101

| | | Human Equivalent Intra- |
|---|---|---|
| | Human Equivalent Subcutaneous | ventricular/Intra-myocardial |
| | or Systemic Dose, including | dose (Multiply Human |
| Mouse Dose | Intra-Coronary (mg/kg) and | Equivalent Intravenous |
| (mg/kg) | Volume (divide mouse dose by 12.3) | Systemic Dose by 0.05) mg/kg |
| 1 | 0.08 | 0.004 |
| 3 | 0.24 | 0.012 |
| 10 | 0.81 | 0.0405 |
| 15 | 1.22 | 0.061 |
| 30 | 2.44 | 0.122 |
| 40 | 3.25 | 0.1625 |
| 50 | 4.06 | 0.203 |
| 60 | 4.89 | 0.2445 |
| 70 | 5.69 | 0.2845 |
| 80 | 6.50 | 0.325 |
| 90 | 7.32 | 0.366 |
| 100 | 8.13 | 0.4065 |

Assumes 60 kg human. The doses can be given as a single administration or multiple administrations. While the preclinical data disclosed herein suggests that in some embodiments, the therapeutic is effective for 1 month when administered at reperfusion, however, in some embodiments, multiple doses may be given systemically or by local intra cardiac or intracoronary injection immediately following an Ischemic event following reperfusion and in the chronic heart failure setting.

TABLE 14
Conversion of Animal Doses to Human for JBT-miR2

| | | | | Human Equivalent |
|---|---|---|---|---|
| | | Human | | Intra-ventricular/Intra- |
| | Mouse Dose | Equivalent | Human | myocardial dose |
| Mouse Dose | Vg/kg | Intravenous | Equivalent | (Multiply Human |
| Vg/ 40 g mouse | (multiply by | Systemic Dose | Intra-Coronary | Equivalent Intravenous |
| range | 25) | Vg/kg | Dose Vg/kg | Systemic Dose by 0.05) |
| 1 × 10¹¹ | 2.5 × 10¹² | 2.5 × 10¹² | 2.5 × 10¹² | 0.125 × 10¹² |
| 1 × 10¹² | 2.5 × 10¹³ | 2.5 × 10¹³ | 2.5 × 10¹³ | 0.125 × 10¹³ |
| 1 × 10¹³ | 2.5 × 10¹⁴ | 2.5 × 10¹⁴ | 2.5 × 10¹⁴ | 0.125 × 10¹⁴ |
| 1 × 10¹⁴ | 2.5 × 10¹⁵ | 2.5 × 10¹⁵ | 2.5 × 10¹⁵ | 0.125 × 10¹⁵ |

Assumes 40 g mouse and 60 kg human. The doses may be given as a single administration or multiple administrations. While the preclinical data disclosed herein suggests that the therapeutic is effective between 2-6 weeks following administration at the time of reperfusion, however, in some embodiments, multiple doses may be given systemically or by local intra cardiac or intracoronary injection immediately following an Ischemic event following reperfusion and in the chronic heart failure setting.

### Applicability of JBT-miR2 and JN-101 for the Treatment of Other Diseases and on Top of Standard of Care

The compositions provided herein can be administered on top of standard of care at the time of reperfusion following a myocardial infraction or on top of established heart failure drugs. One main cause of IHD is a heart attack. Despite a number of treatments that are used to unblock the clot and restore blood flow to the damaged tissue, such as percutaneous coronary interventions (PCI, e.g. balloon dilation, stent implantation) which restore arterial perfusion and in association with thrombolytic and antiplatelet therapy or surgical revascularization (coronary artery bypass grafting, CABG), that have improved the management of patients with IHD (Table 15), recovery of cardiac function and prevention of the transition to HF is unsatisfactory in the majority of these patients because current treatments do not regenerate heart muscle after a MI nor prevent the transition to HF. The compositions and methods provided herein can provide an additive or synergistic improvement in patient outcome when employed in concert with one or more of these standards of care.

TABLE 15
Current Standard of care for IHD

| Intervention | Timing and Use | Limitation(s) |
|---|---|---|
| Percutaneous coronary | Within 90 mins of | Minimal, restores coronary blood flow in 90% of patients |
| interventions | hospitalization | |
| Coronary artery | In failed PCI | Higher risk of mortality than PCI |
| bypass grafting | | |
| Anti-platelets | At signs of symptoms | Can cause Hemorrhage |
| Heparin | Within 48 h | Can Cause Hemorrhage |
| Warfarin | 3 months after an MI | Not normally recommended. Can cause Hemorrhage |
| Fibrinolytics | Use in 1 h | Not used with Intracranial hemorrhage, malignancy, stroke, |
| | | aortic dissection |
| Oxygen | On recognition of an MI | No studies demonstrate reduction of mortality or morbidity |
| Vasodilators | For 48 h | Low BP, headache, and tachyphylaxis |
| Pain Control | At 5 to 15 min intervals | Can mask ischemic symptoms, does not treat disease |
| Beta Blockers | Indefinite use | Bradycardia, HF, bronchospasm, hemodynamic instability |
| ACEI, ARBs | Use in 24 h & indefinite | Can cause hypotension and declining renal function |
| Glycoprotein | During PCI | Can cause headache, back pain, nausea/vomiting, bleeding |
| Antagonist | | |
| Statins | Prior to discharge | Intolerance and pain and inflammation in some subjects |
| Aldosterone | All post-MI patients | Use with ACEI, EF < 40%, creatinine clearance > 30 mL/min |
| Antagonists | | |
| ICDs | Used 40 Days after MI | Not for NYHA functional class IV patients or an EF > 40% |

Based on the RNA Sequencing data degenerated, there are a number of selective potential mRNA target proteins that are upregulated in expression where let-7a/c, miR-99/100 inhibition and treatment of other diseases as tabulated below in Table 16 to increase the expression of compensatory proteins

TABLE 16
Target Proteins

| mRNA target | Disease Indication/Rationale |
|---|---|
| Fh11, Zinc Finger, | The FHL1 gene is responsible for a number of Muscular dystrophy-like muscle disorders, |
| LIM type | ranging from severe, childhood onset diseases through to adult-onset disorders similar to Limb |
| | girdle muscular dystrophy. At present different research groups are using different terminology |
| | for these disorders, which include: |
| | (a) X-linked myopathy with postural muscle atrophy (XMPMA). An adult-onset muscle disorder |
| | known to affect families in Austria and the UK. |
| | (b) Reducing body myopathy (RBM). A rare disorder causing progressive muscular weakness |
| | characterized by aggresome-like inclusions in the myofibrils. The effects of the disorder can be |
| | either severe, with onset of weakness at approximately five years, or adult onset, with weakness |
| | occurring in the late 20s, early 30s. |
| | (c) Scapuloperoneal (SP) syndrome. Another adult-onset muscle disorder, especially affecting |
| | the shoulder girdle and legs. |
| TnnT2, Troponin | Diseases associated with mutations in Troponin: Mutations in this gene have been associated with |
| | familial hypertrophic cardiomyopathy as well as with restrictive and dilated cardiomyopathy. |
| | Transcripts for this gene undergo alternative splicing that results in many tissue-specific isoforms, |
| | however, the full-length nature of some of these variants has not yet been determined. Mutations |
| | of this gene may be associated with mild or absent hypertrophy and predominant restrictive |
| | disease, with a high risk of sudden cardiac death. Advancement to dilated cardiomyopathy may |
| | be more rapid in patients with TNNT2 mutations than in those with myosin heavy chain |
| | mutations. |

### CONCLUSIONS

Ischemic Heart Disease is the largest cause of death in the developed World and can be caused by a myocardial infarction (MI). A major pathologic problem is the failure of human adult cardiomyocytes to regenerate themselves endogenously following a MI, leads to scarring, a decrease in heart function and the development of heart failure. The effective promotion of endogenous cardiomyocyte regeneration in the ischemic heart could potentially offer a new treatment for MI and prevent adverse pathophysiologic consequences.

As described herein, inhibition of a specific combination of four miRNAs (miRNAs); miR-99, miR-100, let-7a and let-7c, is a critical regulator of cardiomyocyte dedifferentiation and proliferation in mammals. As a translational therapeutic approach, this example describes the design and testing of the effects of synthetic oligonucleotide antagomiRs (JN-101) and an adeno associated virus (AAV2/9, JBT-miR2) that can both temporarily inhibit miR-99, miR-100, let-7a and let-7c in the hearts of mice with cardiac ischemic reperfusion (IR) injury.

In extensive double blind studies disclosed in this example, a single dose of 1×10¹¹ vg/mouse of JBT-miR2 administered intravenously at reperfusion is necessary and sufficient to re-activate an underlying cardiac regeneration process in mice with IR injury as determined by reductions in cardiac volumes at 2-weeks (End Diastolic Volume, EDV=63.7±15.5 μl JBT-miR2 vs. 82.9±16.3 μl Control, p=0.051, 23.2% reduction; End Systolic Volume, ESV=39.2±12.9 μl JBT-miR2 vs. 57.4±14.9 μl Control, p=0.037, 31.8% reduction; N=7 confirm that JBT-miR2, N=7 Control) and increased Ejection Fraction (EF) (38±8% JBT-miR2 vs. 31±6% Control, p=0.09, a 22.7% increase). MRI showed similar reductions in EDV (−23.1%), ESV (−34%) and increased EF (23.5%) with significant increases in wall motion in the antero-apical infarction of mice 2-weeks after a single administration of JBT-miR2 compared to Control. JBT-miR2 reduced scarring (47.7%, p=0.039) and increased cardiomyocyte numbers with no obvious safety concerns. When administered 2-weeks after IR, JBT-miR2 reduced fibrosis and levels of blood Urea Nitrogen (p<0.001) and Creatine Kinase (p=0.04) compared to Control virus. RNAseq data showed significant increases in mRNA levels of FHL1A (11-fold)_{log 2} and TNNT2 (10-fold)_{log 2}. ECHO and MRI both showed similar global improvements in mice with IR injury when treated with a single subcutaneous administration of 10 mg/kg JN-101, further validating the target miRNAs.

Global and regional cardiac imaging together with histological and biomarker data show that combined inhibition of miRNAs-99/miR-100 and let-7a/c with two different therapeutic strategies mitigates cardiac muscle injury in mice with cardiac IR injury and emphasize the importance of miRNAs as novel therapeutic targets to treat heart disease.

In this example, a single low dose of JBT-miR2 was administered intravenously to mice with IR injury. An impediment to success in CV viral delivered therapy is in obtaining sufficiently high cardiac uptake to provide a beneficial biological effect. The IV technique is effective and is a simple delivery mode of AAV, since it avoids the risk of an invasive procedure. In humans IV can be implemented with the use of a peripheral or central venous catheter. However, in some embodiments, efficacy can be reduced by the virus becoming sequestered in the lung, liver, spleen, brain or other organs. Moreover, in some embodiments, this route is not amenable for patients with occluded arteries. In some embodiments, for patients presenting with an MI, the ease of delivery following catheter intervention to re-establish coronary flow can make intracoronary delivery appealing as it allows for selective delivery of therapeutics to the myocardial area of interest and can limit risks of systemic toxicity.

The data provided in this example confirm that: (1) a single, low IV dose of JBT-miR2 or SC administered JN-101 that both deliver inhibitors to miR-99, miR-100, let-7a and let-7c can be necessary and sufficient to re-activate an underlying cardiac regeneration process in mice with IR injury as demonstrated by regional wall motion improvement; (2) Greater efficacy as evident with increased heart function and reduced cardiac volumes when JBT-miR2 is administered at the time of reperfusion following transient ischemia in mice; and (3) JBT-miR2 reduces scarring and increases cardiomyocyte numbers, correlated with a decrease in CK and BUN levels weeks after administration. There were no obvious safety concerns in mice with both therapeutic strategies. In some embodiments, local cardiac administration and/or higher dose of the compositions disclosed herein (e.g., virus) may be more effective.

Disclosed herein includes JBT-miR2, a single viral vector comprising two transcribed miR-binding RNAs for let-7a/c and miR-99/100, referred to herein as tough decoys (TuDs). As a single viral vector, JBT-miR2 can be used for multiple drug delivery. In some embodiments, TuDs are artificial single strands of RNA with one antisense miR binding domain (Decoy) or a stabilized stem-loop with two miR binding domains that sequester the miR into stable complexes through complementary base pairing. This exemplary configuration can disable one or more RNAi pathways, for example acting in part, by targeting miRs for destruction by recruiting the tailing and trimming pathway to decrease target miR steady-state abundance. Inserted into the pAV-U6-GFP vector are the two TuDs cloned at the 5′ ends next to the human U6 or H1 promoters that drive their expression.

In some embodiments, JBT-miR2 viral vector is administered intravenously, for example in a therapeutically effective amount, in a subject (e.g., a mammal (e.g., human or mice)). The subject can be, for example, a subject with cardiac ischemic reperfusion injury when administered at the time of reperfusion. Compared to the currently known technique in which two separate AAV2/9 or lenti-viral vectors were used to express modified zip construct inhibitors to the four microRNAs, the viral vectors disclosed herein (for example the JBT-miR2 viral vector) allows delivery of multiple microRNAs in a single viral vector. In the currently known technique, a mouse model of permanent ischemic injury (not ischemic reperfusion) and the two viral vectors were administered by direct intra-myocardial (directly into the heart muscle). The viral vectors disclosed herein, for example JBT-miR2 or JN-101, are effective, in some embodiments, when given as close as possible to the time of the transient ischemic reperfusion injury, at the time of reperfusion as compared to 2-weeks after the cardiac ischemic reperfusion injury. Such results are superior and unexpected. JN-101 consist of equal quantities of JRX0104 and JRX0116 (10 mg/kg of each is given to mice.

The JBT-miR2 TuD sequences are shown in FIG. 1A. As disclosed herein, viral vector JN-101 which comprises two oligonucleotides can be administered by subcutaneous injection to subjects (e.g., mammals, including humans). In some embodiments, intravenous injection results in cardiac thrombi. IV admin of anti-miRs increased mortality to 87% vs. 50% with SC administration. As disclosed herein, one or more of viral vectors JBT-miR2 and JN-101 can significantly decrease cardiac volumes (End Diastolic Volume and End Systolic Volume) and increase ejection fraction (Ejection Fraction) in mammals with a heart attack at 2-weeks after treatment. The therapeutics can be effective 4-8 weeks later. Repeat injections or higher doses may be given. As disclosed herein, the viral vectors disclosed herein can decrease creatine kinase levels reducing cardiac muscle injury and improved kidney function as demonstrated by a decrease in Blood Urea Nitrogen levels in mice with a heart attack. One or more of the viral vectors disclosed herein can, in some embodiments, be used to improve kidney function in mammals and humans. For example, as shown in this example, JBT-miR2 decreases scarring in the left ventricle by 47%. Both treatments improve regional wall motion in the left ventricle. Both treatments could be given two weeks after a heart attack and higher doses will be needed. The human equivalent doses are also provided in this example. In some embodiments, one or more of the viral vectors disclosed herein can increase the number of cardiomyocytes and mRNA encoding proteins that are involved in differentiated cardiomyocyte muscle structure and function and can be applied to other diseases. In some embodiments, one or more of the viral vectors disclosed herein, for example each of JBT-miR2 and JN-101, can increase survival by 20% when administered at a single tested dose when given immediately after ischemia at the time of reperfusion.

In some embodiments, the viral vectors provided herein comprises two tough decoys (TuDs), wherein one of the TuD is a transcribed miR-binding RNAs for let-7a/c and the other TuD is a transcribed miR-binding RNAs for miR-99/100. The viral vector can be for multiple drug delivery. One of the two TuD can comprise an artificial single strands of RNA with one antisense miR binding domain (Decoy) or a stabilized stem-loop with two miR binding domains that sequester the miR into stable complexes through complementary base pairing. The TuD configuration can disable a RNAi pathway. The disabling of the RNAi pathway can comprise targeting miRs for destruction by recruiting the tailing and trimming pathway to decrease target miR steady-state abundance. The two TuDs can be located at the 5′ ends of the viral vector and/or adjacent to a human U6 or H1 promoters that drive their expression. The viral vector can comprise one or more of: (a) TuD (Let-7a/c TuD 1), (b) Let-7a Reverse Complement, (c) miR-99a/100 TuD 2, and (d) miR-99a Reverse Complement, as shown in FIG. 1A.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of preventing, inhibiting, reducing, or treating cardiac ischemic reperfusion injury, comprising administering a therapeutic composition to a subject before, during, and/or after a cardiac ischemic event, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

2. The method of claim 1, comprising reperfusion of ischemic cardiac tissue.

3. A method of increasing heart function, reducing mortality, reducing cardiac volumes and/or reducing scar size following ischemic reperfusion injury, comprising administering a therapeutic composition to a subject before, during, and/or after a cardiac ischemic event, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

4. The method of claim 3, comprising reperfusion of ischemic cardiac tissue.

5. A method of treating myocardial infarction, comprising administering a therapeutic composition to a subject before, during, and/or after reperfusion of ischemic cardiac tissue, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

6. The method of claim 3, wherein myocardial infarction is a cardiac ischemic event.

7. A method of inducing cardiomyocyte regeneration, cardiac repair, vasculogenesis and/or cardiomyocyte differentiation following a cardiac ischemic event, comprising administering a therapeutic composition to a subject before, during, or after reperfusion of ischemic cardiac tissue, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

8. A method of treating a disease or disorder associated with dysregulation of FHL1 and/or TNNT2, comprising administering a therapeutic composition to a subject in need thereof, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

9. A method of treating a kidney condition of a subject and/or protecting a kidney of a subject from injury, the method comprising administering a therapeutic composition to the subject, wherein the therapeutic composition comprises one or more of (a) a composition comprising a plurality of microRNA (miR) antagonists, wherein said plurality of miR antagonists comprises one or more miR-99a antagonists, one or more miR-100-5p antagonists, one or more miR-Let-7a-5p antagonists, and one or more miR-Let-7c-5p antagonists; (b) an expression cassette comprising a nucleotide sequence encoding one or more miR-99a antagonists, one or more miR-100-5p antagonists, one or more miR-Let-7a-5p antagonists, and one or more miR-Let-7c-5p antagonists; and (c) a cloning or expression vector comprising the expression cassette of (b).

10. The method of claim 1, wherein one or more of the followings applies:
(a) at least one of the one or more miR-99a antagonists comprises an anti-miR-99a comprising a nucleotide sequence having at least 80%, 85%, 90%, 95%, 96%, 97, 98%, 99% or 100% identity to a sequence selected from the group consisting of SEQ ID NOs 47, 48, 50, 52, and 54;
(b) at least one of the one or more miR-100-5p antagonists comprises an anti-miR-100-5p comprising a nucleotide sequence having at least 80%, 85%, 90%, 95%, 96%, 97, 98%, 99% or 100% identity to a sequence selected from the group consisting of SEQ ID NOs 46, 49, 51, 53, and 55;
(c) at least one of the one or more Let-7a-5p antagonists comprises an anti-miR-Let-7a-5p comprising a nucleotide sequence having at least 80%, 85%, 90%, 95%, 96%, 97, 98%, 99% or 100% identity to a sequence selected from the group consisting of SEQ ID NOs: 37, 39, and 40-45; and
(d) at least one of the one or more Let-7c-5p antagonists comprises an anti-miR-Let-7c-5p comprising a nucleotide sequence having at least 80%, 85%, 90%, 95%, 96%, 97, 98%, 99% or 100% identity to a sequence selected from the group consisting of SEQ ID NOs: 36, 38, and 40-45.

11. The method of claim 1, wherein one or more of the followings applies:
(a) at least one of the one or more miR-99a antagonists comprises an anti-miR-99a comprising a nucleotide sequence having one or more mismatched nucleobases with respect to a sequence selected from the group consisting of SEQ ID NOs: 47, 48, 50, 52, and 54;
(b) at least one of the one or more miR-100-5p antagonists comprises an anti-miR-100-5p comprising a nucleotide sequence having one or more mismatched nucleobases with respect to a sequence selected from the group consisting of SEQ ID NOs: 46, 49, 51, 53, and 55;
(c) at least one of the one or more Let-7a-5p antagonists comprises an anti-miR-Let-7a-5p comprising a nucleotide sequence having one or more mismatched nucleobases with respect to a sequence selected from the group consisting of SEQ ID NOs: 37, 39, and 40-45; and
(d) at least one of the one or more Let-7c-5p antagonists comprises an anti-miR-Let-7c-5p comprising a nucleotide sequence having one or more mismatched nucleobases with respect to a sequence selected from the group consisting of SEQ ID NOs: 36, 38, and 40-45.

12. The method of claim 1, wherein at least one of the anti-miRs comprises one or more chemical modifications selected from the group consisting of a modified internucleoside linkage, a modified nucleotide, and a modified sugar moiety, and combinations thereof.

13. The method of claim 12, wherein the one or more chemical modifications comprises a modified internucleoside linkage.

14. The method of claim 13, wherein the modified internucleoside linkage is selected from the group consisting of a phosphorothioate, 2′-Omethoxyethyl (MOE), 2′-fluoro, alkylphosphonate, phosphorodithioate, alkylphosphonothioate, phosphoramidate, carbamate, carbonate, phosphate triester, acetamidate, carboxymethyl ester, and combinations thereof.

15. The method of claim 13, wherein the modified internucleoside linkage comprises a phosphorothioate internucleoside linkage.

16. The method of claim 12, wherein at least one of the one or more chemical modifications comprises a modified nucleotide, optionally the modified nucleotide comprises a locked nucleic acid (LNA), and further optionally the locked nucleic acid (LNA) is incorporated at one or both ends of the modified anti-miR.

17. The method of claim 16, wherein the modified nucleotide comprises a locked nucleic acid (LNA) chemistry modification, a peptide nucleic acid (PNA), an arabino-nucleic acid (FANA), an analogue, a derivative, or a combination thereof.

18. The method of claim 12, wherein at least one of the one or more chemical modifications comprises a modified sugar moiety.

19. The method of claim 19, wherein the modified sugar moiety is a 2′-O-methoxyethyl modified sugar moiety, a 2′-methoxy modified sugar moiety, a 2′-O-alkyl modified sugar moiety, a bicyclic sugar moiety, or a combination thereof.

20. The method of claim 18, wherein the modified sugar moiety comprises a 2′-O-methyl sugar moiety.

21. The method of claim 1, wherein the cloning or expression vector is a viral vector.

22. The method of claim 21, wherein the viral vector is a lentiviral vector or an adeno-associated viral (AAV) vector.

23. The method of claim 1, wherein the cloning or expression vector comprises
(a) a nucleotide sequence having at least 80%, 85%, 90%, 95%, 96%, 97, 98%, 99% or 100% identity to each of the nucleotide sequences set forth in SEQ ID NOs: 59-64;
(b) a nucleotide sequence having at least 80%, 85%, 90%, 95%, 96%, 97, 98%, 99% or 100% identity to each of the nucleotide sequences set forth in SEQ ID NOs: 86-89; or
(c) a nucleotide sequence having at least 80%, 85%, 90%, 95%, 96%, 97, 98%, 99% or 100% identity to each of the nucleotide sequences set forth in the SEQ ID NOs indicated in (a) and (b).

24. The method of claim 1, wherein the cloning or expression vector comprises a nucleotide sequence having least 80%, 85%, 90%, 95%, 96%, 97, 98%, 99% or 100% identity to the nucleotide sequence of SEQ ID NO: 85.

25. The method of claim 1, wherein the cloning or expression vector comprises a nucleotide sequence having least 80%, 85%, 90%, 95%, 96%, 97, 98%, 99% or 100% identity to the nucleotide sequence of SEQ ID NO: 101.

26. The method of claim 1, wherein the plurality of miR antagonists are encoded by the same expression cassette or vector.

27. The method of claim 1, wherein the plurality of miR antagonists are encoded by different expression cassettes or vectors.

28. The method of claim 1, wherein the expression cassette comprises a tough decoy (TuD) cassette comprising a nucleotide sequence encoding one or more miR-99a antagonists.

29. The method of claim 28, wherein the TuD cassette comprises one or more promoter sequences operably linked to the nucleotide sequence encoding one or more miR-99a antagonists, optionally the one or more promoter sequences comprise a H1 promoter and/or a U6 promoter.

30. The method of claim 28, wherein the cloning or expression vector comprises two or more TuD cassettes.

31. The method of claim 28, wherein an effective dose of a therapeutic composition comprising a cloning or expression vector comprising two or more TuD cassettes is at least about 1.1-fold less than an effective dose of a therapeutic composition comprising a cloning or expression vector comprising one TuD cassette.

32. The method of claim 28, wherein the TuD cassette comprises a nucleotide sequence having least 80%, 85%, 90%, 95%, 96%, 97, 98%, 99% or 100% identity to the nucleotide sequence of SEQ ID NO: 98.

33. The method of claim 1, wherein the cloning or expression vector comprises a nucleotide sequence having least 80%, 85%, 90%, 95%, 96%, 97, 98%, 99% or 100% identity to the nucleotide sequence of SEQ ID NO: 99.

34. The method of claim 1, wherein the cloning or expression vector comprises a nucleotide sequence having least 80%, 85%, 90%, 95%, 96%, 97, 98%, 99% or 100% identity to the nucleotide sequence of SEQ ID NO: 100.

35. The method of claim 1, wherein the therapeutic composition is a pharmaceutical composition.

36. The method of claim 1, wherein administering the therapeutic composition occurs before the onset of the cardiac ischemic event.

37. The method of claim 1, wherein administering the therapeutic composition occurs during the cardiac ischemic event.

38. The method of claim 1, wherein the therapeutic composition is administered about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, about 72 hours, or about 96 hours prior to reperfusion of ischemic cardiac tissue.

39. The method of claim 1, wherein administering the therapeutic composition occurs concurrent with reperfusion of ischemic cardiac tissue.

40. The method of claim 1, wherein administering the therapeutic composition occurs after reperfusion of ischemic cardiac tissue.

41. The method of claim 1, wherein the therapeutic composition is administered about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, about 72 hours, about 96 hours, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, or about 20 days, after reperfusion of ischemic cardiac tissue.

42. The method of claim 1, wherein the therapeutic composition comprises a plurality of microRNA (miR) antagonists, wherein the administration comprises subcutaneous administration, systemic administration, and/or intra-coronary administration.

43. The method of claim 42, wherein the therapeutic composition is administered at a dose of about 0.08 mg/kg, about 0.24 mg/kg, about 0.81 mg/kg, about 1.22 mg/kg, about 2.44 mg/kg, about 3.25 mg/kg, about 4.06 mg/kg, about 4.89 mg/kg, about 5.69 mg/kg, about 6.50 mg/kg, about 7.32 mg/kg, or about 8.13 mg/kg.

44. The method of claim 1, wherein the therapeutic composition comprises a plurality of microRNA (miR) antagonists, wherein the administration comprises intra-ventricular administration and/or intra-myocardial administration.

45. The method of claim 44, wherein the therapeutic composition is administered at a dose of about 0.004 mg/kg, about 0.012 mg/kg, about 0.0405 mg/kg, about 0.061 mg/kg, about 0.122 mg/kg, about 0.1625 mg/kg, about 0.203 mg/kg, about 0.2445 mg/kg, about 0.2845 mg/kg, about 0.325 mg/kg, about 0.366 mg/kg, or about 0.4065 mg/kg.

46. The method of claim 42, wherein subcutaneous administration of the therapeutic composition yields increased survival and reduced incidence of cardiac thrombus as compared to intravenous administration of the therapeutic composition.

47. The method of claim 1, wherein the therapeutic composition comprises a viral vector, wherein the administration comprises intravenous systemic administration and/or intra-coronary administration, and optionally the therapeutic composition is administered at a dose of about 2.5×10¹² vg (viral genome)/kg, about 2.5×10¹³ vg/kg, about 2.5×10¹⁴ vg/kg, or about 2.5×10¹⁵ vg/kg.

48. The method of claim 1, wherein the therapeutic composition comprises a viral vector, wherein the administration comprises intra-ventricular administration and/or intra-myocardial administration.

49. The method of claim 48, wherein the therapeutic composition is administered at a dose of about 1.0×10⁵ vg/kg to 1.0×10¹⁹ vg/kg, optionally the therapeutic composition is administered at a dose of about 0.125×10¹² vg/kg, about 0.125×10¹³ vg/kg, about 0.125×10¹⁴ vg/kg, or about 0.125×10¹⁵ vg/kg.

50. The method of claim 1, wherein the dose is administered in a single administration.

51. The method of claim 1, wherein the dose is administered over multiple administrations.

52. The method of claim 1, comprising repeated administration of the therapeutic composition to the subject, optionally the repeated administration comprises administration of one or more additional doses of the therapeutic composition to the subject.

53. The method of claim 52, wherein the repeated administration comprises administration of one or more additional doses of the therapeutic composition to the subject about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, about 72 hours, about 96 hours, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, and/or about 20 days, after reperfusion of ischemic cardiac tissue.

54. The method of claim 1, further comprising administrating an effective amount of at least one additional therapeutic agent or at least one additional therapy to the subject for a combination therapy.

55. The method of claim 54, wherein each of the therapeutic composition and the at least one additional therapeutic agent or therapy is administered in a separate formulation or are administered together in a single formulation.

56. The method of claim 54, wherein the therapeutic composition and the at least one additional therapeutic agent or therapy are administered sequentially, are administered concomitantly, and/or are administered in rotation.

57. The method of claim 54, wherein the at least one additional therapeutic agent or therapeutic therapy is selected from the group consisting of Idebenone, Eplerenone, VECTTOR, AVI-4658, Ataluren/PTC124/Translarna, BMN044/PRO044, CAT-1004, microDystrophin AAV gene therapy (SGT-001), Galectin-1 therapy (SB-002), LTBB4 (SB-001), rAAV2.5-CMV-minidystrophin, glutamine, NFKB inhibitors, sarcoglycan, delta (35 kDa dystrophin-associated glycoprotein), insulin like growth factor-1 (IGF-1) expression, genome editing through the CRISPR/Cas9 system, any gene delivery therapy aimed at reintroducing a functional recombinant version of the dystrophin gene, Exon skipping therapeutics, read-through strategies for nonsense mutations, cell-based therapies, utrophin upregulation, myostatin inhibition, anti-inflammatories/anti-oxidants, mechanical support devices, a biologic drug, a gene therapy or therapeutic gene modulation agent, any standard therapy for muscular dystrophy, and combinations thereof.

58. The method of claim 54, wherein the at least one additional therapeutic agent or therapeutic therapy is selected from the group comprising a percutaneous coronary intervention, coronary artery bypass grafting, thrombolytic therapy, anti-platelet therapy, heparin, warfarin, fibrinolytics, oxygen therapy, a vasodilator, pain medication, a beta blocker, an angiotensin-converting enzyme (ACE) inhibitor, an angiotensin receptor blocker (ARB), a glycoprotein antagonist, a statin, an aldosterone antagonist, an implantable cardiac defibrillator (ICD), or any combination thereof.

59. The method of claim 1, wherein reperfusion of ischemic cardiac tissue comprises a percutaneous coronary intervention, coronary artery bypass grafting, thrombolytic therapy, anti-platelet therapy, heparin, warfarin, fibrinolytics, oxygen therapy, a vasodilator, pain medication, a beta blocker, an angiotensin-converting enzyme (ACE) inhibitor, an angiotensin receptor blocker (ARB), a glycoprotein antagonist, a statin, an aldosterone antagonist, an implantable cardiac defibrillator (ICD), or any combination thereof.

60. The method of claim 1, wherein the subject is a mammal, optionally a human.

61. The method of claim 1, the subject has or is suspected of having a cardiac disease, wherein the cardiac disease is myocardial infarction, ischemic heart disease, dilated cardiomyopathy, heart failure (e.g., congestive heart failure), ischemic cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis, aortic stenosis, aortic regurgitation, mitral stenosis, mitral regurgitation, mitral prolapse, pulmonary stenosis, pulmonary regurgitation, tricuspid stenosis, tricuspid regurgitation, congenital disorder, genetic disorder, or any combination thereof.

62. The method of claim 1, wherein the subject is affected by a condition selected from the group comprising alcoholic cardiomyopathy, coronary artery disease, congenital heart disease, nutritional diseases affecting the heart, ischemic cardiomyopathy, hypertensive cardiomyopathy, valvular cardiomyopathy, inflammatory cardiomyopathy, cardiomyopathy secondary to a systemic metabolic disease, dilated cardiomyopathy (DCM), hypertrophic cardiomyopathy (HCM), arrhythmogenic right ventricular cardiomyopathy (ARVC), restrictive cardiomyopathy (RCM), noncompaction cardiomyopathy, supravalvular aortic stenosis (SVAS), vascular scarring, atherosclerosis, chronic progressive glomerular disease, glomerulosclerosis, progressive renal failure, vascular occlusion, hypertension, stenosis, diabetic retinopathy, or any combination thereof.

63. The method of claim 1, wherein the cardiac ischemic reperfusion injury comprises cardiac ischemic damage, cardiac reperfusion injury, or a combination thereof.

64. The method of claim 1, wherein the administration reduces cardiac ischemic damage, cardiac reperfusion injury, or a combination thereof, as compared to a control subject.

65. The method of claim 1, wherein the administration reduces creatine kinase levels as compared to a control subject.

66. The method of claim 1, wherein the cardiac ischemic reperfusion injury comprises injuries caused by the cardiac ischemia event, reperfusion injuries, or a combination thereof.

67. The method of claim 1, wherein the cardiac ischemic event comprises one or more of myocardial infarction, coronary artery bypass grafting (CABG), cardiac bypass surgery, cardiac transplantation, and angioplasty.

68. The method of claim 1, wherein the cardiac ischemic event comprises a vascular interventional procedure employing a stent, laser catheter, atherectomy catheter, angioscopy device, beta or gamma radiation catheter, rotational atherectomy device, coated stent, radioactive balloon, heatable wire, heatable balloon, biodegradable stent strut, a biodegradable sleeve, or any combination thereof.

69. The method of claim 1, wherein the administration results in one or more of (1) increased survival as compared to a control subject, (2) improved kidney function of the subject as compared to a control subject, (3) a decrease in blood urea nitrogen (BUN) levels as compared to a control subject, (4) a reduced scarring in the left ventricle of the subject and/or improved regional wall motion in the left ventricle of the subject as compared to a control subject, (5) a decrease in end diastolic volume and/or end systolic volume as compared to a control subject, (6) an increase in ejection fraction as compared to a control subject, (7) an increase in the number of cardiomyocytes and/or mRNAs encoding proteins that are involved in differentiated cardiomyocyte muscle structure and function as compared to a control subject, (8) an increase in the mRNA levels and/or protein levels of one or more of Ank2, Kdm6a, Grk6, K1h115, Adam22, Pfkp, Gorasp2, Ralgps1, Inppl1, Kdm3a, Kit, Sort1, Dv12, Sema6d, Tead1, B4galnt2, Ltbp4, Osbp19, Nfe2I1, Tnnt2, and Fhl1 as compared to a control subject, and (9) a decrease in the mRNA levels and/or protein levels of one or more of Asph, Map6, Zfp120, Ctnndl, Eya3, Tnnt2, Kdm3a, Myo18a, Ncoa6, Slc25a13, Rpe, Ralgps1, Gimap1, Myo5a, Zeb2, Arap1, Nt5c2, Phldb1, Ttn, Camta2, Mef2c, Slk, Uimc1, Mthfd1I, Mtus1, Ythdc1, and Eif2ak4 as compared to a control subject, and (10) an increase in one of more of cardiomyocyte formation, cardiomyocyte proliferation, cardiomyocyte cell cycle activation, mitotic index of cardiomyocytes, myofilament density, borderzone wall thickness, or any combination thereof, as compared to a control subject.

70. The method of claim 1, wherein the administration induces endogenous cardiomyocyte regeneration.

71. The method of claim 1, wherein the administration enhances cardiac function in the subject as compared to a control subject, wherein enhancing cardiac function comprises one or more of (i) improving left ventricular function, (ii) improving fractional shortening, (iii) improving ejection fraction, (iv) reducing end-diastolic volume, (v) decreasing left ventricular mass, and (v) normalizing of heart geometry, or (vi) a combination thereof.

72. The method of claim 1, wherein the administration has no significant effect on body weight and/or heart weight.

73. The method of claim 1, wherein the administration does not cause one or more of arrhythmia, after contractions (AC), and contraction failure (CF).

74. The method of claim 8, wherein the therapeutic composition increases the mRNA levels and/or protein levels of FHL1 and/or TNNT2.

75. The method of claim 8, wherein the disease or disorder is associated with one or more FHL1 mutations and/or one or more TNNT2 mutations.

76. The method of claim 8, wherein the disease or disorder is a muscular dystrophy disorder or a muscular dystrophy-like muscle disorder, optionally the muscular dystrophy disorder is associated with Amyotrophic Lateral Sclerosis (ALS), Charcot-Marie-Tooth Disease (CMT), Congenital Muscular Dystrophy (CMD), Duchenne Muscular Dystrophy (DMD), Emery-Dreifuss Muscular Dystrophy (EDMD), Inherited and Endocrine Myopathies, Metabolic Diseases of Muscle, Mitochondrial Myopathies (MM), Myotonic Muscular Dystrophy (MMD), Spinal-Bulbar Muscular Atrophy (SBMA), or a combination thereof.

77. The method of claim 8, wherein the disease or disorder is Limb girdle muscular dystrophy, X-linked myopathy with postural muscle atrophy (XMPMA), Reducing body myopathy (RBM), Scapuloperoneal (SP) syndrome, or any combination thereof.

78. The method of claim 8, wherein the disease or disorder is hypertrophic cardiomyopathy (HCM), restrictive cardiomyopathy (RCM), dilated cardiomyopathy (DCM), or any combination thereof, optionally the hypertrophic cardiomyopathy is familial hypertrophic cardiomyopathy.

79. The method of claim 9, wherein the kidney condition is associated with a function of the subject's kidneys.

80. The method of claim 9, wherein the kidney condition is selected from the group consisting of acute kidney diseases and disorders (AKD), acute kidney injury, acute and rapidly progressive glomerulonephritis, acute presentations of nephrotic syndrome, acute pyelonephritis, acute renal failure, idiopathic chronic glomerulonephritis, secondary chronic glomerulonephritis, chronic heart failure, chronic interstitial nephritis, chronic kidney disease (CKD), chronic liver disease, chronic pyelonephritis, diabetes, diabetic kidney disease, fibrosis, focal segmental glomerulosclerosis, Goodpasture's disease, diabetic nephropathy, hereditary nephropathy, interstitial nephropathy, hypertensive nephrosclerosis, IgG4-related renal disease, interstitial inflammation, lupus nephritis, nephritic syndrome, partial obstruction of the urinary tract, polycystic kidney disease, progressive renal disease, renal cell carcinoma, renal fibrosis, graft versus host disease after renal transplant, and vasculitis.

81. The method of claim 9, wherein the injury is associated with one or more of surgery, radiocontrast imaging, radiocontrast nephropathy, cardiovascular surgery, cardiopulmonary bypass, extracorporeal membrane oxygenation (ECMO), balloon angioplasty, induced cardiac or cerebral ischemic-reperfusion injury, organ transplantation, kidney transplantation, sepsis, shock, low blood pressure, high blood pressure, kidney hypoperfusion, chemotherapy, drug administration, nephrotoxic drug administration, blunt force trauma, puncture, poison, or smoking.

82. The method of claim 9, wherein the therapeutic composition is administered in combination with a renal therapeutic agent is selected from the group consisting of dexamethasone, a steroid, budesonide, triamcinolone acetonide, an anti-inflammatory agent, an antioxidant, deferoxamine, feroxamine, a tin complex, a tin porphyrin complex, a metal chelator, ethylenediaminetetraacetic acid (EDTA), an EDTA-Fe complex, dimercapto succinic acid (DMSA), 2,3-dimercapto-1-propanesulfonic acid (DMPS), penicillamine, minocycline, prednisone, azathioprine, mycophenolate mofetil, mycophemolic acid, sirolimius, cyclorsporine, or tacrolimusan antibiotic, an iron chelator, a porphyrin, hemin, vitamin B 12, an Nrf2 pathway activator, bardoxolone, ACE inhibitors, enalapril, glycine polymers, antioxidants, glutathione, N acetyl cysteine, a chemotherapeutic, QPI-1002, QM56, SVT016426 (QM31), 16/86 (third generation ferrostatin), BASP siRNA, CCX140, BIIB023, CXA-10, alkaline phosphatase, Dnmtl inhibitor, THR-184, lithium, formoterol, IL-22, EPO, EPO derivative, agents that stimulate erthyropoietin, epoeitn alfa, darbepoietin alfa, PDGF inhibitor, CRMD-001, Atrasentan, Tolvaptan, RWJ-676070, Abatacept, Sotatercept, an anti-infective agent, an antibiotic, an anti-viral agent, an anti-fungal agent, an aminoglycoside, a nonsteroidal anti-inflammatory drug (NSAID), a diuretic drug, a statin, a senolytic, a corticosteroid, a glucocorticoid, a liposome, renin, angiotensin, ACE inhibitor, mediator of apoptosis, mediator of fibrosis, drug that targets p53, Apaf-1 inhibitor, RIPK1 inhibitor, RIPK3 inhibitor, inhibitor of IL17, inhibitor of IL6, inhibitor of IL23, inhibitor of CCR2, nitrated fatty acids, angiotensin blockers, agonists of the ALK3 receptor, and retinoic acid.

83. The method of claim 9, wherein the therapeutic composition is administered in combination with a renal protective agent or a renal prophylactic agent selected from the group consisting of thiazide, bemetanide, ethacrynic acid, furosemidem torsemide, glucose, mannitol, amiloride, spironolactone, eplerenone, triamterene, potassium canrenoate, bendroflumethiazide, hydrochlorothiazide, vasopressin, amphotericin B, acetazolamide, tovaptan, conivaptan, dopamine, dorzolamide, bendrolumethiazide, hydrochlorothiazide, caffeine, theophylline, theobromine, a statin, a senolytic, navitoclax obatoclax, a corticosteroid, prednisone, betamethasone, fludrocortisone, deoxycorticosterone, aldosterone, cortisone, hydrocortisone, belcometasone, mometasone, fluticasone, prednisolone, methylprednisolone, triamcinolone acetonide, a glucocorticoid, dexamethasone, a steroid, budesonide, triamcinolone acetonide, an anti-inflammatory agent, an antioxidant, a nonsteroidal anti-inflammatory drug (NSAID), deferoxamine, iron, tin, a metal, a metal chelate, ethylenediaminetetraacetic acid (EDTA), dimercap to succinic acid (DMSA), 2,3-dimercapto-1-propanesulfonic acid (DMPS), penicillamine, an antibiotic, an aminoglycoside, an iron chelator, a porphyrin, an Nrf2 pathway activator, bardoxolone, ACE inhibitors, enalapril, glycine polymers, antioxidants, glutathione, N-acetyl cysteine, a PDGF inhibitor, lithium, ferroptosis inhibitors, vitamin B 12QPI-1002, QM56, SVT016426 (QM31), 16/86 (third generation ferrostatin), BASP siRNA, CCX140, BIIB023, CXA-10, alkaline phosphatase, Dnmtl inhibitor, THR-184, lithium, formoterol, IL-22, EPO, EPO derivative, agents that stimulate erthyropoietin, epoeitn alfa, darbepoietin alfa, PDGF inhibitor, CRMD-001, Atrasentan, Tolvaptan, RWJ-676070, Abatacept, Sotatercept, an anti-infective agent, an antibiotic, an anti-viral agent, an antifungal agent, an aminoglycoside, a nonsteroidal anti-inflammatory drug (NSAID), a diuretic drug, a statin, a senolytic, a corticosteroid, a glucocorticoid, a liposome, renin, angiotensin, ACE inhibitor, mediator of apoptosis, mediator of fibrosis, drug that targets p53, Apaf-1 inhibitor, RIPK1 inhibitor, RIPK3 inhibitor, inhibitor of IL17, inhibitor of IL6, inhibitor of IL23, inhibitor of CCR2, nitrated fatty acids, angiotensin blockers, agonists of the ALK3 receptor, SGLT2 modulator, and retinoic acid.

84. The method of claim 9, wherein the therapeutic composition improves one or more markers of kidney function in the subject selected from the group comprising reduced blood urea nitrogen (BUN) in the subject, reduced creatinine in the blood of the subject, improved creatinine clearance in the subject, reduced proteinuria in the subject, reduced albumin:creatinine ratio in the subject, improved glomerular filtration rate in the subject, reduced NAG in the urine of the subject, reduced NGAL in the urine of the subject, reduced KIM-1 in the urine of the subject, reduced IL-18 in the urine of the subject, reduced MCP1 in the urine of the subject, reduced CTGF in the urine of the subject; reduced collagen IV fragments in the urine of the subject; reduced collagen III fragments in the urine of the subject; and reduced podocyte protein levels in the urine of the subject, wherein the podocyte protein is selected from nephrin and podocin, reduced cystatin C protein in the blood of a subject, reduced β-trace protein (BTP) in the blood of a subject, and reduced 2-microglobulin (B2M) in the blood of a subject.
